(19) United States
(12) Patent Application Publication
(10) Pub. No.: US 2025/0241784 A1
(43) Pub. Date: Jul. 31, 2025

(54) APPARATUS AND METHOD FOR DELIVERY AND/OR REMOVAL OF OCCLUSIONS IN THE BODY

(71) Applicant: NEXT Life Sciences, Inc., San Luis Obispo, CA (US)

(72) Inventors: Steven R. BACICH, Half Moon Bay, CA (US); Matthew Thomas YUREK, San Diego, CA (US); Michael HARTSFIELD, Poway, CA (US); Jack GREELIS, Carlsbad, CA (US); Luke FOX, San Luis Obispo, CA (US)

(73) Assignee: NEXT Life Sciences, Inc., San Luis Obispo, CA (US)

(21) Appl. No.: 19/067,505

(22) Filed: Feb. 28, 2025

Related U.S. Application Data
(63) Continuation of application No. PCT/US2024/053266, filed on Oct. 28, 2024.
Application No. PCT/US2024/053266 is a continuation-in-part of application No. 18/792,215, filed on Aug. 1, 2024.
(63) Continuation of application No. 18/936,754, filed on Nov. 4, 2024.
Application No. 18/936,754 is a continuation of application No. PCT/US2024/053266, filed on Oct. 28, 2024.
Application No. PCT/US2024/053266 is a continuation-in-part of application No. 18/792,215, filed on Aug. 1, 2024.
(63) Continuation-in-part of application No. 18/792,215, filed on Aug. 1, 2024.
(60) Provisional application No. 63/593,750, filed on Oct. 27, 2023.
(60) Provisional application No. 63/593,750, filed on Oct. 27, 2023.

(51) Int. Cl. A61F 6/22; A61F 6/20

(52) U.S. Cl. CPC A61F 6/22; A61F 6/20; A61F 6/225

(57) ABSTRACT

Systems, devices, and methods for delivering one or multiple materials into a body space (e.g., to occlude the body space) and/or for removing one or multiple materials from a body space (e.g., to open or reopen the body space) are described. Delivering material into the vas deferens of the male reproductive tract for contraception, removing material from the vas deferens to restore patency of the vas deferens, delivering material into the fallopian tubes of the female reproductive tract for contraception, and removing material from fallopian tubes to restore patency of the fallopian tubes is described. Confirming access to a body space is described. Agitating materials in a body space is described. Creating turbulence in a body space is described.

### CROSS-REFERENCE TO RELATED APPLICATIONS

This application is a continuation of International Patent Application No. PCT/US2024/053266 filed Oct. 28, 2024, which is a continuation-in-part of U.S. Nonprovisional application Ser. No. 18/792,215 filed Aug. 1, 2024, where International Patent Application No. PCT/US2024/053266 filed Oct. 28, 2024 and U.S. Nonprovisional application Ser. No. 18/792,215 filed Aug. 1, 2024 each claim the benefit of priority to U.S. Provisional Application No. 63/593,750 filed Oct. 27, 2023. This application is also a continuation of U.S. Nonprovisional application Ser. No. 18/936,754 filed Nov. 4, 2024, which is a continuation of International Patent Application No. PCT/US2024/053266 filed Oct. 28, 2024 and a continuation-in-part of U.S. Nonprovisional application Ser. No. 18/792,215 filed Aug. 1, 2024, where International Patent Application No. PCT/US2024/053266 filed Oct. 28, 2024 is a continuation-in-part of U.S. Nonprovisional application Ser. No. 18/792,215 filed Aug. 1, 2024, and where International Patent Application No. PCT/US2024/053266 filed Oct. 28, 2024 and U.S. Nonprovisional application Ser. No. 18/792,215 filed Aug. 1, 2024 each claim the benefit of priority to U.S. Provisional Application No. 63/593,750 filed Oct. 27, 2023. This application is also a continuation-in-part of U.S. Nonprovisional application Ser. No. 18/792,215 filed Aug. 1, 2024, which claims the benefit of priority to U.S. Provisional Application No. 63/593,750 filed Oct. 27, 2023. All of the foregoing applications are incorporated herein by reference in their entirety for all purposes.

### BACKGROUND

Systems, devices, and methods for delivering and/or removing occlusive devices (e.g., one or multiple materials) into and/or from body spaces are disclosed, including, for example, systems, devices, and methods for delivering occlusive devices into the vas deferens of the male reproductive tract for contraception, for removing occlusive devices from the vas deferens to restore patency of the vas deferens, for delivering occlusive devices into the fallopian tubes of the female reproductive tract for contraception, for removing occlusive devices from fallopian tubes to restore patency of the fallopian tubes, for occluding and/or reopening a body space for any other purpose, or for any combination thereof. The systems, devices, and methods described herein can be used for occlusive devices in any body space (e.g., body lumens), for example, in any body lumen. The occlusive devices can be, for example, hydrogels. Hydrogels can function as an occlusive device when injected into body spaces due to several key properties. They can conform to the shape of the environment of the body space (e.g., the lumen of a vas deferens or fallopian tube), exhibit know biocompatibility, and form a resilient gel that can withstand dynamic body movements. Their durability can help maintain the occlusive property despite such body movements. After a hydrogel is injected into a body space to form an occlusion (e.g., for contraception), a reversal procedure can be performed to reopen the body space, for example, by removing the occlusion from the body space. For example, removing an occlusive device (e.g., a hydrogel) from the vas deferens or fallopian tubes can result in the return of patency of the vas deferens or fallopian tubes. Removing an occlusive device (e.g., a hydrogel) from the vas deferens or fallopian tubes can, for example, allow the passage of sperm in the vas deferens and the passage of sperm and eggs in the fallopian tubes, which can allow for the return to fertility.

The injection of occlusive devices (e.g., hydrogels) in a reproductive tract is typically done by a physician using a needle system (e.g., a hypodermic needle system) that penetrates the wall of a body space (e.g., body lumen). In practice, the distal end of the needle opening can inadvertently miss the bodily space and result in the delivery of the hydrogel in an unintended location. In such situations, the hydrogel can be ejected into an interstitial space or can miss the body space altogether. Typically, once entry into the body space is achieved, the needle of the needle system is left in place to remain in the lumen throughout the procedure. However, for patients that are awake without general anesthesia, any unintentional movement by the patient or physician can alter the positioning of the needle system, which can cause the sharp distal end of the needle to inadvertently protrude into unintended locations, in particular, outside of the vas deferens, which can cause the patient discomfort and pain. In addition, intended and/or unintended movement of the needle can perforate the vas deferens or penetrate into the posterior wall of the vas deferens, for example, after the initial penetration of the needle through the anterior wall during the injection process. Additionally, the sharp distal end of the needle can become inadvertently embedded within the wall of the vas deferens in an interstitial location. This can be particularly true for the vas deferens with muscular walls and surrounding fascia, and for internal lumens having soft mucosal lining or epithelium within the internal potential body space. For conventional delivery systems, the continued presence of a sharp needle within the body space during the procedure can increase the risk of injecting the hydrogel into an unintended location other than the body space.

Systems, methods, and devices are disclosed which can solve these and other problems associated with conventional delivery procedures.

Systems, methods, and devices are disclosed that can remove occlusive devices (e.g., hydrogels) from body spaces.

Systems, methods, and devices of delivering and/or removing reversible contraception, including, for example, long-lasing, non-hormonal, reversible contraception. In both the male or female applications, the availability of a long-lasting, non-hormonal, reversible contraception that can be delivered and/or removed in a minimally invasive manner meets an important unmet clinical need.

### SUMMARY

A method of confirming intraluminal access of a lumen in a body is disclosed. The method can include inserting a sheath into the body, advancing a probe relative to the sheath by an advancing distance into the lumen, and/or confirming the sheath has access to the lumen if the advancing distance is greater than or equal to a threshold distance.

A device is disclosed that can have a sheath and a probe. The probe can be advanceable from a retracted configuration to an advanced configuration relative to the sheath. When the probe is in the retracted configuration, a first distance can be between the probe and the sheath. When the probe is in the advanced configuration, a second distance greater than the first distance can be between the probe and the sheath. The first distance can be less than a threshold distance. When the second distance is greater than or equal to the threshold distance, the advanced configuration of the probe can be an indicator to a user that the device has access to a target site.

A method of removing a first material from a body lumen is disclosed. The method can include inserting a sheath into the body lumen, advancing a probe into the body lumen relative to the sheath, agitating the first material in the body lumen with the probe, irrigating a second material into the body lumen, and/or removing the first material from the body lumen.

A method of removing a first material from a body lumen is disclosed. The method can include inserting a sheath into the body lumen, inserting a probe into the body lumen, agitating the first material in the body lumen with the probe, irrigating a second material into the body lumen, and/or aspirating the first material and the second material from the body lumen.

A method of removing a first material, a second material, and/or a third material from a body lumen is disclosed. The method can include inserting a sheath into the body lumen, inserting a probe into the body lumen, agitating the first material in the body lumen with the probe, irrigating the second material into the body lumen, and/or removing the first material, the second material, and/or the third material from the body lumen.

A device is disclosed that can have a sheath, a probe, a first material, and/or a first opening. The device can have a first configuration and a second configuration. More of the first material can be distal the first opening when the device is in the second configuration than when the device is in the first configuration. The first material can be closer to a second material when the device is in the second configuration than when the device is in the first configuration. The probe can have a retracted configuration and an advanced configuration. More of the probe can be distal the first opening when the probe is in the advanced configuration than when the probe is in the retracted configuration. When the probe is in the advanced configuration, the probe can be movable from a first position to a second position. When the device is in the second configuration and the probe is in the first position, the probe can be in contact with the first material. When the device is in the second configuration and the probe is in the second position, the probe can be in contact with the first material or a gap can be between the probe and the first material. A distal terminal end of the probe can be the same distance or a different distance from the first opening when the probe is in the second position than when the probe is in the first position.

A device is disclosed that can have a sheath and a probe. The probe can be advanceable relative to the sheath. When the probe is in an advanced position, the probe can be an advanced distance from the sheath. When the advanced distance is greater than or equal to a threshold distance, the advanced position of the probe can be an indicator to a user that the device has access to a target site.

A method of removing a first material, a second material, and/or a third material from a body lumen is disclosed. The method can include inserting a sheath into the body lumen, inserting a probe into the body lumen, agitating the first material in the body lumen with the probe, irrigating the second material into the body lumen, and/or removing the first material, the second material, and/or the third material from the body lumen.

A method of removing a first material from a body space is disclosed. The method can include inserting a sheath into the body space, confirming the sheath has access to the body space by inserting a probe into the body space, irrigating a second material into the body space, and/or aspirating the first material and the second material from the body space.

Mechanisms that can confirm proper placement of the system in the body space prior to the delivery of occlusive material (e.g., hydrogel) that can be advantageous and facilitate better clinical outcomes are disclosed. Systems can have one or more confirmatory mechanisms that can demonstrate that the blockage or occlusion in the body lumen has been achieved following the deposition of the occlusive material.

Removal systems are disclosed that can remove occlusions (e.g., hydrogels) from body spaces in a minimally invasive manner and/or that can confirm the patency of the body space following the removal of the occlusion. The systems disclosed herein can be configured for both ease of use by the healthcare provider and can be designed to provide a confirmation of luminal placement and occlusion at the time of placement. At the time of removal, the systems can confirm intraluminal placement and return to patency once the hydrogel occlusion has been removed.

The system can minimally invasively penetrate body lumens and simplify the number of user steps in the procedure.

Systems for the delivery and removal of occlusive materials are disclosed herein. The systems can have hypodermic needles and syringe systems in which occlusive agents such as hydrogels can be injected into the body lumen. Syringe systems can have handles for greater mechanical action for injecting viscous materials or two-part systems.

Systems for the delivery and removal of occlusive materials can be provided without a needle (e.g., a hypodermic needle), or without part of a needle (e.g., a portion of a hypodermic needle). The delivery and removal systems can be inserted using a conduit (e.g., a catheter, a sheath) once a blunt dissection of the anterior wall of the vas deferens or fallopian tube is performed.

The systems described herein can be applied percutaneously and/or subcutaneously without exposing and isolating the vas deferens.

For the descriptions provided herein, the vas deferens and fallopian tubes can be used or applied interchangeably for the delivery and removal of the occlusive material (e.g., a hydrogel).

The use of a cylindrically shaped needle or hypodermic needle for insertion into the body lumen is disclosed. Hypodermic needles can have hollow, cylindrical tubing with a sharpened bevel at the distal end. These needles can be made from stainless steel with varying degrees of flex depending upon the gauge of needle. The dimensional tubing diameter and wall thickness define the needle gauge. Higher gauge needles are defined by smaller outer diameters. For the injection of hydrogels into the vas deferens for male contraception, the needle gauges can range, for example, from 21 G to 25 G with a 23 gauge as a nominal size insertion needle.

For male contraception, the physician can identify and isolate the vas deferens. This can be accomplished by manually palpating the scrotum until the muscular wall of the vas deferens determined. The location and guidance to the vas deferens can be facilitated by imaging systems such as ultrasonography, computed tomography, and magnetic resonance (MR) imaging to assist in identifying the vas deferens. A ring clamp can be used to retain the vas deferens underneath the scrotal skin and separate from the spermatic cord. In the ‘no scalpel’ technique, sharpened curved dissecting forceps can be used to provide blunt dissection of the scrotal skin with the goal of reaching the retained vas deferens and minimizing bleeding. Curved pinchers can be used for blunt dissection by removing the surrounding fascia from the vas deferens until the appropriate length of the vas deferens is elevated outside the scrotum for access for the remainder of the vasectomy procedure. The curved pinchers can be used for blunt dissection on the anterior wall of the vas deferens to allow for access to the lumen of the vas deferens. Following the ligation or occlusion of the vas deferens, the exposed and isolated portion of the vas deferens can be tucked back into the scrotum and the procedure can be repeated on the contralateral side of the scrotum for the other vas deferens. Using the curved pinchers to apply blunt dissection of the anterior wall of the vas deferens can minimize the amount of the vas deferens that needs to be exposed and isolated. As another example, the procedure can be performed percutaneously. Once the vas deferens procedure has been completed and the absence of bleeding is confirmed, a simple bandage can be applied to the scrotal entry sites.

The following are exemplary descriptions of insertion of the system into the body space.

For the delivery of hydrogels in the vas deferens for male contraception, the use of hydrogels can offer the opportunity of percutaneous delivery through the scrotal skin, thereby minimizing the level of invasiveness for the procedure. By minimizing surgical injury or trauma, patient recovery and comfort can be enhanced.

Variations of delivery and/or removal systems and devices are described herein. The delivery devices can place occlusive devices within the lumen of a reproductive tract and/or deliver removal agents for the return to patency of the reproductive tract. The intended target for the delivery device can be the lumen of the reproductive tract. Unintended placement of the occlusive device or removal agent can render the procedure unsuccessful.

For the delivery of hydrogels in the fallopian tube for female contraception, the use of transcervical approach with a catheter, or a hysteroscope and catheter, can offer a non-incisional insertion into the proximal portion of the fallopian tube. As another example, a hydrogel can be delivered by use of an ultrasonically guided transvaginal needle, or with other imaging modalities such as computed tomography and magnetic resonance (MR) imaging, through the wall of the vagina and directly into the fallopian tube. Another approach can be a laparoscopic entry into the fallopian tube.

When performing a procedure on the vas deferens, ring clamps can be used for lifting or holding the vas deferens tissue. A half-pipe or side-support system can be used to stabilize and maintain the vas deferens in a linearly straight form. The half-pipe or side-support system can be configured to stabilize the vas deferens tissue for injection by a straight penetrating tool such as a needle, or a half needle described below. As another example, for anatomical variations, an inguinal or laparoscopic approach can be used to access the vas deferens.

Holding and isolating the vas deferens with vacuum pressure with suction holder attached to the anterior wall of the vas deferens is disclosed.

A ring clamp for isolating and holding the vas deferens can have an angle miter tool for guiding the specific angle of entry for an access device and catheter.

The system can provide intraluminal insertion into a body lumen with a minimal profile and minimizing user steps. The system can allow for the passage of multiple deviceation within a central lumen of delivery and/or removal system without the need for back exchanges and/or removal of needles or probes from the access system. For example, typically when using a hypodermic needle for insertion and passage of a guidewire, once entry into the body space is established, the hypodermic needle needs to be threaded back in a retrograde direction so that the needle can be removed from the guidewire. Once the hypodermic needle is removed from the guidewire, other deviceation or catheters can be deployed over the guidewire. Eliminating the need for a back exchange (e.g., between hypodermic needle and other deviceation or catheters) can reduce the number of user steps and any instability caused by these additional movements and actions.

A split needle insertion system with two-hemispherical sections is disclosed. The hemispherical sections can be split apart in a radial direction from the co-linear central axis. As the needle is split apart, other deviceation can be passed through the central axis without requiring a back exchange.

The hemispherical sections can be rotated along their central axis to form an opening in the insertion needle. In either configuration, the portions of the split do not have to be equal in size, only cylindrical at insertion and a second configuration with an opening. Multiple sections that split or rotate apart greater than two are possible.

A half-needle insertion system for insertion into a body space is disclosed. The half needle can reduce the profile at entry and shield or have a probe within the half needle section. This can be done with the half needle cut at the equator of its cylindrical shape, the half needle above the equator, or the half needle below the equator. The half needle can have a curved shape, for example, to allow for more oblique angles of insertion or entry into a body lumen.

The system can have a deflecting lancet or trocar for probe insertion. The lancet or trocar can be spring loaded for entry.

The system can enter the vas deferens with a hemi-spherical transection of the vas deferens tube to physically expose the lumen.

The system can enter the vas deferens following a blunt dissection of the anterior wall for access to the lumen of the vas deferens.

For the insertion of occlusive devices into a body space, positive confirmation of body space access (e.g., intraluminal access) can be a significant improvement to the success of the procedure. Intraluminal placement into a body lumen can be confirmed. For example, a spring-loaded retractable probe (e.g., lancet) can ensures a specific depth of penetration into a body lumen. The system can have a spring-loaded probe that can traverse into the lumen of the vessel as a confirmatory method of insertion. As another example, the probe may not be spring-loaded.

A confirmatory method can utilize the injection of media for hydro-distension before and after probe insertion. The injection of media as a confirmation step can be quantified by a specific translation of a probe into the vas deferens. The probe can have a flexible tip and can coil or back out if placed outside the true lumen of the vas deferens. The translation of the probe or guidewire for a predetermined distance can denote or signal true or confirmed body space entry (e.g., lumen entry). This translation distance, also referred to as the distance “L”, the threshold distance, and other similar terms, can be, for example, 5.0 cm in length of probe travel from the end of an access sheath to the distal tip of the probe. The threshold distance can have a range, for example, of 1.0 cm to 15.0 cm.

The probe can be hydrophilic coated for added lubricity, which can reduce friction. The probe can be coated with other lubricity agents, including, for example, silicone oil or MDX solutions. The distal end of the probe can be configured straight in its natural state or can be pre-formed in a “J” shape.

The procedure may not continue, for example, unless the translation distance of the probe by the threshold distance has been accomplished. For example, a hydrogel may not be delivered and/or be deliverable until the probe is advanced by the threshold distance. For example, the delivery and/or removal system handle can have a lock out latch that can only be released once the probe reaches the prescribed translation distance (e.g., the threshold distance). The remaining steps that may not be performed without satisfactorily completing the “L” distance with the probe, including, for example, a) advancing the access sheath over the probe, b) injecting media into the body lumen, and/or c) injecting the occlusive device (e.g., a hydrogel). The “L” distance (e.g., the threshold distance) can provide, for example, a quantifiable threshold for intraluminal confirmation prior to further intervention in the body lumen. The lock out latch mechanism can prevent the end user from defeating the “L” distance threshold. As another example, the delivery and/or removal device may not have the lock out latch mechanism, in which case the user can wait to a) advance the access sheath over the probe, b) inject media into the body lumen, and/or c) inject the occlusive device (e.g., a hydrogel) until the user has confirmed the probe has been advanced by the threshold distance.

The injection of media for hydro-distension before and after probe insertion can be used to confirm intraluminal placement. The amount of force required to inject the media can be quantified as a determinant for true lumen placement. Flow rate and volume can be measured with or without a force measurement.

The injection of media for hydro-distension before and after probe insertion can be visualized by using ultrasound or radiographic means.

The injection of media for hydro-distension before and after probe insertion can be confirmed with lavage that flows through the vas deferens and out through the urethra.

The step of injecting media into the vas deferens or body lumen can be used for clearing the body lumen of material and preparing for subsequent hydrogel insertion.

After the insertion of the occlusive device or hydrogel, the injection of media can assist in the gelation or curing of the hydrogel.

After the occlusive device or hydrogel is placed within the body space, probe insertion with flexible tip can be used for confirming occlusion of the lumen.

A spring mechanism and/or a spring can be used to translate the probe the threshold distance. The spring mechanism and/or a spring can apply a consistent advancement force, for example, to remove the force variability that the end user can apply to the probe. For example, the spring mechanism and/or the spring can prevent a user from advancing the probe with forces that are too high, for example, to force the probe to reach the threshold distance. Forces that are too high can be, for example, forces that can cause the probe to pierce the wall of the body space, forces that can cause the probe to kink inside the body space, forces that can cause the probe to expand or distend the body space, forces that can cause the probe to tear or cut the wall of the body space, or any combination thereof.

The proximal end of the probe can be coupled to a force measurement system to display the amount of force required to traverse the threshold distance to confirm placement of the delivery and/or removal device in the body space.

The sharp distal end of the insertion system can be removed before further manipulation in the body lumen.

After confirmation of placement of the access sheath in the body space, the needle can be split in two-halves in a radial direction. Once split, the two-halves can be retracted, and the access sheath can be passed over the probe without requiring a back exchange.

The two-halves of the split needle can be configured in which one half rotates about the central axis of the cylinder thereby creating an opening in the needle configuration. The remaining section can be retracted and the access sheath advanced over the probe without requiring a back exchange.

A half needle design can minimize the insertion profile and can provide a shape that can be both retracted and minimized for further deviceation in the central lumen of the access system. Once confirmation of intraluminal placement is achieved with the probe, the half needle can be retracted and placed into a location of the handle housing without needing a back exchange or further manipulation by the end user.

The body space can be prepared for delivery of an occlusive device (e.g., a hydrogel) into the body space. The injection of media such as normal saline, phosphate buffered saline, Ringer's lactate, or other media can be used for clearing the body space of remnant materials and fluids. The injection of such media can prepare the body space for insertion of the occlusive device by opening the body space (e.g., the luminal cavity), wetting the body space surfaces (e.g., the intraluminal surfaces), supplying lubricity in addition to the media, mechanical distending the potential space of the lumen, supplying or altering the internal pH, supplying proteins, electrolytes, surfactants, wetting agents, or fluids that improve the bioadhesive properties of the hydrogels and make the body space environment (e.g., the intraluminal environment) more uniform for widespread clinical use and hydrogel implantation, or any combination thereof. The media can be a gas such as CO2 or other gases that are acceptable for use in the body. Gas media can be applied to dry or reduce the amount of fluids in the body space environment (e.g., the intraluminal environment of the vas deferens).

The injection of media can assist in the gelation of the hydrogel after delivery sheath insertion. The media in the intraluminal environment can accelerate the cure rate of the hydrogel in vivo.

The hydrogel can be delivered. The access and delivery system can be configured with a hydrogel cartridge with a plunger. The hydrogel cartridge can have a flexible tip plunger that can allow for the distal tip of the plunger to exit the delivery sheath.

For injection of the hydrogel into the body space, antegrade delivery can be defined as inserting the delivery sheath and injecting the hydrogel forward into the body lumen. Retrograde delivery can be defined by a retraction of the delivery sheath during the injection process. The delivery sheath can be used for retrograde delivery facilitated by simultaneous retraction of the delivery sheath during injection.

Occlusion of the body space can be confirmed after hydrogel delivery and removal of the system from the body space. The injection of media can be used for confirming occlusion using force, pressure, flow rate, and/or volume measurement to confirm occlusion.

The injection of media can be employed after (e.g., immediately after) the injection of the hydrogel for creating space inside the body space to prevent excess material or tail of the hydrogel to exit the body space.

A method for preventing the tail of the hydrogel from exiting the body space can utilize simultaneous rotation of delivery sheath during retraction to prevent the tail of hydrogel from exiting. The simultaneous rotation can be supplied by a motor applying rotary forces to the delivery sheath.

Once an occlusive device (e.g., hydrogel) is inserted into a body space, future identification of the delivery site of the occlusive hydrogel in the body lumen may prove to be difficult. The insertion site may heal with minimal scarring or trauma at the site of entry. For the vas deferens or fallopian tube, a tag or identifier can be applied for future identification of the entry site. The entry site can be tagged with a visual tattoo on the external surface of the vas deferens or fallopian tube.

The site of insertion into the vas deferens or fallopian tube can be tagged with an external clip that is visible via ultrasound or radiographic imaging, for example for future identification of the delivery of the occlusive hydrogel in the body space. The clip may or may not occlude the vas deferens or fallopian tube, and can mark the site of insertion that can be identified in the future.

The site of insertion into the vas deferens or fallopian tube can be tagged with a circumferential band around the vas deferens or fallopian tube, for example, to permanently identify the site of insertion for future identification of the delivery of the occlusive hydrogel in the body space. The circumferential band can be elastomeric (e.g., slightly elastomeric) and may not be restrictive to occlude the body space and may not create tissue necrosis or a chronic inflammatory event.

The occlusion (e.g., hydrogel) can be removed from a body space. At the time of removal of the occlusion, the mode of isolating the body space (e.g., vas deferens) or cannulating the body space (e.g., fallopian tube) can be the same as when the body space was accessed to deliver the occlusion.

To remove the occlusion from the body space, a removal device can be inserted into the body space. At the time of removal, a more proximal entry site may be required for the “L” distance (e.g., the threshold distance) of the probe to be satisfied. The “L” distance can be reduced at the time of removal. For example, the “L” distance at the time of removal can be 1 cm, 2 cm, or 3 cm as opposed to a larger “L” distance at the time of delivery or injection.

At the time of removal, the embodiments for confirmation of intraluminal placement, and the embodiments for placement of intraluminal sheath and removal of the sharp implement, have been described. In this instance, the sheath inserted into the body space can be called the removal sheath and can be configured the same as or different from the delivery sheath.

The removal sheath can have multiple lumens for both irrigation and aspiration. The probe for intraluminal confirmation can be housed or applied in either the irrigation or aspiration lumen.

At the time of removal, media can be delivered to confirm occlusion of the body lumen with the hydrogel using a force, pressure, flow rate, and/or volume measurement system to quantify occlusion.

The probe can be advanced to confirm occlusion in the body lumen by force measurements experienced during the advancement of the probe by encountering the intraluminal occlusion. The prevention of further translation of the probe in the lumen can be used to confirm occlusion in the body space.

The process of removal of the occlusion (e.g., hydrogel) can include instillation of a dissolving media into the body lumen with concurrent irrigation and aspiration. Dissolving media can be, for example, sodium bicarbonate solution composed of 8.4% sodium bicarbonate, or a range of 2% to 10%, or a larger range of 1% to 15% of sodium bicarbonate to dissolving media such as water. The concentration of sodium bicarbonate can range, for example, from 4% to 10%, or a larger range of 1% to 15%. The instillation can be performed with a multi-lumen (e.g., two-lumen), co-linear concentric sheath system.

The two-lumen sheath system can have a central lumen for probe passage and irrigation with an eccentric lumen for aspiration that can be proximal to the distal end of the irrigation lumen.

The sodium bicarbonate can be administered at body temperature (37° C.) or a higher temperature than body temperature to accelerate the reaction to the hydrogel. The elevated temperature can range from 37.1° C. to 44° C., and can be controlled to not create a localized tissue reaction or protein denaturation in the vas deferens.

The irrigation and aspiration can be controlled to ensure that the intraluminal pressure within the body lumen does not exceed 3.0 psi during continuous irrigation and aspiration. The maximum intraluminal pressures can range, for example, from 0.0 psi to 6.0 psi.

The probe can be configured to provide mechanical agitation to the occlusive hydrogel and/or to fluid and/or solids in the body space during the irrigation and aspiration process to accelerate the mechanical breakdown and surface contact causing dissolution of the hydrogel by the sodium bicarbonate solution.

Mechanical agitation can be provided by an ultrasonic generation source connected to the proximal end of the probe, by a vibratory generation source connected to the proximal end of the probe, by a motor providing rotational motion and is connected to the proximal end of the probe, or by any combination thereof.

The probe can zero, one, or multiple agitator features, including, for example, protrusions, bristles, brushes, indentations, coils, loops, angulations, or any combination thereof, for example, that can be configured to agitate the occlusion (e.g., hydrogel). The agitator features can, for example, enhance the mechanical agitation that the probe can provide to the occlusion. For example, mechanical agitation can be enhanced by protrusions, bristles, brushes, indentations, coils, loops, and angulations of the probe to enhance the mechanical agitation of the probe on the occlusion (e.g., hydrogel). The agitator features can enhance the agitation and action of the dissolving media (e.g., sodium bicarbonate solution).

An external vibratory source can be applied to the body lumen during instillation of the dissolving media (e.g., sodium bicarbonate solution) to enhance the agitation and action of the dissolving media.

The removal system can supply a lavage of sodium bicarbonate at the completion of the removal step. Other lavage media options include, for example, saline, phosphate buffered saline, Ringer's lactate, and other biocompatible media suitable for the reproductive tract.

The removal system can have pressure relief valves, for example, to control the intraluminal pressure of the vas deferens. Examples of pressure ranges can be, for example, from 1.0 psi to 10.0 psi (e.g., 3.0 psi as a nominal value).

The irrigation and aspiration function of the removal system can be monitored as a function of time. Examples of time durations are 1 minute to 10 minutes of irrigation and aspiration, including every 1 second increment within this range (e.g., 4 minutes as a nominal time duration).

The irrigation and aspiration function of the removal system can be performed using a predetermined volume of sodium bicarbonate solution, of other dissolving solution, and terminating the step when the fluid volume is exhausted. Examples of fluid volumes include, for example, 10 cc to 250 cc (e.g., 50 cc as a nominal fluid volume).

At the conclusion of the irrigation and aspiration function of the removal system, a final irrigation lavage can be performed with the visual confirmation of effluent from the patient's urethra to signify a patent vas deferens.

The advancement of the removal system probe (e.g., guidewire) past the hydrogel implantation site can be an indicator that provides confirmation that the occlusive hydrogel has been removed.

The advancement of the removal system catheter past the hydrogel implantation site over the wire can be an indicator that provides confirmation that the occlusive hydrogel has been removed.

The removal system can be configured to have a force or flow measurement system to confirm or quantitate the restoration of patency in the body space during lavage.

The delivery or removal systems can be introduced into the lumen of the reproductive tract by a wrap around sheath designed to penetrate the vas deferens or fallopian tube. The wrap around sheath can have a needle or other sharp implement to facilitate penetration into the lumen. Once entry into the lumen is achieved and confirmed by the advancement of the probe, the wrap around sheath can be retracted, peeled away, or removed from the delivery or removal system.

### BRIEF DESCRIPTION OF THE DRAWINGS

The drawings shown and described are exemplary variations and non-limiting. Like reference numerals indicate identical or functionally equivalent features throughout.

FIG. 1A illustrates a cross-sectional view of the male reproductive tract.

FIG. 1B illustrates the vas deferens positioned in a clamp for percutaneous insertion.

FIG. 1C illustrates an isolated vas deferens that is visible through the scrotal skin.

FIG. 2A illustrates a cross-sectional view of the female reproductive tract.

FIG. 2B is a schematic illustration showing the position of a hysteroscopically positioned catheter at the os of a fallopian tube prior to insertion of materials (e.g., hydrogels) in the fallopian tube for female contraception.

FIG. 3 is a schematic illustration of a variation of a system for accessing and/or isolating the vas deferens.

FIG. 4 is a schematic illustration showing a device for isolating the vas deferens with vacuum (e.g., by holding the anterior wall of the vas deferens).

FIG. 5A illustrates a variation for isolating the vas deferens with a hand and a clamp.

FIG. 5B is a cross-sectional view of the features in FIG. 5A along lines 5B-5B.

FIG. 6 illustrates a variation of the device of FIG. 3 and a variation of an angle miter.

FIG. 7 is a schematic illustration of a transcervically positioned catheter for intrauterine placement into the os of the fallopian tube.

FIG. 8 is a schematic illustration of using transvaginal ultrasound to deliver material directly into the fallopian tube.

FIG. 9A illustrates that a variation of a device with a split needle.

FIG. 9B1 illustrates a cross-sectional view of the split needle of FIG. 9 taken along the line 9B1-9B1 in a non-split configuration.

FIG. 9B2 illustrates the split needle of FIG. 9B1 in a radially split configuration.

FIG. 9C1 illustrates a cross-sectional view of the split needle of FIG. 9 taken along the line 9C1-9C1.

FIG. 9C2 illustrates the split needle of FIG. 9C1 in a rotationally split configuration.

FIG. 10 is a perspective view a variation of a device with a needle attached to a conduit or a probe.

FIG. 11A is schematic illustration of a variation of a device with a needle attached to a conduit or a probe.

FIG. 11B is schematic illustration of a side view of the device of FIG. 11A.

FIG. 12A is schematic illustration of a variation of a device with a needle attached to a conduit or a probe.

FIG. 12B is schematic illustration of a side view of the device of FIG. 12A.

FIG. 13A is schematic illustration of a variation of a device with a needle attached to a conduit or a probe.

FIG. 13B is schematic illustration of a side view of the device of FIG. 13A.

FIG. 14A is schematic illustration of a variation of a device with a needle attached to a conduit or a probe.

FIG. 14B is schematic illustration of a side view of the device of FIG. 14A.

FIG. 15 is schematic illustration of a variation of a cutting tool having a variable guillotine depth for transection of a body space for probe insertion.

FIGS. 16A-16E illustrate a variation of a spring-loaded lancet system that can be used to confirm access to a body space.

FIG. 17A illustrates a variation of a device before and after insertion of a probe into a body space.

FIG. 17B illustrates a close-up view of the device in section 17B of FIG. 17A.

FIG. 17C illustrates a close-up view of the device in section 17C of FIG. 17B.

FIG. 17D illustrates the device of FIG. 17A with a probe in an advanced position in the body space.

FIG. 17E illustrates a close-up view of the device in section 17E of FIG. 17D.

FIG. 17F illustrates that the device of FIG. 17A can be used in combination with ultrasound or radiographic imaging for confirmation of intraluminal placement in the body space.

FIG. 17G illustrates a variation of a method for the injection of media for hydro-distension before and after probe insertion by confirming lavage through the urethra.

FIG. 17H illustrates a variation of a method for the injection of a media for assisting in the gelation of the hydrogel.

FIG. 18 is a schematic illustration of a variation of a probe having multiple sections.

FIG. 19 is a schematic illustration of a variation of a probe having a J-angle flexible tip.

FIG. 20 is a schematic illustration of a variation of confirming placement of a device in a body space.

FIG. 21 is a schematic illustration of a variation of the probe with a spring mechanism for translation and for confirming placement of the device in a body space.

FIG. 22 is a schematic illustration of a probe with a deflection force measurement for confirming placement of a device in a body space.

FIGS. 23A-23B illustrate a method of removing a split needle from a body space and/or of advancing a conduit over a probe in a body space.

FIGS. 23-23C illustrate a method of removing a half needle from a body space and/or of advancing a conduit over a probe in a body space.

FIG. 24A illustrates a variation of confirming access to a body space with a probe.

FIG. 24B illustrates a variation of retracting a probe from a body space back into a handle.

FIG. 24C1 illustrates a variation of a device.

FIG. 24C2 illustrates a close-up perspective view of the device in section 24C2 of FIG. 24C1.

FIG. 24D1 illustrates the device of FIG. 24C1 in a body space with a needle and a connector in an advanced configuration.

FIG. 24D2 illustrates a close-up view of the device in section 24D2 of FIG. 24D1.

FIG. 24D3 illustrates the device of FIG. 24C1 in the body space with the needle and the connector in a partially retracted configuration.

FIG. 24D4 illustrates the device of FIG. 24C1 in the body space with the needle and the connector in a fully retracted configuration.

FIG. 24D5 illustrates the device of FIG. 24C1 in the body space and the delivery of a material (e.g., a hydrogel) to the body space while the needle and the connector are in a fully retracted configuration and while the probe is in a fully retracted configuration.

FIG. 24E1 illustrates a variation of the device.

FIG. 24E2 illustrates a close-up view of the device in section 24E2-24E2 of FIG. 24E1.

FIG. 24F1 illustrates a side cross-sectional view of the device of FIG. 24E1 taken along the line 24F1-24F1.

FIG. 24F2 illustrates a variation of the device of FIG. 24F1 in section 24F2-24F2.

FIG. 24G illustrates a cross-sectional view of the device of FIG. 24E2 taken along the line 24G-24G.

FIG. 24H1 illustrates a variation of a distal end of the device with a connector and a needle in an advanced configuration.

FIG. 24H2 illustrates a close-up perspective view of the device in section 24H2 of FIG. 24H1.

FIG. 24H3 illustrates a variation of the distal end of the device of FIG. 24H1 with the probe in an advanced configuration and with the needle in the advanced configuration of FIG. 24H1.

FIG. 24H4 illustrates a close-up perspective view of the device in section 24H4 of FIG. 24H3.

FIG. 24H5 illustrates a close-up view of the device in section 24H5 of FIG. 24H3.

FIG. 24H6 illustrates a variation of the distal end of the device of FIG. 24H1 with the probe in an advanced configuration and with the needle and the connector in a retracted configuration.

FIG. 24H7 illustrates a close-up view of the device in section 24H7 of FIG. 24H6.

FIG. 24H8 illustrates a variation of the distal end of the device of FIG. 24H1 with the probe in a retracted configuration and with the needle and the connector in the retracted configuration of FIGS. 24H6-24H7.

FIG. 24H9 illustrates a close-up view of the device in section 24H9 of FIG. 24H8.

FIG. 24H10 illustrates a variation of advancing a cartridge into the distal end of the device of FIG. 24H1.

FIG. 24H11 illustrates a close-up view of the device in section 24H11 of FIG. 24H10.

FIG. 24H12 illustrates a variation of delivering a material (e.g., hydrogel) from the device of FIG. 24H1.

FIG. 24I1 illustrates a variation of the device of FIG. 24C1.

FIG. 24I2 illustrates a close-up perspective view of the device in section 2412 of FIG. 24I1 in a body space with a needle and a connector in an advanced configuration.

FIG. 24I3 illustrates a close-up perspective view of the device in section 24I3 of FIG. 24I1.

FIG. 24I4 illustrates the device of FIG. 24I1 in the body space with the needle and the connector in an advanced configuration and with a probe in an advanced configuration.

FIG. 24I5 illustrates the device of FIG. 24I1 in the body space with the needle and the connector in a fully retracted configuration, with the probe in an advanced configuration in the body space, and with a conduit advanced over the probe in the body space.

FIG. 24I6 illustrates the device of FIG. 24I1 in the body space with the needle and the connector in a fully retracted configuration and with the probe in a fully retracted configuration.

FIG. 24I7 is a cross-sectional view of the features in FIG. 24I6 along lines 24I7-24I7 showing the delivery of a material (e.g., a hydrogel) to the body space.

FIGS. 25A-25B show variations for preparing the body lumen for hydrogel delivery. FIG. 25A illustrates the injection of media for clearing the body lumen of material and preparing for hydrogel insertion after delivery sheath insertion. FIG. 25B illustrates injection of media for assisting in the gelation of the hydrogel after delivery sheath insertion.

FIGS. 26A-26D illustrate a variation of the device for the delivery of the hydrogel using a hydrogel cartridge with plunger.

FIG. 27 illustrates a hydrogel cartridge with flexible tip plunger.

FIG. 28 illustrates antegrade delivery of the hydrogel in the vas deferens.

FIG. 29 illustrates a variation configured to provide retrograde delivery of the hydrogel in the vas deferens with simultaneous retraction of the delivery sheath.

FIGS. 30A-30B show variations for confirming occlusion of the body lumen after hydrogel delivery and removal of the system from the body lumen. FIG. 30A illustrates the injection of media, gas, or air for confirming occlusion using a force measurement system. The injection of media can also be used for initiating gelation of the hydrogel. FIG. 30A illustrates the injection of media for confirming occlusion using a force measurement device. FIG. 30B illustrates injection of media for confirming occlusion using volume measurement system.

FIG. 31 illustrates the injection of media for creating space from body lumen insertion site and/or for preventing a tail of hydrogel from exiting the vas deferens.

FIG. 32 illustrates the removal of the delivery sheath with simultaneous rotation of sheath to prevent a tail of hydrogel from exiting the vas deferens.

FIGS. 33A-33C show systems for future identification of the delivery site of the occlusive hydrogel in the body lumen. FIG. 33A illustrates a system where the site of insertion into the vas deferens can be tagged with a visual tattoo on the external surface of the vas deferens. FIG. 33A illustrates a vas deferens tagged with a tattoo. FIG. 33A illustrates a vas deferens tagged with an external clip. FIG. 33B illustrates a system for future identification of the delivery of the occlusive hydrogel in the body lumen where the site of insertion into the vas deferens can be tagged with an external clip that is also visible via ultrasound or radiographic imaging. The clip may not occlude the vas deferens, but only marks the site of insertion that can be identified in the future. FIG. 33C illustrates a vas deferens tagged with a circumferential band. FIG. 33C illustrates a system for future identification of the delivery of the occlusive hydrogel in the body lumen where the site of insertion into the vas deferens can be tagged with a circumferential band around the vas deferens to permanently identify the site of insertion. The circumferential band is slightly elastomeric and is not restrictive to occlude the body lumen and does not create tissue necrosis.

FIGS. 34A1-35 show variations for a dual lumen removal system for the removal of a hydrogel occlusion from a body lumen with an intraluminal sheath, termed the removal sheath, a rotating knob, and can be configured the same as the previously described delivery sheath. FIGS. 34A1-34A2 illustrates the removal system with irrigation and aspiration holes. FIG. 34B illustrates the handle of the removal system in a cross-sectional view. FIGS. 34C1-34C3 illustrate a variation of the dual lumen removal system of FIGS. 34A1-34A2. FIG. 34C3 illustrates a cross-sectional view of the device of FIG. 34C2 taken along the line 34C3-34C3.

FIG. 35 illustrates a variation where the removal sheath has multiple lumens for both irrigation and aspiration. The probe for intraluminal confirmation can be housed or applied in either the irrigation or aspiration lumen.

FIG. 36 demonstrates that at the time of removal, media can be delivered to confirm occlusion of the body lumen with the hydrogel using a force or pressure measurement.

FIG. 37 illustrates that a probe can be advanced or translated to confirm occlusion in the body lumen by force measurements collected on the proximal end. Force measurements experienced from the advancement of the probe interacting with the intraluminal occlusion can be measured and recorded. The prevention of further translation of the probe in the lumen can be used to confirm occlusion in the body lumen.

FIG. 38 illustrates a variation of the process of removal of the hydrogel with the instillation of a dissolving media into the body lumen with concurrent irrigation and aspiration. Dissolving media can be sodium bicarbonate solution composed, for example, of 8.4% sodium bicarbonate. The concentration of sodium bicarbonate can range, for example, from 4% to 10%. The installation can be performed with a two-lumen, co-linear concentric sheath system.

FIG. 39 illustrates that the two-lumen sheath system can have a central lumen for probe passage and irrigation with an eccentric lumen for aspiration that is proximal to the distal end of the irrigation lumen.

FIG. 40 illustrates that a heating source where the sodium bicarbonate can be administered at body temperature (37° C.), or a higher temperature than body temperature to accelerate the dissolution action on the hydrogel. The elevated temperature can range, for example, from 37.1° C. to 44° C. and is controlled to not create tissue necrosis or protein denaturation.

FIG. 41 illustrates that irrigation and aspiration can be controlled by a pressure regulator to ensure that the intraluminal pressure within the body lumen may not exceed 3.0 psi during continuous irrigation and aspiration. The maximum intraluminal pressures can range, for example, from 0.1 psi to 6.0 psi.

FIG. 42 illustrates a probe that can be configured to provide mechanical agitation to the occlusive hydrogel during the irrigation and aspiration process to accelerate the surface contact and dissolution of the hydrogel by the sodium bicarbonate solution. Manual agitation could be accomplished by forward and backward motion, rotary motion, or a combination of both motions on the probe. The proximal end of the probe can be configured with a handle to facilitate the user in applying these motions to the probe. Mechanical agitation can also be provided by an ultrasonic generation source connected to the proximal end of the probe.

FIG. 43 illustrates that mechanical agitation can be provided by a vibratory generation source connected to the proximal end of the probe.

FIG. 44 illustrates that mechanical agitation can be provided by a motor providing rotational motion and is connected to the proximal end of the probe.

FIGS. 45A-45G show mechanical agitation that can be furthered by protrusions (FIG. 45A), bristles (FIG. 45B), brushes (FIG. 45C), indentations (FIG. 45D), coils (FIG. 45E1-45E3), loops (FIG. 45F), and/or angulations (FIG. 45G) of the probe to further enhance the mechanical agitation of the probe on the occlusive hydrogel.

FIG. 46 illustrates that an external vibratory source can be applied to the body lumen (e.g., the body space) during instillation of a material (e.g., dissolving media and/or sodium bicarbonate solution) to enhance the agitation and action of the dissolving media.

FIG. 47 illustrates that the removal device can supply a lavage of sodium bicarbonate at the completion of the removal step. Additional or alternative lavage media options can include, for example, saline, phosphate buffered saline, Ringer's lactate, and other biocompatible media suitable for the reproductive tract. The removal system can be configured to have a force or flow measurement system to confirm or quantitate the restoration of patency in the body space during lavage. The lavage can be visible as it goes through the vas deferens and exits the urethra. FIG. 47 illustrates applying lavage at the end with force or flow measurement, for example, to confirm patency of the body space.

FIGS. 48A-48E illustrate a delivery or removal system with a penetrating sheath configured to wrap around the probe and intraluminal catheter. The wrap around sheath and be retracted or peeled away from the handle once entry into the lumen has been established. FIG. 49A illustrates the wrap around penetrating sheath on a delivery or removal handle. FIG. 48B illustrates a close-up view of the wraparound penetrating sheath. FIG. 48C illustrates the probe through the distal opening of the wrap around penetrating sheath. FIG. 48D illustrates the wrap around penetrating sheath slightly retracted with both the probe and intraluminal catheter through the distal opening of the wrap around penetrating sheath. FIG. 48E illustrates the wrap around penetrating sheath completely retracted and separate from the delivery or removal handle.

FIGS. 49A-49C illustrate a wrap around penetrating sheath that can have a needle configured at the distal end. FIG. 49A illustrates the delivery or removal handle with wrap around penetrating sheath with needle. FIG. 49B illustrates a close-up view of the wrap around penetrating sheath with needle. FIG. 49C illustrates the wrap around penetrating sheath with needle completely retracted and separate from the delivery or removal handle.

FIGS. 50A-50B illustrate a removal system with alternating lumens for the irrigation and aspiration of the hydrogel. FIG. 50A illustrates the removal handle with a transparent section identifying the alternating lumens mechanism. FIG. 50B illustrates the removal system in cross-section with a close-up illustration of the rotation wheel for alternating the lumens.

FIGS. 51A-51B show the distal end of the removal system and dual-lumen catheters. FIG. 51A illustrates in cross-section the distal end of the dual-lumen catheter. FIG. 51B illustrates a side-view of the distal end of the removal catheter with the aspiration hole and irrigation side holes.

FIGS. 52A-52C illustrates a variation of a delivery system.

FIG. 52D1 illustrates a perspective view of the sheath assembly of FIGS. 52A-52C.

FIG. 52D2 illustrates a top view of the sheath assembly of FIG. 52D1.

FIG. 52D3 illustrates an end view of the sheath assembly of FIG. 52D2.

FIG. 52D4 illustrates a cross-sectional view of the sheath assembly of FIG. 52D3 taken along the line 52D4-52D4.

FIG. 52E1 illustrates a top view of the probe assembly of FIGS. 52A-52C.

FIG. 52E2 illustrates an end view of the probe assembly of FIG. 52E1.

FIG. 52E3 illustrates a cross-sectional view of the probe assembly of FIG. 52E2 taken along the line 52E3-52E3.

FIG. 52E4 illustrates a close-up view of the probe assembly in section 54E4 of FIG. 52E3.

FIG. 52F1 illustrates a side view of the handle assembly of FIGS. 52A-52C.

FIG. 52F2 illustrates an end view of the probe assembly of FIG. 52F1.

FIG. 52F3 illustrates a cross-sectional view of the probe assembly of FIG. 52F2 taken along the line 52F3-52F3.

FIG. 52F4 illustrates a close-up view of the probe assembly in section 54F4 of FIG. 52F3.

FIG. 52F5 illustrates a close-up view of the probe assembly in section 54F5 of FIG. 52F3.

FIG. 52F6 illustrates a close-up view of the probe assembly in section 54F6 of FIG. 52F3.

FIG. 52F7 illustrates a perspective view of the probe assembly of FIG. 52F1.

FIG. 52G illustrates a close-up perspective view of a variation of the cartridge of FIG. 52C.

FIG. 52H1 illustrates Table 1 showing Vasalgel® properties.

FIG. 52H2 illustrates an exemplary synthesis of Vasalgel®.

FIG. 52I1 illustrates a variation of a package.

FIG. 52I2 illustrates a variation of a tray of an open package.

FIG. 52I3 illustrates a variation of a package holder.

FIGS. 53A1-53F2 illustrate a variation of a process of accessing a body space (e.g., a lumen of a vas deferens) and deploying a material 160 into the body space.

FIG. 54A illustrates a variation of a removal system.

FIG. 54B illustrates a close-up view of the removal system in section 54B of FIG. 54A.

FIG. 54C illustrates a close-up view of the removal system in section 54C of FIG. 54A.

FIG. 55A illustrates a variation of the removal system of FIG. 54A.

FIG. 55B illustrates a close-up view of the removal system in section 55B of FIG. 55A.

FIGS. 56A-56B illustrate a variation of a process of accessing a body space (e.g., a lumen of a vas deferens) and removing an occlusion from the body space.

### DETAILED DESCRIPTION

Variations of a system 48 are disclosed, as are variations of methods of using the same. The system 48 can, for example, access a body space 8, deliver one or multiple materials 160 to the body space 8, remove one or multiple materials 160 from the body space 8, temporarily or permanently occlude (e.g., block) the body space, or any combination thereof. For example, the system 48 can access a body space 8, deliver a material 160a to the body space 8, remove the material 160a from the body space 8, deliver a material 160b to the body space 8, remove the material 160b from the body space 8, deliver and/or remove one or more other materials (e.g., material 160c, material 160d) to and/or from the body space, temporarily or permanently occlude the body space, or any combination thereof. The body space 8 can be a space in a body and/or a lumen in a body, for example, in a person or an animal. The body space 8 can be, for example, a body lumen or any other space. The terms body space and body lumen (or any other similar terms) can be used interchangeably herein. For example, any use of the term body lumen herein can be replaced with the term body space and vice versa. For example, the body space 8 can be a space and/or a lumen of an organ and/or a reproductive tract. The reproductive tract can be a male or a female reproductive tract. For example, the body space 8 can be the lumen 8v of a vas deferens 3. As another example, the body space 8 can be the space inside of a fallopian tube 10. As yet additional examples, the body space 8 can be a lumen of a blood vessel (e.g., an artery or a vein), a lumen of the gastrointestinal tract, a lumen of the lymphatic system, or a space or a cavity in any organ.

The material 160a can be delivered to the body space 8, for example, to occlude the body space 8. The material 160a can form an occlusion (e.g., a blockage) in the body space 8 that can temporarily or permanently occlude (e.g., block) the body space 8. The material 160a can be, for example, a hydrogel that can be deployed into the body space 8 to temporarily or permanently occlude (e.g., block) the body space 8. The material 160a can partially occlude (e.g., partially block) the body space 8 or can fully occlude (e.g., fully block) the body space 8. Fully occluding (e.g., fully blocking) the body space 8 can be desirable, for example, male and/or female contraception. As another example, an occlusive device comprising the material 160a can be delivered to the body space 8 to occlude the body space 8. As another example, an occlusive device and the material 160a can be delivered to the body space 8 to occlude the body space 8.

The material 160b can be delivered to the body space 8, for example, to de-occlude the body space 8. The material 160b can be delivered to the body space 8, for example, to reopen the body space 8. The material 160b can be delivered to the body space 8, for example, to restore partial or full patency of the body space 8. The material 160b can be, for example, an agent that can physically and/or chemically break down the material 160a. The product of the interaction between the material 160a and the material 160b can include, for example, the material 160a (e.g., pieces or chunks of the material 160a), the material 160b, a material chemically different from the material 160a and the material 160b, or any combination thereof. The material 160b can be delivered into the body space 8, for example, to break down the material 160a in the body space 8 to restore patency of the body space 8. The material 160a can be broken down to reopen the body space 8, for example, to reverse the occlusive effect of the material 160a in the body space 8.

The material 160a, the material 160b, and/or the material the product of the interaction between the material 160a and the material 160b can be removed from the body space 8. For example, after the material 160b is delivered into a body space 8 that has the material 160a, the material 160a, the material 160b, and/or the material the product of the interaction between the material 160a and the material 160b can be actively and/or passively removed from the body space 8. Active removal can involve, for example, suctioning the material 160a, the material 160b, and/or the material the product of the interaction between the material 160a and the material 160b out of the body space 8. Passive removal can involve, for example, a person or an animal naturally passing the material 160a, the material 160b, and/or the material the product of the interaction between the material 160a and the material 160b through their body, for example, through their urethra.

The material 160a can be, for example, a first material. The material 160b can be, for example, a second material. The product of the interaction between the material 160a and the material 160b can be, for example, a third material, whereby the third material can comprise, for example, a mixture of the material 160a and the material 160b, a material chemically different from the material 160a and the material 160b, or both. The one or other materials referred to above can comprise, for example, the third material.

The system 48 can have an occlusion delivery system 48D (also referred to as the system 48D), an occlusion removal system 48R (also referred to as the system 48R), or both the system 48D and the system 48R. The system 48D can have, for example, an occlusion delivery device 50 (also referred to as the device 50). The system 48R can have for example, an occlusion removal device 180 (also referred to as the device 180). The device 50 can be, for example, an access device configured to access the body space 8 and deliver the material 160a to the body space 8. The device 180 can be, for example, an access device configured to access the body space 8 and remove the material 160a from the body space 8.

The figures illustrate various exemplary variations of the system 48, various exemplary devices that the system 48 can have, various exemplary uses for the system 48, various exemplary methods of using the system 48, and various exemplary anatomies that the system 48 can be used with. The system 48 can have any combination of the features and/or devices described and/or illustrated herein. The system 48 can be used, for example, with any of the anatomy shown in any of the figures. The systems, devices, and/or features described and/or illustrated herein can be combined with each other in any combination such that the system 48 can have any combination of the features and/or devices described and/or illustrated herein. For example, the system 48 can include the occlusion delivery device 50, the occlusion removal device 180, the occlusion delivery device 50 and the occlusion removal device 180, or a single device having any combination of features of the occlusion delivery system 48D (e.g., the device 50) and the occlusion removal system 48R (e.g., the device 180). As another example, the occlusion delivery device 50 can have any of the features of the occlusion removal device 180 or vice versa.

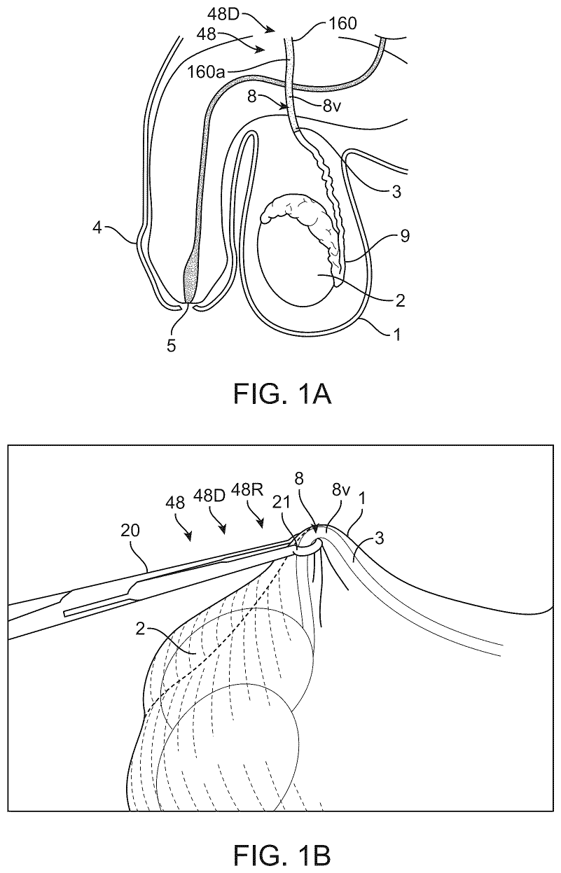

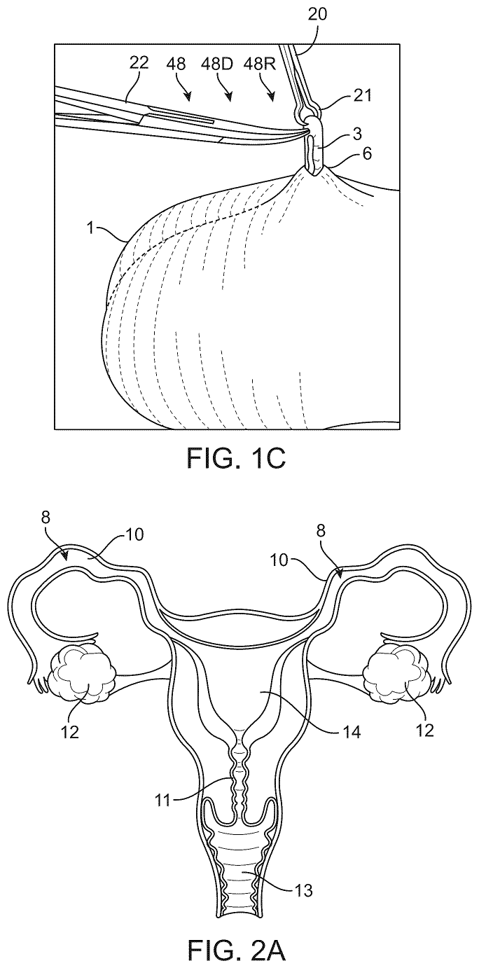

FIGS. 1A-1C illustrate an anatomical location of the vas deferens 3 in the male reproductive tract.

FIG. 1A illustrates a cross-sectional view of a typical anatomical location of a vas deferens 3 in the male reproductive tract; however, anatomical variations are possible. FIG. 1A illustrates the location of a vas deferens 3 in a scrotum 1. Sperm produced in the testes 2 and matured in the epididymis 9 traverse the vas deferens 3. During a normal ejaculation, for example, prior to a vasectomy procedure, sperm will be ejected out of the penis 4 through the urethra 5. For male sterilization, vasectomy procedures can ligate the vas deferens 3 or can occlude the vas deferens 3 to prevent the passage of sperm into the urethra 5. For example, FIG. 1A illustrates that the material 160a can be injected into the lumen 8v of the vas deferens 3 to prevent the passage of sperm into the urethra 5.

The material 160a can comprise one or multiple hydrogels. For example, the material 160a can comprise 1-5 hydrogels, including, for example, every 1 hydrogel increment within this range (e.g., 1 hydrogel, 2 hydrogels, 3 hydrogels, 4 hydrogels, 5 hydrogels). For example, FIG. 1A illustrates that the material 160a can be one hydrogel. The material 160a can be, for example, a hydrogel. The hydrogel can be, for example, Vasalgel®. The material 160a can be porous. The material 160a can be non-porous. The material 160a can have a porous region and a non-porous region. For example, on the one hand, the porosity of the material 160a (e.g., a hydrogel) can allow fluid to pass into the urethra 5, and on the other hand, the matrix of the material 160a (e.g., a hydrogel) can prevent sperm from permeating through the material 160a (e.g., through the hydrogel) into the urethra 5.

The material 160a can be designed for removal from the body space 8 (e.g., from the vas deferens lumen 8v), for example, with the introduction of the material 160b into the body space 8. The material 160b can comprise one or multiple agents (also referred to as removal agents). For example, the material 160b can comprise 1-5 or more agents, including, for example, every 1 agent increment within this range (e.g., 1 agent, 2 agents, 3 agents, 4 agents, 5 agents). The material 160b can be deployed into the body space 8, for example, to interact with the material 160a. The material 160b can physically and/or chemically break down the material 160a. For example, the material 160b can break down the material 160a by dissolving the material 160a. The material 160b can comprise, for example, sodium bicarbonate, dimethyl sulfoxide, or both. The material 160b can be or can comprise a one or multiple solutions. For example, the material 160b can be or can comprise solutions such as sodium bicarbonate solution, dimethyl sulfoxide solution, or other solutions. The material 160b can break down (e.g., dissolve and/or disassociate) the material 160a in the body space 8. Once the material 160a is broken down by the material 160b, patency within the vas deferens lumen 8v can be restored for a return to fertility for the male. Once the material 160a is broken down by the material 160b and/or as the material 160a is being broken down by the material 160b, the material 160a, the material 160b, and/or the product of the interaction between the material 160a and the material 160b can be actively and/or passively removed from the body space 8, for example, by suctioning it out of the body space 8 and/or by allowing it to pass into the urethra 5 be discharged through the urethral opening at the tip of the penis 4, or any combination thereof.

FIG. 1B illustrates that the system 48 (e.g., the system 48D and/or the system 48R) can have a clamp 20. The clamp 20 can have a holder 21. The holder 21 can be, for example, a distal end of the clamp 20. The clamp 20 can be, for example, a ring clamp. The holder 21 can be, for example, a holding ring. FIG. 1B illustrates a variation of isolating the vas deferens 3 from the scrotum 1 by the placing and positioning of the clamp 20 and the holder 21. The physician can palpate the scrotum 1 to identify and isolate (e.g., elevate) the vas deferens 3, and can use the holder 21 of the clamp 20 to grasp the vas deferens 3. Additionally or alternatively, the vas deferens 3 can be identified and isolated via one or multiple imaging modalities. For the insertion of the material 160a and/or an implant comprising the material 160a into the vas deferens lumen 8v, the clamp 20 can be used as a stabilizer for the percutaneous insertion of the material 160a and/or an implant comprising the material 160a. The material 160a and/or the implant comprising the material 160a can be inserted into the lumen 8v of the vas deferens 3 to occlude the lumen 8v.

FIG. 1C illustrates that the system 48 (e.g., the system 48D and/or the system 48R) can have a forceps 22. The forceps 22 can be, for example, a dissecting forceps. FIG. 1C illustrates an isolated vas deferens 3 that is visible through the scrotum 1 through an opening 6 in the scrotal skin (also referred to as the scrotal skin opening 6). FIG. 1C illustrates that the vas deferens 3 can be lifted through the opening 6 by the holder 21 of the clamp 20. Fascia and other tissues can be removed by the forceps 22 to further isolate the vas deferens 3. At this juncture (i.e., the juncture shown in FIG. 1C), non-percutaneous and/or direct access to the vas deferens 3 can be possible via the system 48D and/or via the system 48R.

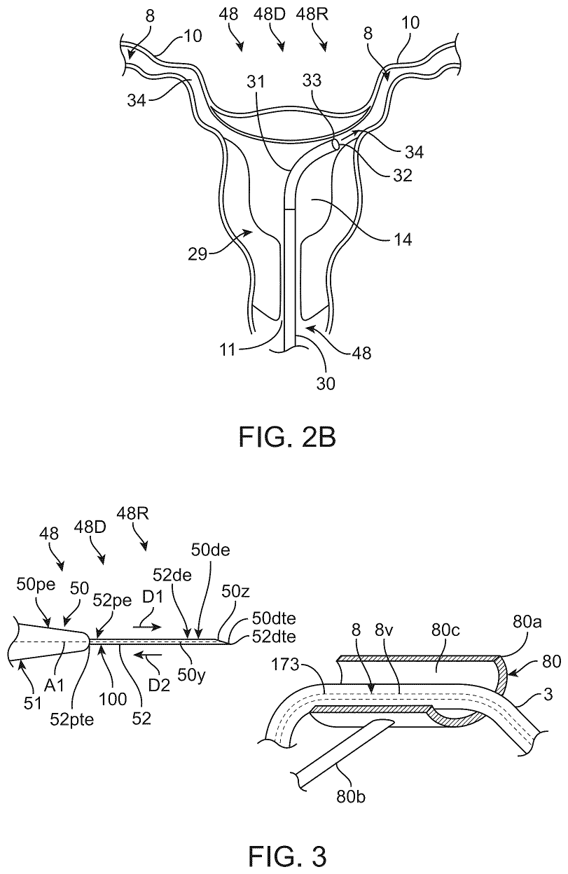

FIG. 2A illustrates a schematic cross-sectional view of a location of the fallopian tubes 10 in the female reproductive tract. The external opening of the uterus 14 is through the cervix 11 which is located in the vagina 13. At the distal end of the fallopian tube 10 is the ovary 12 in which follicles mature and eggs are emitted. Sperm from the male fertilize the egg within the fallopian tube 10. For female sterilization, the fallopian tubes 10 can be ligated or occluded. For female contraception that is designed to be reversable, the material 160a (e.g., a hydrogel) can be placed within the fallopian tubes 10 to inhibit the passage of sperm in the fallopian tubes 10 to prevent the fertilization of an egg.

FIG. 2B illustrates that the system 48 (e.g., the system 48D and/or the system 48R) can have a hysteroscope 30 and/or a catheter 31. The catheter 31 can be used to delivery and/or remove one or multiple materials 160 (e.g., the material 160a) to and/or from the fallopian tubes 10. For example, FIG. 2B illustrates an exemplary position of the hysteroscope 30 for the positioning of the catheter 31 at the os 34 of the fallopian tube 10 prior to insertion of the material 160a for female contraception. The hysteroscope 30 can provide an internal image of the uterus 14 and identify the os 34 for direct insertion of the material 160a within the fallopian tube 10. FIG. 2B illustrates the uterine wall 29. Additionally or alternatively, the fallopian tube 10 can be accessed directly through a trans-abdominal approach involving laparoscopy or a surgical incision. The fallopian tube 10 on the left side of FIG. 2B can be, for example, the right fallopian tube of the patient, and the fallopian tube 10 on the right side of FIG. 2B can be, for example, the left fallopian tube of the patient. The distal end of the hysteroscope 30 can have illumination source and visualization optics 32 for imaging the uterine cavity. The illumination source and visualization optics can be, for example, a camera chip with LED lighting, rigid lens optics, or any combination thereof. The distal end of hysteroscope 30 can have a distal end opening lumen 33 for the delivery of catheters, sheaths, media, and materials.

FIG. 3 illustrates that the system 48 (e.g., the occlusion delivery system 48D) can have an occlusion delivery device 50. FIG. 3 illustrates a variation of the system 48, for example, of the device 50.

FIG. 3 illustrates that the device 50 can have a handle 51, a conduit assembly 100, or both the handle 51 and the conduit assembly 100. The conduit assembly 100 can have a conduit 52. The conduit 52 can be, for example, a shaft, a sheath, a tube, a needle, or any combination thereof. The needle can be, for example, a full needle (e.g., a hypodermic needle), a portion of a needle (e.g., a half needle), a split needle, or any other type of needle. For example, FIG. 3 illustrates that conduit 52 can be a sheath with a beveled end. FIG. 3 illustrates that the device 50 may not have a needle, whereby the conduit 52 can be inserted into the body space 8, for example, after puncturing the wall of the body space 8 (e.g., using a scalpel, the forceps 22, or a needle). The conduit 52 can be flexible or rigid. For example, FIG. 3 illustrates that the conduit 52 can be flexible. The distal end of the of the conduit 52 can be blunt, sharp, beveled, non-beveled, or any combination thereof. For example, FIG. 3 illustrates that the distal end of the conduit 52 can be beveled, for example, to have a pointed tip to help push the conduit 52 through the puncture (e.g., hole) created in the wall of the body space 8 (e.g., via the scalpel, the forceps 22, or the needle) to access the body space 8 (e.g., the vas deferens lumen 8v) with the conduit 52. The distal end of the conduit 52 can have a beveled edge, a non-beveled edge, or both. As another example, the conduit assembly 100 can have a needle separate from the conduit 52 that can be attached to or integrated with the device 50. As another example, the conduit 52 can be a needle. FIG. 3 illustrates that the conduit 52 can have a conduit proximal end 52pe and a conduit distal end 50de. FIG. 3 illustrates that the conduit proximal end 52pe can have a conduit proximal terminal end 52pte, and that the conduit distal end 52de can have a conduit distal terminal end 52dte. The device 50 can have a device proximal end 50pe and a device distal end 50de. FIG. 3 illustrates that the device distal end 50de can have a device distal terminal end 50dte. The device distal terminal end 50dte can be the same as or different from the conduit distal terminal end 52dte. The device proximal end 50pe can be or can comprise, for example, the handle 51, the conduit 52 (e.g., the conduit proximal end 52pe), or both. The device distal end 50de can be or can comprise, for example, the conduit 52, the handle 51, or both. FIG. 3 illustrates that the device distal end 52ee can be or can comprise the conduit distal terminal end 52dte.

FIG. 3 illustrates that the device 50 can have one or multiple lumens 50y (also referred to as the lumen 50y and the lumens 50y). For example, the device 50 can have 1-10 lumens 50y, or more narrowly, 1-5 lumens 50y, or more narrowly still, 1-3 lumens 50y, including, for example, every 1 agent increment within these ranges (e.g., 1 lumen, 2 lumens, lumens, 10 lumens). For example, FIG. 3 illustrates that the device 50 can have the lumen 50y. The lumens 50y can extend through, for example, the conduit 52 and/or the handle 51. For example, FIG. 3 illustrates that the lumens 50y can extend through the conduit 52 and the handle 51.

FIG. 3 illustrates that the device 50 can have one or multiple openings 50z (also referred to as the opening 50z and the openings 50z). For example, the device 50 can have 1-30 or more openings 50z, or more narrowly, 1-20 openings 50z, or more narrowly still, 1-10 openings 50z, including every 1 opening increment within these ranges (e.g., 1 opening, 2 openings, 5 openings, 10 openings, 20 openings, 30 openings, 40 openings). For example, FIG. 3 illustrates that the distal end 50de can have the opening 50z. The openings 50z can be one or more side openings of the device 50 and/or one or multiple distal terminal openings of the device 50. For example, the opening 50z can be a distal terminal opening of the device 50, for example, of the conduit 52. As another example, the opening 50z can be a side opening of the device 50, for example, an opening on the side of the conduit 52 (e.g., that extends through the wall of the conduit 52). For example, FIG. 3 illustrates that the opening 50z can be a distal terminal opening of the conduit 52. FIG. 3 illustrates, for example, that the conduit distal end 52de can have the opening 50z and/or can be the tip of the conduit 52. FIG. 3 illustrates that the openings 50z can be ports of the lumens 50y. Each lumen 50y can have one or multiple openings 50z. For example, FIG. 3 illustrates that the lumen 50y can terminate at the opening 50z.

FIG. 3 illustrates that the lumens 50y and the openings 50z can be, for example, for the delivery and/or removal of the material 160a into and/or from the body space 8, for the delivery and/or removal of the material 160b into and/or from the body space 8, for the removal of the product of the interaction between the material 160a and the material 160b from the body space 8, or any combination thereof. For example, FIG. 3 illustrates that the material 160a can be delivered to the body space 8 through one or more of the lumens 50y and openings 50z, the material 160a can be removed from the body space 8 through one or more of the lumens 50y and openings 50z, the material 160b can be delivered to the body space 8 through one or more of the lumens 50y and openings 50z, the material 160b can be removed from the body space 8 through one or more of the lumens 50y and openings 50z, the product of the interaction between the material 160a and the material 160b can be removed from the body space 8 through one or more of the lumens 50y and openings 50z, or any combination thereof.

FIG. 3 illustrates that the device 50 can be advanced in direction D1 and/or retracted in direction D2 toward and away from the body space 8, respectively. The direction D1 and the direction D2 can be, for example, opposite to each other. The direction D1 and the direction D2 can be parallel, for example, to the center longitudinal axis A1 of the conduit 52 (also referred to as the conduit center longitudinal axis A1). The conduit center longitudinal axis A1 can be, for example, the center longitudinal axis of the lumen 50y and/or of the opening 50z.

FIG. 3 illustrates that the device 50 can be advanced in direction D1 to access a target site 173 with the device distal end 50de (e.g., with the conduit 52). The target site 173 can be, for example, the body space 8 that is being targeted (e.g., the vas deferens lumen 8v, the space in a fallopian tube 10). The target site 173 can be a delivery site, a removal site, or a delivery site and a removal site, for example, for the material 160a, the material 160b, the product of the interaction between the material 160a and the material 160b, other materials, or any combination thereof. FIG. 3 illustrates that the target site 173 can be the body space 8. For example, FIG. 3 illustrates that the target site 173 can be the vas deferens lumen 8v. FIG. 3 illustrates, for example, that the device 50 can be moved in direction D1 to advance the conduit 52 into the vas deferens lumen 8v. FIG. 3 illustrates that the device distal end 50de (e.g., the conduit distal end 52de) can pierce tissue (e.g., the anterior surface of vas deferens 3) to access the body space 8 (e.g., the vas deferens lumen 8v). The device 50 can have access to the target site 173, for example, when one or more of the openings 50z (e.g., the opening 50z shown in FIG. 3) have access (e.g., are in) the target site 173. For example, FIG. 3 illustrates that the device 50 can have access to the target site 173 when the opening 50z is in the vas deferens lumen 8v.

FIG. 3 illustrates that the device 50 can be retracted in direction D2 to withdraw the device 50 (e.g., the device distal end 50de) from the target site 173. FIG. 3 illustrates, for example, that the device 50 can be moved in direction D2 to withdraw the conduit 52 from the vas deferens lumen 8v.

FIG. 3 illustrates that the system 48 (e.g., the system 48D and/or the system 48R) can have a support system 80. The support system 80 can have a support 80a, an arm 80b, and/or a surface 80c. FIG. 3 illustrates that the support 80a can, support, isolate, and/or stabilize the vas deferens 3, for example, so that the device 50 can be advanced directly into the target site 173 (e.g., as opposed to through the scrotum 1). FIG. 3 illustrates that the support 80a can be attached to or integrated with the arm 80b. The arm 80b can be, for example, a rod that can be used to manipulate (e.g., move) the support 80a into a position (e.g., into the position shown in FIG. 3). FIG. 3 illustrates that the support 80a can be a platform having a surface 80c. FIG. 3 illustrates that the surface 80c can support the vas deferens 3. FIG. 3 illustrates that the vas deferens 3 can be in contact with the surface 80c. The platform can be flat and/or curved. For example, the surface 80c can be flat and/or curved. The support 80a can be, for example, a tube (also referred to as a pipe) or a portion of a tube. For example, FIG. 3 illustrates that the support 80a can be a portion of a tube. FIG. illustrates that the portion can be half of a pipe (e.g., a transverse half of a pipe) such that FIG. 3 illustrates that the support 80a can be a half-pipe. However, any portion of a pipe is appreciated, including for example, an eighth-pipe or a quarter-pipe. The support 80a can surround (e.g., circumferentially surround) the vas deferens 3 so that direct access with the device 50 can be made without rotation of the tubular structure of the vas deferens 3. For example, FIG. 3 illustrates that the support 80a can partially circumferentially surround the vas deferens 3. As another example, the support 80a can be a flat platform (e.g., the surface 80c can be flat) on the posterior portion of the vas deferens 3 which can, for example, facilitate a linear insertion of the device distal end 50de (e.g., the opening 50z) into the target site 173.

FIG. 3 illustrates that isolating the vas deferens 3 from the scrotum 1 can be performed with the support system 80. For example, the vas deferens 3 can be raised and lowered relative to the scrotum 1 (e.g., through the scrotal skin opening 6), for example, by raising and lowering the arm 80b, respectively. For example, FIG. 3 illustrates that the support system 80 can facilitate insertion of the device distal end 50de through an anterior surface of vas deferens 3. While the vas deferens 3 is positioned on and/or within the support 80a, the device distal end 50de (e.g., the opening 50z) can access the body space 8, for example, by being advanced in direction D1. FIG. 3 illustrates, for example, that the support system 80 can isolate and/or position the vas deferens 3 so that the device 50. As another example, the device 50 can pierce the scrotum 1 to indirectly access the target site 173 (e.g., through the scrotum 1). When the target site 173 is indirectly accessed, the support system 80 may or may not be used.

FIG. 3 illustrates that accessing the body space 8 can include, for example, penetrating tissue such as a wall (e.g., an anterior wall) of the vas deferens 3 by advancing the device 50 in direction D1. The body space 8 can be accessed by the device distal end 50de (e.g., via the opening 50z), for example, for the delivery and/or removal of the material 160a, for the delivery and/or removal of the material 160b, for the removal of the product of the interaction between the material 160a and the material 160b, or any combination thereof. The device 50 can be, for example, a delivery device, a removal device, or both a delivery device and a removal device, for example, for the material 160a and/or the material 160b. The device 50 can be used, for example, to deliver the material 160a, to deliver an implant comprising the material 160a, to remove the material 160a, to remove an implant comprising the material 160a, to deliver a removal agent (e.g., the material 160b), to remove a removal agent (e.g., the material 160b), to remove the product of the interaction between the material 160a and the material 160b, or any combination thereof. The device 50 can be, for example, a delivery and/or removal device (e.g., a delivery device, a removal device, or both a delivery and removal device).

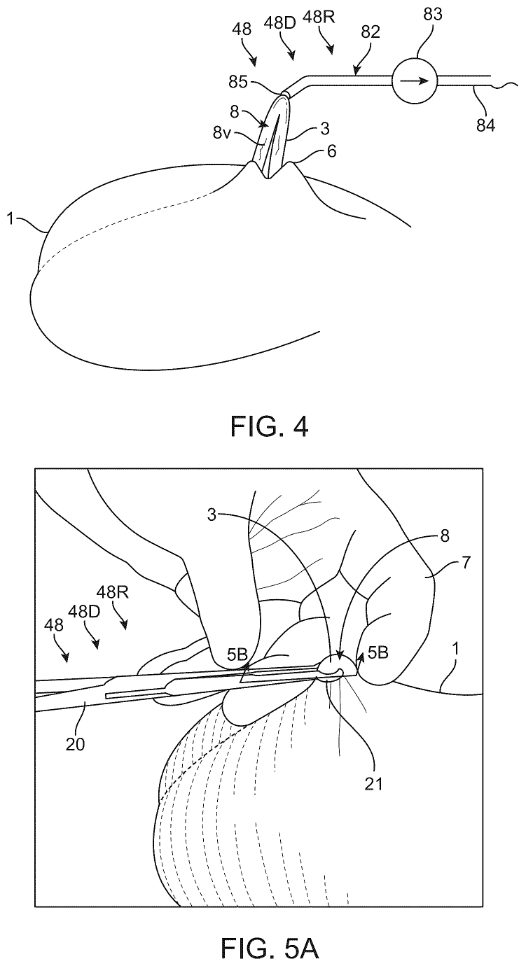

FIG. 4 illustrates that the system 48 (e.g., the system 48D and/or the system 48R) can have a holding device 82. The holding device 82 can have a vacuum source 83, an aspiration conduit 84, and an opening 85. The opening 85 can be, for example, at the distal end of the aspiration conduit 84. For example, FIG. 4 illustrates that the opening 85 can be a distal terminal opening of the aspiration conduit 84. FIG. 4 illustrates that the holding device 82 can apply negative pressure to the vas deferens 3 through the opening 85 via negative pressure provided by the vacuum source 83 through the aspiration conduit 84. FIG. 4 illustrates that the vas deferens 3 can be attached to the holding device 82 at the opening 85 via negative pressure, for example, to isolate the vas deferens 3 from the scrotum 1 by pulling the vas deferens 3 out of the scrotum 1 through the scrotal skin opening 6 (e.g., to the position shown in FIG. 4). Through the negative pressure holding force, the vas deferens 3 can be stabilized for subsequent delivery and/or removal of the material 160a into and/or from the body space 8, for the subsequent delivery and/or removal of the material 160b into and/or from the body space 8, for subsequent removal of the product of the interaction between the material 160a and the material 160b from the body space 8, or any combination thereof.

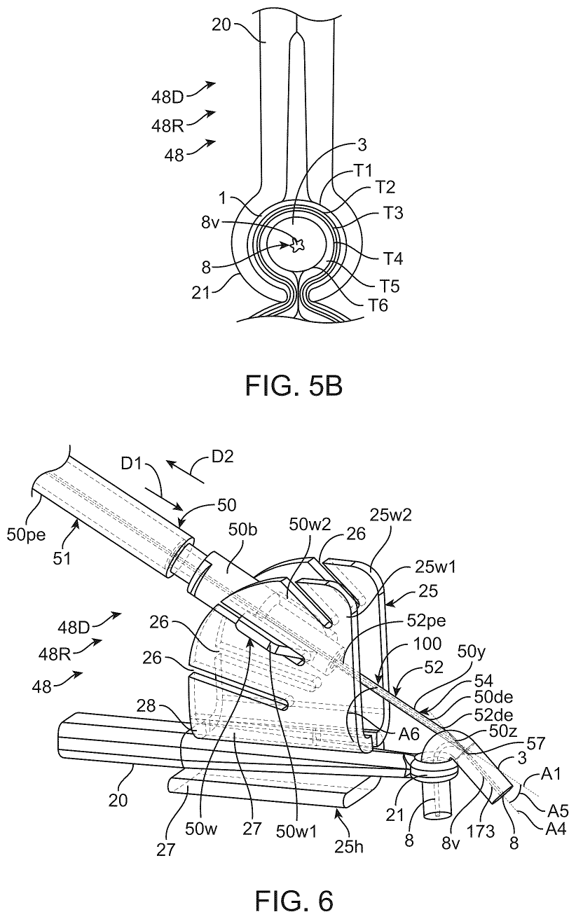

FIGS. 5A 5B illustrate a technique for isolating the vas deferens 3 with a hand and/or the clamp 20 for vasectomy procedures (e.g., for traditional vasectomy procedures), for example, in preparation for direct or indirect access via the system 48 (e.g., via the device 50 and/or via the device 180). Additional imaging modalities can be employed for identification and isolation of the vas deferens 3.

FIG. 5A illustrates that the physician's fingers 7 can palpate the vas deferens 3 from the scrotum 1, for example, to access the vas deferens via the system 48 (e.g., via device 50 and/or via the device 180). For example, FIG. 5A illustrates that the physician can palpate the scrotum 1 to identify and isolate (e.g., elevate) the vas deferens 3, and can use the holder 21 of the clamp 20 to grasp the vas deferens 3 from outside of the scrotum 1. For example, FIG. 5A illustrates the vas deferens 3 and the scrotum 1 clamped by the holder 21.

FIG. 5B illustrates in a cross-sectional view the structure of the vas deferens 3 within the scrotum 1 and held by the holder 21 of the clamp 20. FIG. 5B illustrates the vas deferens lumen 8v within the vas deferens 3. The body space 8 (e.g., a vas deferens lumen 8v, the space inside a fallopian tube 10) can be the intended target site 173 for placement of the device 50.

When the device distal end 50de is positioned inside of the body space 8 (e.g., inside of a vas deferens lumen 8v or a space in a fallopian tube 10), the device 50 can have access to the target site 173 (e.g., to the body space 8). The device 50 can be considered to have access to the target site 173 when the conduit distal terminal end 52dte and/or an opening 50z has access (e.g., is inside) the body space 8. When the device 50 has access to the body space 8, the device 50 can be considered to be in a correctly placed position, for example, for the delivery and/or removal of material from the body space 8. For males, when the device distal end 50de is in a correctly placed position, the device distal end 50de (e.g., the conduit distal terminal end 52dte and/or an opening 50z) can be inside of the vas deferens lumen 8v.

When the device distal end 50de is positioned outside of the body space 8 (e.g., outside of the vas deferens lumen 8v or a space in the fallopian tube 10), the device 50 may not have access to the target site 173 (e.g., to the body space 8). The device 50 can be considered to not have access to the target site 173 when the conduit distal terminal end 52dte and/or an opening 50z is outside of the body space 8. When the device 50 does not have access to the body space 8, the device 50 can be considered to be in an incorrectly placed position, for example, for the delivery and/or removal of material from the body space 8. For males, when the device distal end 50de is in an incorrectly placed position, the device distal end 50de (e.g., the conduit distal terminal end 52dte and/or an opening 50z) can be outside of the vas deferens lumen 8v, for example, in other portions of the vas deferens 3 wall, fascia, or interstitial spaces, including, for example, in the skin T1 of the scrotum 1, in the dartos T2, in the external spermatic fascia T3, in the cremasteric fascia T4, in the cremaster muscle T5, or in the internal spermatic facia T6. An incorrect placement of the device distal end 50de can render subsequent delivery and/or removal of the material 160a, the material 160b, and/or the product of the interaction between the material 160a and the material 160b unsuccessful given that the material 160a and/or the material 160b may not be deployed into the body space 8 (e.g., the vas deferens lumen 8v) during delivery and/or removal. An incorrect placement of the device 50 can damage the structure of the vas deferens 3 and/or can prevent the potential for reversing the occlusion formed by the material 160a and a return to fertility in the future. Hence, the proper identification and placement of the device 50 (e.g., the device distal end 50de) within the lumen 8v can be a requirement for both contraception and reversal in the future. An incorrect placement of the device 50 can be unintentional.

FIG. 6 illustrates that when the device 50 is inserted into the body space 8, the device 50 can be aligned with the body space 8. FIG. 6 illustrates that when the device 50 is aligned with the body space 8 (e.g., with vas deferens lumen 8v), the conduit center longitudinal axis A1 can be aligned with the body space 8 (e.g., with the vas deferens lumen 8v) such that the conduit center longitudinal axis A1 intersects the body space 8 (e.g., the vas deferens lumen 8v), for example, at an alignment angle A5. The alignment angle A5 can be measured, for example, between the conduit center longitudinal axis A1 and the center longitudinal axis A4 of the body space 8 (also referred to as the body space center longitudinal axis A4). FIG. 6 illustrates that the body space longitudinal axis A4 can be, for example, the center longitudinal axis of the vas deferens lumen 8v (also referred to as the vas deferens center longitudinal axis). FIG. 6 illustrates that when the device 50 is aligned with the body space 8, the alignment angle A5 can be, for example, 0-45 degrees, or more narrowly, 0-30 degrees, or more narrowly still, 0-20 degrees, including every 1 degree increment within these ranges (e.g., 0 degrees, 5 degrees, 10 degrees, 15 degrees, 20 degrees, 30 degrees, 45 degrees). An alignment angle A5 of 0 degrees can indicate that the conduit center longitudinal axis A1 and the body space center longitudinal axis A4 are parallel to or collinear with each other. FIG. 6 illustrates that the device 50 (e.g., the conduit 52) can be inserted into the body space 8 when the alignment angle is at the alignment angle A5, including, for example, when the alignment angle A5 is greater than 0 degrees (e.g., as shown in FIG. 6).

FIG. 6 illustrates that the device 50 can have a body 50b. FIG. 6 illustrates that the body 50b can have one or multiple device wings 50w, including, for example, a device first wing 50w1 and a device second wing 50w2. FIG. 6 illustrates that the device first wing 50w1 and the device second wing 50w2 can be, for example, extensions that extend laterally and/or radially away from the body 50b. The handle 51 can be and/or can comprise the body 50b, for example, as shown in FIG. 6. As another example, the body 50b can be between the handle 51 and the conduit 52.

FIG. 6 illustrates that the system 48 (e.g., the system 48D and/or the system 48R) can have an angle miter 25. FIG. 6 illustrates that the angle miter 25 can allow for the controlled insertion of the system 48 (e.g., the device 50 and/or the device 180) into the body space 8 by aligning the device distal end 50de (e.g., the conduit assembly 100) with the body space 8 (e.g., the vas deferens lumen 8v). The angle miter 25 can have the arrangement of features shown. For example, FIG. 6 illustrates that the angle miter 25 can have angle miter slots 26, an angle miter first wing 25w1, an angle miter second wing 25w2, an angle miter holder 25h, an angle miter base 27, an angle miter groove 28, or any combination thereof. The angle miter 25 can have, for example, 2-20 or more slots 26 in each of the angle miter first wing 25w1 and the angle miter second wing 25w2, or more narrowly, 2-10 slots 26 in each of the angle miter first wing 25w1 and the angle miter second wing 25w2, including every 1 slot increment within these ranges (e.g., 2 slots, 3 slots, 10 slots, 20 slots, 30 slots). The angle miter first wing 25w1 and the angle miter second wing 25w2 can have the same number of slots 26. FIG. 6 illustrates, for example, that the angle miter first wing 25w1 and the angle miter second wing 25w2 can each have 3 slots 26. The device first wing 50w1 can be inserted into and withdrawn from any of the slots 26 in the angle miter first wing 25w1, and the device second wing 50w2 can be inserted into and withdrawn from any of the slots 26 in the angle miter second wing 25w2, or vice versa. The device first wing 50w1 and the device second wing 50w2 can be inserted (e.g., slid) into the slot 26 in the angle miter first wing 25w1 and the slot 26 in the angle miter second wing 25w2, respectively, that aligns (e.g., that best aligns) the device distal end 50de with the body space 8 (e.g., with the vas deferens lumen 8v). For example, the device first wing 50w1 and the device second wing 50w2 can be inserted (e.g., slid) into the slot 26 in the angle miter first wing 25w1 and the slot 26 in the angle miter second wing 25w2, respectively, that minimizes the alignment angle A5. Minimizing the alignment angle A5 can include choosing the slots 26 that result in the alignment angle A5 being 0 degrees or the closest to 0 degrees.

FIG. 6 illustrates that each of the slots can be at an angle relative to the angle miter base 27. FIG. 6 illustrates, for example, that the slots 26 can each be at an angle within in an angle range A6. The angle range A6 can include, for example, angles measured between the slots 26 and the angle miter base 27. The angle range A6 can be, for example, 0-90 degrees, or more narrowly, 0-60 degrees, or more narrowly still, 0-45 degrees, including every 1 degree increment within these ranges. Each of the slots 26 can have any angle within the angle range A6, including, for example, 0 degrees, 10 degrees, 15 degrees, 30 degrees, 45 degrees, 60 degrees, 90 degrees. For example, FIG. 6 illustrates that the angle miter 25 can have a 15 degree slot 26, a 30 degree slot 26, and a 45 degree slot 26. The slots 26 can be evenly or unevenly spaced, for example, in 5 degree, 10 degree, 15 degree, 20 degree, 25 degree, and/or 30 degree increments. For example, FIG. 6 illustrates that the slots 26 can be evenly spaced in 15 degree increments.

FIG. 6 illustrates that the device 50 (e.g., the device wings 50w of the device 50) can be placed in the slots 26, for example, to set, stabilize, and/or control the alignment angle A5. FIG. 6 illustrates, for example, that the angle miter 25 can stabilize the device 50 such that the alignment angle A5 can be stabilized (e.g., held constant) while the device 50 is inserted into the body space 8, while the device 50 is maintained in the body space 8 (e.g., during delivery and/or removal of materials), while the device 50 is withdrawn from the body space 8, or any combination thereof. FIG. 6 illustrates, for example, the device 50 (e.g., the device wings 50w) can be placed in the 30 degree slot 26 of the angle miter 25, for example, to set, stabilize, and/or control the alignment angle A5. The alignment angle A5 can be set, stabilized, and/or controlled with or without the angle miter 25. When the angle miter 25 is not used, the alignment angle A5 can be set, stabilized, and/or controlled, for example, by hand. For example, FIG. 3 illustrates that the alignment angle A5 can be set, stabilized, and/or controlled via one or both of a person's hands, and FIG. 6 illustrates that the alignment angle A5 can be set, stabilized, and/or controlled via the angle miter 25.

FIG. 6 illustrates that the vas deferens 3 can be isolated and held by the holder 21 with the clamp 20 removably attached to the angle miter 25 for the controlled insertion of the device distal end 50de (e.g., the opening 50z) into the vas deferens lumen 8v. FIG. 6 illustrates that the holder 25h can be, for example, a connector that the clamp 20 can be removably attached to. The holder 25h can be, for example, a clip. For example, the angle miter 25 can be clipped onto the clamp 20, or vice versa, by placing the clamp 20 into a groove 28 in the angle miter base 27. The holder 21 can, for example, stabilize the vas deferens 3 while the clamp 20 is attached to the holder 25h. FIG. 6 illustrates that the device distal end 50de can be placed through a wall (e.g., the anterior wall) of the vas deferens 3 and into the lumen 8v by advancing the device 50 in direction D1 while in the slots 26. The device 50 can be used for the placement of the material 160a and/or the material 160b. In either situation, the angle miter 25 can facilitate the proper placement of the device 50 into the body space (e.g., the vas deferens lumen 8v). For example, FIG. 6 illustrates the device 50 fully advanced in direction D1 in the slots 26 with the device distal end 50de (e.g., the opening 50z) in the vas deferens lumen 8v. FIG. 6 illustrates the device 50 in a position in which the device 50 has access to the vas deferens lumen 8v such that one or more materials (e.g., the material 160a, the material 160b, and/or the product of the interaction between the material 160a and the material 160b) can be delivered into and/or removed from the vas deferens lumen 8v through the device distal end 50de (e.g., through the opening 50z).

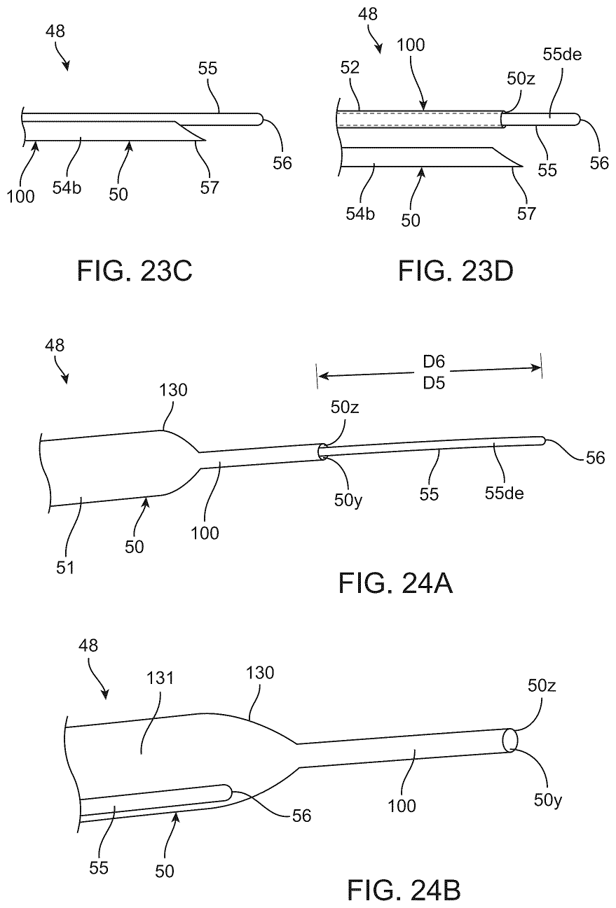

FIG. 6 illustrates that that the conduit assembly 100 can have a needle 54. The needle 54 can be configured to penetrate the wall of the body space 8, for example, as shown in FIG. 6. FIG. 6 illustrates that the conduit 52 can be the needle 54. As another example, the needle 54 can be different from the conduit 52. The conduit 52 can be, for example, a sheath that can house the needle 54 or vice versa. The conduit 52 can be, for example, advanced and/or retracted relative to the needle 54 or vice versa. As another example, the device 50 can have two needles, whereby the conduit 52 can be a first needle and the needle 54 can be a second needle. As another example, the device 50 can have a sheath and the needle 54, whereby the conduit 52 can be the sheath (e.g., as shown in FIGS. 23A-23D). The needle 54 can be any type of needle, including, for example, a full needle (e.g., a conventional needle, a non-split needle), a portion of a needle (e.g., a half needle), a split needle, or any combination thereof. FIG. 6 illustrates that the needle 54 can have a tip 57 (also referred to as the needle tip 57). FIG. 10 illustrates that the needle tip 57 can be a distal tip of the needle 54. The needle tip 57 can be sharp, for example, so that the needle 54 can penetrate tissue and access the target space 173 (e.g., the body space 8).

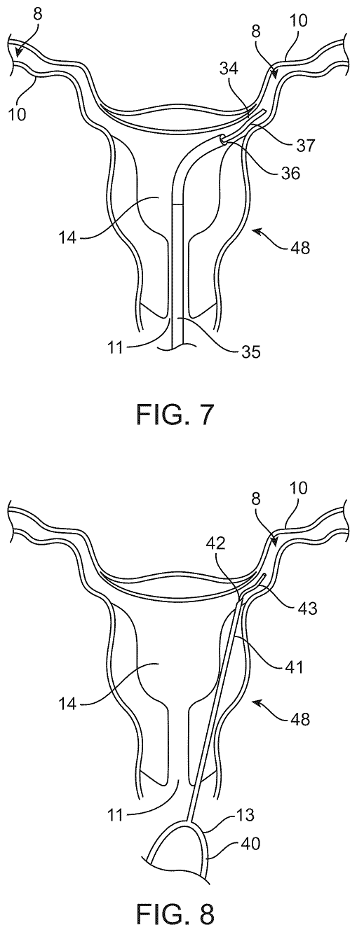

FIG. 7 illustrates that the system 48 (e.g., the system 48D and/or the system 48R) can have a catheter 35. FIG. 7 illustrates that the body space 8 can be the space inside of a fallopian tube 10. FIG. 7 illustrates that the catheter 35 can be transcervically positioned for intrauterine placement into the os 34 of the fallopian tube 10. The catheter 35 can be placed through cervix 11 into the uterus 14. Through a distal end opening 36 of the catheter 35, a delivery device 37 can intubate fallopian tube 10. Once positioned within the body space 8 (e.g., within the fallopian tube 10), the delivery and/or removal of one or more materials (e.g., the material 160a, the material 160b, and/or the product of the interaction between the material 160a and the material 160b) can be performed, for example, via the device 50 and/or via the device 180.

FIG. 8 illustrates that the system 48 (e.g., the system 48D and/or the system 48R) can have a transvaginal ultrasound transducer 40, a transvaginal needle 41, a delivery catheter 43, or any combination thereof. FIG. 8 illustrates delivering the occlusive device (e.g., the material 160a) or the removal agent (e.g., the material 160b) directly into the fallopian tube 10 with transvaginal ultrasound. FIG. 8 illustrates that the transvaginal ultrasound transducer 40 can be placed into the vagina 13 with the transvaginal needle 41. FIG. 8 illustrates that a distal tip 42 of the transvaginal needle 41 can penetrate through the uterus 14 and directly into the fallopian tube 10. FIG. 8 illustrates that the delivery catheter 43 can directly access the fallopian tube 10 by being advanced through the transvaginal needle 41. For example, FIG. 8 illustrates that the delivery catheter 43 can exit the distal tip 42 of the transvaginal needle 41 directly into the fallopian tube 10. The penetration of the transvaginal needle 41 and the location of the fallopian tube 10 can be visualized, for example, with the aid of ultrasonic imaging (ultrasound console and monitor not shown).

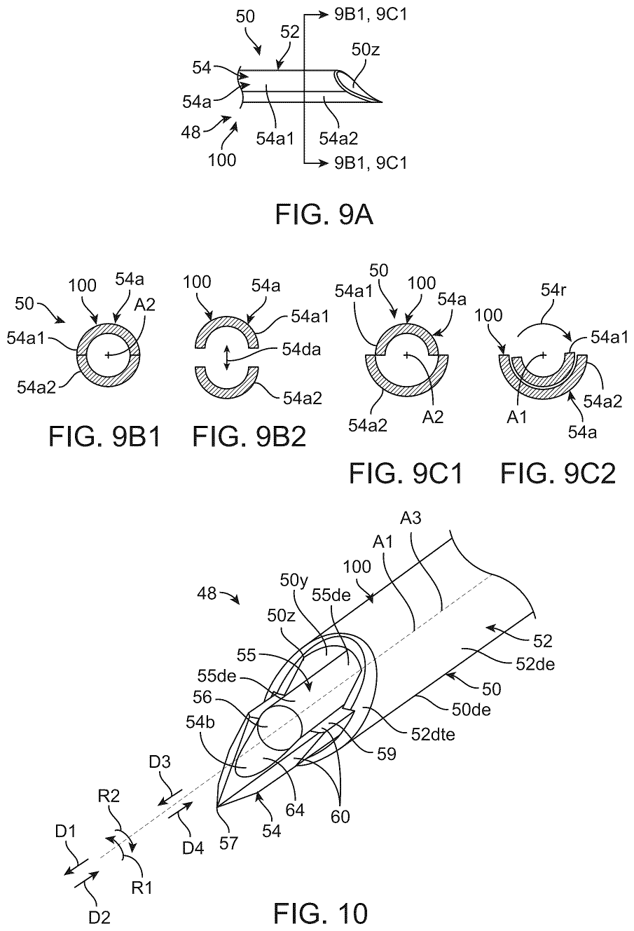

FIGS. 9A-9C2 illustrate that the needle 54 can be a split needle 54a. The split needle 54a can split radially and/or circumferentially. FIGS. 9A-9C2 illustrate that the split needle 54a can have a needle first portion 54al (also referred to as a split needle first portion 54a1) and a needle second portion 54a2 (also referred to as a split needle second portion 54a2). The needle first portion 54a1 can be, for example, a needle first half. The needle second portion 54a2 can be a needle second half. For example, FIGS. 9A-9C2 illustrate that the needle first portion 54a1 can be a first hemispherical section and that the needle section portion 54a2 can be a second hemispherical section. The needle 54 can be constructed, for example, from thin-walled tubing. The needle 54 can be made, for example, with stainless steel, nitinol, titanium, other biocompatible materials, or any combination thereof. The needle 54 can be, for example, a hollow, cylindrical needle tubing with a continuous circumferential wall. As another example, the needle 54 can be the split needle 54a with a breaks, or discontinuities, in the circumferential wall so that the needle portions (e.g., the needle first portion 54al and the needle second portion 52a2) can separate from and/or be moved relative to each other. The split needle 54a can be configured to penetrate the wall of the body space 8. FIGS. 9B1-9C2 illustrate that after the split needle 54a penetrates through the wall and gains access to the body space 8, the split needle 54a can separate into two halves from a non-split configuration (e.g., as shown in FIGS. 9A, 9B1, 9C1) to a split configuration (e.g., as shown FIGS. 9B2, 9C2). The split needle 54a can be considered to have gained access to the body space 8 when, for example, the opening 50z is in the body space 8. The split needle 54a can split radially and/or rotationally. For example, FIGS. 9B1-9B2 illustrate that the split needle 54a can separate radially into the needle first portion 54al and the needle second portion 54a2, for example, away from the center longitudinal axis A2 of the split needle 54a (e.g., of the conduit 52) in opposing radial directions as indicated, for example, by the double arrow 54da in FIG. 9B2. FIGS. 9B1-9B2 illustrate, for example, that the needle first portion 54a1 and the needle second portion 54a2 can be split apart in a radial direction from a collinear central axis (e.g., the axis A2). FIGS. 9B1-9B2 illustrate, for example, that the needle first portion 54al can be the anterior half of the split needle 54a and that the needle second portion 54a2 can be the posterior half of the split needle 54a. After the split needle 54a is radially separated (e.g., as shown in FIG. 9B2), the split needle 54a can be removed from the system 48 (e.g., from the device 50) without requiring an exchange or back loading from other components that occupy the central axis of the system 48. As another example, FIGS. 9C1-9C2 illustrate that the split needle 54a can be separated by rotating the needle first portion 54al clockwise or counterclockwise relative to and/or onto the needle second portion 54a2, for example, about the center longitudinal axis A2 of the split needle 54a (e.g., of the conduit 52) as indicated, for example, by the rotation arrow 54r in FIG. 9C2 (e.g., which indicates a clockwise rotation). As another example, FIGS. 9C1-9C2 illustrate, for example, that the needle first portion 54a1 and/or the needle second portion 54a2 can be rotated along their central axis to form an opening in the split needle 54a. After the body space 8 is penetrated by the split needle 54a, rotation of the split needle 54a can eliminate the continuity of the cylindrical wall of the split needle 54a (e.g., of the conduit 52), which can thereby allow other elements of the delivery or removal system to be introduced without a back exchange. The needle first portion 54a1 and the needle second portion 54a2 can have the same size as each other or can have different sizes. The needle first portion 54al and the needle second portion 54a2 can have needle first configuration during insertion (e.g., a closed configuration), and can have a needle second configuration after insertion (e.g., an open configuration with one or multiple openings or gaps). The split needle 54a can have more than two needle sections.

FIG. 10 illustrates that the system 48 (e.g., the device 50 and/or the device 180) can have a probe 55. FIG. 10 illustrates a variation of the device distal end 50de. FIG. 10 illustrates that the needle 54 can be attached to and/or extend from the device distal end 50de. For example, FIG. 10 illustrates that the needle 54 can be attached to and/or extend from the conduit distal end 52de. FIG. 10 illustrates, for example, that the needle 54 can be attached to and/or extend from the conduit distal terminal end 52te. As another example, the needle 54 can be integrated with the conduit 52, for example, as shown in FIGS. 3 and 6. FIG. 10 illustrates that the opening 50z can be a port for the probe 55 as defined by the conduit 52 and/or the needle 54.

FIG. 10 illustrates that the needle 54 can be a half needle 54b. For example, FIG. 10 illustrates that the half needle 54b can have a needle channel 64. When the probe 55 is proximal the distal terminal end of the half needle 54b (e.g., as shown in FIG. 10), the half needle 54b can be the device distal terminal end 50dte. The probe 55 can be, for example, a rod, a wire, a tube, or any combination thereof. For example, FIG. 10 illustrates that the probe 55 can be a wire (e.g., a guidewire). FIG. 10 illustrates that the probe 55 can have a probe distal end 55de having a tip 56 (also referred to as the probe tip 56). The probe tip 56 can be, for example, the distal terminal end of the probe 55. The probe tip 56 can be blunt and/or sharp. For example, FIG. 10 illustrates that the probe tip 56 can be blunt (e.g., rounded). The probe 55 can comprise (e.g., be made from) nitinol, stainless steel, other biocompatible materials or polymers, or any combination thereof.

FIG. 10 illustrates that the device 50 can house the probe 55. For example, FIG. 10 illustrates that the probe 55 can extend through the conduit 52 and/or the needle 54. The probe 55 can be translatable and/or rotatable relative to the device 50, for example, relative to the handle 51, the body 50b, the conduit 52, the half needle 54b, or any combination thereof. The probe 55 can be translatable and/or rotatable in the handle 51 (e.g., in a lumen that extends through the handle 51 such as the lumen 50y), in the body 50b (e.g., in a lumen that extends through the body 50b such as the lumen 50y), in the conduit 52 (e.g., in a lumen that extends through the conduit 52 such as the lumen 50y), in the needle 54 (e.g., in a lumen that extends through the needle 54 such as the lumen 50y), in the half needle 54b (e.g., in the needle channel 64), or any combination thereof. The probe 55 can be translatable through the opening 50z, can be rotatable in the opening 50z, or both.

FIG. 10 illustrates, for example, that the probe 55 can be translated (e.g., advanced) in direction D3, translated (e.g., retracted) in direction D4, rotated in direction R1, rotated in direction R2, or any combination thereof. The direction D3 and the direction D4 can be opposite to each other. The direction D3 and the direction D4 can be a first translational direction and a second translational direction, respectively, that the probe 55 can be translated in, or vice versa. The direction D3 and the direction D4 can be parallel, for example, to the conduit center longitudinal axis A1. FIG. 10 illustrates that the direction D3 can be the same as (e.g., parallel to or collinear with) the direction D1. As another example, the direction D3 can be a different direction than the direction D1. FIG. 10 illustrates that the direction D4 can be the same as (e.g., parallel to or collinear with) the direction D2. As another example, the direction D4 can be a different direction than the direction D2. The direction R1 and the direction R2 can be opposite to each other. The direction R1 and the direction R2 can be a first rotational direction and a second rotational direction, respectively, that the probe 55 can be rotated in, or vice versa. For example, FIG. 10 illustrates that the direction R1 can be clockwise rotation of the probe 55 and that the direction R2 can be counterclockwise rotation of the probe 55 about the center longitudinal axis A3 of the probe (also referred to as the probe center longitudinal axis A3). FIG. 10 illustrates that the conduit center longitudinal axis A1 and the probe center longitudinal axis A3 can be parallel to each other and/or collinear with each other. As another example, the conduit center longitudinal axis A1 and the probe center longitudinal axis A3 may not be parallel with each other and/or may be offset from each other. FIG. 10 illustrates that the probe 55 can be translated and/or rotated within the conduit 52 and/or the needle 54, for example, within the needle channel 64.

FIG. 10 illustrates that the half needle 54b can have one or multiple cut-away portions 60 at the mid-section 59 (also referred to as the equator 59) of the half needle 54b to form the half needle 54b into a needle half-cylinder having the needle channel 64. The needle tip 57 can provide a minimal penetration implement in which the probe 55 can be housed in. For example, from the position shown in FIG. 10, the probe 55 can be advanced into the 8 body space once the penetration into the body space by the half needle 54b is accomplished, for example, by advancing the half needle 54b into the body space 8 by advancing the device 50 (e.g., the conduit 52) in direction D1. For example, FIG. 10 can illustrate the probe 55 in a retracted position. The retracted position of the probe 55 in FIG. 10 can be, for example, a partially retracted position or a fully retracted position. The probe 55 can be advanced and/or retracted, for example, to confirm whether or not the device has access to the body space 8. FIG. 10 illustrates, for example, a half-needle insertion system that can house the probe 55 within the half needle section with the cut-away portion 60 at the mid-section 59.

As another example, the half needle 54b can be attached to or integrated with the probe 55. In such a case, FIG. 10 illustrates that the half needle 54b can extend from the distal end 55de of the probe 55de (also referred to as the probe distal end 55de) such that the probe 55 and the half needle 54b can be translated (e.g., in direction D3 and/or D4) and/or rotated (e.g., in direction R1 and/or R2) in unison. In such a variation, the probe 55 and/or the half needle 54b can translate and/or rotate inside the delivery device 50 (e.g., in the lumen 50y) through distal end opening of the device 50 (e.g., through the opening 50z). FIG. 10 illustrates, for example, that the half needle 54b can be attached to the probe 55 such that when the probe 55 is advanced and retracted, the half needle 54b can be advanced and retracted by advancing and retracting the probe 55, respectively. The probe 55 can be advanced, for example, to translate the half needle 54b through tissue to access the body space 8. For example, the needle tip 57 can provide a minimal penetration implement from and/or through which the probe 55 can be advanced and/or retracted. The probe 55 and the probe tip 56 can be, for example, advanced into the body space 8 through the needle 54 (e.g., through the needle channel 54c) once the needle tip 57 penetrates the body space (e.g., once penetration into the lumen is accomplished by the needle tip 57). The device 50 can be configured such that the probe 55 and the half needle 54b can translate inside the device 50 (e.g., inside the lumen 50y) through a distal opening of the device 50 (e.g., through the opening 50z).

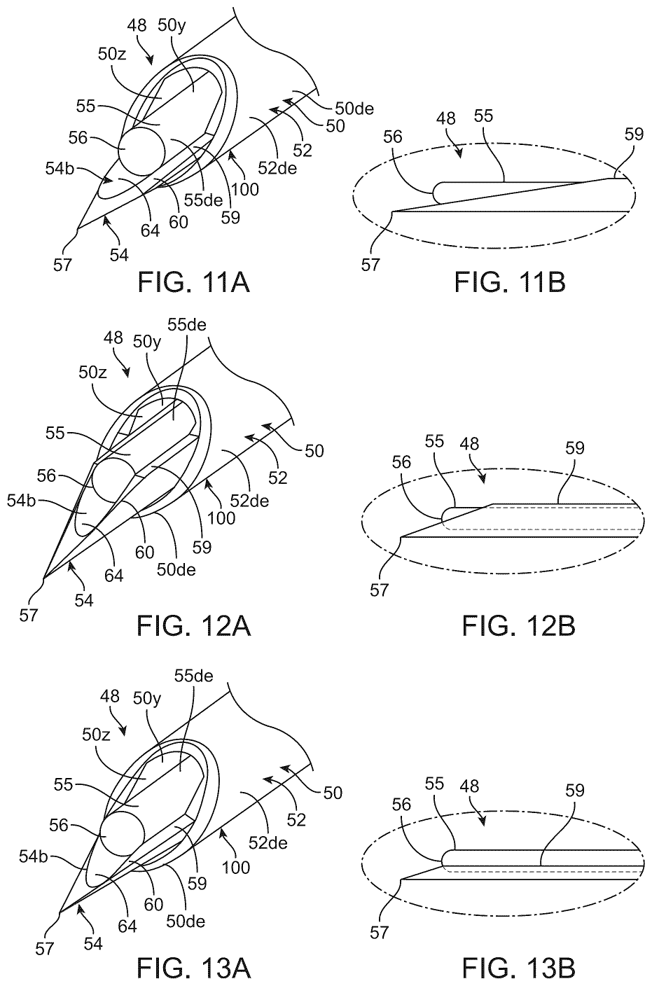

FIG. 11A illustrates that the half needle 54b can have a larger cut-away portion 60 such that the needle tip 57 can be more pointed and taper up at the mid-section 59, for example, as compared to the half needle 54b in FIG. 10. FIG. 11A illustrates, for example, that the half needle 54b can have a cut-away portion 60 that extends below the equator 59 of the half needle 54b and/or the conduit 52 so that a smaller portion of the probe 55 can be in the half needle 54b (e.g., in the needle channel 64), for example, as compared to the half needle 54b in FIG. 10. FIG. 11A illustrates that the needle channel 64 can be less than a half cylinder, for example, a one-fourth cylinder or a one-fifth cylinder. FIG. 11A illustrates that the half needle 54b can have a smaller wall (e.g., a smaller needle channel 64) for housing the probe 55, for example, as compared to the half needle 54b in FIG. 10. FIG. 11B illustrates a side view of the features shown in FIG. 11A, whereby FIG. 11B illustrates that the half needle 54b can have a sharper tip in relation to the probe 55, for example, as compared to the half needle 54b in FIG. 10. FIGS. 11A and 11B illustrate, for example, that the half needle 54b can be more pointed at the distal end of the half needle 54b, for example, as compared to the half needle 54b in FIG. 10. FIGS. 11A and 11B illustrate that the half needle 54b can be a half needle with the arrangement of features shown, whereby FIGS. 11A and 11B illustrate that the half needle 54b can be a half needle with a cut away portion 60 that extends below the equator 59.

FIG. 12A illustrates that the cut-away portion 60 can be located above the mid-section 59 of the half needle 54b and/or the conduit 52 so that a greater portion of the probe 55 can be in the half needle 54b (e.g., in the needle channel 64), for example, as compared to the half needle 54b in FIG. 10. FIG. 12A illustrates that the half needle 54b can have a larger wall (e.g., a larger needle channel 64) for housing the probe 55, for example, as compared to the half needle 54b in FIG. 10. FIG. 12B illustrates a side view of the arrangement of features shown in FIG. 12A, whereby FIG. 12B illustrates that the half needle 54b can be a half needle with a cut away portion 60 above the equator 59.

FIG. 13A illustrates that the cut-away portion 60 can be located below the mid-section 59 of the half needle 54b and/or the conduit 52. FIG. 13B illustrates a side view of the arrangement of features shown in FIG. 13A, whereby FIG. 13B illustrates that the half needle 54b can be a half needle with a cut away portion 60 below the equator 59.

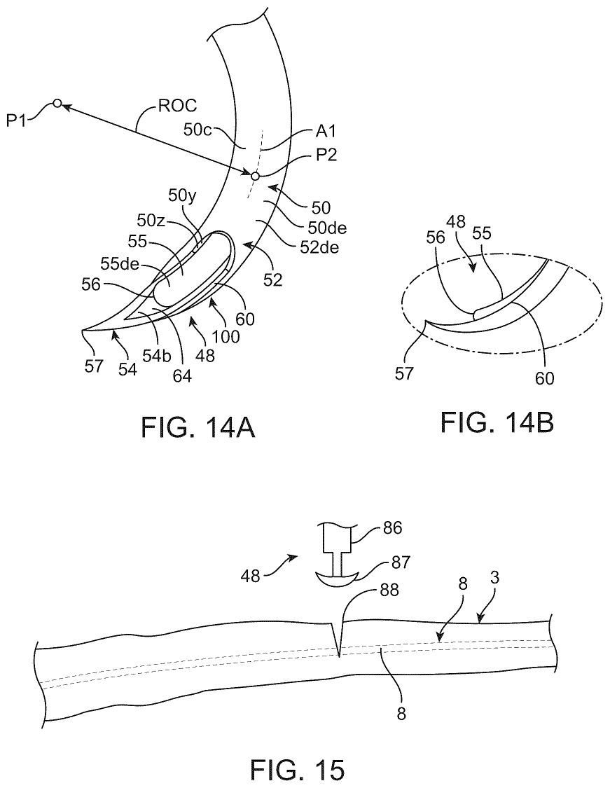

FIG. 14A illustrates the device distal end 50de can have a curve 50c having a radius of curvature ROC as measured, for example, between point P1 and point P2 in FIG. 14A. Point P1 can be a center of the curvature, and point P2 can be a point along the curve 50c, for example, a point along the conduit center longitudinal axis A1. Figure For example, FIG. 14A illustrates that the conduit distal end 52de) and/or the half needle 54b can have the curve 50c. FIG. 14A illustrates, for example, that the half needle 54b can be a curved half needle. The curvature of the device distal end 50de can be configured for insertion of the probe 55 insertion into the body space 8. The curvature of the device distal end 50de (e.g., of the conduit distal end 60sde and/or of the half needle 54b) can force the probe 55 to ride along the inside arc or curve of the device distal end 50de (e.g., of the conduit distal end 60sde and/or of the half needle 54b). For example, FIG. 14A illustrates that the curvature of the device distal end 50de can force the probe 55 to ride along the inside of the arc or curve of the needle channel 64 shown in FIG. 14A. The curvature of the device distal end 50de (e.g., of the conduit distal end 60sde and/or of the half needle 54b) can allow for an oblique entry into the body space 8 from the device 50. FIG. 14B illustrates a side view of the arrangement of features shown in FIG. 14A, whereby FIG. 14B illustrates, for example, the curved needle cut away portion 60 and the needle tip 57 in relation to the probe 55 and the probe tip 56.

FIG. 15 illustrates that the system 48 (e.g., the system 48D and/or the system 48R) can have a cutting tool 87. FIG. 15 illustrates that the cutting tool 87 can be used to gain access to the body space 8 (e.g., to the vas deferens lumen 8v). FIG. 15 illustrates that the cutting tool 87 can have a variable guillotine depth 86 for transection 88 of the vas deferens 3 through which the probe 55 can be inserted. The transection can be hemi-spherical. For variations in which the cutting tool 87 is used, the device distal end 50de may or may not have a needle. For example, the conduit distal end 52de may not be a needle and/or the conduit distal end 52de may not have the half needle 54b. The hemi-spherical transection 88 can provide direct access to the body space 8 (e.g., the vas deferens lumen 8v).

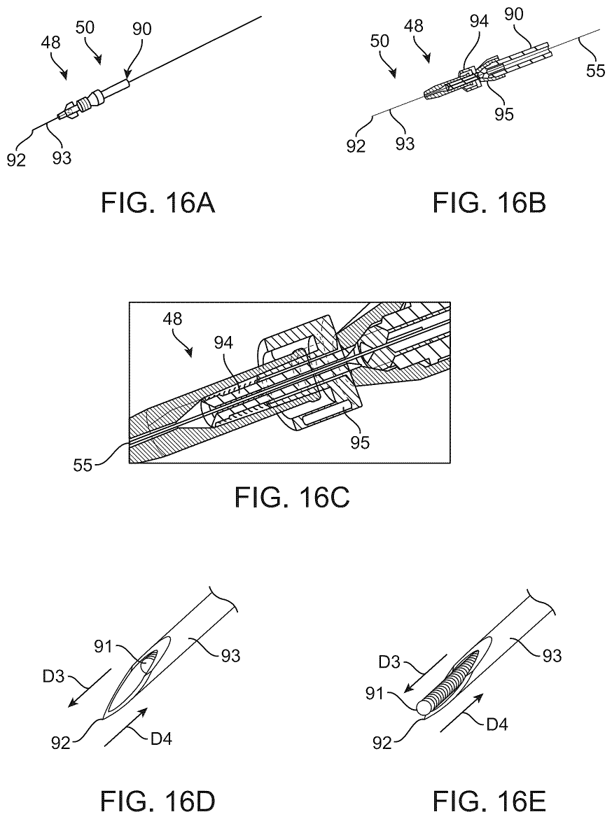

FIGS. 16A-16E illustrate that the system 48 (e.g., the device 50 and/or the device 180) can have a spring-loaded lancet system 90. FIGS. 16A-16E illustrate that the spring-loaded lancet system 90 can be used, for example, to confirm placement of the system 48 in the body space 8 and/or to confirm that the system 48 has access to the body space 8. The spring-loaded lancet system 90 can have a probe 91, a needle 93, a spring 94, a housing 95, or any combination thereof. The needle 93 can have a needle tip 92. The needle tip 92 can be a sharp distal tip of the needle 93 so that the needle 93 can penetrate tissue to access the target site 173 (e.g., the body space 8). The needle tip 92 can be used to penetrate tissue and access the body space 8. The probe 91 can be spring loaded with the spring 94. The probe 91 can advance automatically via the spring 94 once the needle tip 92 reaches a point of less resistance. The point of less resistance can be defined as when the needle tip 92 and/or the opening 50z of the needle tip 92 enters the body space 8 such that once entry of the needle tip 92 and/or the opening 50z of the needle tip 92 into the body space 8 is achieved, the probe 91 can advance automatically into the body space 8 via the spring 94. The automatic advance of the probe 8 can indicate to the user that the spring-loaded lancet system 90 is in the body space 8 and/or indicate to the user that the spring-loaded lancet system 90 has gained access to the body space 8. FIGS. 16A-16E illustrate that the spring-loaded mechanism can include the spring 94. FIGS. 16A-16E illustrate that the spring 94 can be in the housing 95, whereby the spring 94 can be under compression when the probe 91 is in a retracted position within the needle 93 (e.g., as shown in FIG. 16A-16D). FIGS. 16A-16D illustrate a variation of a retracted position of the probe 91, for example, before the system 48 (e.g., the spring-loaded lancet system 90) has reached the point of less resistance. FIG. 16E illustrates a variation of an advanced position of the probe 91, for example, after the system 48 (e.g., the spring-loaded lancet system 90) has reached the point of less resistance and the probe 91 has advanced automatically (e.g., to the position shown) via the spring 94. The spring-loaded lancet system 90 can be a different device than the device 50. For example, the spring-loaded lancet system 90 and the device 50 can be used separately. For example, once the spring-loaded lancet system 90 indicates to the user that the spring-loaded lancet system 90 is in the body space 8 and/or that the spring-loaded lancet system 90 has gained access to the body space 8, the user can withdraw the spring-loaded lance 90 from the body space 8 and the device 50 (e.g., with a blunt or a sharp tip at the conduit distal end 52de) can be inserted into the body space 8 in its place. The spring-loaded lancet system 90 can be attached to the device 50 or vice versa. As another example, the device 50 can be and/or can comprise the spring loaded lancet system 90. In such variations, the device 50 can have any of the features of the spring-loaded lance 90 or vice versa. For example, in such variations (e.g., in which the device 50 is and/or comprises the spring loaded lancet system 90), the probe 91 can be, for example, the probe 55 or vice versa, the probe 516 or vice versa, the probe 620 or vice versa, the device 50 can have both the probe 55 and the probe 91, the needle 93 can be, for example, the conduit 52 and/or the needle 54 or vice versa, the device 50 can have both the needle 54 and the needle 93, or any combination thereof. The probe 516 and/or the probe 620 can be advanceable, for example, via a spring, including, for example, the spring of the spring-loaded lancet system 90.

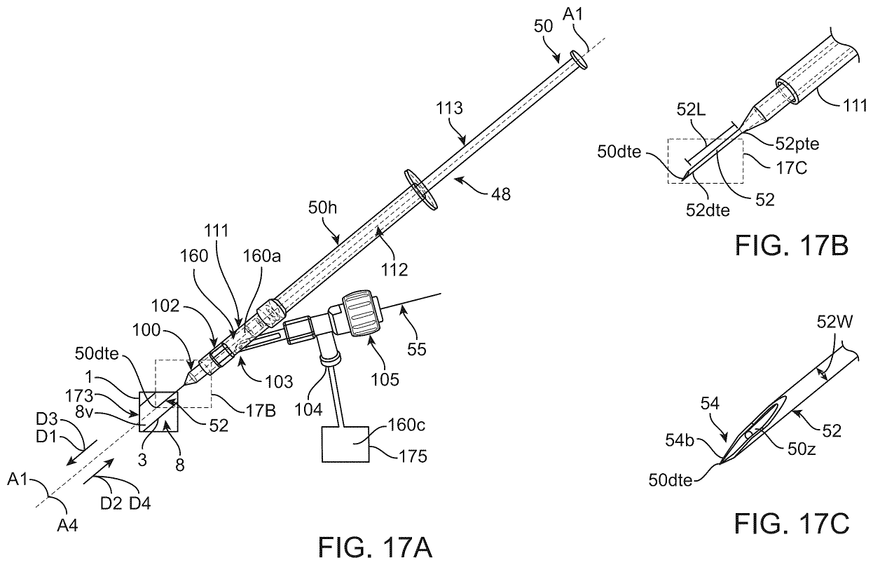

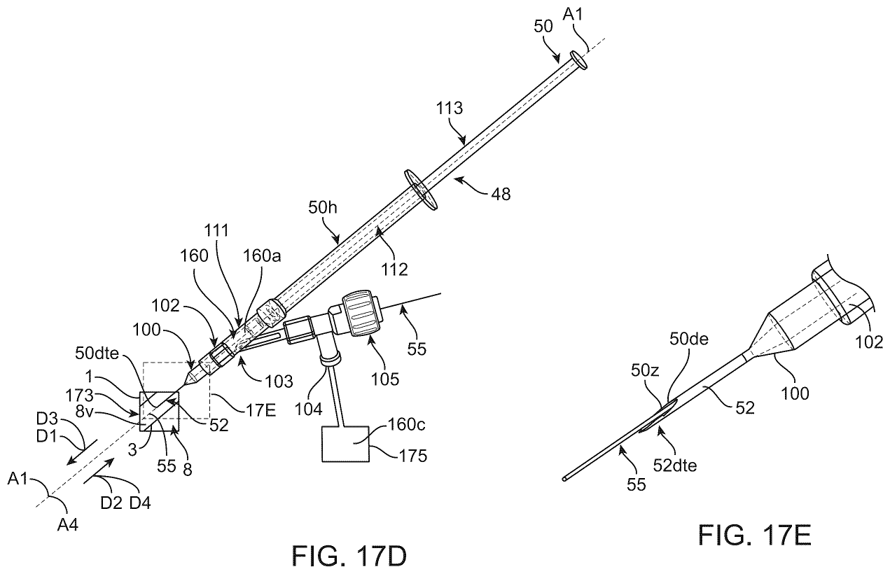

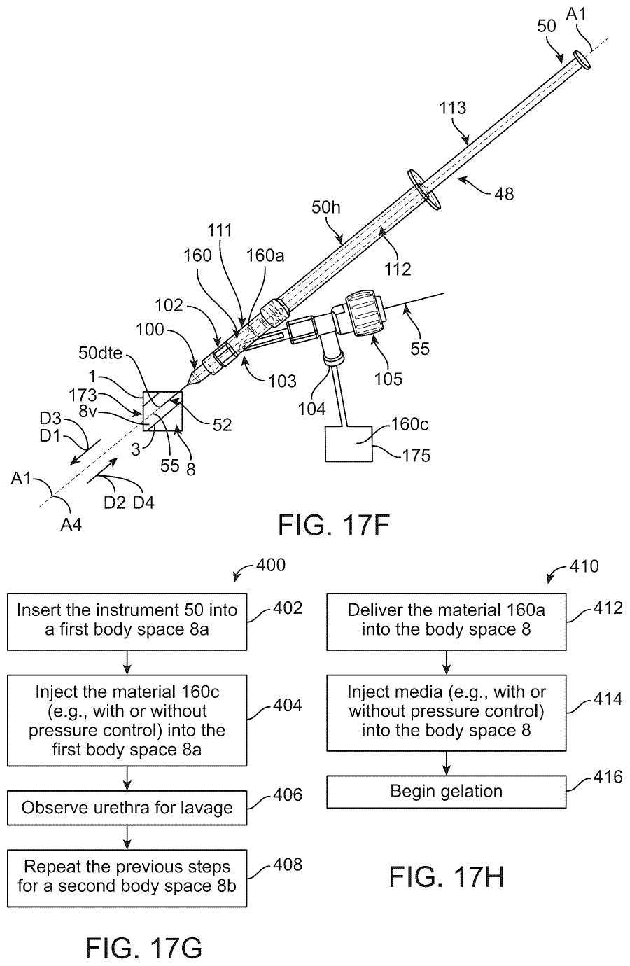

FIGS. 17A-17E illustrate that the system 48 (e.g., the system 48D and/or the system 48R) can have the arrangement of features shown. For example, FIGS. 17A-17E illustrate that a device (e.g., the device 50) can have the arrangement of features shown. FIGS. 17A-17E can illustrate, for example, a variation of the device 50. For example, the device 50 can be and/or can comprise the device shown in FIGS. 17A-17E. FIGS. 17A-17F illustrate, for example, that the system 48 (e.g., the device 50) can deliver and/or remove the materials 160 into and/or from the body space 8, respectively. The materials 160 can include, for example, the material 160a, the material 160b, the product of the interaction between the material 160a and the material 160b, one or more other materials (e.g., the material 160c). For example, FIGS. 17A-17F illustrate that the material 160a can be delivered to the body space 8. As another example, FIGS. 17A-17F illustrate that the material 160c can be injected into the body space 8 to clean the body space 8 and/or for hydro-distension of the body space 8 (e.g., for hydro-distension of the vas deferens 3 or the fallopian tube 10). The material 160a can be delivered into the body space 8 before or after the material 160c is delivered to the body space 8. For example, FIGS. 17A-17F illustrate that the material 160a can be delivered into the body space 8 after the material 160c has been injected into the body space 8.

FIG. 17A illustrates that the device 50 can have the features shown, including, for example, in the arrangement shown. For example, FIG. 17A illustrates that the device 50 can have the conduit 52, the probe 55, the conduit assembly 100, a needle control 102, a fitting 103, a port 104, a connector 105, the handle 51, a cartridge 111, a rod 112, a control 113, or any combination thereof. The conduit assembly 100 can have the conduit 52 and/or a needle (e.g., the needle 54). The needle control 102 can be, for example, a needle control slide. The fitting 103 can be, for example, a Y-fitting. The port 104 can be, for example, a fluid delivery port. The connector 105 can be, for example, a guidewire Touhy connector. The cartridge 111 can comprise, for example, one or multiple materials 160, including, for example, the material 160a, the material 160b, the material 160c, or any combination thereof. For example, FIG. 17A illustrates that the cartridge 111 can comprise the material 160a. The cartridge 111 can have the material 160a, for example, in a compartment or a reservoir in the cartridge 111. The cartridge 111 can be, for example, inside the conduit assembly 100 and/or the fitting 103. The cartridge 111 can, for example, be inside the sheath and the fitting 103. The rod 112 can be, for example, a push rod. The control 113 can be, for example, a plunger. FIG. 17A illustrates that the handle 51 can comprise, for example, any of the features proximal the conduit 52, including, for example, the rod 112 and the control 113. Moving (e.g., pushing) the control 113 in direction D1 can force the material 160a out of the cartridge 111 so that the material 160a is discharged from the device distal end 50de into the body space 8. For example, moving the control 113 in direction D1 can force (e.g., push) the material 160a out of the cartridge, through the lumen 50y of the conduit 52, out of the opening 50z of the conduit 52, and into the body space 8. FIG. 17A illustrates that the probe 55 may not have a needle (e.g., the needle 54). FIG. 17A illustrates that conduit center longitudinal axis A1 and the body space center longitudinal axis A4 can be parallel or collinear with each other (e.g., such that the alignment angle A5 is 0 degrees).

FIG. 17A illustrates that the device 50 can deliver the material 160c to the target site (e.g., the body space 8), for example, to lavage the body space 8, for example, from a source 175 in fluid communication with the port 104. The source 175 can have the material 160c. The source 175 can be the source of the material 160c. The source 175 can be, for example, a syringe having the material 160c. FIG. 17A illustrates that the source 175 can be, for example, removably attached to the port 104. FIG. 17A illustrates that the material 160c can be delivered to the body space 8 from the source 175, for example, by pressing the plunger of the syringe holding the material 160c (e.g., when the source 175 comprises a syringe). FIG. 17A illustrates that the material 160c can be injected into the body space 8 (e.g., the vas deferens lumen 8v) before and after insertion of the probe 55 into the body space 8, for example, to lavage the body space 8. The material 160c can be injected into the body space 8, for example, to clear the body space 8 of material and prepare for the delivery of the material 160a into the body space 8.

The material 160c can be, for example, a fluid comprising a liquid and/or a gas. The material 160c can be, for example, saline, phosphate buffered saline, Ringer's lactate, other biocompatible liquids, CO2, other gases, or any combination thereof. The material 160c can be used to lavage the body space 8 to clear the body space 8 of material and bodily fluids, to stabilize the pH of the body space 8, to control intraluminal temperature of the body space 8, to prepare the body space 8 for insertion of the material 160a, or any combination thereof. For example, for the vas deferens 3, the material 160c can wash the vas deferens lumen 8v of remnant sperm and fluids to establish a uniform environment so the material 160a can form an occlusion in the vas deferens lumen 8v when the material 160a is delivered to the vas deferens lumen 8v.

FIG. 17B illustrates that the conduit 52 can be a sheath. FIG. 17B illustrates that the conduit 52 can have a conduit length 52L. The conduit length 52L can be, for example, measured from the conduit proximal terminal end 52pte to the conduit distal terminal end 52dte, for example, along the conduit center longitudinal axis A1. The conduit length 52L can be, for example, 1.5 cm-30.0 cm, or more narrowly, 1.5 cm-10.0 cm, or more narrowly, 1.0 cm-5.0 cm, including every 0.1 cm increment within these ranges (e.g., 1.0 cm, 1.5 cm, 3.0 cm, 5.0 cm, 10.0 cm, 30.0 cm).

FIG. 17C illustrates that the conduit 52 can have a smooth bevel sheath distal tip.

FIG. 17C illustrates that the device 50 can have the needle 54. For example, FIG. 17C illustrates that the needle 54 can be the half needle 54b. FIG. 17C illustrates that the conduit assembly can have the needle 54. FIG. 17C illustrates, for example, that the needle 54 can be in the lumen 50y of the conduit 52.

FIG. 17C illustrates that the needle 54 can be an advanceable and/or a retractable, for example, relative to the conduit 52 and/or to the handle 51. For example, FIG. 17C illustrates that the needle 54 can be advanced and/or retracted via the needle control 102 in direction D1 and/or in direction D2, respectively. FIG. 17C illustrates that the needle 54 can be retractable into the conduit assembly 100 via the needle control 102. For example, FIG. 17C illustrates that the needle 54 can be retractable into the conduit 52, for example, in the lumen 50y of the conduit 52 along the conduit center longitudinal axis A2 via the needle control 102. For example, FIGS. 17A-17C illustrate the needle 54 in a pre-retracted position (also referred to as an advanced position, a non-retracted configuration), and FIGS. 17D-17E illustrate the needle 54 in a retracted positioned. FIGS. 17A-17E illustrate that the needle 54 can be advanced and/or retracted via the needle control 102, for example, by pressing, sliding, and/or twisting the needle control 102. For example, the needle control 102 can be a control that can be moved (e.g., slid) from a needle control first position (e.g., as shown in FIG. 17A) to a needle control second position (e.g., as shown in FIG. 17D) in direction D2. When the needle control 102 is in the needle control first position, the needle 54 can be in a fully advanced position (e.g., in the advanced position shown in FIGS. 17A-17C). When the needle control 102 is in the needle control second position, the needle 54 can be in a fully retracted position (e.g., in the retracted position shown in FIGS. 17D-17E). The needle 54 can be advanced from the retracted position to the advanced position by moving (e.g., sliding) the needle control 102 from the needle control second position to the needle control first position. As another example, the needle 54 may be retractable but not advanceable.

FIG. 17C illustrates that the conduit 52 can have a conduit width 52W. The conduit width 52W can be, for example, an outer diameter of the conduit 52. The conduit width 52W can be, for example, 0.5 mm to 2.5 mm, including every 0.1 mm increment within this range (e.g., 0.5 mm, 1.0 mm, 2.5 mm). For example, FIG. 17C illustrates that the conduit width 52W can be 1.0 mm.

FIGS. 17A-17C illustrate the probe 55 in a retracted position.

FIG. 17D illustrates the probe 55 in an advanced position (e.g., in a fully advanced position). FIG. 17D illustrates that the probe 55 can be moved (e.g., translated) from the retracted position to the advanced position, for example, to confirm that the device distal end 50de has access to the body space 8. Once the probe 55 is advanced, for example, to the advanced position shown in FIG. 17D, the advanced position of the probe 55 can be an indication to the user that the device 50 has access to the body space 8. The device 50 can have access to the body space 8, for example, as described above, including, for example, if the device distal end 50de (e.g., the opening 50z, the conduit distal end 52de, the needle 54) is in the body space 8. The probe 55 can be advanced (e.g., translated), for example, to the position shown in FIG. 17D if the device distal end 50de has access to the body space 8. If the device distal end 50de does not have access to the body space 8, the probe 55 may not be advanceable, in which case the inability to advance the probe 55 can indicate to the user that the device distal end 50de does not have access to the body space 8. Additionally or alternatively, access to the body space 8 can be confirmed by a force, a flow rate, or a volume measurement of the material 160c (the force, the flow rate, and the volume measuring device is not shown in FIG. 17D). In practice, the flow of the material 160c in the body space 8 can require less force, have a higher flow rate, or allow for a greater volume if the device distal end 50de (e.g., the opening 50z, the conduit distal end 52de, the needle 54) is located within the body space 8.

FIG. 17E illustrates a closeup of the needle 54 in a retracted position in the lumen 50y of the conduit 52, the probe 55 in an advanced position, and details of the beveled tip of the conduit 52. FIG. 17E illustrates that the conduit 52 can be flexible and that the needle 54 can be rigid. FIG. 17E illustrates that the conduit distal end can have a bevel tip.

FIGS. 17A-17E illustrate that the material 160a can be injected into the body space 8 by pushing the control 113 (e.g., the plunger) in the direction D1, which can be done when the needle 54 in an advanced position (e.g., as shown in FIG. 17A) or when the needle 54 is in a retracted position (e.g., as shown in FIGS. 17D-17E). FIGS. 17A-17C illustrate that when the needle 54 is in the advanced position, the device distal terminal end 50dte can be the needle tip 57. FIGS. 17D-17E illustrate that when the needle 54 is in the retracted position, the device distal terminal end 50dte can be the tip of the conduit 52 (e.g., the tip of the sheath) and/or the probe 55. The needle 54 can be withdrawn within distal sheath 100 after insertion into the body space 8. The material 160a and/or the material 160c can be delivered to the body space 8 when the needle 54 is in the advanced position or when the needle is in the retracted position.

FIGS. 17A-17E illustrate that the probe 55 can be inserted through the lumen (e.g., a lumen 50y) in fluid communication with the port 104, for example, through the connector 105.

FIG. 17F illustrates that injection of the material 160c before and after insertion of the probe 55 into the body space 8 can be performed in combination with ultrasound or radiographic imaging (imaging equipment not shown) for confirmation of intraluminal placement in the body space 8.

FIG. 17G illustrates a variation of a method 400 that can be performable using the system 48 (e.g., the device 50 and/or the device 180). FIG. 17G illustrates that the method 400 can involve, for example, step 402, step 404, step 406, step 408, or any combination thereof. The steps of method 400 can be executed in any order, for example, in the order shown in FIG. 17G. Any of the steps can be omitted. For example, step 408 can be omitted. FIG. 17G illustrates that the device 50 can be inserted into multiple body spaces 8, including, for example, into a first body space 8a and/or into a second body space 8b. For example, the first body space 8a can be a first vas deferens 3 of a patient, and the second body space 8b can be a second vas deferens 3 of the patient. As another example, the first body space 8a can be a first fallopian tube 10 of a patient, and the second body space 8b can be a second fallopian tube 10 of the patient. FIG. 17G illustrates that step 402 can include inserting the device 50 into the first body space 8a, that step 404 can including injecting the material 160c (e.g., with or without pressure control) into the first body space 8a, that step 406 can include observing the urethra 5, and that step 408 can involve repeating steps 402, 404, and 406 for the second body space 8b. FIG. 17G illustrates that the device 50 can be used for the injection of media (e.g., the material 160c) for hydro-distension before and after probe 55 insertion by confirming lavage through the vas deferens 3 or fallopian tube 10 of a reproductive tract. For the vas deferens 3, the device distal end 50de can be delivered into the vas deferens lumen 8v, followed by the injection of the media (e.g., the material 160c). The fluid pressure, flow rate, and fluid volume of the media (e.g., the material 160c) can be monitored or governed by pressure control, flow rate control, and fluid volume measurement devices. While the media (e.g., the material 160c) slightly distends the potential space of the vas deferens lumen 8v during injection, the excess lavage can exit the reproductive tract through the urethra 5 to confirm patency. These steps can be repeated for the contralateral side (e.g., the other vas deferens 3 or the other fallopian tube 10). The method 400 illustrates, for example, that lavage through the urethra 5 can be confirmed.

FIG. 17H illustrates a variation of a method 410 that can be performable using the system 48 (e.g., the device 50 and/or the device 180). FIG. 17H illustrates that the method can involve, for example, step 412, step 414, step 416, or any combination thereof. The steps of method 410 can be executed in any order, for example, in the order shown in FIG. 17H. Any of the steps can be omitted. FIG. 17H illustrates, for example, that the delivery of the material 160a and/or of a media. FIG. 17H illustrates, for example, the injection of a media for assisting in the gelation of the hydrogel. The media can be used as a primer for assisting in the gelation of the material 160a (e.g., a hydrogel) in the body space 8. The media can be a fluid comprising a liquid and/or a gas. The media can wash the body space 8 of excess materials, provide greater lubricity, deliver proteins for accelerate gelation of the hydrogel, provide for a uniform pH, or a pH beneficial to gelation. FIG. 17H illustrates that step 412 can include delivering (e.g., injecting) the material 160a into the body space 8, that step 414 can include injecting the media (e.g., with or without pressure control) into the first body space 8a, and that step 416 can include beginning gelation. The media can be, for example, the material 160c or a different material.

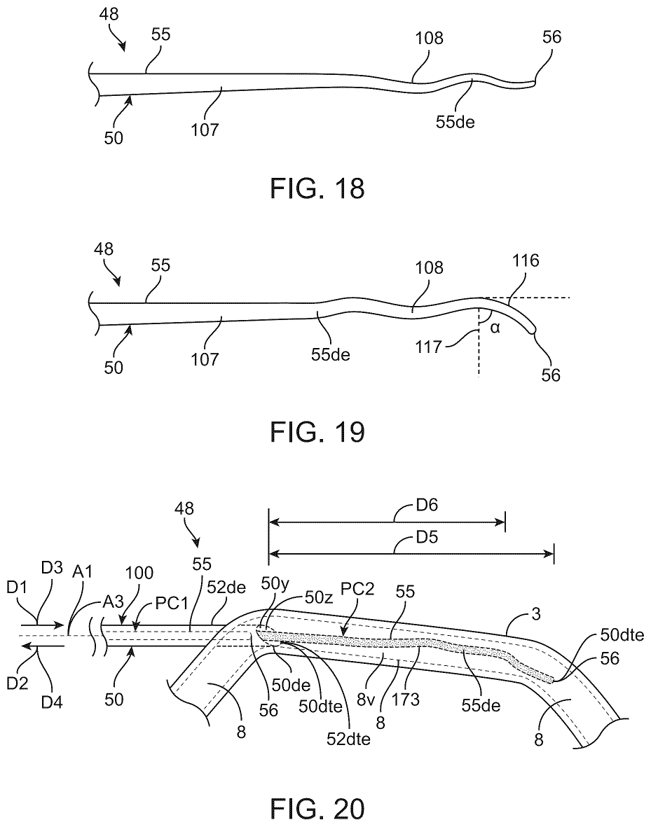

FIG. 18 illustrates that the probe distal end 55de can be flexible and/or have a flexible section. For example, FIG. 18 illustrates that the probe 55 can be configured for improved insertion, for example, by having probe first section 107 and a probe second section 108. The probe first section 107 can be more rigid than the probe second section 108, and the probe second section 108 can be more flexible than the probe first section 107. For example, the probe first section 107 can be rigid, and the probe second section 108 can be flexible. The probe proximal end 55pe and/or the probe distal end 55de can have the probe first section 107 and/or the probe second section 108. For example, FIG. 18 illustrates that the probe distal end 55de can have both the probe first section 107 and the probe second section 108. FIG. 18 illustrates that the probe second section 108 can be near the probe tip 56, with the probe 55 becoming gradually stiffer toward the probe first section 107 in the more proximal direction.

FIG. 19 illustrates that the probe 55 can have the probe first portion 117, the probe second portion 108, and a preformed J-angle 117 near the probe tip 56. FIG. 19 illustrates that the probe distal end 55 can have a curve 116. The curve 116 can, for example, facilitate the identification of the body space 8 and/or facilitate confirming access to the body space 8.

FIG. 20 illustrates that the probe 55 can be moved (e.g., translated) relative to the device 50, for example, relative to the conduit 52 and the body space 8. FIG. 20 illustrates that the probe 55 can be moved into and/or out of the conduit 52, for example, into and/or out of the lumen 50y, into and/or out of the opening 50z, or any combination thereof. For example, FIG. 20 illustrates that the probe 55 can be moved into and/or out of the conduit through the opening 50z, into and/or out of the body space 8 through the opening 50z, or any combination thereof. FIG. 20 illustrates that the probe 55 can be moved into and/or out of the body space 8. FIG. 20 illustrates, for example, that the probe 55 can be moved from a probe first configuration PC1 to a probe second configuration PC2 in direction D3, and/or that the probe 55 can be moved from the probe second configuration PC2 to the probe first configuration PC1 in direction D4. The probe 55 can be moved in directions D3 and D4 while the position of the device 50 is held constant (e.g., in the position shown in FIG. 20) and/or while the device 50 is moved (e.g., advanced and/or retracted in directions D1 and/or D2, respectively). In FIG. 20, the probe first configuration PC1 of the probe 55 is illustrated with dashed lines, and the probe second configuration PC2 of the probe 55 is illustrated with the solid lines. FIG. 20 illustrates that the conduit 52 can have the same position when the probe 55 is in the probe first configuration PC1 and when the probe 55 is in the probe second configuration PC2.

The probe first configuration PC1 can be, for example, any configuration of the probe 55 in which the probe tip 56 is closer to the handle 51 than when the probe 55 is in the probe second configuration PC2, any configuration in which the probe tip 56 is closer to the conduit distal end 52de than when the probe 55 is in the probe second configuration PC2, any configuration of the probe 55 in which the probe 55 has not yet been advanced, any configuration of the probe 55 in which the probe tip 56 is in the conduit 52, or any combination thereof. For example, the probe first configuration PC1 can be any configuration of the probe 55 in which the probe tip 56 is in the device 50 (e.g., is in the conduit 52), is flush with the conduit distal terminal end 52dte, is flush with the opening 50z, is proximal the conduit distal terminal end 52dte, is proximal the opening 50z, is distal the conduit distal terminal end 52dte, is distal the opening 50z, or any combination thereof. For example, FIG. illustrates that when the probe 55 is in the probe first configuration PC1, the probe tip 56 can be proximal the probe distal terminal end 52dte and the opening 50z.

The probe first configuration PC1 can be, for example, a probe neutral configuration, a probe retracted configuration, or a probe advanced configuration. The probe second configuration PC2 can be, for example, a probe advanced configuration, a more advanced configuration of the probe 55 than the probe first configuration PC1, a partially advanced configuration of the probe 55, a fully advanced configuration of the probe 55, or any combination thereof. For example, the probe first configuration PC1 can be a probe neutral configuration and the probe second configuration PC2 can be a probe advanced configuration. As another example, For example, the probe first configuration PC1 can be a probe retracted configuration and the probe second configuration PC2 can be a probe advanced configuration. As another example, the probe first configuration PC1 can be a probe first advanced configuration and the probe second configuration PC2 can be a probe second advanced configuration, whereby the probe first advanced configuration can be a first partially advanced configuration of the probe 55 and the probe second advanced configuration can be a second partially advanced configuration of the probe 55 or whereby the probe first advanced configuration can be a partially advanced configuration of the probe 55 and the probe second advanced configuration can be a fully advanced configuration of the probe 55.

FIG. 20 illustrates that when the probe 55 is in the probe first configuration PC1, the device distal terminal end 50dte can be, for example, the conduit distal terminal end 52dte. FIG. 20 illustrates that when the probe 55 is in the probe second configuration PC2, the device distal terminal end 50dte can be, for example, the probe tip 56.

FIG. 20 illustrates that the probe 55 can be moved (e.g., advanced) a distance D5 (also referred to as the deployed distance D5, the advanced distance D5, the advancing distance D5, or any other similar term) beyond the conduit 52. The distance D5 can be, for example, the distance that the probe 55 is advanced beyond the distal end opening (e.g., the opening 50z) of the device 50. The distance D5 can be, for example, the distance that the probe 55 is advanced beyond the opening 50z to position the probe 55 in the probe second configuration PC2. The distance D5 can be measured, for example, from the probe distal end 55de to the conduit distal end 52de. The distance D5 can be measured, for example, from the probe tip 56 to the conduit distal terminal end 52dte and/or from the probe tip 56 to the opening 50z (e.g., the opening 50z that the probe 55 can be advanced through). For example, FIG. 20 illustrates that the distance D5 can be measured from the probe tip 56 to the longitudinal center of the opening 50z. The distance D5 can be, for example, measured along the probe center longitudinal axis A3. The distance D5 can be, for example, 0.0 cm-30.0 cm, or more narrowly, 0.0 cm-20.0 cm, or more narrowly, 0.0 cm-15.0 cm, or more narrowly, 0.0 cm-10.0 cm, or more narrowly, 0.0 cm-5.0 cm, including, for example, every 0.1 cm increment within these ranges (e.g., 0.0 cm, 5.0 cm, 10.0 cm, 15.0 cm, 20.0 cm, 30.0 cm). The upper bound of these ranges for the distance D5 (e.g., 30.0 cm, 20.0 cm, 10.0 cm, 5.0 cm) can be, for example, the maximum distance that the probe 55 can be advanced out of the conduit 52. As another example, there may not be a maximum distance that the probe 55 can be advanced out of the conduit 52, in which case the upper bound of the above ranges for the distance D5 may not be the maximum distance that the probe 55 can be advanced out of the conduit 52. For example, the distance D5 can be the distance that the user advances the probe 55 beyond and/or out of the conduit 52, whereby the distance D5 may or may not also be the maximum distance that the probe 55 is capable of being advanced beyond and/or out of the conduit 52.

FIG. 20 illustrates that the probe 55 can be moved beyond and/or out of the conduit (e.g., through the opening 50z), for example, to confirm whether the device 50 has access to the target site 173 (e.g., the body space 8). FIG. 20 illustrates, for example, that the probe 55 can be moved from proximal the conduit distal terminal end 52dte (e.g., from the probe first configuration PC1) to distal the conduit distal terminal end 52dte (e.g., to the probe second configuration PC2), for example, through the opening 50z, to confirm whether the device 50 has access to the body space 8. When the distance D5 is greater than or equal to a threshold distance D6, the configuration of the probe 55 can be an indicator that the device 50 has access to the body space 8. When the distance D5 is greater than or equal to the threshold distance D6, the configuration of the probe 55 can be an indicator that the device 50 has enough access to deliver and/or remove one or multiple materials 160 (e.g., the material 160a, the material 160b) to and/or from the body space 8, respectively. When the distance D5 is less than the threshold distance D6, the configuration of the probe 55 can be an indicator that the device 50 does not have access to the body space 8. When the distance D5 is less than the threshold distance D6, the configuration of the probe 55 can be an indicator that the device 50 does not have enough access to deliver and/or remove one or multiple materials 160 (e.g., the material 160a, the material 160b) to and/or from the body space 8, respectively. The distance D6 can be measured, for example, the same way or in a different way as the distance D5.

The device 50 can be considered to have access to the target site 173 (e.g., the body space 8) when the conduit distal terminal end 52dte and/or an opening 50z has access (e.g., is inside) the body space 8. For example, the advanced distance D5 being greater than or equal to the threshold distance D6 can be an indicator that the opening 50z has access to the body space 8. As another example, the advanced distance D5 being greater than or equal to the threshold distance D6 can be an indicator that the device 50 has sufficient access to the body space 8 to deliver and/or remove one or multiple materials 160 (e.g., the material 160a, the material 160b) to and/or from the body space 8, respectively. The device 50 can be considered to not have access to the target site 173 (e.g., the body space 8) when the conduit distal terminal end 52dte and/or an opening 50z does not have access (e.g., is outside) the body space 8. For example, the advanced distance D5 being less than the threshold distance D6 can be an indicator that the opening 50z does not have access to the body space 8. As another example, the advanced distance D5 being less than the threshold distance D6 can be an indicator that the device 50 does not have sufficient access to the body space 8 to deliver and/or remove one or multiple materials 160 (e.g., the material 160a, the material 160b) to and/or from the body space 8, respectively.

FIG. 20 illustrates a variation in which the probe 55 has been advanced to a distance D5 that is greater than the threshold distance D6. FIG. 20 illustrates, for example, that achieving the threshold distance D6 for advancement of the probe 55 can indicate that the conduit 52 and the distal opening (e.g., the opening 50z) are within the body space 8 (e.g., in the vas deferens lumen 8v). The threshold distance D6 can be the confirmation test of intraluminal placement of the device 50. FIG. 20 illustrates, for example, that the device 50 has access to the vas deferens lumen 8v since the probe 55 was able to reach the threshold distance D6.

The threshold distance D6 can be, for example, 1.0 cm to 15.0 cm, or more narrowly, 1.0 cm-12.0 cm, or more narrowly, 2.0 cm-12.0 cm, or more narrowly, 2.0 cm-10.0 cm, or more narrowly, 2.0 cm-5.0 cm, including every 0.1 cm increment within these ranges (e.g., 1.0 cm, 2.0 cm, 5.0 cm, 10.0 cm, 12.0 cm, 15.0 cm). The threshold distance D6 can be, for example, 2.0 cm to 12.0 cm with a 5.0 cm translation.

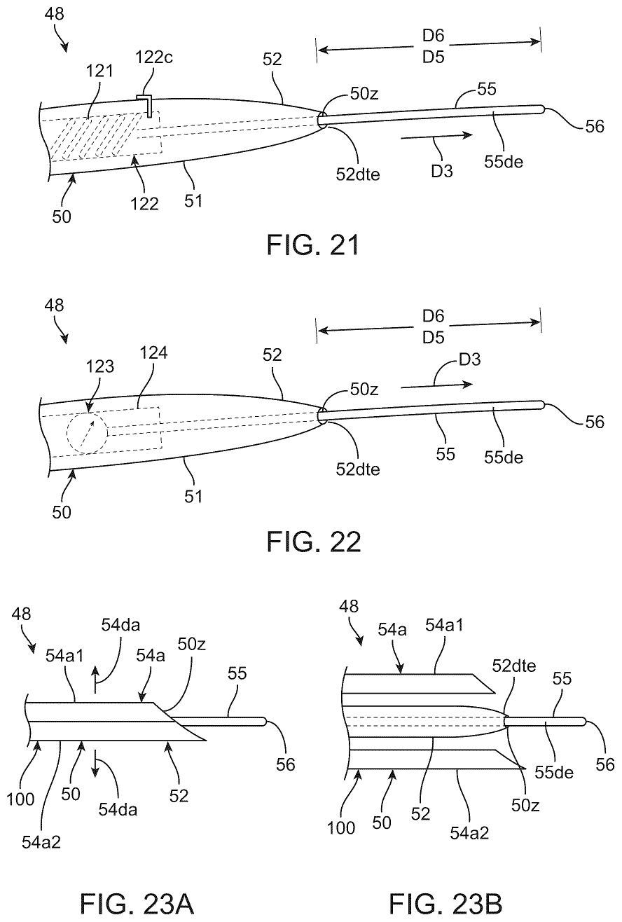

FIG. 21 illustrates that the probe 55 can be connected to a spring mechanism 122 for translation of the probe 55 and for confirmation of placement of the device 50 in the body space 8. FIG. 21 illustrates that the spring mechanism 122 can have a spring 121 for providing a translation of the probe 55 and for confirmation of placement in the body space 8. The spring mechanism 122 can, for example, advance the probe 55 the threshold distance D6 in direction D3. FIG. 21 illustrates, for example, that that the spring can push the probe 55 out of the device distal end 50de such that the advanced distance D5 equals the threshold distance D6. FIG. 21 illustrates a variation of the probe second configuration PC2. FIG. 21 illustrates, for example, the probe 55 in the probe second configuration PC2. FIG. 21 illustrates that the spring mechanism 122 can be triggered by the user, for example, via a release latch 122c connected to the spring 121.

FIG. 22 illustrates that the probe 55 can be connected to a deflection force measurement device 123 for placement confirmation of the device 50 in the body space 8. FIG. 22 illustrates that the deflection force measurement device 123 within a housing 124 (e.g., a compartment) of the system 48 (e.g., the system 48D and/or the system 48R). For example, FIG. 22 illustrates that the deflection force measurement device 123 and the housing 124 can be in the handle 51. Upon advancement of the probe 55, the deflection force measurement device 123 can record the amount of force, and/or can limit the amount of force, required to advance the probe 55 the threshold distance D6. Achieving a force below a predetermined threshold for the advancement of the probe 55 at the threshold distance D6 can be used as a confirmation for placement of the device 50 in the body space 8. FIG. 22 illustrates a variation of the probe second configuration PC2. FIG. 22 illustrates, for example, the probe 55 in a probe second configuration PC2 when the advanced distance D5 is equal to the threshold distance D6.

FIGS. 23A-23B illustrate that that the sharp distal end of the device 50 can be removed before further manipulation of the device 50 in the body space 8. FIGS. 23A-23B illustrate, for example, that the conduit assembly 100 can have the conduit 52 and the split needle 54a such that the conduit 52 can be advanced over the probe 55 after the split needle 54a is in the split configuration and/or after the split needle 54a is retracted or otherwise removed from the body space 8. FIGS. 23A-23B illustrate that the sharp distal end of the device 50 can be the split needle 54a shown in FIGS. 9B1-9B2. FIG. 23A illustrates a side view of the non-split configuration shown in FIG. 9B1. FIG. 23B illustrates a side view of the split configuration shown in FIG. 9B2. FIGS. 23A-23B illustrate, for example, that the split needle 54a can radially separate after the probe 55 is advanced into the body space 8 and access to the body space 8 has been confirmed. Once the needle first portion 54al and the needle second portion 54a2 are separated (e.g., as shown in FIG. 23B), the conduit 52 can be advanced over the probe 55 (e.g., as shown in FIG. 23B), for example, for the delivery and/or removal of one or multiple materials 160 through the conduit 52.

FIGS. 23C-23D illustrate that that the sharp distal end of the device 50 can be removed before further manipulation of the device 50 in the body space 8. FIGS. 23C-23D illustrate, for example, that the conduit assembly 100 can have the conduit 52 and a half needle (e.g., the half needle 54b) such that the conduit 52 can be advanced over the probe 55 after the half needle 54b is withdrawn from the body space and/or moved away from the probe 55 (e.g., as shown in FIG. 23D). Once the conduit 52 is advanced over the probe 55 and into the body space, the device can deliver and/or remove one or multiple materials 160 to and/or from the body space 8, respectively. FIG. 23 illustrates that the probe 55 can advanced beyond the needle tip 57.

For variations of the device 50 in which the conduit assembly 100 has a sheath (e.g., the conduit 52) and a needle (e.g., the needle 54) that is separate from the sheath (e.g., as shown in FIGS. 23A-23D), the probe 55 can be advanced relative to the needle 54 just as the probe 55 can be advanced relative to the conduit 52 as described herein, mutatis mutandis, for example, where the opening 50z can be the distal opening of the needle 54 (e.g., of a conventional needle as shown in FIG. 6, the split needle 54a, the half needle 54b). For example, when the needle 54 is the split needle 54a, the opening 50z can be the distal opening of the split needle 54a when the needle is in the non-split configuration. For example, when the needle 54 is the half needle 54b, the opening 50z can be the end of the needle channel 64 and/or the distal terminal opening (e.g., the opening 50z) of the conduit 52.

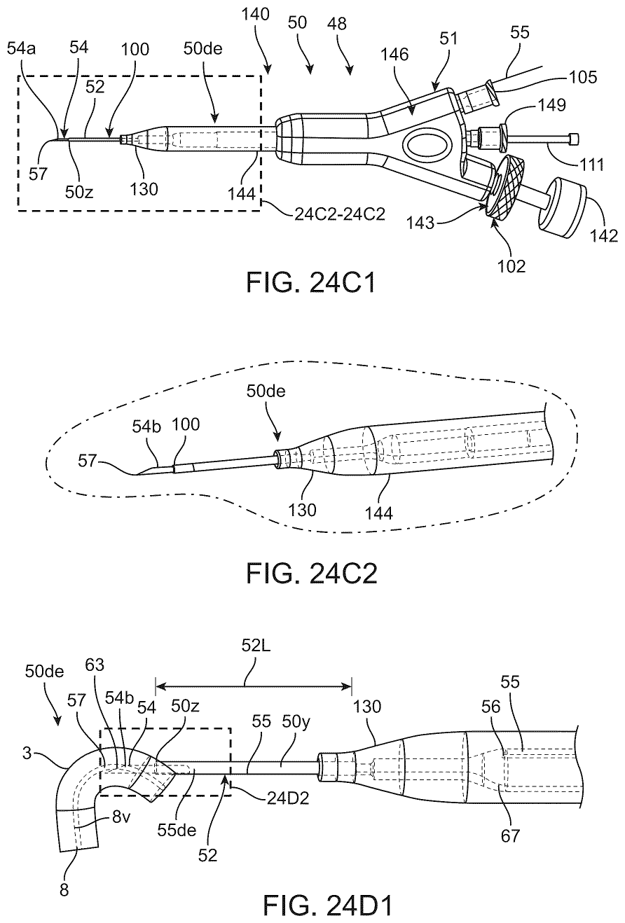

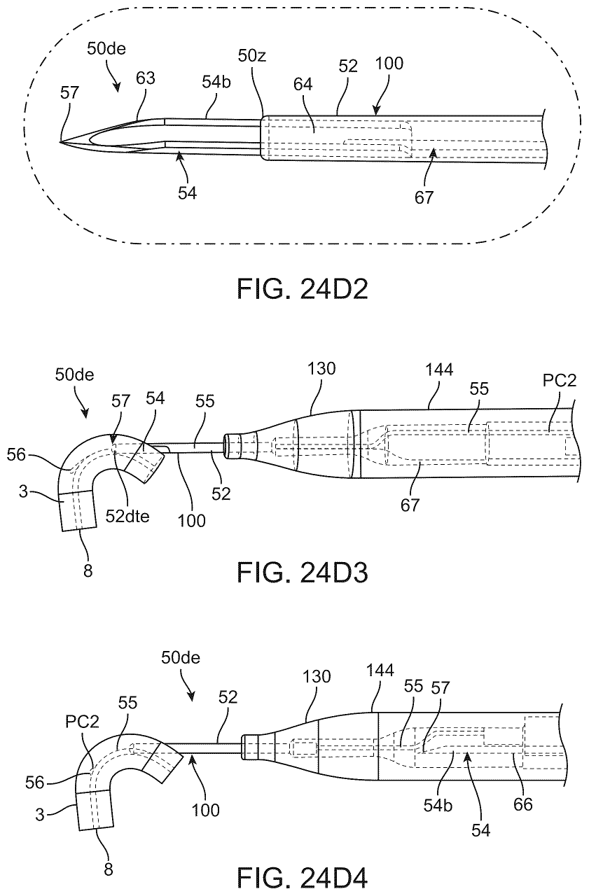

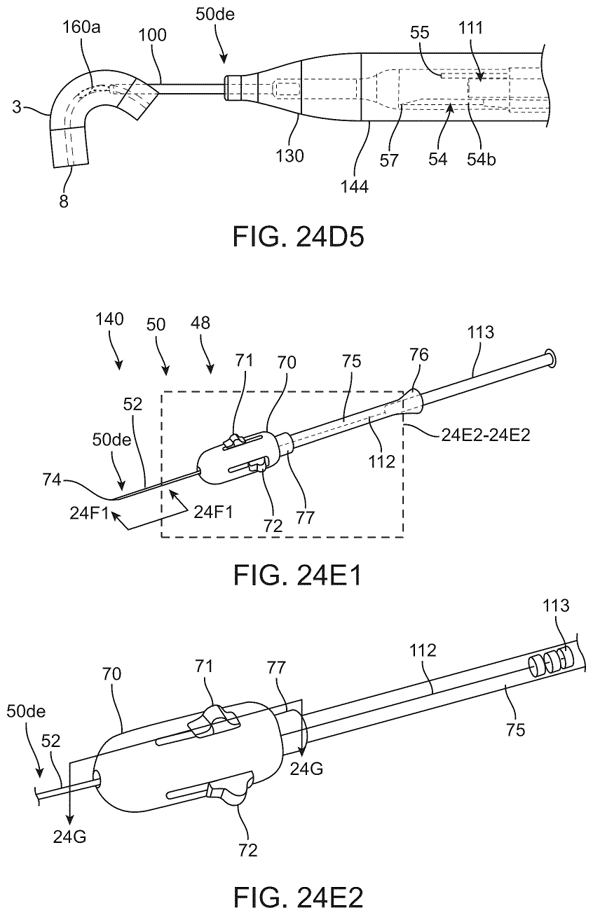

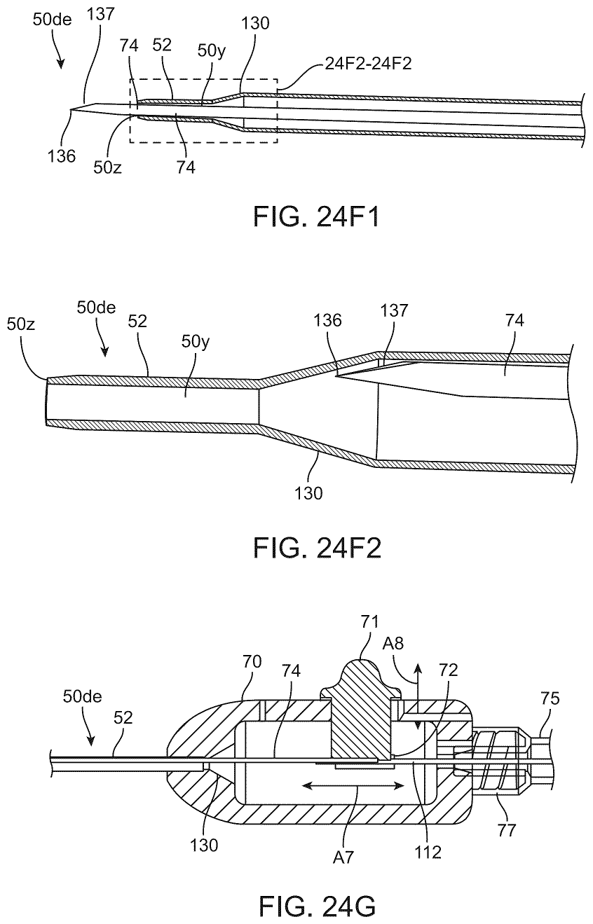

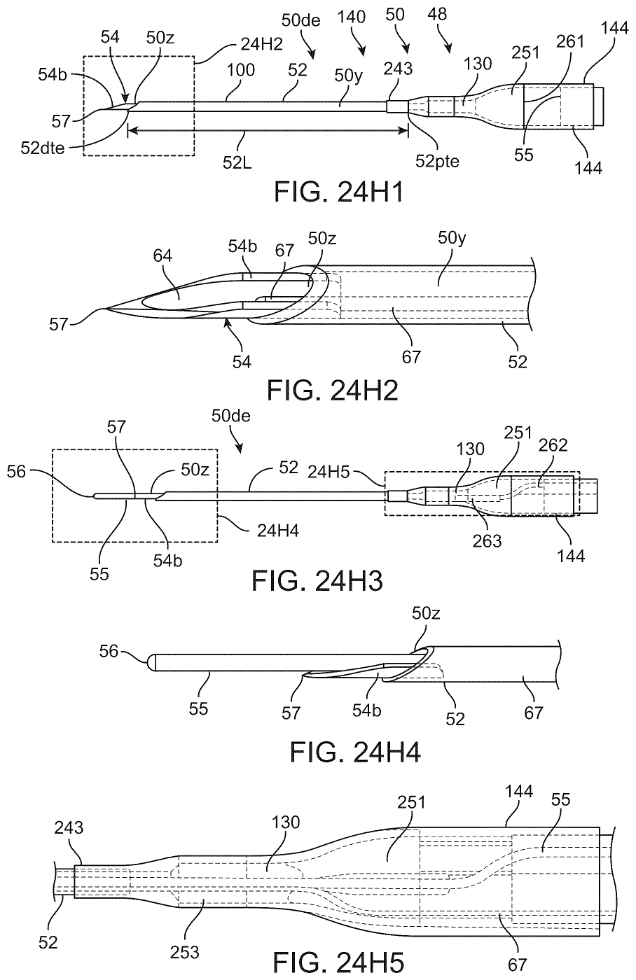

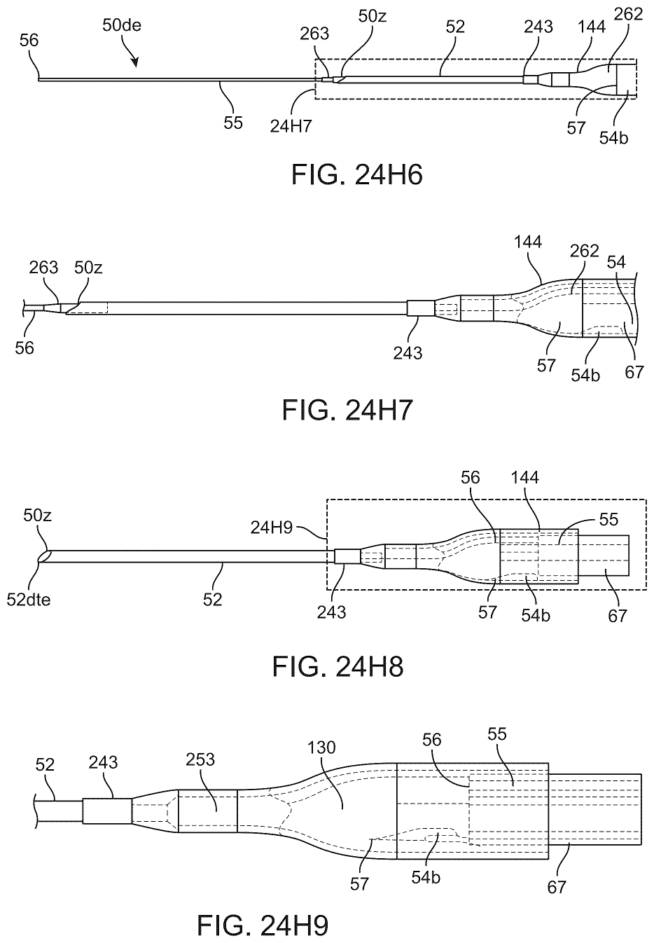

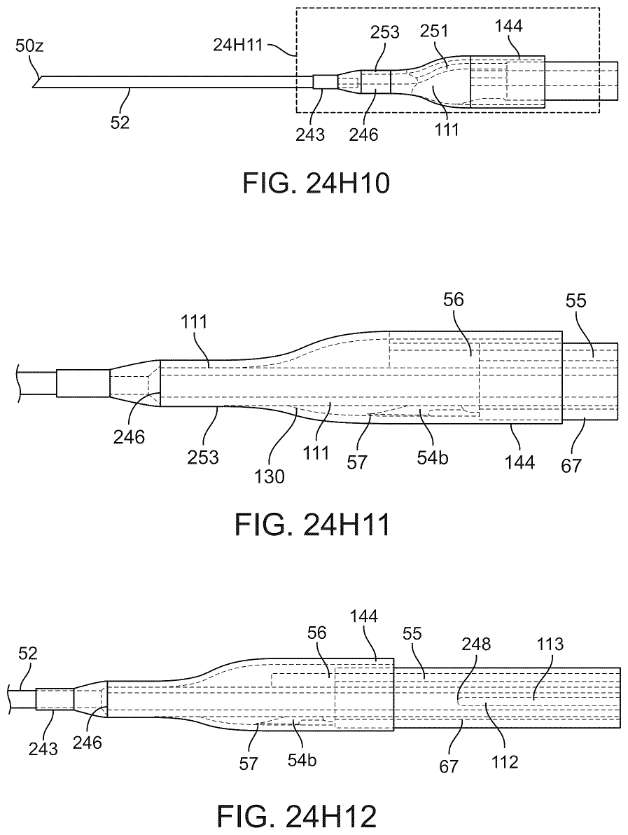

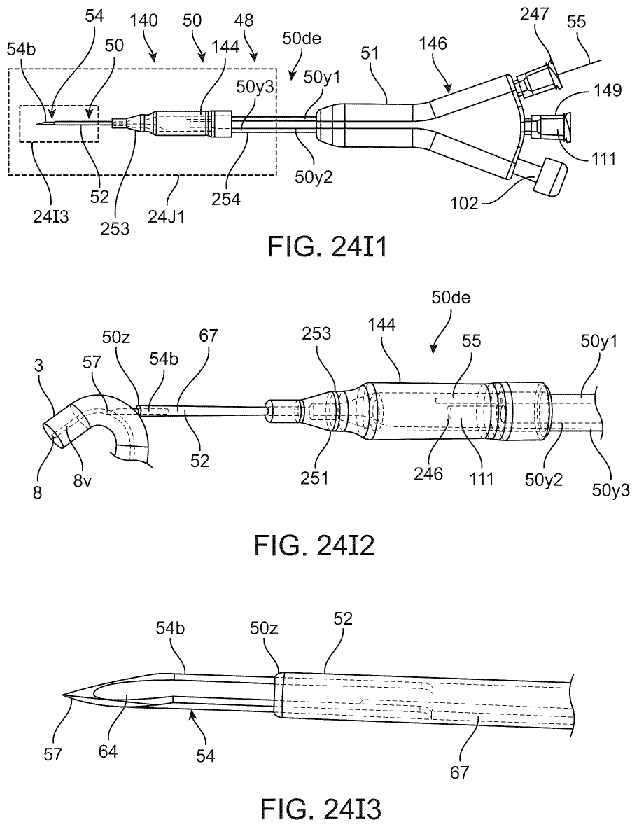

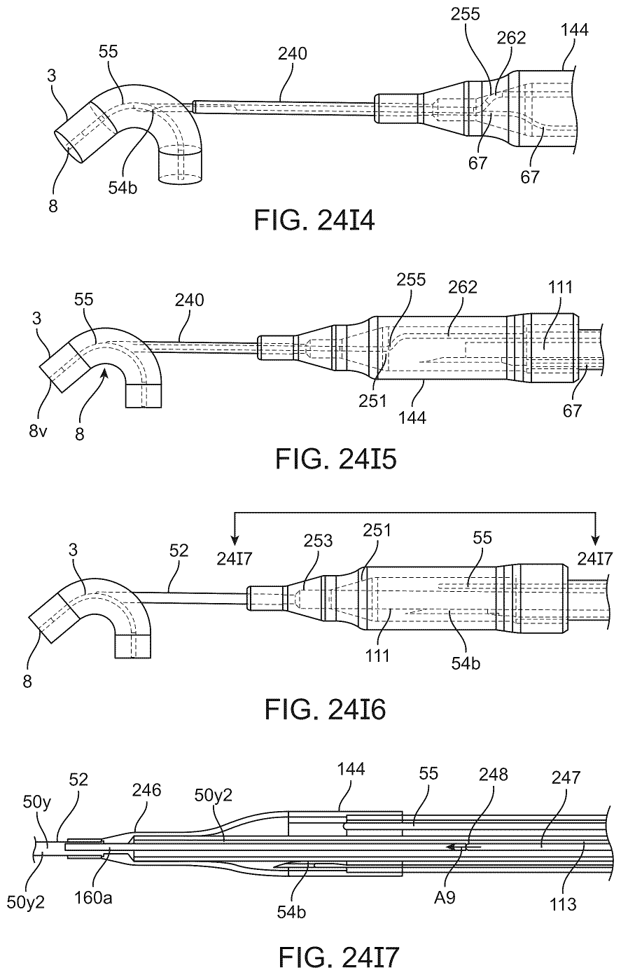

FIG. 24A to 24K illustrate various variations of the device 50 and various processes of placing the device 50 in the body space 8.

FIG. 24A illustrates that to confirm placement of the device 50 in the body space 8, the probe 55 can be advanced the threshold distance D6 into the body space 8.

FIG. 24B illustrates the retraction of the probe 55 back into handle 51 and proximal to a connector 130. FIG. 24B illustrates that the connector 130 can have a nose cone. FIG. 24B illustrates that the connector 130 can be a nose cone. The connector 130 can be attached to and/or integrated with the handle 51. Retraction of probe 55 into the connector 130 can drop the probe distal end 55de to a location below the conduit center longitudinal axis A1, for example, to provide an unobstructed lumen 50y in the conduit assembly 100. FIG. 24B illustrates that the lumen 50y can be the central lumen of the conduit assembly 100. FIG. 24B illustrates that the probe 55 can be retracted out of the lumen 50y.

FIG. 24C1 illustrates a variation of the system 48 (e.g., the system 48D and/or the system 48R). FIG. 24C1 illustrates, for example, that the device 50 can be a multi-lumen (e.g., a three-lumen) system 140 having the arrangement of features shown. FIG. 24C1 illustrates that the device 50 can have the handle 51, the conduit 52, the needle 54, the connector 130, a probe port 105 for the probe 55, a cartridge port 149 for the cartridge 111 with the material 160a (e.g., a hydrogel 150), the control 113 (e.g., a plunger), the needle control 102 comprising a penetration adjuster 143 (e.g., the penetration adjuster 143 (also referred to as the adjuster 143) can control the advancement and retraction of the needle 52 to control the depth of the needle in the body space 8), a removal hub 142, a housing 144, a fitting 146 (e.g., a Y-fitting having three lumens), or any combination thereof. FIG. 24C1 illustrates that the conduit 52 can be a sheath (e.g., an access sheath). FIG. 24C1 illustrates that the needle 54 can be a half needle (e.g., the half needle 54b). FIG. 24C1 illustrates that the needle can be a 21-gauge half needle, that the probe 55 can be a 0.018 inch guidewire, that the housing 144 can be a 3-lumen extrusion, or any combination thereof. FIG. 24C1 illustrates the probe 55 in the probe port 105. The housing 144 may or may not have the connector 130. The connector 130 can, for example, merge two lumens into one lumen. As another example, the connector 130 can connect a first lumen and a second lumen to a third lumen. The housing 144 can have the connector 130.

FIG. 24C2 illustrates the housing 144 in a transparent view, and that the connector can terminate at the proximal opening of the conduit 52. The half needle 54b can be sized as one-half of, for example, a 21-gauge needle, a 22-gauge needle, a 23-gauge needle, a 24-gauge needle, or a smaller or larger gauge needle.

FIGS. 24D1-24D5 illustrate a variation of the device 50 and a process for accessing the body space 8 (e.g., the vas deferens lumen 8v) with the device 50 and delivering a material (e.g., the material 160a) to the body space 8 with the device 50.

FIG. 24D1 illustrates that the device 50 can have a connector 67 (e.g., a pull wire) attached to the needle 54 (e.g., to the half needle 54b). The connector 67 can be used, for example, to advance and retract the needle 54 (e.g., the half needle 54b). The needle 54 (e.g., the half needle 54b) can be advanced and/or retracted, for example, by pushing and/or pulling the proximal end of the connector 67 that extends proximally from one of the ports in the handle 51. As another example, the proximal end of the connector 67 can be connected to the needle control 102 such that the needle 54 can be advanced by moving the needle control 102 in a first direction (e.g., by pushing the adjuster 143 toward the handle 51 or by rotating the adjuster 143 in a clockwise direction) and can be retracted by moving the needle control 102 in a second direction (e.g., by pulling the adjuster 143 away from the handle 51 or by rotating the adjuster 143 in a counterclockwise direction). FIG. 24D1 illustrates, for example, that the needle 54 (e.g., the half needle 54b) can be advanced in the conduit assembly 100 through the lumen 50y of the conduit 52 via the needle control 102 such that the needle 54 protrudes beyond distal end opening 50z of the conduit 52.

FIG. 24D1 illustrates that the conduit 52 can be positioned such that as the needle 54 (e.g., the half needle 54b) is advanced out of the sheath 52, the needle 54 (e.g., the half needle 54b) can be advanced into the body space 8. For example, FIG. 24D1 illustrates the half needle 54b entering the vas deferens lumen 8v as the needle 54 is advanced via the needle control 102. FIG. 24D1 illustrates the half needle 54b in the vas deferens lumen 8v

As another example, when the needle 54 (e.g., the half needle 54b) is in a position in which the needle 54 protrudes from the opening 50z of the conduit 52, the half needle 54b can be advanced into the body space 8 by pushing the device 50 as a whole (e.g., in direction D1) via the handle 51.

FIG. 24D1 illustrates that the conduit length 52L can be, for example, 3.0 cm, and that the conduit width 50W can be an outer diameter of 1 mm. FIG. 24D1 illustrates that the needle 54 (e.g., the half needle 54b) can have a distal bevel 63.

FIG. 24D2 illustrates that the connector 67 can be attached to the half needle 54b, for example, somewhere along a surface of the needle channel 64. FIG. 24D2 illustrates that the connector 67 can be, for example, a 0.008 inch diameter wire or a 0.008 inch flattened wire. FIG. 24D2 illustrates that the half needle 54b can be, for example, a 21-gauge lancet needle.

FIG. 24D3 illustrates that the needle 54 can be retracted into the conduit 52, for example, via the needle control 102, that the probe 55 can be advanced into the body space 8, and that that the conduit 52 can be advanced into the body space 8.

FIG. 24D3 illustrates that the probe 55 can be advanced and/or retracted, for example, by pushing and/or pulling the proximal end of the probe 55 that extends proximally from the probe port 105 (e.g., as shown in FIG. 24C1).

FIG. 24D3 illustrates that after the probe 55 is advanced into the body space 8 (e.g., by pushing the proximal end of the probe 55 through the probe port 105), the probe 55 can be advanced into the body space 8. The conduit 52 can then be advanced over the probe 55 into the body space 8 (e.g., by advancing the device in direction D1).

FIG. 24D3 illustrates, for example, the needle 54 (e.g., the half needle 54b) being withdrawn from the vas deferens lumen 8v, and the conduit 52 and the probe 55 entering the vas deferens lumen 8v. FIG. 24D3 illustrates the conduit 52 and the probe 55 in the vas deferens lumen 8v.

FIG. 24D3 illustrates the needle 54 (e.g., the half needle 54b) in a partially retracted position in the conduit 52, with the needle tip 57 in the body space 8 distal the opening 50z of the conduit 52.

FIG. 24D3 illustrates the probe 55 in an advanced configuration. The advanced configuration shown can be, for example, the probe second configuration PC2.

As another example, after the conduit 52 has been advanced over the needle 54 (e.g., by moving the device 50 in direction D1), the probe 55 can be advanced into the body space 8, for example, to confirm that the conduit 52 (e.g., the opening 50z) has access to the body space 8. Advancing the conduit 52 over the needle 54 can result in the needle 54 having a retracted configuration.

FIG. 23D3 illustrate that the probe 55 and the connector 67 can extend through the device 50 as shown.

FIG. 23D4 illustrates the needle 54 (e.g., the half needle 54b) in a fully retracted position

FIG. 23D4 illustrates the needle 54 (e.g., the half needle 54b) in a fully retracted position and the probe in a retracted configuration. The retracted configuration of the probe 55 shown can be, for example, the probe first configuration PC1. FIG. 23DE illustrates that the probe first configuration PC1 can be a fully retracted configuration of the probe 55.

FIG. 23D5 illustrates that the cartridge 111 can be in the housing 144 or can be pushed into the housing 144 through the cartridge port 149. FIG. 23D5 illustrates that the control 113 (e.g., a plunger) can be advanced to deliver the material 160a to the target space 8.

FIGS. 24E1 and 24E2 illustrate another variation of features that the device 50 can have. For example, FIGS. 24E1 and 24E2 illustrate another variation of the multi-lumen (e.g., three-lumen) system 140. FIGS. 24E1 and 24E2 illustrate that the multi-lumen system 140 can have the arrangement of features shown. FIGS. 24E1 and 24E2 illustrate that the device 50 can have the handle 51, the conduit 52, a housing 70, a trocar slide button 71, a guidewire slide button 72, a trocar 74, a holder 75 for the cartridge 111, a stopper 76 (e.g., for the control 113), a cartridge connector 77, or any combination thereof. FIGS. 24E1 and 24E2 illustrate that the handle 51 can have the housing 70, and that the housing 70 can have the trocar slide button 71 and the guidewire slide button 72. The trocar slide button 71 can control linear and/or vertical movement of the trocar 74. The trocar slide button 71 can, for example, advance and retract the needle 54 (e.g., the half needle 54b) through the conduit 52. The guidewire slide button 72 can advance and retract the probe 55 (not shown) through the conduit 52. The device 50 can have the cartridge connector 77 with the cartridge holder 75 in the arrangement shown. The cartridge holder 75 can hold the material 160a (e.g., a hydrogel). On the proximal end, the control 113 (e.g., a plunger) can advance the rod 112 (e.g., a plunger push rod) to eject the material 160a (e.g., a hydrogel) from the cartridge holder 75. FIG. 24E1 illustrates the trocar slide button 71 in an advanced position, and FIG. 24E2 illustrates the trocar slide button 71 in a retracted position. FIGS. 24E1 and 24E2 illustrate the guidewire slide button 72 in a retracted position. The guidewire slide button 72 can be, for example, be connected to the probe 55 or to the connector 67.

FIGS. 24F1-24F2 illustrate that the trocar 74 can have a sharp distal tip 136 and a bevel 137. FIGS. 24F1-24F2 illustrate that the trocar 74 can be removed from the lumen 50y and/or be retracted to a retracted position that does not obstruct the opening 50z, for example, prior to advancement of the probe 55. FIG. 24F1 illustrates the trocar 74 advanced beyond the distal end opening of the of conduit 52, for example, beyond the opening 50z. FIG. 24F1 illustrates that the trocar 74 is ready for insertion into the vas deferens 3. FIG. 24F2 illustrates the trocar 74 retracted into connector 130 with the trocar 74 (e.g., the trocar tip 136) housed away from (e.g., above) the lumen 50y of the conduit 52. For example, FIG. 24F2 illustrates the trocar 74 pulled back into the handle 51. FIGS. 24F1-24F2 illustrate that the lumen 50y shown can be the central lumen of the device 50.

FIG. 24G illustrates a variation of the trocar slide button 71 for advancing and retracting the trocar 74, for example, out of and into the handle 51 (e.g., out of and into the housing 70). FIG. 24G illustrates that the trocar slide button 71 can be attached to the trocar 74 and can provide movement both horizontally and vertically by spring loading. The trocar slide button 71 can be moved back and forth as indicated by double headed arrow A7 and up and down as indicated by double headed arrow A8 (e.g., the trocar slide button 71 can be spring loaded).

FIGS. 24H1-24H6 illustrate a variation of a process of accessing the body space 8 (e.g., within the vas deferens lumen 8v) with the device 50 and deploying the material 160a to the body space 8. For example, FIGS. 24H1-24H2 illustrate the steps of advancing the probe and delivering the material 160a into the vas deferens 3.

FIG. 24H1 illustrates a variation of features that the device distal end 50de can have. For example, FIG. 24H1 illustrates a variation of the housing 144 and the arrangement of features shown. FIG. 24H1 can be, for example, a variation of the device 50 shown in FIGS. 24D1-24D5. FIG. 24H1 illustrates that the housing 144 can have a distal terminal end 243. FIG. 24H1 illustrates the device 50, for example, ready to be advanced into the body space 8. FIG. 24H1 illustrates the connector 130 can have a proximal terminal end 261. FIG. 24H1 illustrates that the device 50 can have three lumen junction 251, which can be the mating point for the multiple components.

FIG. 24H2 illustrates a variation of the device distal end 50de with the needle tip 57 protruding beyond the distal opening 50z of the conduit 52.

FIGS. 24H3-24H5 illustrate the probe 55 advanced beyond the needle tip 57 and beyond the distal opening 50z of the conduit 52. FIGS. 24H3-24H5 illustrate that the device can have the arrangement of features in the relative positions shown. For example, FIGS. 24H3-24H5 illustrate that when the probe 55 is in an advanced configuration, the probe 55 can extend through the needle channel 64 of the half needle 54b. FIG. 24H5 illustrates that the housing 144 can have a cartridge docking area 253 for the cartridge 111. FIGS. 24H3-24H5 illustrate a configuration that the device distal end 50de can have when the probe 55 is in an advanced position in the body space (e.g., the vas deferens lumen 8v). FIG. 24H3 illustrates that a pull wire 262 can be attached to the probe 55, for example, at a probe pull wire connection 263. FIG. 24H3 illustrates that the probe pull wire connection 263 can be the junction of the probe 55 and probe pull wire 262. As another example, the device 50 may not have the pull wire 262, in which case the probe 55 can be pulled directly.

FIGS. 24H6-24H7 illustrate the probe 55 in an advanced configuration and the needle 54 in a retracted configuration. FIG. 24H6 illustrates the probe 55 advanced beyond the conduit 52 by the distance D5. FIG. 24H6 illustrates, for example, the probe 55 advanced the distance D5 beyond the opening 50z of the conduit 52. The distance D5 shown in FIG. 24H6 can be, for example, 4.0 cm. As another example, FIG. 24H6 illustrates that the distance between the probe tip 56 can be 4.0 cm from the needle tip 57. FIGS. 24H6-24H7 illustrate that when the needle 54 is in a retracted position, the needle 54 and the distal end of the connector 67 can be in the housing 144. FIGS. 24H6-24H7 illustrate, for example, the probe 55 in a fully advanced configuration. FIGS. 24H6-24H7 illustrate, for example, the needle 54 and the connector 67 in a fully retracted position.

FIGS. 24H8-24H9 illustrate the probe 55 in a retracted configuration and the needle 54 in a retracted configuration. FIGS. 24H8-24H9 illustrate the needle 54 and the probe 55 can be completely retracted and clear of the lumen 50y of the conduit 52. FIGS. 24H8-24H9 illustrate that the lumen 50y of the conduit 52 can be the central lumen of the device distal end 50de. FIGS. 24H6-24H7 illustrate that when the probe 55 is in a retracted configuration, the probe 55 (e.g., the probe tip 56) can be in the housing 144. FIG. 24H9 illustrates that the connector 130 (also referred to as a transition piece) can be clear of both the half needle 54b and the probe 55 after they are retracted from the conduit 52.

FIGS. 24H10-24H11 illustrate that when the probe 55 and the needle 54 are in retracted configurations (e.g., in fully retracted configurations), the cartridge 111 can be advanced into the cartridge docking area 253. For example, FIGS. 24H10-24H11 illustrate that the cartridge 111 can have a connector 246 that can mate with the cartridge docking area 253 of the connector 130. The cartridge 111 can releasable mate with the connector 130, for example, to provide the material 160a into the body space 8. FIGS. 24H10-24H11 illustrate that the cartridge 111 can be placed within the central lumen of the housing 144, translated through connector 130, and coupled (e.g., releasably coupled) with the cartridge docking area 253. FIG. 24H11 illustrates the arrangement of features shown.

FIG. 24H12 illustrates that when the cartridge 111 is in the docking area 253, the control 113 (e.g., a plunger) can be advanced through cartridge 111 to force the material 160a out of the cartridge and into the body space 8 (e.g., the vas deferens lumen 8v). FIG. 24H12 illustrates the control 113 and/or the rod 112 pushing the material 160a (not shown) through the cartridge 111. The distal end 248 of the control 113 and/or the rod 112 can extend through traverse distal end opening of the cartridge 111 and through access sheath 240 to expel hydrogel in the vas deferens (not shown) or body lumen.

FIGS. 24I1-24I7 illustrate a variation of the device 50 and a process for accessing the body space 8 (e.g., the vas deferens lumen 8v) with the device 50 and delivering a material (e.g., the material 160a) to the body space 8 with the device 50. FIGS. 24I1-24I7 illustrate that the device 50 can have the arrangement of features shown.

FIG. 24I1 illustrates a variation of the multi-lumen system 140. FIG. 24I1 illustrates that the handle 51 (e.g., a proximal end of the handle 51) can have the irrigation connector 247 and the probe 55, the cartridge port 149 and cartridge with hydrogel 245, and the needle control 102. FIG. 24I1 illustrates that the needle control 102 can be, for example, a D-shaped needle handle for advancing and retracting the half needle 54b via the connector 67. FIG. 24I1 illustrates that the device 50 can have a multi-lumen piece 254. FIG. 24I1 illustrates that the connector 144 can connect to the multi-lumen piece 254. FIG. 251 illustrates that the multi-lumen piece 254 can have 3 lumens 50y, for example, a first lumen 50y1, a second lumen 50y2, and a third lumen 50y3. FIG. 24I1 illustrates that the second lumen 50y2 can be, for example, the central lumen of the device 50. FIG. 24I1 illustrates that the distal opening of the conduit 52 (e.g., the opening 50z) can be the distal opening of the second lumen 50y2. FIG. 24I1 illustrates that the probe 55 can be advanceable and retractable in the first lumen 50y1 and the second lumen 50y2, that the cartridge 111 can be advanceable and retractable in the second lumen 50y2, that the needle 54 (e.g., the half needle 54b) and the connector 67 can be advanceable and retractable in the second lumen 50y2 and the third lumen 50y3. FIG. 24I1 illustrates a configuration that the device 50 can have when packaged, for example, for shipping.

FIG. 24I2 illustrates the conduit 52 ready to advance into the vas deferens 3, showing the half needle 54b entering the vas deferens lumen 8v.

FIG. 24I3 illustrates the half needle 54b and the connector 67 attached to each other.

FIG. 24I4 illustrates that the probe 55 can be advanced into the vas deferens lumen 8v. FIG. 24I4 illustrates that the device 50 (e.g., the connector 144) can have a pull wire latch release 255 that can provide, for example, a positive opening for the cartridge 111 (shown in the next step in FIG. 24I5). The pull wire latch release 255 can be a lock-out that can prevent the cartridge 111 from being deployed. Deployment of the cartridge 111 and delivery of the material 160a (e.g., a hydrogel) can be done after the translation of probe 55 is satisfied, which can include confirming that the device 50 has access to the body space 8, for example, by advancing the probe 55 by the distance D5 and confirming that the device 50 has access if the distance D5 is greater than or equal to the threshold distance D6. In practice, deployment of the cartridge 111 and delivery of the material 160a (e.g., a hydrogel) can be prevented until the translation of probe 55 is satisfied.

FIG. 24I5 illustrates the half needle 54b in a fully retracted configuration in the housing 144. FIG. 24I5 illustrates that the conduit 52 can be advanced, for example, by pushing the conduit 52 over the probe 55 (e.g., by moving the handle 51 in direction D1 toward the body space 8) while the half needle 54b is in a retracted position (e.g., a fully retracted position) in the housing 144. The advancement of the probe 55 within the body space 8 can open the central lumen (e.g., lumen 50y2) within the housing 144 by the pull wire latch release 255 reaching a threshold distance.

FIG. 24I6 illustrates the half needle 54 and the probe 55 completely retracted and the cartridge 111 in an advanced configuration with the connector 246 mated with the cartridge docking area 253 with the conduit 52 in the body space 8. The cartridge 111 can be coupled to the connector 144 in the cartridge docking area 253. A larger outer diameter taper can be added to the probe 55, for example, to aid in accessing the vas deferens lumen 8v. FIG. 24I6 illustrates that the material 160a in the cartridge 111 is ready to be delivered to the body space 8.

FIG. 24I7 illustrates that the control 113 (e.g., a plunger) can be advanced through the cartridge 111 and the material 160a (e.g., a hydrogel) can be injected into the vas deferens lumen 8v (see FIG. 24I6). FIG. 24I7 illustrates the that the material 160a can be deployed into the body space 8 when the cartridge 111 is in the docking area 253. FIG. 24I7 illustrates that the rod 112 attached to or integrated with the control 113 can advance through the central lumen (e.g., the lumen 50y2) and into the conduit 52 through the cartridge 111 to push the material 160a into the body space 8 as shown by arrow A9. The direction of arrow A9 can be, for example, the same as direction D1. FIG. 24I7 illustrates the material 160a being pushed out of the cartridge 111 into the conduit 52 via the rod 112.

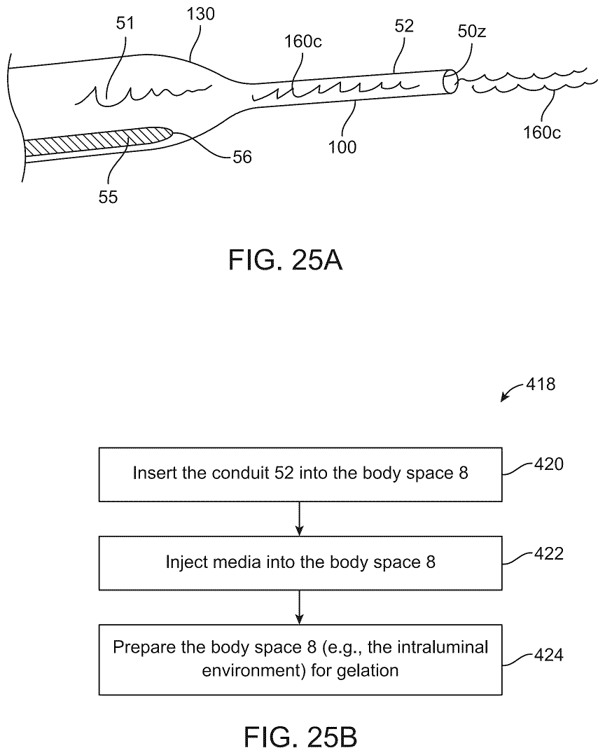

FIGS. 25A-25B illustrate a variation for preparing the body space 8 for delivery of the material 160c. FIG. 25A illustrates the injection of the material 160c from the device distal end 50de for clearing the body space 8 of material and preparing for hydrogel insertion after delivery sheath 100 insertion, for example, as described above with respect to the FIGS. 17A-17H.

FIG. 25B illustrates a variation of a method 418 that can be performable using the system 48 (e.g., the device 50 and/or the device 180). FIG. 25B illustrates that the method 418 can involve, for example, step 420, step 422, step 424, or any combination thereof. The steps of method 418 can be executed in any order, for example, in the order shown in FIG. 25B. Any of the steps can be omitted. FIG. 25B illustrates that the method 418 can be performed using the device 50 for the injection of the material 160c for assisting in the gelation of the material 160a after the conduit 52 has been inserted into the body space 8 and the opening 50z of the conduit 52 has access to the body space. FIG. 25B illustrates, for example, that the injection of a media the can be used as a primer for assisting in the gelation of the material 160a (e.g., a hydrogel) in the body space 8. As a primer, the media can wash the body space 8 of excess materials, provide greater lubricity, deliver proteins to accelerate gelation of the hydrogel, provide for a uniform pH, or a pH beneficial to gelation. Step 420 can include, for example, inserting the conduit 52 into the body space 8. Step 422 can include, for example, injecting the material 160c into the body space 8. The material 160c can be, for example, phosphate buffered saline, saline, Ringer's lactate or other biocompatible fluid with a pH or property that opens the lumen, wets or changes the lubricity, affects the pH, or washes away materials in the lumen of the vas deferens 3. Injection of the material 160c can be pressure regulated, flow rate regulated, volume controlled, or any combination thereof. Step 424 can include, for example, preparing the environment of the body space 8 (also referred to as the body space environment and the intraluminal environment) for gelation of the material 160a (e.g., a hydrogel). After the injection of the material 160c, the intraluminal environment can be uniform in terms of lubricity, pH, or without remnant sperm and proteins.

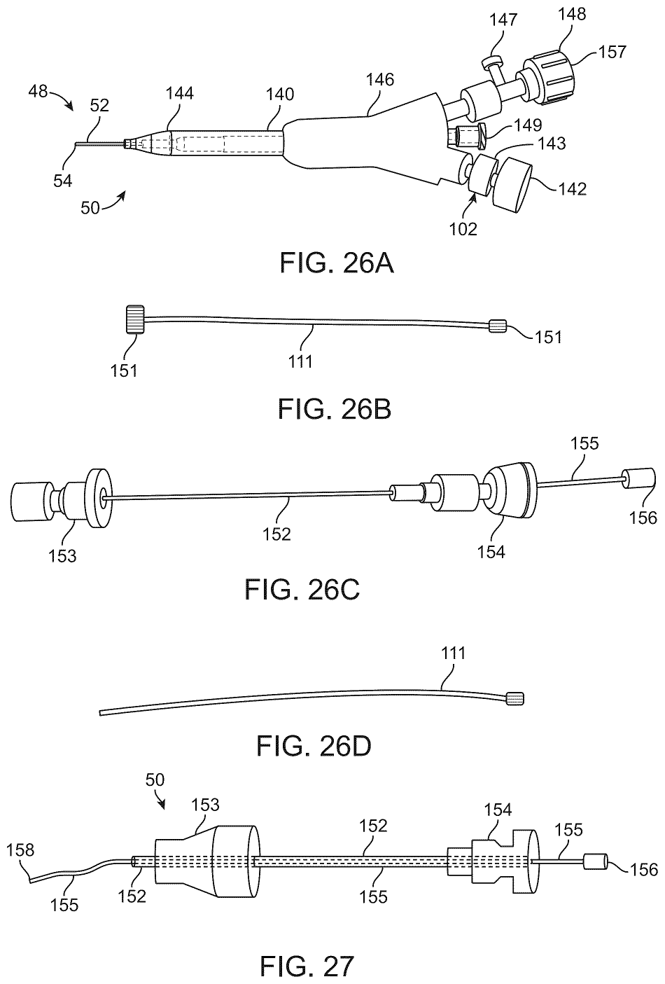

FIGS. 26A-26D illustrate a variation for the delivery of the material 160a using the cartridge 111 with plunger 152.

FIG. 26A illustrates components of that the device 50 can have, including, for example, the conduit 52, a Touhy connector 148 for the probe 55 with a probe opening 157 and an irrigation flush port 147. The proximal end of the device 50 can have, for example, a cartridge port 149, a needle removal hub 142 (e.g., for the needle 54), the penetration control 143, or any combination thereof.

FIG. 26B illustrates that the cartridge 111 can have the material 160a (e.g., a hydrogel) and can have cartridge caps 151 on both ends.

FIG. 26C illustrates that the device can have a plunger 152 with a push rod connector 153, a push rod hub 154, a distal mini push rod 155 and a distal mini push rod hub 156.

FIG. 26D illustrates a variation of the cartridge 111. The mini push rod 155 can be configured to pass through the conduit 52 to deliver the material 160a.

FIG. 27 illustrates a hydrogel cartridge with flexible tip plunger. FIG. 27 illustrates a hydrogel cartridge with an alternative plunger 152 with the mini push rod 155 and mini push rod flexible tip 158. The mini flexible tip 158 can be configured to adapt to the curvature of the vas deferens or body lumen during delivery of the hydrogel.

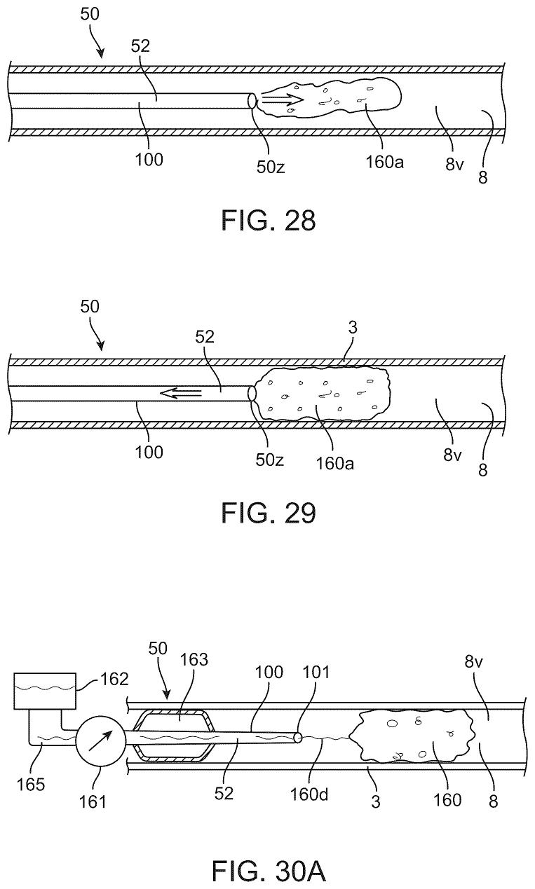

FIG. 28 illustrates in a cross-sectional view antegrade delivery with the conduit 52 placed within the vas deferens lumen 8v and injecting the material 160a (e.g., a hydrogel) within the vas deferens lumen 8v.

FIG. 29 illustrates in a cross-sectional view retrograde delivery of the material 160a (e.g., a hydrogel) within the vas deferens lumen 8v. During this delivery, the ejection of material 160a can occur simultaneously with the retraction of the conduit 52.

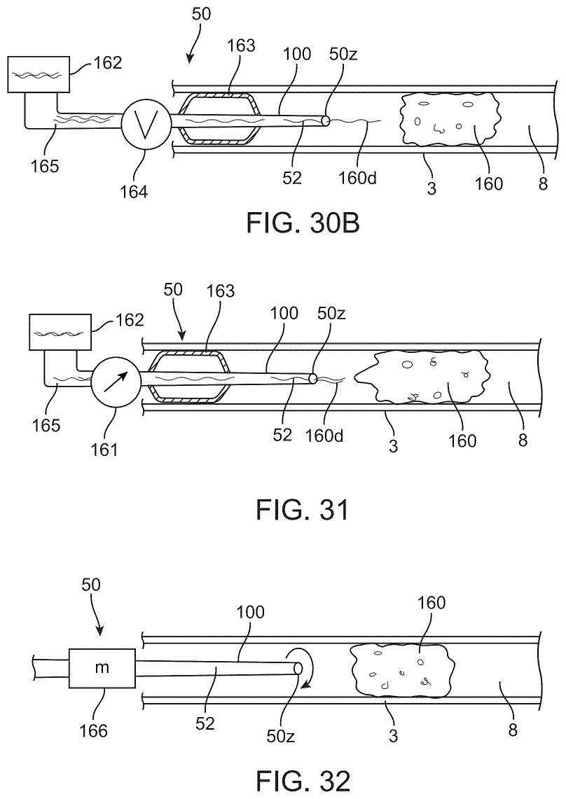

FIGS. 30A-30B show a variation for confirming occlusion of the body lumen after hydrogel delivery and removal of the system from the body space 8.

FIG. 30A illustrates the injection of a media 160d (e.g., gas, air) for confirming occlusion using a force measurement system 161. The injection of media can also be used for initiating gelation of the material 160a (e.g., the hydrogel). In practice, media supply 162 can be connected to the media force measurement device 161 via media conduit 165 and can be connected to the conduit 52 within the vas deferens lumen 8v. The media 160d can be instilled through distal end opening 50z and can meet the occlusive device (e.g., the material 160a). The conduit 52 can have a proximal occlusion balloon 163 (balloon lumen and balloon inflation device not shown) to prevent media pressure from passing retrograde of the system.

FIG. 30B illustrates injection of the media 160d for confirming occlusion using a volume measurement device 164 with a similar configuration as the media force measurement system 161. In practice, a predetermined volume of the media 160d can instilled into the body space 8 (e.g., of the vas deferens lumen 8v) to determine when a threshold volume of the media 160d can be and/or is delivered into the body space 8 from the conduit 52 and occlusive device or hydrogel 160a. In practice, a predetermined volume of the media 160d can instilled into the body space 8 (e.g., of the vas deferens lumen 8v) to determine when a threshold volume of the media 160d has been delivered into the body space 8 from the conduit 52 and occlusive device or hydrogel 160a. The conduit 52 can have a proximal occlusion balloon 163 (balloon lumen and balloon inflation device not shown) to prevent media flow in the retrograde direction.

FIG. 31 illustrates a variation with the injection of the media 160d for creating space in the body space 8 from the distal end opening 50z of the conduit 52 toward the material 160a with the intention of preventing a back flow, tail, or remnant hydrogel from exiting the vas deferens 3. In practice, after (e.g., immediately after) or near the final injection of the material 160a, the media 160d can be injected to prevent the back flow, tail, or remnant material 160a (e.g., remnant hydrogel) from flowing in a retrograde direction.

FIG. 32 illustrates a technique for the removal of the conduit 52 after the injection of the material 160a with simultaneous rotation of the conduit 52 around its central axis during the injection of the material 160a, and removal of the conduit 52, for example, to prevent a tail of the material 160a from exiting the vas deferens 3 as the conduit 52 is removed from the vas deferens 3. FIG. 32 illustrates that a rotational motor 166 at the proximal end of the conduit 52 can facilitate uniform and constant rotation of the conduit 52. The rotational motor 166 can be employed during just the removal of the conduit 52 from the vas deferens 3. As another example, the rotational motor 166 can be replaced by non-motorized gearing (not shown) that automatically rotates the conduit 52 during withdrawal. As another example, the user can manually rotate the conduit 52 during removal of the conduit 52 from the body space 8.

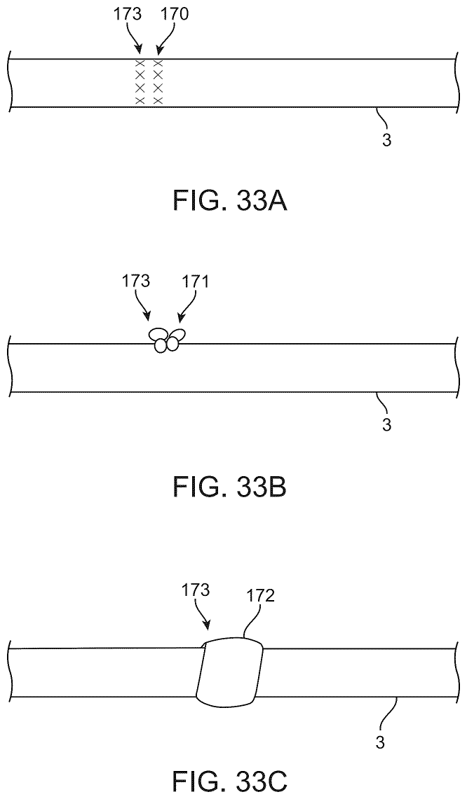

FIGS. 33A-33C show systems for future identification of the target site 173 (e.g., delivery site) of the material 160a (e.g., the occlusive hydrogel) in the body space 8.

FIG. 33A illustrates a system where the target site 173 (e.g., delivery site) of insertion into the vas deferens 3 can be tagged with a visual tattoo 170 on the external surface of the vas deferens 3.

FIG. 33B illustrates a system for future identification of the target site 173 (e.g., delivery site) of the material 160a (e.g., occlusive hydrogel) in the body space 8 where the site of insertion into the vas deferens 3 can be tagged with an external clip 171 that can be visible via ultrasound or radiographic imaging. The external clip 171 may not occlude the vas deferens 3, but can mark the target site 173 (e.g., delivery site) of insertion that can be identified in the future.

FIG. 33C illustrates a system for future identification of the target site 173 (e.g., delivery site) of the material 160a (e.g., occlusive hydrogel) in the body space 8 where the site of insertion into the vas deferens 3 can be tagged with a circumferential band 172 to permanently identify the site of insertion. The circumferential band 172 can be slightly elastomeric and may not be restrictive or compressive enough to occlude the vas deferens 3 and/or may be restrictive enough to not create tissue necrosis.

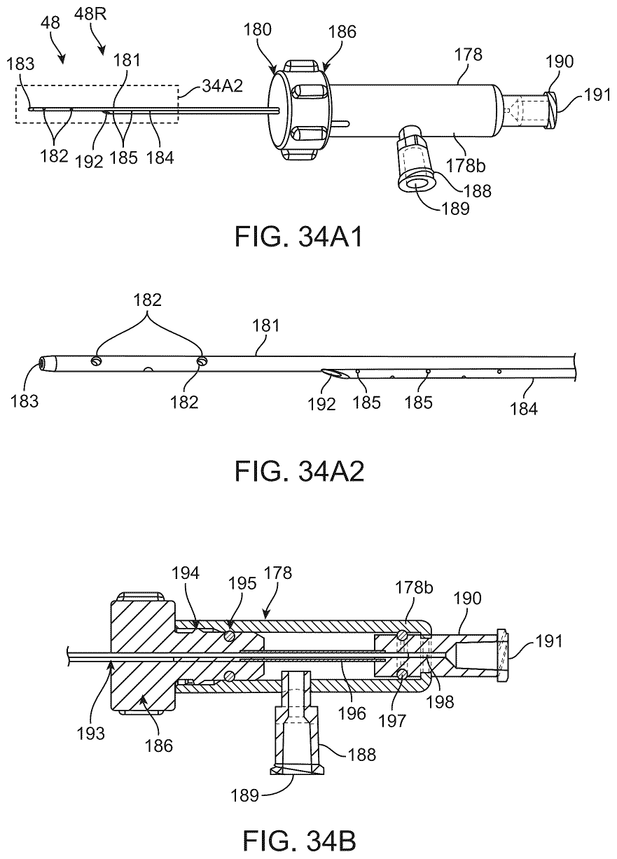

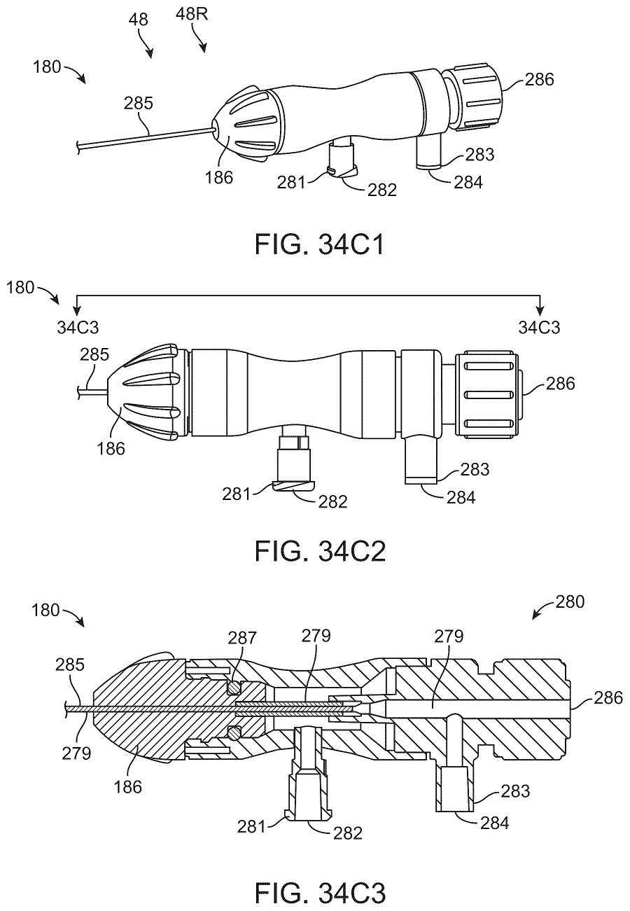

FIGS. 34A1-34C3 illustrate that the system 48 (e.g., the occlusion removal system 48R) can have an occlusion removal device 180. FIGS. 31A1-34C3 illustrate a variation of the system 48, for example, a variation of the device 180.

FIG. 34A1 illustrates that the device 180 can have a handle 178. The handle 178 can be rotatable. The device 180 can remove the material 160a (e.g., a hydrogel) from the body space 8 with an intraluminal removal sheath 179 (also referred to as the removal sheath 179), a control 186 (e.g., a rotatable knob), and can be configured in a similar fashion with a probe (e.g., the probe 55) and can require translation of the probe 55 the threshold distance D6 as confirmation of intraluminal access as previously described with the device 50. FIG. 341 illustrates that the handle 178 can have a handle body 178b. The handle body 178b can be, for example, a housing of the handle 178. The handle body 178b can be, for example, a stationary or a rotatable handle housing. FIG. 34A1 illustrates that the control 186 can be rotated relative to the handle body 178b. FIG. 34A1 illustrates that the device 180 can have irrigation holes 182 in the wall of an irrigation lumen 181 and can have aspiration holes 185 in the wall of an aspiration lumen 184. The irrigation lumen 181 can be connected to an irrigation connector 190 with irrigation connector opening 191 for the instillation of media such as sodium bicarbonate solution or other media that affect, dissolve, break cross-links, change the pH or properties of the material 160a (e.g., the occlusive device or hydrogel) for removal from the body space 8. Such media can be, for example, the material 160b. The material 160b can be and/or can comprise, for example, sodium bicarbonate solution or other media that affect, dissolve, break cross-links, change the pH or properties of the material 160a (e.g., the occlusive device or hydrogel) for removal from the body space 8. The irrigation or source of the material 160b is not shown. The material 160b can exit the irrigation lumen 181 from irrigation holes 182. The irrigation hole 182 at the distal end of the irrigation lumen can be, for example, the distal end opening 183 of the irrigation lumen 181. The aspiration lumen 184 can be connected to vacuum port 188 and vacuum port opening 189 for the application of negative pressure within the body space 8 to remove excess material 160b and loose portions of the material 160a (e.g., the occlusive device or hydrogel). Vacuum source is not shown. FIG. 34A1 illustrates that the irrigation lumen 181 can have an outer diameter, for example, of 0.8 mm.

FIG. 34A2 shows irrigation lumen 181 with irrigation holes 182 and aspiration lumen 184 with distal end opening of aspiration lumen 192 and additional aspiration holes 185. For both irrigation and aspiration, the number of lumen holes can be expanded or reduced.

FIG. 34B illustrates that the handle 178 can have a rotatable seal 195, a rotatable seal 197, the vacuum port 188, and the irrigation port 190. The handle 178 can allow for the concurrent application of irrigation of media (e.g., the material 160b) and aspiration of excess media (e.g., excess material 160b) and dissolved material 160a (e.g., dissolved hydrogel) from the body space 8 during the removal process. FIG. 34B illustrates that the handle body 178b can be held stationary while the control 186, which can be permanently fixed to the dual lumen, is rotated. The center of axis can be the irrigation lumen. This can remain concentric to the control 186 and the handle body 178b while the vacuum or aspiration lumen rotates around the central axis. FIG. 34B illustrates that the overall length of the handle 178 can be, for example, about 60 mm. FIG. 34B illustrates that the device 180 can have an adhesive bond 193, a snap fit 194, a stiffening tube 196, and irrigation media 198.

FIGS. 34C1-34C3 illustrates two views of a variation of the device 180. FIGS. 34C1-34C3 illustrate that the device 180 can have the arrangement of features shown, for example, an aspiration port 281, an aspiration opening 282, an irrigation port 283, an irrigation opening 284, a proximal end opening 286, and a dual lumen sheath 285. The dual lumen sheath 285 can be connected to the device 180 with a central lumen (e.g., as shown in FIG. 34C3) connected to a guidewire or probe port 286 (e.g., a port for the probe 55) on the proximal for the placement of a probe (e.g., the probe 55). FIG. 34C3 illustrates the device 180 in a cross-sectional view, showing a rotatable seal 287, and a central lumen 279 that traverses from the probe port 286 on the proximal end through the dual lumen sheath 285.

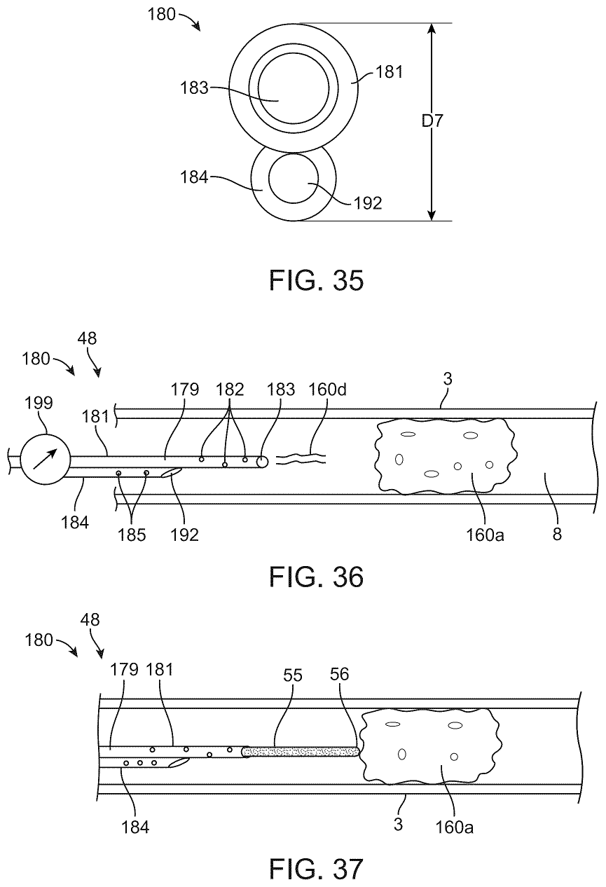

FIG. 35 illustrates the device 180 with eccentric lumens in an end-on view from the distal end opening of the irrigation lumen 183 and distal end opening of the aspiration lumen 192. The probe for intraluminal confirmation (e.g., the probe 55) can be housed or applied in either the irrigation or aspiration lumen. FIG. 35 illustrates that the inner diameter of the irrigation lumen 183 can be, for example, 1.4 mm, that the inner diameter of the aspiration lumen 184 can be, for example, 0.8 mm or 0.9 mm, and that the distance D7 can be, for example, 1.27 mm.

FIG. 36 illustrates an occlusion measurement or identification system for use at the time of removal of an occlusive device (e.g., the material 160a). With intraluminal placement in a body lumen, media (e.g., the material 160d) can be delivered to confirm occlusion of the body lumen or vas deferens 3 by a hydrogel using a force or pressure measurement device 199. The media can be, for example, saline, phosphate buffered saline, Ringer's lactate, or other biocompatible fluid. The media can be a gas like CO2 as well. In practice, the dual lumen sheath 179 with irrigation lumen 181 can be used for the delivery of media, and the pressure measurement device 199 can confirm the presence of the occlusion in the vas deferens 3.

FIG. 37 illustrates that a probe (e.g., the probe 55) can be advanced or translated to confirm occlusion in the body space 8 (e.g., the vas deferens) by force measurements collected on the proximal end of the probe 55 by a force measurement system. Force measurements experienced from the advancement of the probe 55 interacting with the intraluminal occlusion (e.g., the material 160a) can be recorded.

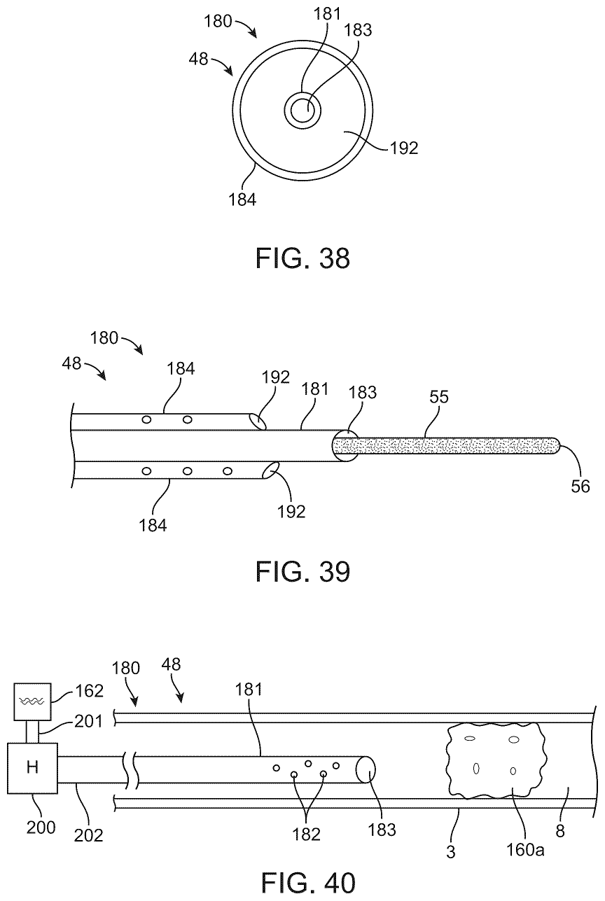

FIG. 38 illustrates instillation of the material 160b (e.g., sodium bicarbonate). FIG. 38 illustrates in an end-on view of the distal end opening for the dual lumen removal sheath of the device 180 with concentric lumens. The central lumen shows the distal end opening of the irrigation lumen 183 of the irrigation lumen 181, with the outer lumen having the aspiration lumen 184 and distal end opening of the aspiration lumen 192. The concentric design can be used for the removal of the material 160a (e.g., the hydrogel) with the instillation of a dissolving media (e.g., the material 160b) into the body space 8 with concurrent irrigation and aspiration. The dissolving media can be, for example, sodium bicarbonate solution comprising 8.4% sodium bicarbonate. The concentration of sodium bicarbonate can range, for example, from 4% to 10%. The instillation of the sodium bicarbonate solution can be performed with a two-lumen, co-linear concentric sheath system.

FIG. 39 illustrates that the dual lumen sheath system can have a central lumen 181 with distal end opening 183 for probe 55 passage and irrigation, with eccentric aspiration lumens 184 on the anterior and posterior surface of the irrigation lumen 181 for removal of excess media and dissolved hydrogel. FIG. 39 illustrates that the device 180 can have a central lumen for the probe 55 and eccentric lumens for aspiration.

FIG. 40 illustrates that the removal of the material 160a (e.g., an occlusion device or hydrogel can be with a heating source 200 (e.g., an additional heating source) where media or sodium bicarbonate solution (e.g., the material 160b) can be administered at body temperature (37° C.), or a slightly higher temperature than body temperature but not enough create tissue necrosis, to accelerate the dissolution action on the hydrogel. The elevated temperature can range from 37.1° C. to 40° C. Media supply 162 can be placed through heating source 200 to elevate media temperature and can be directed through the media heater connector 202 to the irrigation lumen 181 and into the body lumen through irrigation holes 182 and irrigation distal end opening 183.

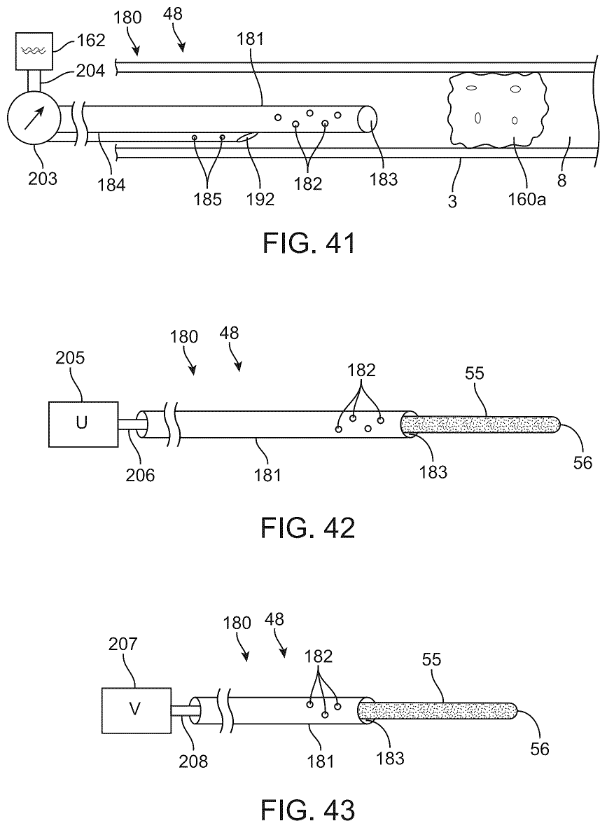

FIG. 41 illustrates that irrigation and aspiration can be controlled by a pressure regulator. FIG. 41 illustrates that the removal of an occlusion device or hydrogel (e.g., the material 160a) can be with irrigation and aspiration controlled by a pressure regulator 203 and the intraluminal pressure within the body lumen can be kept equal to or below 3.0 psi during continuous irrigation through irrigation lumen 181 and aspiration through aspiration lumen 184. Pressure regulation can minimize the amount of media distension within the vas deferens 3. The maximum intraluminal pressures can range, for example, from about 0.1 psi to about 6.0 psi.

FIG. 42 illustrates that the removal of an occlusion device or hydrogel (e.g., the material 160a) can be with a probe (e.g., the probe 55) that can be configured to provide mechanical agitation with an ultrasonic generator source 205 during the irrigation and aspiration process to accelerate the surface contact, break up, and dissolution of the hydrogel (e.g., the material 160a) by the sodium bicarbonate solution (e.g., the material 160b) delivered through irrigation lumen 181 and irrigation holes 182. Mechanical agitation can be by an ultrasonic generation source connected with ultrasonic generator connector 206 to the proximal end of the probe 55. FIG. 42 illustrates mechanical agitation by an ultrasonic generator.

FIG. 43 illustrates that the removal of an occlusion device or hydrogel (e.g., the material 160a) can be with a probe (e.g., the probe 55) that can be configured to provide mechanical agitation with a vibratory source 207 during the irrigation and aspiration process to accelerate the surface contact, break up, and dissolution of the hydrogel (e.g., the material 160a) by the sodium bicarbonate solution (e.g., the material 160b) delivered through irrigation lumen 181 and irrigation holes 182. Mechanical agitation can be provided by a vibratory source connected with vibration connector 208 to the proximal end of the probe 55. FIG. 42 illustrates mechanical agitation by vibratory source.

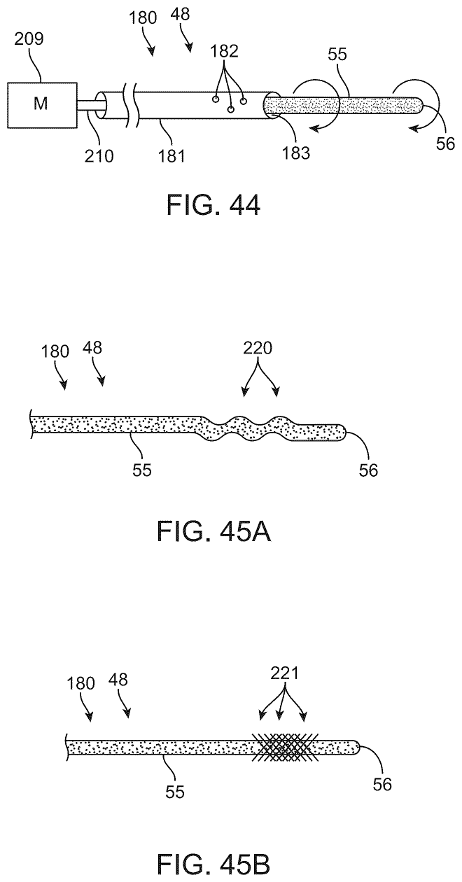

FIG. 44 illustrates a variation of mechanical agitation that can be provided by a rotary motion source 209 providing rotational motion and is connected by rotary motion source connector 210 to the proximal end of the probe 55. FIG. 44 illustrates that the probe can be rotated by a rotational motor.

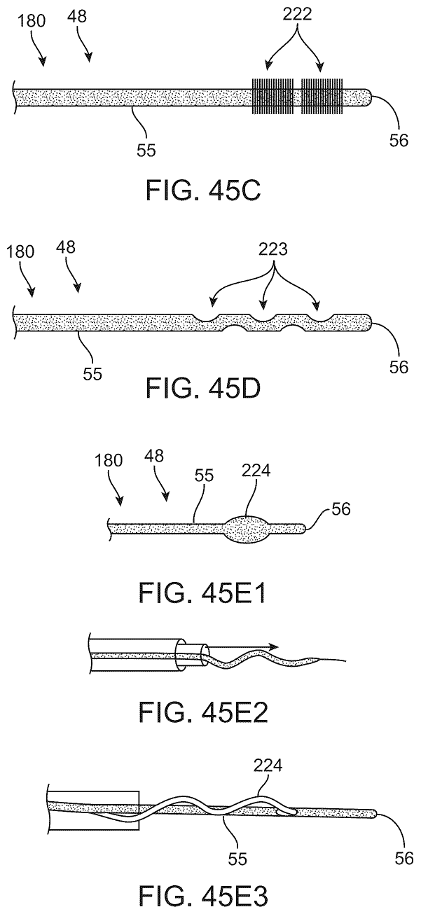

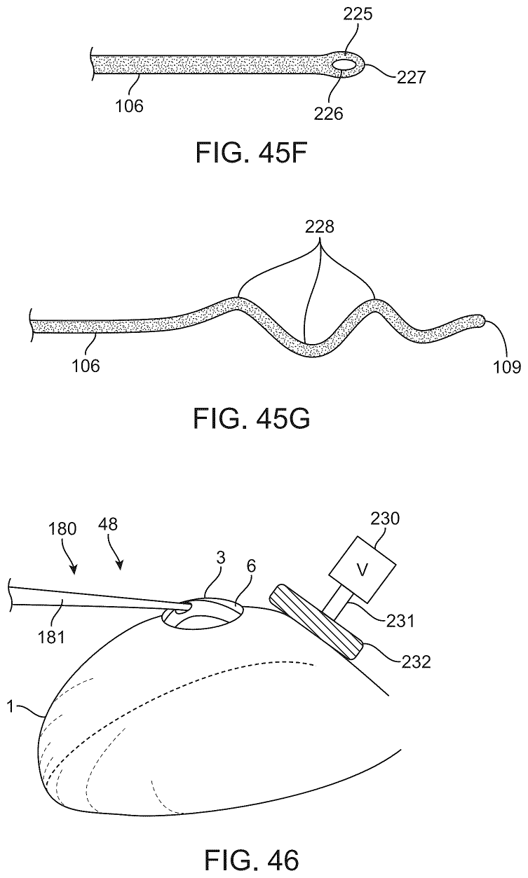

FIGS. 45A-45G show a variation for enhanced mechanical agitation from the probe 55 within a body space 8 for accelerated dissolution of an occlusion device (e.g., the material 160a).

FIG. 45A demonstrates that the mechanical action of probe 55 can be enhanced by protrusions 220 on the probe 55, for example, near the probe tip 56.

FIG. 45B illustrates that the probe 55 can have bristles 221. The bristles 221 can be attached to the probe 55, for example, proximal and/or near the probe tip 56. The bristles 221 can be designed to agitate the occlusive device (e.g., the material 160a) during removal.

FIG. 45C illustrates that the probe 55 can have brushes 222 attached to the probe 55, for example, proximal and/or near the probe tip 56. The brushes 222 can be configured to agitate the occlusive device during removal.

FIG. 45D illustrates that the probe 55 can have indentations 223. The indentations can be, for example, near the probe tip 56 as shown, and can be configured to agitate the occlusive device during removal.

FIGS. 45E1-45E3 illustrates that the probe 55 can have coils 224. The coils 224 can be attached to the probe 55, for example, proximal and/or near the probe tip 56. The coils 224 can be designed to agitate the occlusive device (e.g., the material 160a) during removal.

FIG. 45F illustrates that the probe 55 can have a loop 225 with an opening 226 that extends partially or completely through the probe 55. FIG. 45F illustrates that the opening 226 can extend completely through the probe 55. The loop 225 can be proximal and/or near the probe tip 56. The loop 225 can be configured to agitate the occlusive device during removal. The probe 55 can have multiple loops near the distal end.

FIG. 45G illustrates that the probe 55 can have angulations 228. The angulations 228 can be proximal and/or near the probe tip 56. The angulations 228 can be configured to agitate the occlusive device during removal. The angulations 228 can be pre-formed or designed to retain their shape on the probe 55 to provide mechanical action on the occlusive device.

FIG. 46 illustrates that an external vibratory source 230 can be applied to external portion of the body space 8 during instillation of the dissolving media or sodium bicarbonate solution (e.g., the material 160b) to enhance the agitation and action of the dissolving media (e.g., the material 160b). In practice, a vibratory source pad 232 can be applied on the scrotum 1 to apply vibration forces during the irrigation and aspiration application for removal by sheath 181 in the vas deferens 3 that is exposed through the scrotal skin opening 6.

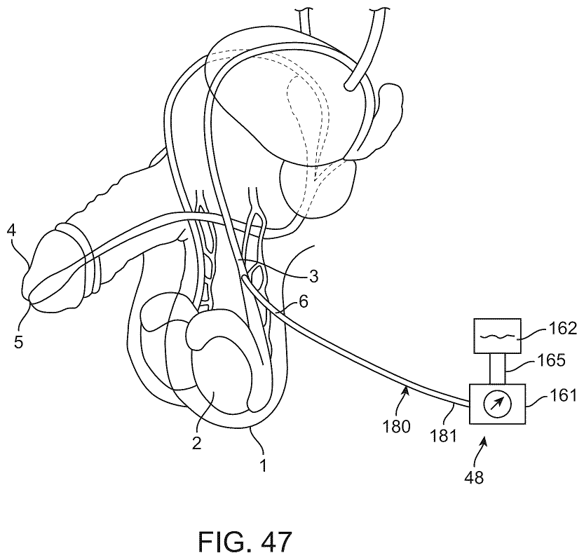

FIG. 47 illustrates that the device 180 can supply a lavage of sodium bicarbonate (e.g., the material 160b) at the completion of the removal step. Other lavage media options for the material 160b can include, for example, saline, phosphate buffered saline, Ringer's lactate, and other biocompatible media suitable for the reproductive tract. The removal system can have a media supply 162 (e.g., a supply of the material 160b) connected to media conduit 165 and force or flow measurement system 161 to confirm or quantitate the restoration of patency in the body space 8 during lavage. The media for lavage (e.g., the material 160b) can be supplied to the vas deferens 3, for example, through scrotal skin opening 6 and/or percutaneously. Excess lavage can traverse the reproductive tract and exit the urethra 5.

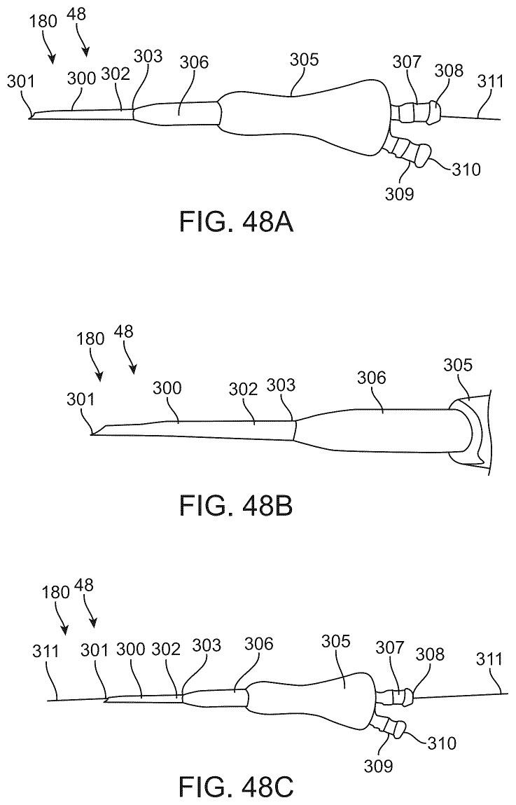

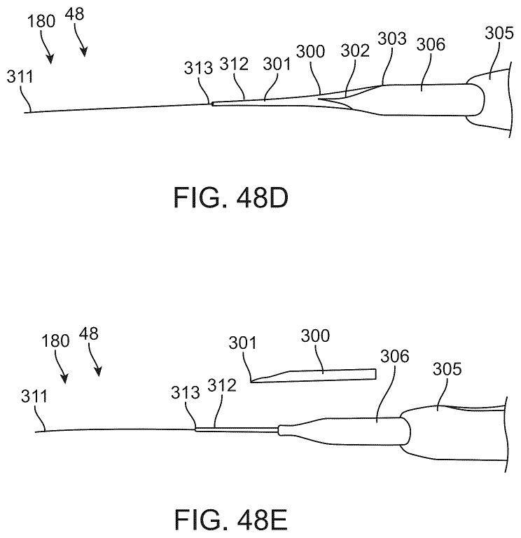

FIGS. 48A-48E illustrate a variation of the device 180, for example, with a penetrating sheath configured to wrap around the probe and intraluminal catheter. The wrap around sheath can replace the need for a needle, a half needle, blunt dissection using the curved forceps, lancet, or trocar. After entry to the body space 8 has been established, the wrap around sheath can be retracted, peeled away, or removed from the handle once entry into the body space 8 has been established (e.g., confirmed). The wrap around sheath can facilitate the exchange of deviceation without increasing the outer diameter of the entry site into the body space 8. Column strength of the wrap around penetrating sheath by the tightness of the circular wraps of sheath material like a tight funnel shape. The wrap around penetrating sheath can be sized to perform the following steps: a) enter the body space 8, b) expand enough to only allow the probe (e.g., the probe 55) to pass through its center and into the body space 8, c) expand further to only allow the access sheath to pass through its center and over the probe (e.g., the probe 55), and/or d) fully expand at its split to be retracted onto the delivery or removal handle or be retracted and peeled away from the entire handle assembly.

Wrap around penetrating sheaths can be made from thin wall polymers such as PEEK, PET, nylon and other biocompatible polymers that have strength in thin wall sections. Other material choices include, for example, stainless steel, titanium, nitinol, or other biocompatible metals.

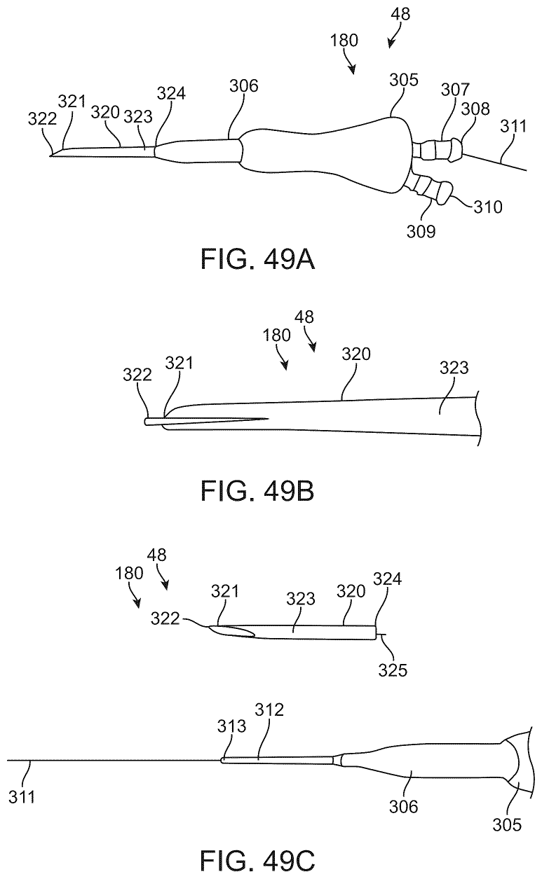

FIG. 49A illustrates that the device 180 can have the wrap around penetrating sheath 300 at the distal end of a delivery or removal handle 305. FIG. 49A illustrates that the device 180 can have a connector 307 having a probe connector port 308 and a connector 309 having an irrigation and/or aspiration connector port 310 on the proximal end of the delivery or removal handle 305. The conduit 52 can be, for example, the wrap around penetrating sheath 300.

FIG. 48B illustrates a close-up view of the wrap around penetrating sheath 300 with sharp distal tip of wrap around penetrating sheath 301, the pre-made split within the wrap around penetrating sheath 302, and proximal end of wrap around penetrating sheath 303 positioned on nose cone of delivery or removal handle 306. In this configuration, the distal tip of wrap around penetrating sheath 301 can be introduced into the body space 8 be penetrating the anterior wall.

FIG. 48C illustrates the configuration of the wrap around penetrating sheath 300 once entry into the body space 8 has been established and probe 311 is advanced through the distal opening of the wrap around penetrating sheath 300.

FIG. 48D illustrates the wrap around penetrating sheath 300 slightly retracted with both the probe 311 and intraluminal catheter 312 through the distal opening of the wrap around penetrating sheath 300. To accommodate the retraction process, the split within wrap around penetrating sheath 302 can expand onto the nose cone of the delivery or removal handle 306.

FIG. 48E illustrates the wrap around penetrating sheath 300 completely retracted and separate from the delivery or removal handle 305. The split within wrap around penetrating sheath 302 can extend the entire length of the wrap around penetrating sheath 300, for example, to enable complete retraction or peel away from delivery or removal handle 305.

FIGS. 49A-49C illustrate a variation of a wrap around penetrating sheath with a sharp pointed needle or lancet configured at the distal end to enhance entry and penetration into the body space 8. The needle can be placed axially within the wrap around sheath and can provide greater column strength or structure to the wrap around sheath for penetration.

FIG. 49A illustrates the delivery or removal handle 305 with a wrap around penetrating sheath with needle 320.

FIG. 49B illustrates a close-up view of the wrap around penetrating sheath with needle 320 with sharp needle tip 322 located at the distal tip of the wrap around penetrating sheath 321. An axial split within the wrap around penetrating sheath 323 can run the entire length of sheath.

FIG. 49C illustrates the wrap around penetrating sheath with needle 320 completely retracted and separate from the delivery or removal handle 305 with the extended probe 311 and access sheath 312. The probe 312 can be, for example, the probe 55.

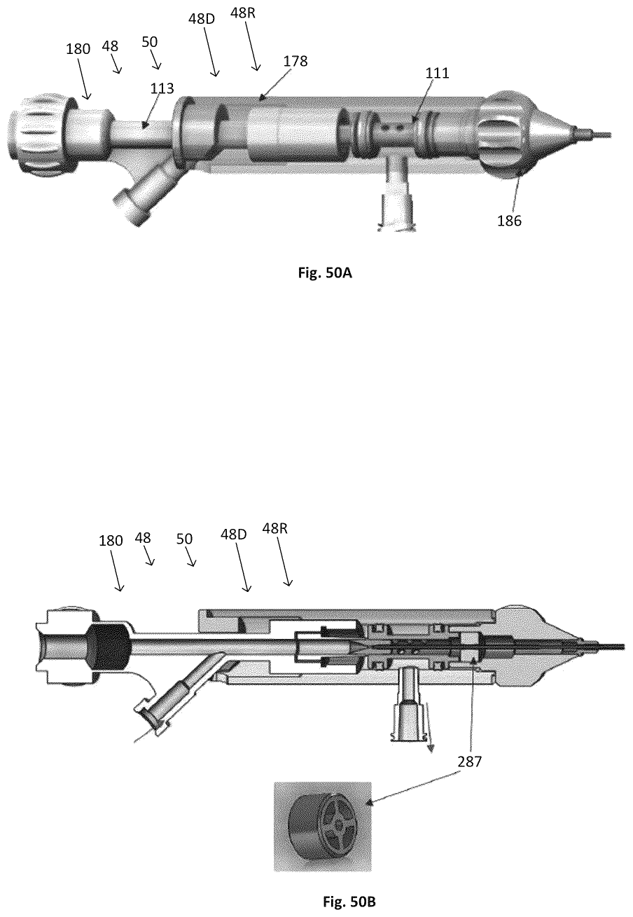

FIGS. 50A-50B illustrate a removal system with alternating lumens for the irrigation and aspiration of the hydrogel. FIG. 50A illustrates the removal handle with a transparent section identifying the alternating lumens mechanism. FIGS. 50A-50B illustrate that the irrigation and aspiration tubings can have pressure relief valves to ensure that the intraluminal pressure of the vas deferens 3 can be controlled. Examples of pressure ranges can be, for example, from 1.0 psi to 10.0 psi, including every 0.1 psi increment within this range (e.g., 3.0 psi as a nominal value). The irrigation and aspiration of the hydrogel can be monitored as a function of time. Examples of time durations can be, for example, 1 minute to 10 minutes of irrigation and aspiration, including every 1 second increment within this range (e.g., 4 minutes as a nominal time duration). Alternatively or additionally, the irrigation and aspiration step can be performed using a predetermined volume of sodium bicarbonate solution, of other dissolving solution, and terminating the step when the fluid volume is exhausted. Examples of fluid volumes can include, for example, 10 cc to 250 cc, or 50 cc as a nominal fluid volume. At the conclusion of the irrigation and aspiration step, a final irrigation lavage can be performed with the visual confirmation of effluent from the patient's urethra to signify a patent vas deferens. Additionally, the advancement of the removal catheter past the hydrogel implantation site is another confirmation that the occlusive hydrogel has been removed. FIG. 50B illustrates the removal system in cross-section with a close-up illustration of the rotation wheel for alternating the lumens. FIG. 50B illustrates that the rotatable seal 287 (e.g., a rotating seal) can be a wheel that seals the inner lumens and allow the delivery instrument 50 to rotate axially around the removal instrument with the rotatable handle 178.

FIGS. 50A-50B illustrate that the features of the device 50 and the features of the device 180 can be combined with each other in any combination. FIGS. 50A-50B illustrate that the system 48 can include the device 50, a device 50 that has any of the features of the device 180, the device 180, a device 180 with any of the features of the device 50, the device 50 and the device 180, or any combination thereof. As another example, the device 50 can be, for example, a first device, and the device 180 can be, for example, a second device or vice versa. As another example, a single device can be a delivery and removal device such that a single device can have any combination of features disclosed herein.

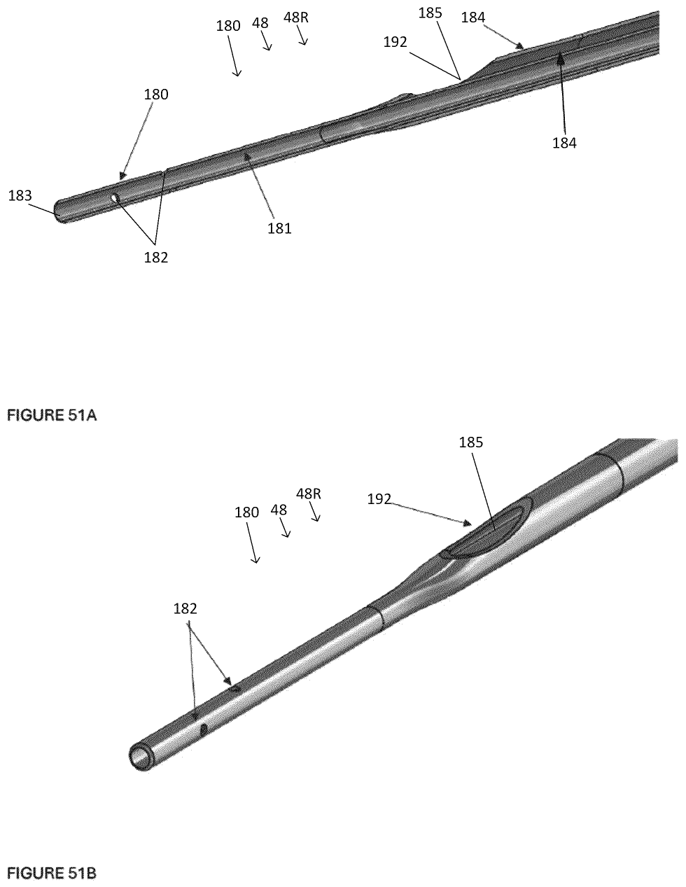

FIGS. 51A-51B illustrate another removal catheter system with the distal end of the removal system and dual-lumen catheters. FIG. 51A illustrates in cross-section the distal end of the dual-lumen catheter. FIG. 51B illustrates a side-view of the distal end of the removal catheter with the aspiration hole and irrigation side holes.

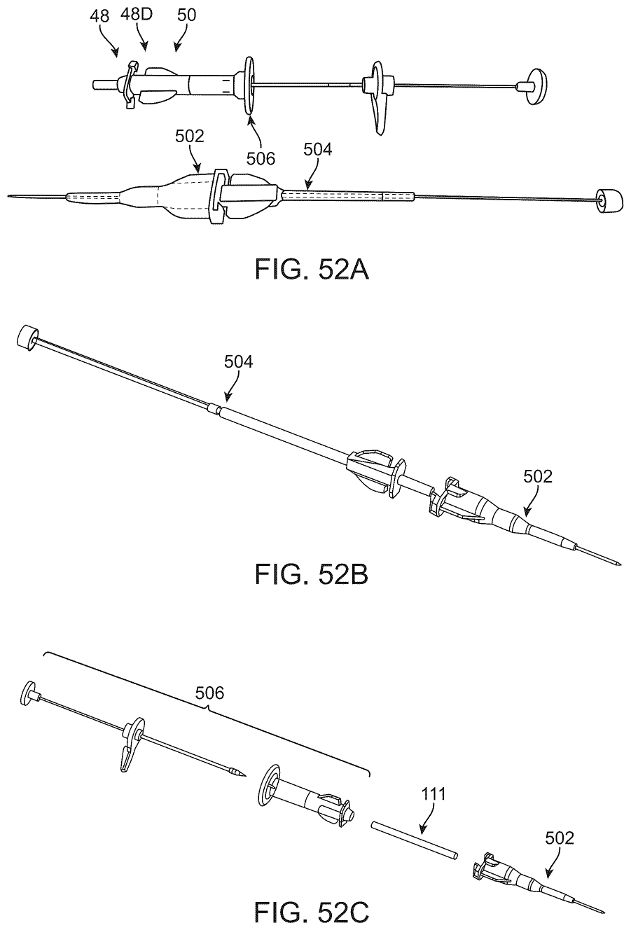

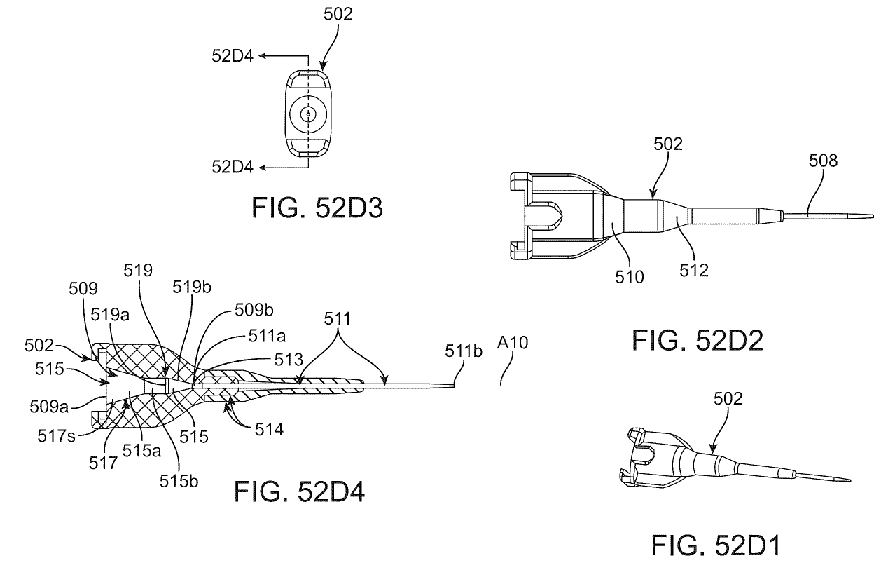

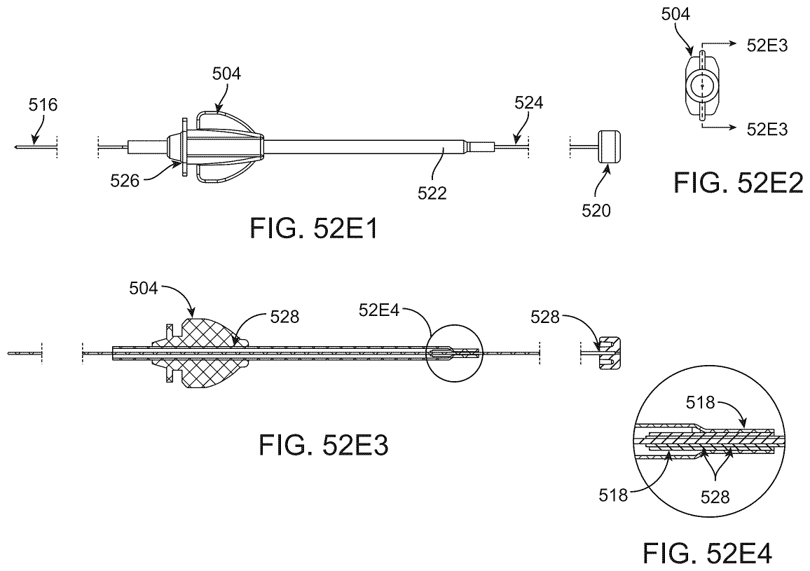

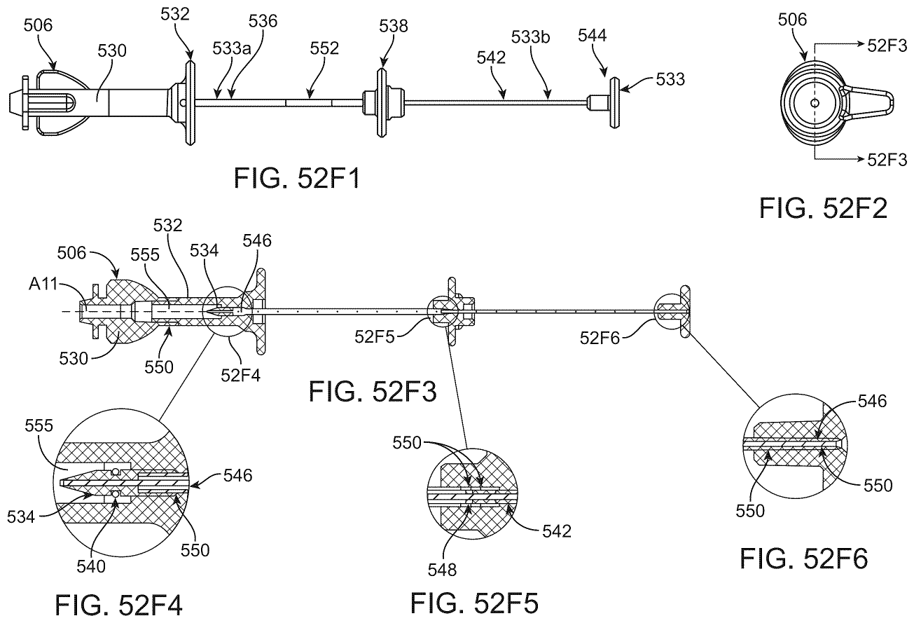

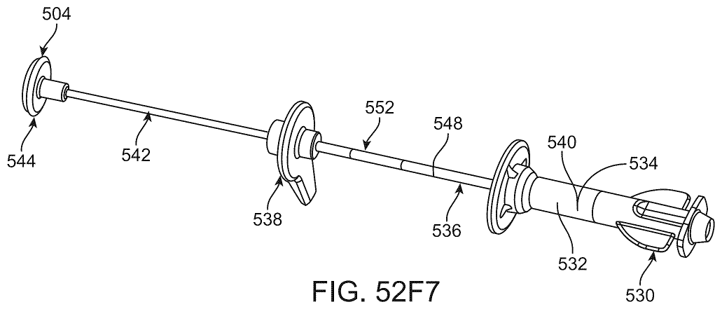

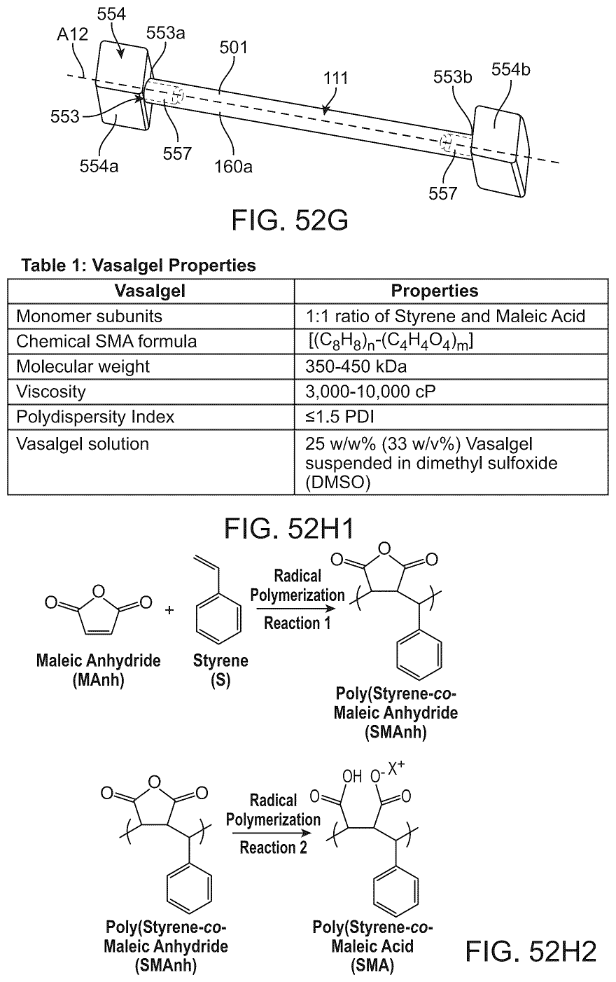

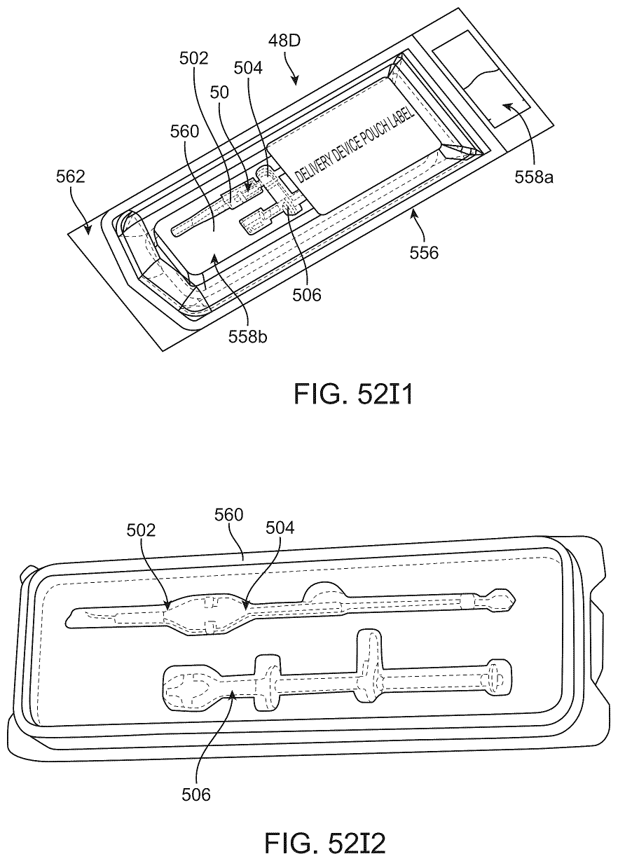

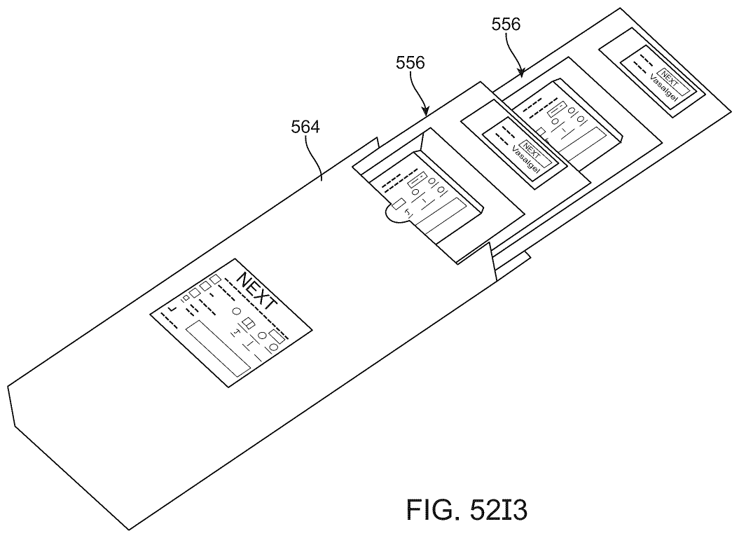

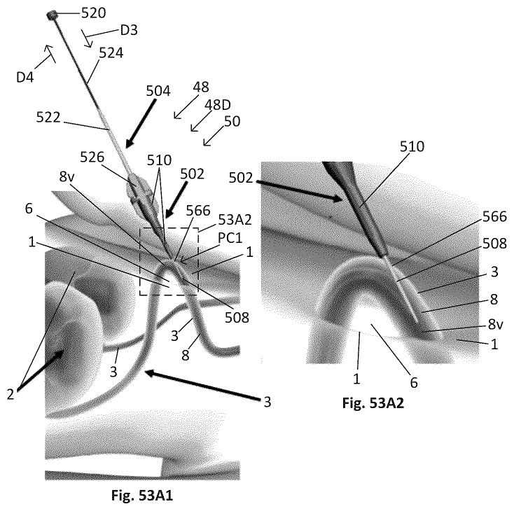

FIG. 52A illustrates that the system 48D can have a device 50 with the arrangement of features shown. FIG. 52A illustrates that the device 50 can have a sheath assembly 502, a probe assembly 504, and a handle assembly 506. FIG. 52A illustrates the probe assembly 504 can be attached to the sheath assembly 502. FIG. 52A illustrates, for example, the sheath assembly 502 attached to (e.g., loaded with) the probe assembly 504. The sheath assembly 502 can be, for example, a 2F sheath assembly. The probe assembly 504 can be, for example, a guidewire assembly (e.g., a 0.014 inch guidewire assembly). The device 50 can deliver the material 160 into the body space 8 (e.g., into the vas deferens lumen 8v).

FIG. 52B illustrates a perspective view of the sheath assembly 502 and the probe assembly 504, for example, with the arrangement of features shown.

FIG. 52C illustrates that the system 48D can have the cartridge 111. The cartridge 111 can have the material 160a. The cartridge 111 can be, for example, a hydrogel (e.g., Vasalgel®) filled cartridge. FIG. 52C illustrates that the handle assembly 506 can be loaded with the cartridge 111. FIG. 52 illustrates an exploded view of the sheath assembly 502, the handle assembly 504, and the cartridge 111.

FIGS. 52D1-52D4 illustrate that the sheath assembly 502 can have a sheath 508, a sheath hub 510, a strain relief, 512, an adhesive 514, or any combination thereof, for example, in the arrangement shown. FIG. 52D4 illustrates that the sheath assembly 502 can have a sheath assembly space 509 and/or a sheath assembly lumen 511. FIG. 52D4 illustrates that the sheath assembly lumen 511 can have a center longitudinal axis A10. The center longitudinal axis A10 of the access lumen 511 can be, for example, the center longitudinal axis of the sheath assembly 502. FIGS. 52D2 and 52D4 illustrate that the sheath assembly 504 can have the dimensions shown in parentheses (e.g., units can be inches). These dimensions can vary, with values greater or less than those shown.

The sheath assembly space 509 can be a space in the sheath hub 510. For FIG. 52D4 illustrates that the assembly space 509 can be a space in a proximal end of the sheath 510. The sheath assembly space 509 can have a space proximal opening 509a and a space distal opening 509b. The space proximal opening 509a can be, for example, the proximal terminal opening of the sheath assembly space 509, and/or the space distal opening 509b can be, for example, the distal terminal opening of the sheath assembly space 509. FIG. 52D4 illustrates that the space proximal opening 509a can be, for example, the proximal terminal opening of the sheath assembly 502 (e.g., of the sheath hub 510).

The sheath assembly lumen 511 can extend through the sheath 508 and/or the sheath hub 510. For example, FIG. 52D4 illustrates that the sheath assembly lumen 511 can extend through the sheath 508 and the sheath hub 510. FIG. 52D4 illustrates that the sheath assembly lumen 511 can have a lumen proximal opening 511a and a lumen distal opening 511b. The lumen proximal opening 511a can be, for example, the proximal terminal opening of the sheath assembly lumen 511, and/or the lumen distal opening 511b can be, for example, the distal terminal opening of the sheath assembly lumen 511. FIG. 52D4 illustrates that the lumen distal opening 511b can be, for example, the distal terminal opening of the sheath assembly 502 (e.g., of the sheath 508). The lumen distal opening 511b can be, for example, the opening 50z.

FIG. 52D4 illustrates that the space distal opening 509b and the lumen proximal opening 511a can be inside the sheath hub 510. FIG. 52D4 illustrates that the space distal opening 509b and the lumen proximal opening 511a can be the same opening (e.g., as indicated by the opening 513 in FIG. 52D4), whereby the sheath assembly space 509 and the sheath assembly lumen 511 can merge at the opening 513. The opening 513 can be, for example, the merge point of the sheath assembly space 509 and the sheath assembly lumen 511. As another example, that the space distal opening 509b and the lumen proximal opening 511a can be different openings.

FIG. 52D4 illustrates that the sheath assembly space 509 can have one or multiple sections 515 (e.g., 1-5 sections 515, including every 1 section increment within this range, for example, 1 section, 2 sections, 5 sections). For example, FIG. 52D4 illustrates that the sheath assembly space 509 can have three sections 515. For example, FIG. 52D4 illustrates that the sheath assembly space 509 can have a space first section 515a, a space second section 515b, and/or a space third section 515, for example, in the arrangement shown. For example, the space first section 515a can be the proximal-most section, the space second section 515b can be between the space first section 515a and space the third section 515c, and the space third section 515c can be the distal-most section. The sections 515 can have any shape, including, for example, tapered and/or non-tapered shapes. For example, FIG. 52D4 illustrates that the space first section 515a and the space third section 515c can be defined by tapered surfaces and that the space second section 515b can be defined by a non-tapered surface. For example, the space first section 515a can have the shape of a truncated cone, the space second section 515b can have the shape of a cylinder, and the space third section 515c can have the shape of a cone with an opening (e.g., the opening 513) at the apex. FIG. 52D4 illustrates that the space sections 515 can be defined by one or multiple interior walls of the sheath hub 510.

FIG. 52D4 illustrates that the sheath hub 510 can have a guide 517 having a tapered surface 517s. FIG. 52D4 illustrates that the space first section 515a can be, for example, a guide space defined by the tapered surface 517s. The guide 517 can be configured, for example, to guide the cartridge 111 into the space second section 515b.

FIG. 52D4 illustrates that the sheath hub 510 can have one or multiple stops 519 (e.g., 1-5 stops 519, including every 1 stop increment within this range, for example, 1 stop, 2 stops, 5 stops). For example, FIG. 52D4 illustrates that the sheath hub 510 can have a first stop 519a and/or a second stop 519b.

The first stop 519a can be, for example, a cartridge stop. The first stop 519a can be configured, for example, to prevent the cartridge 111 from being advanced distally beyond the first stop 519a in the sheath hub 510. For example, FIG. 52D4 illustrates that the first stop 519a can prevent the cartridge 111 (e.g., the distal end terminal end of the cartridge 111) from being advanced into the space third section 515c from the space second section 515b. For example, FIG. 52D4 illustrates that the first stop 519a can prevent the cartridge 111 from entering the space third section 515c. The first stop 519a can be, for example, a narrowing of the sheath assembly space 509 to a size smaller than the width (e.g., diameter) of the cartridge 111. The first stop 519a can be, for example, a step 523 in the portion of the wall of the sheath hub 510 that defines the distal end of the space second section 515b. The step 523 can extend into the sheath assembly space 509, for example, radially into the space second section 515b. The step 523 can, for example, define the distal terminal end of the space section 515b. As another example, the first stop 519a can be the proximal end of the second stop 519b. FIG. 52D4 illustrates that the second stop 519b can be a tapered surface, whereby the first stop 519a can be, for example, the proximal end of the tapered surface.

The second stop 519b can be, for example, a plunger tip stop. The second stop 519b can be configured, for example, to prevent the plunger tip 534 (see e.g., FIGS. 52F1-52F7) from being advanced into the sheath 508 and/or into the sheath assembly lumen 511. FIG. 52D4 illustrates, for example, that the second stop 519b can prevent the plunger tip 534 (see e.g., FIGS. 52F1-52F7) from being advanced into the sheath 508 and into the sheath assembly lumen 511. For example, FIG. 52D4 illustrates that the second stop 519b can prevent the plunger tip 534 (see e.g., FIGS. 52F1-52F7) entering the portion of the sheath assembly lumen 511 in the sheath hub 510 and the portion of the sheath assembly lumen 511 in the sheath 508.

The sheath assembly 502 (e.g., the sheath 508) can enter a wall of the body space 8. For example, the sheath 508 can enter a wall (e.g., the anterior wall) of the vas deferens 3. In practice, the wall of the vas deferens 3 (e.g., the anterior wall) can be opened by the physician with the distal tip of dissecting forceps (e.g., the forceps 22), for example, in the same fashion that the scrotal skin is opened via blunt dissection in a no-scalpel technique, and then the sheath 508 can be inserted through the wall of the body space 8 and into the body space 8.

FIGS. 52E1-52E4 illustrate that the probe assembly 504 can have a probe 516 (e.g., a guidewire), a probe stop extrusion 518 (e.g., a guidewire stop extrusion), a probe knob 520 (e.g., a guidewire knob), a distal tube extrusion 522, a probe proximal tube 524 (e.g., a guidewire proximal tube), a probe hub 526 (e.g., a guidewire hub), an adhesive 528, or any combination thereof, for example, in the arrangement shown. The probe 516 can be, for example, the probe 55, a guidewire, or both. FIGS. 52E1-52E4 illustrate that a proximal end of the probe 516 can be connected to the probe proximal tube 524. FIGS. 52E1-52E4 illustrate that the probe 516 and the probe proximal tube 524 can extend through the distal tube extrusion 522. As another example, the probe proximal tube 524 can be a proximal end of the probe 516. FIGS. 52E1-52E4 illustrate that the probe 516 can be translated (e.g., pushed) through the distal tube extrusion 522 and the probe hub 526, for example, by pushing probe knob 518 toward the probe hub 526. The probe hub 526 can be removably attached to the sheath hub 510 or vice versa. FIGS. 52E1 and 52E3 illustrate that the probe assembly 504 can have the dimensions shown in parentheses (e.g., units can be inches). These dimensions can vary, with values greater or less than those shown.

Once the tip of the sheath 508 is in the body space 8 (e.g., the vas deferens lumen 8v), the probe 516 can be used to confirm placement of the tip of the sheath 508 in the body space 8, for example, in the intraluminal space of the vas deferens 3. The probe 516 can be advanced the threshold distance D6 (e.g., a minimum of 5 cm) into the body space 8, for example, to signify proper location of the sheath 508 (e.g., the tip of the sheath 508) in the body space 8. After translation by the threshold distance D6 (e.g., a minimum of 5 cm) is accomplished with the probe 516 without restriction, the sheath 518 can be advanced over-the-probe (e.g., over-the-wire) to a distance of 0.5 cm-3.0 cm into the vas deferens lumen 8v, or more narrowly, 1.0 cm-1.5 cm into the vas deferens lumen 8v, including every 0.1 cm increment within these ranges (e.g., 0.5 cm, 1.0 cm, 1.5 cm, 2.0 cm, 3.0 cm).

FIGS. 52A-52B show the probe 516 in a retracted configuration (e.g., in a fully retracted configuration). When the probe 516 is in a fully retracted configuration, the distal end of the probe 516 can be in the sheath 508, for example, in a lumen of the sheath (e.g., lumen 50y). For example, FIGS. 52A-52B illustrate that when the probe 516 is in a fully retracted configuration, the distal end of the probe 516 can be inside the distal end of the sheath 508. FIGS. 52A-52B illustrate that the probe 516 can be advanced out of the sheath 508 relative to the sheath assembly 502, for example, by moving (e.g., pushing) the probe 516 toward the probe hub 526 via the probe knob 520 and/or the probe proximal tube 524. The probe 516 can be retracted from the sheath assembly 502, for example, by moving (e.g., pulling) the probe 516 away from the sheath assembly 502 by pulling the probe knob 520 and/or the probe proximal tube 524, by disconnecting the probe assembly 504 from the sheath assembly 502, or any combination thereof. The probe 516 can be retracted and removed from the vas deferens lumen 8v, for example leaving the sheath 508 providing access to the vas deferens lumen 8v.

FIGS. 52F1-52F7 illustrate that the handle assembly 506 can have a distal cap 530, a housing 532, a plunger assembly 533, an adhesive 550, a space 555 (also referred to as the housing space 555), a laser mark 552 or other markings as indicia to provide the user a visual indicator of the amount of travel or delivery, or any combination thereof, for example, in the arrangement shown. The distal cap 530 can be attached to or integrated with the housing 532. For example, FIGS. 52F1-52F7 illustrate that the distal cap 530 can be a distal end of the housing 532. FIGS. 52F1-52F7 illustrate that the housing 532 can have a housing flange 535. FIGS. 52F1-52F7 illustrate that the housing 532 can have an opening 531. The opening 531 can be, for example, the distal opening of the handle assembly 506 (e.g., of the housing 532), for example the distal terminal opening of the housing 532 (e.g., the distal cap 530). The cartridge 111 can be loaded into and/or unloaded from the space 555, for example, through the opening 531. As another example, the cartridge 111 can be loaded into the space 555 but may not be unloaded from the space 555. The space 555 can be, for example, a cartridge docking area (e.g., the cartridge docking area 253). The space 555 can be, for example, a space of the handle assembly 506. FIG. 52F3 illustrates that the space 555 can have a center longitudinal axis A11. The center longitudinal axis A11 of the space 555 can be, for example, the center longitudinal axis of the probe assembly 504.

FIGS. 52F1-52F7 illustrate that the plunger assembly 533 can have a first plunger 533a, a second plunger 533b, a plunger tip 534, a seal 540 (e.g., e.g., an O-Ring), a plunger rod 546, or any combination thereof. The seal 540 can be, for example, a 0.039 in.×0.22 in. O-Ring. FIGS. 52F1-52F7 illustrate that the first plunger 533a can have a first plunger tube 536 and/or a first plunger knob 538. FIGS. 52F1-52F7 illustrate that the second plunger 533b can have a second plunger tube 542, a second plunger knob 544, and/or a second plunger stop 548. FIGS. 52F1-52F7 illustrate that the first plunger 533a and the second plunger 533b can both connected to the plunger tip 534, for example, via the plunger rod 546 such that both the first plunger 533a and the second plunger 533b can move the plunger tip 534. The first plunger 533a and the second plunger 533b can be separate plungers, for example, connected to the same plunger tip 534 and/or to the same plunger rod 546. As another example, the first plunger 533a and the second plunger 533b can be the same plunger, for example, whereby the first plunger 533a can be a first portion of the plunger and the second plunger can be a second portion of the plunger. FIG. 52F1 illustrates that the handle assembly 506 can have the dimensions shown in parentheses (e.g., units can be inches). These dimensions can vary, with values greater or less than those shown.

When the cartridge 111 is loaded into the space 555 and the handle assembly 506 is attached to the sheath assembly 502, the plunger assembly 533 (e.g., the plunger tip 534 and/or the plunger rod 546) can be advanced relative to the cartridge 111 and the space 555 by advancing the first plunger 533a and/or the second plunger 533b to move the plunger tip 534 and/or the plunger rod 546 through the cartridge 111 and the space 555. Advancing the plunger tip 534 through the cartridge 111 and the space 555 can force (e.g., push) the material 160a out of the cartridge 111 and the space 555 and into the sheath assembly 502, for example, through the opening 531 and into the sheath 508. As another example, advancing the plunger tip 534 through the cartridge 111 and the space 555 can force (e.g., push) the material 160a out of the cartridge 111 and the space 555 and into the sheath assembly 502, for example, through the opening 531, into the sheath 508, and into the body space 8. When the cartridge 111 is loaded into the space 555 and the handle assembly 506 is attached to the sheath assembly 502, the plunger assembly 533 (e.g., the plunger tip 534 and/or the plunger rod 546) can be advanced out of the opening 531 and into the sheath assembly 502, for example, to force (e.g., push) the material 160a in the sheath assembly 502 (e.g., in the sheath 508) out of the sheath 508 and into the body space 8.

FIG. 52F4 illustrates that the seal 540 can prevent the material 160a from being drawn into the plunger assembly 533, for example, into the channel that the plunger rod 546 is movable in (e.g., translatable in) as the first plunger 533a is advanced and/or retracted relative to the housing 532 and/or as the second plunger 533b is advanced and/or retracted relative to the housing 532.

FIG. 52G illustrates that the cartridge 111 can have openings 553, for example, a cartridge first opening 553a on a first end of the cartridge 111 and a cartridge second opening 553b on a second end of the cartridge 111. The cartridge 111 can be a hollow cylinder that can house the material 160a. For example, FIG. 52G illustrates that the cartridge 111 can be a hollow tube. FIG. 52G illustrates that the cartridge 111 can have a reservoir 501 (also referred to as the cartridge space 501) that can have the material 160a. The cartridge space 501 can be, for example, the lumen of the cartridge 111. The reservoir 501 can be, for example, a lumen that extends through the cartridge 111 from the cartridge first opening 553a to the cartridge second opening 553b. The system 48D can include, for example, the device and the cartridge 111.

FIG. 52G illustrates that the cartridge 111 have caps 554. The caps 554 can be, for example, removably attachable to the cartridge 111. The caps 554 can be used, for example, to removably plug the openings 553. FIG. 52G illustrates, for example, that the caps 554 can be attached to the cartridge 111 to keep the material 160a inside the cartridge space 501. FIG. 52G illustrates that a cap 554 can be attached to each end of the cartridge 111. For example, FIG. 52G illustrates that a first cap 554a can be attached to a first end of the cartridge 111, and that a second cap 554b can be attached to a second end of the cartridge 111. Each of the caps 554 can have an extension 557 that can be inserted into the cartridge space 501. FIG. 52G illustrates that the first cap 554a can be attached to the cartridge 111 by inserting the extension 557 of the first cap 554a into the cartridge space 501 through the cartridge first opening 553a, and illustrates that the second cap 554b can be attached to the cartridge 111 by inserting the extension 557 of the second cap 554b into the cartridge space 501 through the cartridge second opening 553b. When the caps 554 are attached to the cartridge 111 and the extensions 557 are in the cartridge space 501 (e.g., as shown in FIG. 52G), the extensions 557 can be, for example, seals that can prevent the material 160a from flowing or leaking out of the cartridge 111. The caps 554 can be attached to the cartridge 111, for example, with a screw fit, a snap fit, a friction fit, or an combination thereof, for example, between the extensions 557 and the cartridge 111. For example, FIG. 52G illustrates that the extensions can have a friction fit with the cartridge 111. The caps 554 can be attached and removed from the cartridge 111, for example, by pushing and pulling the caps 554 onto and off of the cartridge 111, respectively.

FIG. 52G illustrates that after the cartridge 111 is loaded with the material 160a, the caps 554 can be attached to the cartridge 111. FIG. 52G illustrates the material 160a (e.g., a hydrogel such as Vasalgel®) can be packaged in the cartridge 111 (e.g., in the cartridge space 501). The caps 554 can keep the material 160a in the cartridge 111, for example, until the cartridge 111 is loaded into the handle assembly 506, at which point the caps 554 can be removed (e.g., as shown in FIG. 52C). The cartridge 111 can be vacuum sealed in a pouch (e.g., in the first pouch 558 as shown in FIG. 52I1). Once the cartridge 111 is removed from the pouch and the caps 554 are removed, the cartridge 111 can be inserted into the handle assembly 506 (e.g., into the space 555 of the housing 532). The cartridge 111 and/or the caps 554 can be made, for example, from polyethylene, polypropylene, PEEK, and/or other biocompatible material. For example, the cartridge 111 and/or the caps 554 can be made from and/or comprise a high-density polyethylene (HDPE). For example, the cartridge 111 can be a HDPE capped cartridge. FIG. 52G illustrates that the cartridge space 501 can have a center longitudinal axis A12. The center longitudinal axis A12 of the cartridge space 501 can be, for example, the center longitudinal axis of the cartridge 111.

FIGS. 52A-52G illustrate that the device 50 can have the sheath assembly 502, the probe assembly 504, and the handle assembly 506 loaded with the cartridge 111. The cartridge 111 can hold a volume of the material 160a (e.g., a hydrogel such as Vasalgel®), for example, in the cartridge space 501. The volume of the material 160a that the that the cartridge 111 can hold (e.g., in the cartridge space 501) can range, for example, from 10 μl to 250 μl, or more narrowly, 25 μl to 150 μl, including, for example, every 1 μl increment within these ranges (e.g., 10 μl, 25 μl, 50 μl, 100 μl, 150 μl, 250 μl). The volume of the material 160a that the that the cartridge 111 can hold can be, for example, the total volume of the cartridge space 501. As another example, the total volume of the cartridge space 501 can be greater than the volume of the material 160a that the cartridge 111 can hold, for example, to accommodate the volume of the cartridge space 501 that the caps 554 (e.g., the extensions 557) can occupy when the caps 554 are attached to the cartridge 111 (e.g., as shown in FIG. 52G). The total volume of the cartridge space 501 can be, for example, for example, 5 μl to 50 μl greater than the volume of the material 160a that the cartridge 111 can hold. The total volume of the cartridge space 501 can range, for example, from 15 μl to 300 μl, for more narrowly, 30 μl to 200 μl, including, for example, every 1 μl increment within these ranges (e.g., 15 μl, 30 μl, 55 μl, 60 μl, 75 μl, 100 μl, 105 μl, 150 μl, 155 μl, 200 μl, 255 μl, 300 μl). Dimensionally, the cartridge 111 can be sized and shaped to fit into a 1 mL syringe. For example, the cartridge 111 can have a cylindrical shape having an outer diameter of 2 mm and a length of 6 cm. Other outer diameters and lengths of the cylindrical shape are possible. In practice, the plunger assembly (e.g., the first plunger 533a and/or the second plunger 533b) can be advanced to pass the plunger tip 534 through the cartridge 111 (e.g., through the plunger space 501) to deliver the material 160a through the handle assembly 506, through the sheath assembly 502, and into the body space 8. The cartridge 111 can have any size and shape, including, for example, cylindrical, spherical, or rectangular. The cartridge 111 can be a blister (e.g., a foil-wrapped blister) that can house (e.g., hold) the material 160a. In practice, the cartridge 111 (e.g., a blister, a hollow cylinder) can be pierced for the delivery of the material 160a with the handle assembly 506. For example, advancing the plunger tip 534 via the first plunger 533a or the second plunger 533b can pierce the cartridge 111 (e.g., a blister, a hollow cylinder). When the cartridge 111 is a hollow cylinder, for example, as shown in FIG. 52G, piercing the cartridge 111 can include, for example, advancing the plunger tip into the first opening 553a or the second opening 553b (whichever of the openings 553 was inserted first into the housing space 555 in the handle assembly 506). As another example, the cartridge 111 can have a foil, or multiple foil wrappings, that can be pierced by the plunger assembly 533 (e.g., by the plunger tip 534) to deliver the material 160a through the handle assembly 506. The plunger tip 534 can be advanced to pierce the foil, or multiple foil wrappings, for example, by advancing the plunger tip 534 via the first plunger 533a and/or via the second plunge 533b to deliver the material 160a through the handle assembly 506.

FIGS. 52A-52G illustrate once the caps 554 are removed from the cartridge 111, the cartridge 111 can be inserted into the handle assembly 506 (e.g., see FIG. 52C). The handle assembly 506 having the cartridge 111 can then be connected onto the sheath hub 510. Once connected, the first plunger 533a can be advanced to deliver the material 160a into the body space (e.g., the vas deferens lumen 8v). The second plunger 533b can be used to expel the final portion of material 160a into the vas deferens lumen 8v. After delivery of the material 160a into the vas deferens lumen 8v, the device 50 can be removed and the contralateral vas deferens 3 can accessed with a new, second device 50, whereby two identical devices can be used, a first device 50 (e.g., as shown in FIGS. 52A-52F7) for a first vas deferens 3 and a second device 50 (e.g., as shown in FIGS. 52A-52F7) for a second vas deferens 3 (e.g., the contralateral vas deferens 3).

The material 160a can be, for example, a hydrogel such as Vasalgel®. Vasalgel® is a polymer comprised of a 1:1 ratio of styrene and maleic acid monomeric subunits (SMA). The chemical formula is [(C₈H₈)ₙ—(C₄H₄O₄)ₘ]. The SMA is dissolved in dimethyl sulfoxide (DMSO) at a ratio of 25% w/w SMA and 75% w/w DMSO. Vasalgel® has a molecular weight range of 350-450 KDaltons and it is sterilized using electron beam irradiation. FIG. 52H1 illustrates Table 1 which details the properties of Vasalgel®.

FIG. 52H2 illustrates that the synthesis of Vasalgel® is achieved by a two-step process. The first step is a free radical solution copolymerization of styrene and maleic anhydride producing the styrene/maleic anhydride copolymer. The second step involves the hydrolysis of the anhydride groups to yield the styrene/maleic acid copolymer (SMA). Under nitrogen blanketing, the SMA material is then dissolved in DMSO and packaged in a capped cartridge (e.g., as shown in FIG. 52G) and vacuum sealed in a foil pouch (e.g., as shown in FIG. 52I1).

FIG. 52I1 illustrates that the occlusion delivery system 48D can be packaged as separate components in a package 556. For example, 52I1 illustrates that the cartridge 111 having the material 160a, the sheath assembly 502, the probe assembly 504, and the handle assembly 506 can be packaged in the package 556. The package 556 can have one or multiple holders 558 (e.g., 1-5 holders 558, including every 1 holder increment within this range, for example, 1 holder, 2 holders 5 holders), a tray 560, or any combination thereof. FIG. 52I1 illustrates, for example, that the package 556 can have a first holder 558a for the cartridge 111 having the material 160a, and can have a second holder 558b for the sheath assembly 502, the probe assembly 504, and the handle assembly 506. The holders 558 can be, for example, pouches. For example, FIG. 52I1 illustrates that the first holder 558a can be a first pouch and the second holder 558b can be a second pouch. FIG. 52I1 illustrates that the material 160a can be placed into the cartridge 111, the cartridge 111 can be capped with the caps 554, and the capped cartridge can be vacuum sealed in the first holder 558a and e-beam sterilized separately. The first holder 558a can be, for example, a foil pouch. FIG. 52I1 illustrates that the sheath assembly 502, the probe assembly 504, and the handle assembly 506 can be placed in the tray 560, and can be sealed inside of the second holder 558b and sterilized by e-beam separately. The tray 560 can be, for example, a polyethylene terephthalate glycol (PETG) tray. The second pouch 558b can be, for example, a Tyvek pouch. FIG. 52I1 illustrates that when the device 50 is in the package 556, the probe assembly 504 can be attached to the sheath assembly 502. The sterile material 160a in the first pouch 558a can be kitted with the sterile components in the second pouch 558b. For example, FIG. 52I1 illustrates that the first pouch 558a can be externally adhered to the second pouch 558b.

FIG. 52I2 illustrates that the tray 560 can be removed from the package 556. FIG. 5212 illustrates that the tray 560 can have recesses for the sheath assembly 502, the probe assembly 504, and the handle assembly 506. For example, FIG. 52I2 illustrates that the tray can have a first recess for the sheath assembly 502 and the probe assembly 504, and can have a second recess for the handle assembly 506.

FIG. 52I3 illustrates that two of the kitted occlusion delivery systems 48D can be put into a package holder 564 (e.g., a shelf carton), for example, so that the physician can have two devices 50, one for a first vas deferens 3 and another for the contralateral vas deferens 3.

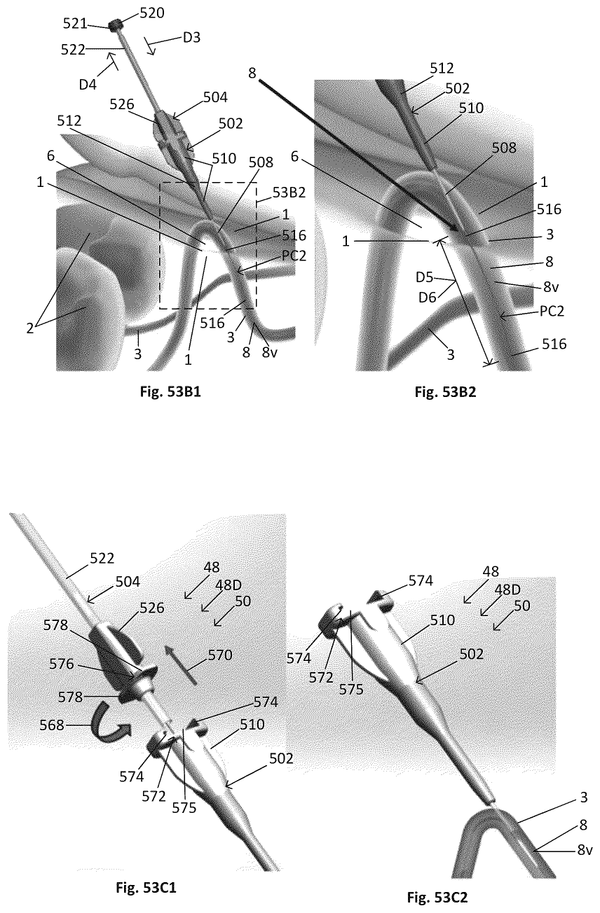

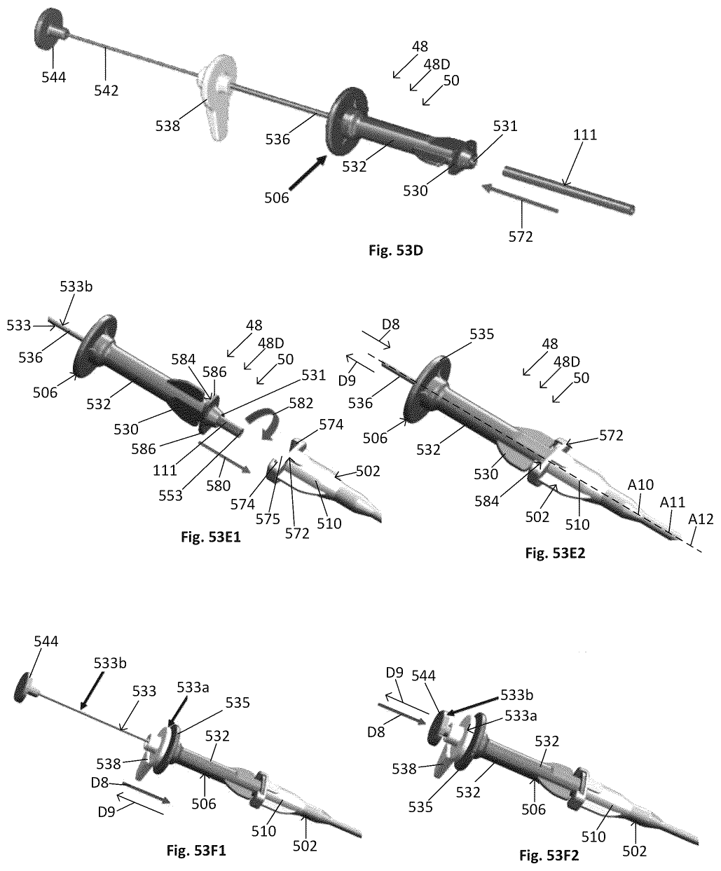

FIGS. 53A1-53F2 illustrate a variation of a process of accessing the body space 8 (e.g., the vas deferens lumen 8v) with the device 50 and deploying the material 160a into the body space 8. For example, FIGS. 53A1-53F2 illustrate the steps shown to deliver the material 160a into the vas deferens lumen 8v.

FIGS. 53A1-53A2 illustrate that the body space 8 (e.g., the vas deferens lumen 8v) can be accessed using the sheath assembly 502 and the probe assembly 504 of the device 50, for example, while the probe assembly 504 is attached to the sheath assembly 502.

FIGS. 53A1-53A2 illustrate that using standard no-scalpel vasectomy procedures, the vas deferens 3 can be locally anesthetized and exposed outside the scrotum 1 through a scrotal skin opening 6. Using the forceps 22 (e.g., as shown in FIG. 1C), FIGS. 53A1-53A2 illustrate that a puncture 566 (e.g., a small puncture) can be placed in a wall of the vas deferens 3 (e.g., the vas deferens anterior wall) for access to the vas deferens lumen 8v (e.g., as shown in FIG. 1C). FIGS. 53A1-53A2 illustrate, for example, that the device 50 may not have a needle (e.g., the needle 54). As another example, the device 50 (e.g., the device 50 shown in FIGS. 53A1-53A2) can have a needle (e.g., the needle 54).

FIGS. 53A1-53A2 illustrate that the sheath 508 can be pushed through the puncture 566 into the vas deferens lumen 8v. The portion of the sheath 508 in the vas deferens lumen 8v shown in FIGS. 53A1-53A2 may or may not have the probe 516. For example, FIGS. 53A1-53A2 illustrate that the portion of the sheath 508 in the vas deferens lumen 8v can have the probe 516 such that FIGS. 53A1-53A2 illustrate that the sheath 508 (e.g., the tip of the sheath 508) and the probe 516 (e.g., the tip of the probe 516) can be advanced through the puncture 566 together (e.g., at the same time) while the probe 516 is in the lumen of the sheath 508. FIGS. 53A1-53A2 illustrate, for example, that the sheath 508 and the probe 516 can be simultaneously pushed through the puncture 566 and into the vas deferens lumen 8v. FIGS. 53A1-53A2 illustrate that the sheath 508 and the probe 516 can be pushed through the puncture 566 while the probe 516 is in a retracted configuration (e.g., in a fully retracted configuration). FIGS. 53A1-53A2 illustrate that when the probe 516 is in a fully retracted configuration, the distal end of the probe 516 can be inside a distal end of the sheath 508. FIG. 53A1 illustrates that during insertion of the sheath assembly 502 and the probe assembly 504 into the body space 8, the sheath hub 510 and the probe hub 526 can be removably connected to each other.

FIGS. 53B1-53B2 illustrate that the probe 516 can be advanced a distance D5 out of the sheath 508 by moving (e.g., pushing) the probe knob 520 and/or the probe proximal tube 524 toward the sheath assembly 502. FIGS. 53B1-53B2 illustrate that the probe 516 can be advanced out of the sheath 508 a distance D5 less than or equal to the threshold distance D6 (e.g., 5 cm) into the body space 8. FIGS. 53B1-53B2 illustrate that the probe 516 can be advanced into the body space a distance D5 that equals the threshold distance D6 to confirm that the sheath 508 is in the body space 8 (e.g., the vas deferens lumen 8v). FIGS. 53B1-53B2 illustrate that the fully advanced position of the probe 516 can correspond to an advanced distance D5 that equals the threshold distance D6, for example, as measured from a distal terminal end of the sheath 508. FIGS. 53B1-53B2 illustrate, for example, that the threshold distance D6 can be the maximum distance D5 that the probe 516 can be advanced out of the sheath 508. FIGS. 53B1-53B2 illustrate, for example, that when the probe 516 is in a fully advanced configuration, the probe knob 520 can abut (e.g., can contact) the distal tube extrusion 522 at location 521. As another example, the maximum distance D5 that the probe 516 can be advanceable out of the sheath 508 can be greater than the threshold distance D6. FIGS. 53B1-53B2 illustrate that for the probe 516 to reach the threshold distance D6 as the probe 516 is advanced into the body space 8 from the distal opening of the sheath 508, the probe 516 can travel freely in the body space 8 without resistance and/or with minimal resistance out of the sheath 508, for example, along the center of the vas deferens lumen 8v and/or along an inner wall of the vas deferens 3 that defines the vas deferens lumen 8v. FIGS. 53B1-53B2 illustrate that when the probe 516 is in an advanced configuration (e.g., a fully advanced configuration), the sheath 508 and the probe 516 can be in the body space 8. After confirmation, the sheath 508 can be advanced over the probe 516 into the body space 8 (e.g., the vas deferens lumen 8v). In practice, the sheath 508 can be inserted (e.g., fully inserted) into the body space 8 until the insertion site of the body space 8 (e.g., wall of the vas deferens 3 adjacent the puncture 566 through the wall of the vas deferens 3) is contacted by the strain relief 512 (e.g., by the larger diameter portion of the strain relief 512). For example, FIGS. 53A1-53B2 illustrate the strain relief 512 (e.g., the distal end of the strain relief 512) in contact with the insertion site (e.g., the tissue adjacent the puncture 566). The distance of advancement of the sheath assembly 502 over the probe 516 can be, for example, 0.5 cm to 1.5 cm over the probe 516 until the insertion site contacts the larger diameter portion of the sheath hub 510 (e.g., the strain relief 512). The advancement of the sheath 508 over the probe 516 can facilitate placement into the body space 8 (e.g., into the vas deferens lumen 8v). The profile of the sheath 508 and the sheath hub 510 can be configured as a cone that can promote engagement of the insertion site to occlude the opening that provides access to the body space 8 (e.g., the puncture 5666 in the wall of the vas deferens 3). By occluding the insertion site (e.g., the puncture 566 in the wall of the vas deferens 3), the possibility of the material 160a leaking around the sheath hub 510 from the insertion site can be reduced or prevented.

FIGS. 53A1-53B2 illustrate, for example, the probe 516 in a probe first configuration PC1 (e.g., as shown in FIGS. 53A1-53A2) and the probe 516 in a probe second configuration PC2 (e.g., as shown in FIGS. 53B1-53B2). FIGS. 53A1-53A2 illustrate that the probe first configuration PC1 can be a fully retracted configuration of the probe 516. FIGS. 53A1-53A2 illustrate that the fully retracted configuration of the probe 516 can be the configuration of the probe 516 before the probe 516 is advanced. FIGS. 53B1-53B2 illustrate that the probe second configuration PC2 can be a fully advanced configuration of the probe 516. Relative to the probe second configuration PC2 in FIGS. 53B1-53B2, the probe can have a partially advanced configuration when the advanced distance D5 is less than the threshold distance D6. FIGS. 53A1-53B2 illustrate that the probe 516 can be advanced and/or retracted, for example, in direction D3 and/or direction D4, respectively.

FIGS. 53C1-53C2 illustrate that the sheath assembly 502 and the probe assembly 504 can be disconnected from each other, for example, while the sheath 508 is in the body space 8. FIGS. 53C1-53C2 illustrate, for example, that the probe assembly 504 can be disconnected from the sheath assembly 502 while the sheath 508 is in the body space 8. FIGS. 53C1 illustrates, for example, that the probe assembly 504 can be removed from the sheath assembly 502 by rotating (e.g., twisting) the probe hub 526 and/or moving (e.g., pulling) the probe assembly 504, including the probe 516, away from the sheath hub 510. The rotating is shown by arrow 568 in FIG. 53C1 and can be clockwise rotation or counterclockwise rotation, and the translating (e.g., pulling) is shown by arrow 570 in FIG. 53C1. FIG. 53C1 illustrates, for example, that the sheath assembly 502 can have a sheath assembly connector 572, and that the probe assembly 504 can have a probe assembly connector 576. The sheath assembly connector 572 can have, for example, zero, one, or multiple grooves 574 (e.g., 0-4 grooves 574, including every 1 groove increment within this range, for example, 0 grooves, 1 groove, 2 grooves, 4 grooves) and zero, one, or multiple gaps 575 around the perimeter of the sheath assembly connector 572. The grooves 574 can be, for example, grooves 574 in the sheath hub 510. For example, FIG. 53C1 illustrates that the sheath assembly connector 572 can have two grooves 574, one on each lateral side of the sheath hub 510, and one gap 575 that extends cross the sheath assembly connector 572 between the two grooves 574. The probe assembly connector 576 can have, for example, zero, one, or multiple extensions 578 (e.g., 0-4 extensions 578, including every 1 extension increment within this range, for example, 0 extensions, 1 extension, 2 extensions, 4 extensions). For example, FIG. 53C1 illustrates that the probe assembly connector 576 can have two extensions 578 that extend in opposite directions. FIG. 53C1 illustrates that the extensions 578 can extend laterally and/or radially from the probe hub 526. The extensions 578 can be moved (e.g., slid) into and out of the grooves 574 for example, when the probe assembly 504 is attached to and detached from the sheath hub 510, respectively. For example, FIGS. 53A1 and 53B1 illustrate that when the probe assembly 504 is in an attached configuration with the sheath assembly 502, each one of the extensions 578 can be in one of the grooves 574. FIG. 53C1 illustrates that when the probe assembly 504 is removed from the sheath assembly 502, the probe assembly 504 can be rotated in direction 568 (e.g., a counterclockwise direction) to rotate the extensions 578 out of the grooves 574 and into the gap 575 so that the probe assembly 504 can be moved (e.g., pulled) away from the sheath assembly 502. As another example, when the probe assembly 504 is attached to the sheath assembly 502, the probe assembly 504 can be rotated relative to the sheath assembly 502 so that each one of the extensions 578 can be rotated into one of the grooves 574, for example, into the positions shown in FIGS. 53A1 and 53B1. When the extensions 578 are rotated into a fully attached position into the grooves 574, the extensions 578 can, for example, snap into place and/or have a friction fit with the sheath hub 510 in the grooves 574. FIGS. 53C1-53C2 illustrate that the rotation indicated by the arrow 568 can be, for example, any fraction of a full rotation of the handle assembly 506 relative to the sheath assembly 502, including, for example, a sixteenth of a turn, an eighth of a turn, a quarter of a turn in order to disengage the probe assembly connector 576 from the sheath assembly connector 572 to remove the probe assembly 504 from the sheath assembly 502. For example, FIGS. 53C1-53C2 illustrate that the probe assembly 504 can be removed from the sheath assembly 502 with a quarter of a turn of the probe assembly 504 relative to the sheath assembly 502. As another example, the probe assembly 504 can be attached to and detached from the sheath assembly 502 with only translational motion, whereby the probe assembly 504 (e.g., the probe hub 526 and/or the probe assembly connector 576) can have, for example, a snap fit and/or a friction fit with the sheath assembly 502 (e.g., with the sheath hub 510). FIG. 53C2 illustrates that when the probe assembly 504 is removed from the sheath assembly 502, the sheath 508 can be left in the body space 8 (e.g., the vas deferens lumen 8v). FIGS. 53A1-53C2 illustrate, for example, that the extensions 578 and the grooves 574 can form a tongue and groove connection between the sheath assembly 502 and the probe assembly 504.

FIG. 53D illustrates that the cartridge 111 (e.g., the capped cartridge 111 full of the material 160a) can be removed from the package 556 (e.g., from the first pouch 558). FIG. 53D illustrates that the caps 554 can be removed from the cartridge 111, for example, by pulling them off the ends of the cartridge by hand. FIG. 53D illustrates that the cartridge can be inserted into the handle assembly 506 as indicated by arrow 572. FIG. 53D illustrates that the cartridge 111 can be inserted into the space 555 in the housing 532, for example, by inserting the cartridge into the space 555 through the opening 531. FIG. 53D illustrates, for example, that the cartridge 111 can be inserted into the housing 532 through the opening 531. FIG. 53D illustrates that either end of the cartridge 111 can be inserted first into the handle assembly 506 through the opening 531.

FIGS. 53E1-53E2 illustrate that the handle assembly 506 can be attached to the sheath assembly 502, for example, while the sheath assembly 502 (e.g., the sheath 508) remains in the body space 8. FIG. 53E1 illustrates, for example, that the handle assembly 506 can be attached to the sheath assembly 502 by pushing the distal cap 530 into the sheath hub 510 and then rotating the handle assembly 506 relative to the sheath hub 510. FIGS. 53E1 illustrates, for example, that the handle assembly 506 can be attached to the sheath assembly 502 by translating (e.g., pushing) the handle assembly 506 toward the proximal end of sheath hub 510 until the distal cap 530 engages with (e.g., makes contact with) the sheath hub 510 and then rotating the handle assembly 506 until it engages with the sheath hub 510. The translating (e.g., pushing) is shown by arrow 580 in FIG. 53E1, and the rotating is shown by arrow 582 in FIG. 53E1, which can be clockwise or counterclockwise rotation. FIG. 53E1 illustrates, for example, that the handle assembly 506 can have a handle assembly connector 584 that can removably engage with the sheath assembly connector 572. The distal cap 530 can have and/or can be the handle assembly connector 584. For example, the handle assembly connector 584 can be attached to or integrated with the distal cap 530 and/or with the housing 532. FIG. 53E1 illustrates, for example, that the handle assembly connector can be integrated with the distal cap 530. The handle assembly connector 584 can have, for example, zero, one, or multiple extensions 586 (e.g., 0-4 extensions 586, including every 1 extension increment within this range, for example, 0 extensions, 1 extension, 2 extensions, 4 extensions). For example, FIG. 53E1 illustrates that the handle assembly connector 584 can have two extensions 586 that extend in opposite directions. FIG. 53E1 illustrates that the extensions 586 can extend laterally and/or radially from the distal cap 530 and/or the housing 532. The extensions 586 can be moved (e.g., slid) into and out of the grooves 574 of the sheath hub 510 when the handle assembly 506 is attached to and detached from the sheath hub 510, respectively. For example, FIG. 53E2 illustrates that when the handle assembly 506 is in an attached configuration with the sheath assembly 502, each one of the extensions 586 can be in one of the grooves 574. FIG. 53E1 illustrates that when the handle assembly 506 is attached to the sheath assembly 502, the handle assembly 506 can be rotated in direction 582 (e.g., a counterclockwise direction) to rotate the extensions 586 out of the gap 585 and into of the grooves 574 so that the handle assembly 506 can be moved (e.g., pulled) away from the sheath assembly 502. As another example, when the handle assembly 506 is removed from the sheath assembly 502, the handle assembly 506 can be rotated relative to the sheath assembly 502 so that each one of the extensions 586 can be rotated out of the grooves 574 and into the gap, for example, so that the handle assembly 506 can be moved (e.g., pulled) away from the sheath assembly 502. When the extensions 586 are rotated into a fully attached position into the grooves 574, the extensions 586 can, for example, snap into place and/or have a friction fit with the sheath hub 510 in the grooves 574. FIGS. 53E1-53CE illustrate that the rotation indicated by the arrow 582 can be, for example, any fraction of a full rotation of the handle assembly 506 relative to the sheath assembly 502, including, for example, a sixteenth of a turn, an eighth of a turn, a quarter of a turn in order to engage the handle assembly connector 584 to the sheath assembly connector 572 to attach the handle assembly 506 to the sheath assembly 502. For example, FIGS. 53E1-53E2 illustrate that the handle assembly 506 can be attached to the sheath assembly 502 with a quarter of a turn of the handle assembly 506 relative to the sheath assembly 502. As another example, the handle assembly 506 can be attached to and detached from the sheath assembly 502 with only translational motion, whereby the handle assembly 506 (e.g., the distal cap 530) can have, for example, a snap fit and/or a friction fit with the sheath assembly 502 (e.g., with the sheath hub 510). FIGS. 53E1-53E2 illustrate that when the sheath 508 is in the body space 8 (e.g., the vas deferens lumen 8v), the handle assembly 506 can be attached to the sheath assembly 502. For example, FIGS. 53E1-53E2 illustrates that the sheath 508 can be left in the body space 8 (e.g., the vas deferens lumen 8v) after the probe assembly 504 is removed such that the sheath 508 does not need to be removed from the body space 8 to attach the handle assembly 506 to the sheath assembly 502. FIGS. 53E1-53E2 illustrate, for example, that the extensions 586 and the grooves 574 can form a tongue and groove connection between the sheath assembly 502 and the handle assembly 506.

FIG. 53E1 illustrates that when the cartridge 111 is in the fully inserted configuration in the handle assembly 506 (e.g., in the housing 532), the end of the cartridge that is in the space 555 can be in contact with the plunger tip 534. When the cartridge 111 is in the fully inserted configuration in the housing 532, the plunger tip 534 may or may not be in the cartridge 111. For example, FIG. 53E1 illustrates that when the cartridge 111 is in the fully inserted configuration 532, the plunger tip 534 can be in the proximal end of the cartridge 111 which can, for example, hold the cartridge 111 in place in the space 555 while the handle assembly 506 is attached to the sheath assembly 502. As another example, when the cartridge 111 is in the fully inserted configuration in the housing 532, the end of the cartridge 111 that is in the space 555 may or may not be in contact with the plunger tip 534 and the plunger tip 534 may not be in the cartridge 111, whereby the cartridge 111 can be held in place in the space 555 while the handle 506 is attached to the sheath assembly 502, for example, via a connection (e.g., a friction fit and/or a snap fit) between the cartridge 111 and the housing 532 when the cartridge 111 is in the fully inserted configuration in the housing 532.

FIG. 53E1 illustrates that when the cartridge 111 is fully inserted into the housing 532, a first end (e.g., a proximal end) of the cartridge 111 can be inside the housing 532 and that a second end (e.g., a distal end) of the cartridge 111 can be outside the housing 532. For example, FIG. 53E1 illustrates that when the cartridge 111 is fully inserted into the housing 532, the cartridge 111 can extend out of the housing 532, for example, through the opening 531. For example, FIG. 53E1 can illustrate the cartridge in a fully loaded configuration (also referred to as a fully inserted configuration) once the loading of the cartridge 111 into the handle assembly 506 (e.g., into the housing 532) is complete, and FIG. 53D can illustrate the cartridge 111 in a fully unloaded configuration, for example, during the loading process before the first end (e.g., the proximal end) of the cartridge 111 passes through the opening 531 in the distal end the housing 532. FIGS. 53D-53E1 illustrate that the cartridge 111 can be attached to the handle assembly 506. For example, FIG. 53E1 illustrates that when the cartridge 111 is attached to the housing 532 by inserting the cartridge 111 into the space 555. FIG. 53E1 illustrates that when the cartridge 111 is attached to the housing 555 (e.g., when the cartridge 111 is in an attached configuration with the housing 532), the cartridge 111 can be in the fully inserted configuration. FIG. 53E1 illustrates that when the cartridge 111 is fully inserted into the housing 532, the material 160a in the first end (e.g., the proximal end) of the cartridge 111 can be inside the housing 532 (e.g., proximal the opening 531 and inside the space 555) and that the material 160a in the second end (e.g., the distal end) of the cartridge 111 can be outside the housing 532. FIG. 53E1 illustrates, for example, that when the cartridge 111 is fully inserted into the housing 532, the material 160a in the first end (e.g., the proximal end) of the cartridge 111 can be proximal the opening 531 and inside the space 555, and that the material 160a in the second end (e.g., the distal end) of the cartridge 111 can be distal the opening 531 and outside of the space 555.

FIGS. 53E1-53E2 illustrate that the guide 517 can guide the cartridge 111 into the sheath hub 510, for example, through the space first section 515a of the sheath assembly space 509 toward and/or into the space second section 515b of the sheath assembly space 509, for example, as the handle assembly 506 is moved (e.g., pushed) toward the sheath assembly 502 as indicated by arrow 580.

FIGS. 53E1-53E2 illustrate that the cartridge 111 can be an aligner (e.g., a guide) that can be used to align (e.g., guide) the handle assembly 506 with the sheath assembly 502, for example, while the handle assembly 506 is attached to the sheath assembly 502. FIGS. 53E1-53E2 illustrate, for example, that advancing the cartridge 111 into the sheath second space 515b can align the handle assembly connector 584 with the sheath assembly connector 572, for example, so that the extensions 586 can be pushed into the gap 575 and then be engaged with (e.g., rotated into) the grooves 574. FIGS. 53E1-5E2 illustrate that when the cartridge 111 is in the fully inserted configuration in the handle assembly 506, the cartridge 111 can be moved into the sheath assembly space 509 by moving (e.g., pushing) the handle assembly 506 toward the sheath assembly 502 while the cartridge 111 is in the fully inserted configuration in the handle assembly 506.

FIGS. 53E1-53E2 illustrate that the material 160a can have a viscosity that can prevent the material from flowing or leaking out of the cartridge 111, for example, out of the distal opening 553 shown in FIG. 53E1, before the plunger tip 534 is advanced through the cartridge 111 (e.g., through the cartridge space 501) via plunger assembly 533. FIGS. 53E1-53E2 illustrate that the viscosity of the material 160a can prevent the material from flowing or leaking out of the cartridge 111, for example, out of the distal opening 553 shown in FIG. 53E1, as the handle assembly 506 is attached to the sheath assembly 502.

FIGS. 53E1-53E2 illustrate that attaching the handle assembly 506 to the sheath assembly can include loading the cartridge 111 (e.g., the distal end of the cartridge 111) into the sheath hub 510, for example, into the sheath assembly space 509.

FIG. 53E2 illustrates a fully attached configuration between the sheath assembly 502 and the handle assembly 506. FIG. 53E2 illustrates that when the sheath assembly 502 and the handle assembly 506 are attached to each other, the cartridge 111 can be in the sheath assembly space 509 and in the handle assembly space 555. For example, FIG. 53E2 illustrates that when the sheath assembly 502 and the handle assembly 506 are connected to each other, the distal end of the cartridge 111 can be in the sheath assembly space 509 and the proximal end of the cartridge 111 can be in the handle assembly space 555. For example, the housing space 555 can be a cartridge first space and the sheath assembly space 509 can be a cartridge second space or vice versa. FIG. 53E2 illustrates that when the handle assembly is attached to the sheath assembly 502, the cartridge 111 can be in the cartridge first space (e.g., in the housing space 555) and in the cartridge second space (e.g., in the sheath assembly space 509). FIG. 53E2 illustrates that when the device 50 is in a loaded configuration (e.g., in a fully loaded configuration), the cartridge 111 can be in the cartridge first space (e.g., in the sheath assembly space 509) and in the cartridge second space (e.g., in the housing space 555). FIG. 53E2 illustrates that when the device 50 has the cartridge 111, the cartridge 111 can be the cartridge first space (e.g., in the sheath assembly space 509) and in the cartridge second space (e.g., in the housing space 555).

The cartridge 111 can provide structural support to the connection between the sheath assembly 502 and the handle assembly 506. For example, FIG. 53E2 illustrates that when the sheath assembly 502 and the handle assembly 506 are in a connected configuration (e.g., the fully connected configuration shown in FIG. 53E2), the cartridge 111 can stabilize and/or strengthen the connection between the sheath assembly 502 and the handle assembly 506, for example, by extending through both assemblies and reducing the stress on the joint between the sheath assembly connector 572 and the handle assembly connector 584. As another example, the cartridge 111 may not provide structural support to the connection between the sheath assembly 502 and the handle assembly 506.

FIG. 53E2 illustrates that that when the sheath assembly 502 and the handle assembly 506 are in a connected configuration (e.g., the fully connected configuration shown in FIG. 53E2), the distal end of the cartridge 111 (e.g., the distal terminal end of the cartridge 111) can be in contact with the first stop 519a.

FIG. 53E2 illustrates that that when the sheath assembly 502 and the handle assembly 506 are in a connected configuration (e.g., the fully connected configuration shown in FIG. 53E2), the space 555, the lumen of the cartridge 111, and the sheath assembly lumen 511 can be aligned with each other. FIG. 53E2 illustrates, for example, that when the sheath assembly 502 and the handle assembly 506 are in a connected configuration (e.g., the fully connected configuration shown in FIG. 53E2), the space 555, the lumen of the cartridge 111, and the sheath assembly lumen 511 can be longitudinally aligned with each other such that the center longitudinal axis A10 of the sheath assembly lumen 511, the center longitudinal axis of the space 555, the center longitudinal axis A11, and the center longitudinal axis A12 of the cartridge space 501 can be parallel and/or collinear with each other.

When the sheath assembly 502 and the handle assembly 506 are in a connected configuration (e.g., the fully connected configuration shown in FIG. 53E2), the material 160a can be forced (e.g., pushed) out of the cartridge 111 and into the sheath assembly lumen 511, for example, by forcing (e.g., pushing) the plunger tip 534 through the cartridge 111. The plunger tip 534 can be pushed, for example, through the cartridge space 501 and into the sheath assembly space 9. The plunger tip 534 can be advanced in the sheath hub 510, for example, until the plunger tip 534 engages (e.g., comes into contact with) the second stop 519b. The second stop 519b can prevent the plunger tip 534 (see e.g., FIGS. 52F1-52F7) from being advanced into the sheath assembly lumen 511.

FIGS. 53E1-53E2 illustrate that the handle assembly 506 can be attached to the sheath assembly 502 when the plunger assembly 533 is in a retracted configuration, for example, when the first plunger 533a is in a fully retracted configuration and when the second plunger 533b is in a fully retracted configuration. The fully retracted configuration of the first plunger 533a and the fully retracted configuration of the second plunger 533b is shown, for example, in FIGS. 52F1-52F7 and 53D-56E2. The fully retracted configurations of the first plunger 533a and the second plunger 533b can be, for example, the configurations of the first plunger 533a and the second plunger 533b before the first plunger 533a and the second plunger 533b are advanced or after the first plunger 533a and the second plunger 533b have been fully retracted (e.g., from an advanced configuration). The retracted configurations of the first plunger 533a and the second plunger 533b can be, for example, the neutral configurations (e.g., packaged configurations) of the first plunger 533a and the second plunger 533b.

FIGS. 53E2-53F2 illustrate that the plunger assembly 533 can be advanced to deliver the material 160a into the body space 8. The first plunger 533a and/or the second plunger 533b can be advanced to force (e.g., push) the material 160 into the body space 8. For example, FIGS. 53E2-53F2 illustrate that the first plunger 533a and the second plunger 533b can be advanced to deliver the material 160a to the body space 8. The material 160a can be delivered to the body space 8, for example, by pushing the material 160a with the plunger head 534. FIGS. 52F1-52F7 and 53E2-53F2 illustrate, for example, that advancing the first plunger 533a can push the plunger tip 534 into the material 160a, and that advancing the second plunger 533b can push the plunger tip 534 into the material 160a.

FIGS. 52F1-52F7 and 53E2-53F2 illustrate, for example, the plunger tip 534 can be pushed through the cartridge space 501 and the housing space 555 and into the sheath assembly space 509 by advancing the first plunger 533a and/or the second plunger 533b.

FIGS. 52F1-52F7 and 53E2-53F2 illustrate, for example, that the material 160a can be pushed (e.g., via the plunger tip 534) through cartridge space 501, out of the cartridge space 501, out of the housing space 555, into the sheath assembly space 509, and/or into the sheath assembly lumen 511 by advancing the first plunger 533a and/or the second plunger 533b.

The first plunger 533a and the second plunger 533b can be advanced separately and/or simultaneously. The first plunger 533a and the second plunger 533b can be advanced and/or or retracted in any order. For example, FIGS. 53E2-53F2 illustrate that the first plunger 533a can be advanced before the second plunger 533b is advanced, and that the second plunger 533b can be advanced after the first plunger 533a is advanced. Advancing the first plunger 533a can, for example, deliver a first portion of the material 160a into the body space 8 and/or into the sheath assembly lumen 511, and advancing the second plunger 533b can, for example, deliver a second portion of the material 160a into the body space 8 and/or into the sheath assembly lumen 511. As another example, the second plunger 533b can be advanced before the first plunger 533a is advanced, and that the first plunger 533a can be advanced after the second plunger 533b is advanced. In such a case, advancing the second plunger 533b can, for example, deliver a first portion of the material 160a into the body space 8 and/or into the sheath assembly lumen 511, and advancing the first plunger 533a can, for example, deliver a second portion of the material 160a into the body space 8 and/or into the sheath assembly lumen 511.

FIGS. 53E2-53F1 illustrate that the first plunger 533a can be advanced and/or retracted, for example, by pushing and/or pulling the first plunger knob 538 in directions D8 and/or D9, respectively. Direction D8 can be toward the sheath assembly 502, and direction D9 can be away from the sheath assembly 502. Directions D8 and D9 can be opposite to each other.

FIGS. 53E2-53F1 illustrate that the first plunger 533a can be advanced from a retracted configuration to an advanced configuration by pushing the first plunger knob 538 in direction D8. FIGS. 53E2-53F1 illustrate, for example, that the first plunger 533a can be advanced (e.g., pushed) to the fully advanced configuration shown in FIG. 53F1.

FIG. 53E2 illustrates the first plunger 533a in a first plunger fully retracted configuration, and FIG. 53F1 illustrates the first plunger 533a in a first plunger fully advanced configuration.

FIGS. 52F1-52F7 and 53E2-53F1 illustrate that the first plunger 533a can be advanced until the first plunger knob 538 makes contact with the housing flange 535. For example, FIG. 53F1 illustrates that when the first plunger 533a is a fully advanced position, the first plunger knob 538 can be in contact with the housing flange 535. The first plunger knob 538 coming into contact with the housing flange 535 can be an indicator (e.g., can indicate to the user) that the first plunger 533a has been fully advanced (e.g., is in a fully advanced configuration). The housing flange 535 can be, for example, a third stop 519c that can stop the forward motion of the plunger tip 534.

FIGS. 53F1-53F2 illustrate that the second plunger 533b can be advanced and/or retracted, for example, by pushing and/or pulling the second plunger knob 544 in directions D8 and/or D9, respectively.

FIGS. 53E2-53F1 illustrate that the second plunger 533b can be advanced from a retracted configuration to an advanced configuration by pushing the second plunger knob 544 in direction D8. FIGS. 53E2-53F2 illustrate, for example, that the second plunger 533b can be advanced (e.g., pushed) to the fully advanced configuration shown FIG. 53F2.

FIG. 53E2 illustrates the second plunger 533b in a second plunger fully retracted configuration, FIG. 53F1 illustrates the second plunger 533b in the second plunger fully retracted configuration, and FIG. 53F2 illustrates the second plunger 533b in a fully advanced configuration. FIGS. 53E2-53F1 illustrate that the second plunger 533b can be moved (e.g., translated) relative to the sheath assembly 502 while in a retracted configuration and/or while in an advanced configuration. For example FIGS. 53E2-53F1 illustrate that the second plunger 533b can be moved toward the sheath assembly 502 while the second plunger 533b is in the second plunger fully retracted configuration. FIGS. 53E2-53F1 illustrate that advancing the first plunger 533a can move (e.g., simultaneously advance) the second plunger 533b and the plunger head 534, for example, relative to the sheath assembly 502. FIGS. 52F1-52F7 and 53E2-53F1 illustrate, for example, that when the second plunger 533b is in the second plunger fully retracted configuration, the second plunger 533b can have a second plunger first position (e.g., as shown in FIGS. 52F1-52F7 and 53E2) and a second plunger second position (e.g., as shown in FIG. 53F1). FIGS. 52F1-52F7 and 53E2-53F1 illustrate that the second plunger 533b can be closer to the sheath assembly 502 when the second plunger 533b is in the second plunger second position than when the second plunger 533b is in the second plunger first position. The configuration of the second plunger 533b when the second plunger 533b is in the second plunger first position can be the same as or different from the configuration of the second plunger 533 when the second plunger 533b is in the second plunger first position. For example, FIGS. 53E2-53F2 illustrate that the second plunger 533b can have the same configuration (e.g., the second plunger fully retracted configuration) when the second plunger 533b is in the second plunger first position and when the second plunger 533b is in the second plunger second position. FIGS. 52F1-52F7 and 53E2-53F1 illustrate, for example, that the fully retracted configuration of the second plunger 533b can be closer to the sheath assembly 502 when the second plunger 533b is in the second plunger second position than when the second plunger 533b is in the second plunger first position.

FIGS. 53E2-53F1 illustrate that advancing the first plunger 533a can move the second plunger 533b from the second plunger first position to the second plunger second position.

The second plunger 533b can be advanced when the second plunger 533b is in the second plunger first position and/or when the second plunger 533b is in the second plunger second position. For example, FIGS. 53E2-53F2 illustrate that when the second plunger 533b is in the second plunger second position (e.g., as shown in FIG. 53F1), the second plunger 533b can be advanced to the second plunger fully advanced configuration (e.g., as shown in FIG. 53F2).

The second plunger 533b can be advanced to deliver material 160a to body space 8, for example, when the second plunger 533b is in the second plunger first position and/or when the second plunger 533b is in the second plunger second position. The second plunger 533b can be advanced, for example, when the first plunger 533a is in the first plunger fully retracted configuration or when the first plunger 533a is in the first plunger fully advanced configuration. For example, FIGS. 53F1-53F2 illustrate that the second plunger 533b can be advanced to deliver the material 160a (e.g., a second portion of the material 160a) to the body space 8 when the first plunger 533a is in a first plunger advanced configuration (e.g., the first plunger fully advanced configuration).

FIGS. 52F1-52F7 and 53F1-53F2 illustrate that the second plunger 533b can be advanced until the second plunger knob 544 makes contact with the first plunger knob 538 and/or until plunger tip 534 makes contact with the second stop 519b. For example, FIG. 53F2 illustrates that when the second plunger 533b is a fully advanced configuration, the second plunger knob 544 can abut the first plunger knob 538. The second plunger knob 544 coming into contact with the first plunger knob 538 can be an indicator (e.g., can indicate to the user) that the second plunger 533b has been fully advanced (e.g., is in a fully advanced configuration). The first plunger knob 538 can be, for example, a fourth stop 519d that can stop the forward motion of the plunger tip 534. As another example, the first plunger knob 538 may not be a stop such that the second plunger 533b can be advanced until the plunger tip 534 makes contact with the second stop 519b in the sheath hub 510. FIG. 53F2 illustrates, for example, that that when the second plunger 533b is in a fully advanced configuration, the plunger tip 534 can be in contact with the second stop 519b. FIG. 53F2 illustrates, for example, that that when the second plunger 533b is in a fully advanced configuration, the second stop 519b can prevent the plunger tip 534 from entering the sheath assembly lumen 511.

FIGS. 53E2-53F2 illustrate that the first plunger 533a and the second plunger 533b can be moved relative to each other and/or can move each other.

FIGS. 53E2-53F2 illustrate that the first plunger 533a can be advanced and retracted independently of the second plunger 533b.

FIGS. 53E2-53F2 illustrate that the second plunger 533b can be advanced and retracted independently of the first plunger 533a.

The first plunger 533a (e.g., the position of the first plunger 533a) can be translated by advancing and/or retracting the second plunger 533b, and/or the second plunger 533b (e.g., the position of the second plunger 533b) can be translated by advancing and/or retracting the first plunger 533a.

FIGS. 53E2-53F2 illustrate, for example, that the second plunger 533b (e.g., the position of the second plunger 533b) can be translated by advancing and/or retracting the first plunger 533a.

Advancing the first plunger 533a to from the first plunger fully retracted configuration to the first plunger fully advanced configuration can deliver a first portion of the material 160a to the body space 8.

Advancing the second plunger 533b to from the second plunger fully retracted configuration to the second plunger fully advanced configuration can deliver a second portion of the material 160a to the body space 8.

FIGS. 52E2-52F2 illustrate that the material 160a can be delivered to the body space 8 in multiple stages, for example, in a first stage and a second stage. Advancing the first plunger 533a can be the first stage, and advancing the second plunger 533b can be the second stage. As another example, the first stage and the second stage can be performed in one continuous stage, for example, in one continuous push of the first plunger 533a and the second plunger 533b.

The device 50 can be removed from the body space 8, for example, after some or all of the material 160a in the cartridge 111 has been delivered to the body space 8. For example, once the material 160a has been delivered to the body space 8, the sheath 508 can be removed from the body space 8 by moving (e.g., pulling) the sheath assembly 502 and the handle assembly 506 away from the body space 8 while the sheath assembly 502 and the handle assembly 506 are connected to each other. As another example, the sheath 508 can be removed from the body space 8 after the handle assembly 506 is removed from the sheath assembly 502.

Read through disclosure again and start going away! 3 hours finish! Then 1 hour clean up!

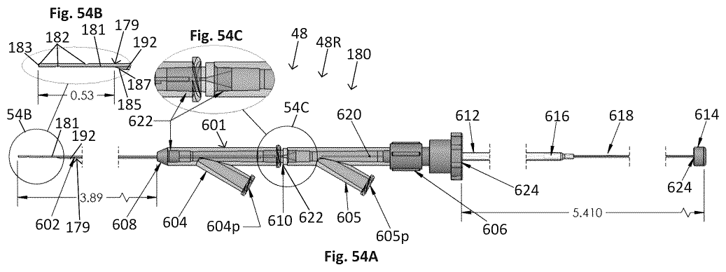

FIGS. 54A-54C illustrate that the system 48R can have a device 180 with the arrangement of features shown. FIGS. 54A-54C illustrate that the device 180 can have the removal sheath 179, a body 601, an aspiration connector 604 (e.g., a Y-connector), an irrigation connector 605 (e.g., a Y-connector), a fitting 606 (e.g., a Tuohy-Borst fitting), a distal connector 608, a proximal connector 610, a distal tubing 612, a probe knob 614 (e.g., a guidewire knob), a probe stop 616 (e.g., a guidewire stop), a probe proximal tube 618 (e.g., a guidewire proximal tube), a probe 620 (e.g., a guidewire), an adhesive 622 (e.g., Loctite 4310), an adhesive 624 (e.g., Loctite 4310), or any combination thereof, for example, in the arrangement shown. FIGS. 54A-54C illustrate that the aspiration connector 604 can have an aspiration port 604p, and that the irrigation connector 605 can have an irrigation port 605p. FIGS. 54A-54C illustrate that the device 180 can have the dimensions shown (e.g., units can be inches). These dimensions can vary, with values greater or less than those shown.

The removal sheath 179 can have one or multiple lumens, including for example, one or multiple irrigation lumens 181, one or multiple aspiration lumens 192, or any combination thereof. For example, FIGS. 54A-54C illustrate that the removal sheath 179 can have an inflation lumen 181 (e.g., one inflation lumen 181) and an aspiration lumen 192 (e.g., one aspiration lumen 192. FIGS. 54A-54C illustrate that the removal sheath 179 can be, for example, an extrusion 602 (e.g., a two lumen extrusion). The sheath 179 can be, for example, a catheter.

The inflation lumen 181 can cave one or multiple irrigation holes 182 (e.g., 1-10 irrigation holes 182, including, for example, every 1 irrigation hole increment within this range, for example, 1 irrigation hole, 2 irrigation holes, 10 irrigation holes). For example, FIGS. 54A-54C illustrate that the inflation lumen 181 can have three irrigation holes 182 (e.g., two side holes 182 and a distal end hole 182). The distal end hole 182 of the irrigation lumen 181 can be, for example, the distal end opening 183 of the irrigation lumen 181.

The aspiration lumen 192 can cave one or multiple aspiration holes 185 (e.g., 1-10 aspiration holes 185, including, for example, every 1 aspiration hole increment within this range, for example, 1 aspiration hole, 2 aspiration holes, 10 aspiration holes). For example, FIGS. 54A-54C illustrate that the aspiration lumen 192 can have one aspiration hole 185 (e.g., a distal end hole 185). The distal end hole 185 of the aspiration lumen 192 can be, for example, a distal end opening 187 of the aspiration lumen 192. For example, FIGS. 54A-54C illustrate that the aspiration hole 185 can be the distal opening 187 of the aspiration lumen 192.

FIGS. 54A-54C illustrate that suction can be supplied to the aspiration lumen 192, for example, through the aspiration connector 604 (e.g., through the aspiration port 604p) from a suction source. FIGS. 54A-54C illustrate, for example, that suction can be supplied to the aspiration lumen 192 and the aspiration hole 185 from the aspiration connector 604.

FIGS. 54A-54C illustrate that the material 160b, the material 160c, and/or the material 160d can be supplied to the irrigation lumen 181, for example, through the irrigation connector 605 (e.g., through the irrigation port 605p) from an irrigation source (e.g., a source of the material 160b, a source of the material 160c, and/or a source of the material 160d). FIGS. 54A-54C illustrate, for example, that the material 160b can be supplied to the to the irrigation lumen 181 and the irrigation holes 182 from the irrigation connector 605.

The probe 620 can be, for example, the probe 55, the probe 516, a guidewire, or any combination thereof or vice versa. The probe 620 can be, for example, a 0.014 inch diameter guidewire. FIGS. 54A-54C illustrate that a proximal end of the probe 620 can be connected to the probe proximal tube 618. FIGS. 54A-54C illustrate that the probe 620 and the probe proximal tube 618 can extend through the probe stop 618. As another example, the probe proximal tube 618 can be a proximal end of the probe 620. The probe 620 can be pushed through the irrigation lumen 181 and/or through the aspiration lumen 192. For example, FIGS. 54A-54C illustrate that the probe 620 can be translated (e.g., pushed) in the irrigation lumen 181. The probe 620 can be translated a distance D5 out the irrigation lumen 181 (e.g., out of the distal end hole 182). When the distance D5 is greater than or equal to the threshold distance D6, the user can confirm that the irrigation lumen 181 is in the body space 8. As another example, the probe 620 can be translated (e.g., pushed) in the aspiration lumen 192. The probe 620 can be translated a distance D5 out the aspiration lumen 192 (e.g., out of the distal end hole 185). When the distance D5 is greater than or equal to the threshold distance D6, the user can confirm that the irrigation lumen 181 and the aspiration lumen 192 are in the body space 8. The probe 620 can be pushed through the irrigation lumen 181 and/or through the aspiration lumen 192, for example, until the probe knob 614 engages (e.g., contacts) the probe stop 616. The probe knob 614 can engage with the probe stop 616, for example, when the distance D5 equals the threshold distance D6. The probe knob 614 can engage with the probe stop 616, for example, when a proximal surface 626 of the probe knob 614 contacts the probe stop 616. FIGS. 54A-54C illustrate that the probe 620 can be advanced out of the irrigation lumen 181 and/or the aspiration lumen 192, for example, by pushing probe knob toward the body 601.

The portion of the irrigation lumen 181 distal the body 601 can have an irrigation lumen length, and the portion of the aspiration lumen 192 distal the body 601 can have an aspiration lumen length. The irrigation lumen length can be less than, equal to, or greater than the aspiration lumen length. For example, FIGS. 54A-54C illustrate that the irrigation lumen length can be greater than the aspiration lumen length.

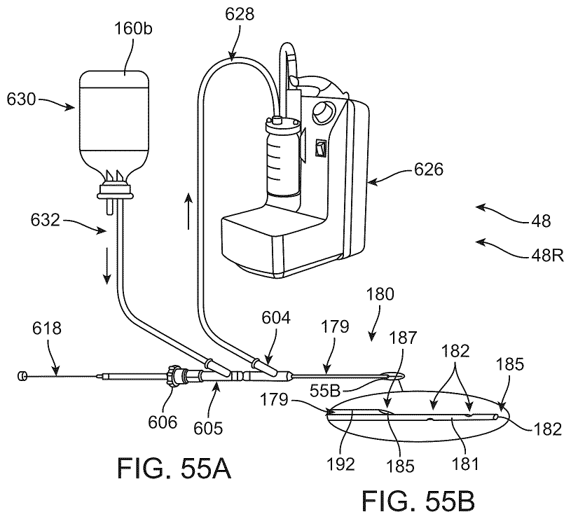

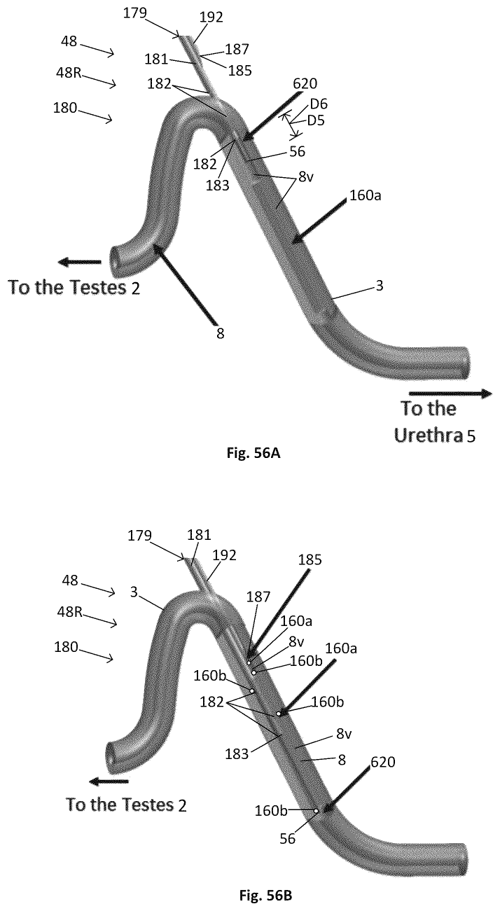

FIGS. 55A-55B illustrate that the removal system 48R can have an aspiration source 626, an aspiration tubing 628, an irrigation source 630, an irrigation tubing 632, or any combination thereof.

FIGS. 55A-55B illustrate that the device 180 can be connected to the aspiration source 626, for example, via the aspiration tubing 628. A first end of the aspiration tubing 628 can be connected to the aspiration source 626, and a second end of the aspiration tubing 628 can be connected to the aspiration connector 604. FIGS. 55A-55B illustrate that the aspiration source 626 can be, for example, a negative pressure pump.

FIGS. 55A-55B illustrate that the device 180 can be connected to the irrigation source 630, for example, via the irrigation tubing 632. A first end of the irrigation tubing 632 can be connected to the irrigation source 630, and a second end of the irrigation tubing 632 can be connected to the irrigation connector 605. The irrigation source 630 can be, for example, a source of the material 160b, a source of the material 160c, a source of the material 160d, or a source of another material. The irrigation source 630 can be connected to the irrigation lumen 181, for example, via the irrigation tubing 632 so that the material in the irrigation source 630 can be supplied to the irrigation lumen 181 and the irrigation holes 182, for example, through the irrigation connector 605. FIGS. 55A-55B illustrate, for example, that the irrigation source 630 can be a source of the material 130b. FIGS. 55A-55B illustrate that the material 160b can be and/or can comprise a sodium bicarbonate solution (e.g., an 8.4% sodium bicarbonate solution). The irrigation source 630 can be, for example, a bottle (e.g., a 50 cc bottle) of the material 160b. The removal system 45R can have a source of the material 160b (e.g., the irrigation source 630), a source of the material 160c, and/or a source the material 160d.

FIGS. 55A-55B illustrate that the device 180 can be connected to the aspiration source 626 and/or to the irrigation source 630.

The material 160a (e.g., a hydrogel such as Vasalgel®) can dissolve and become a liquid when exposed to the material 160b. The material 160b can be, for example, sodium bicarbonate (e.g., to 8.4% USP), sterile for injection sodium bicarbonate solution). The material 160a can dissolve and become a liquid when exposed to the material 160b, for example, due to a change in pH from about 4.5 to over 7.0. Mechanically, the viscosity of the material (e.g., a hydrogel such as Vasalgel®) can change from a soft solid to about the viscosity of water (1.0 to 1.3 centipoise) after exposure to the material 160b (e.g., 8.4% USP, sterile for injection sodium bicarbonate solution). The device 180 can be used, for example, to deliver the material 160b (e.g., the sodium bicarbonate) to the body space 8 (e.g., the vas deferens 3).

Removing of the material 160a (e.g., a hydrogel such as Vasalgel®) from the body space 8 with the device 180 can include any of the following steps in any order, for example, in the order listed: (1) expose the vas deferens 3 via no-scalpel vasectomy technique, (2) advance the device 180 (e.g., the removal sheath 179 over the probe 620 into the vas deferens lumen 8v 1.0 cm-1.5 cm, (3) connect the irrigation tubing 632 to the irrigation port 605 and spike to the irrigation source 630 (e.g., a 8.4% USP, sterile for injection sodium bicarbonate bottle), (4) connect the aspiration tubing 628 to the aspiration port 604 and to the aspiration source 626, (5) apply irrigation of the material 160b (e.g., 8.4% USP, sterile for injection sodium bicarbonate solution) through the irrigation lumen 181 with concurrent aspiration through the aspiration lumen 192, (6), apply irrigation and aspiration for a minimum time period, for example, of 1-10 minutes (e.g., for 4 minutes), (7) confirm vas deferens patency by free movement of the probe 620 and the removal sheath 179 within the vas deferens 3, (8) repeat on contralateral side, or any combination thereof. Any of these steps can be modified and/or omitted. The maximum irrigation pressure can be, for example, 1.0 psi to 6.0 psi, or more narrowly, 2.0 psi to 4.0 psi, including every 0.1 psi increment within these ranges (e.g., 3.0 psi).

The irrigation tubing can be made from flexible polyvinyl chloride (PVC) and can have a male luer connector distally for connection to the irrigation port of the device 180 (e.g., to the port of irrigation connector 605) and a spike for connection to the irrigation source 630 (e.g., for a bottle having 8.4% USP, sterile for injection sodium bicarbonate solution).

The aspiration tubing 628 can be made from and/or can comprise flexible PVC and can have a male luer connector distally for connection to the aspiration port of the device 180 (e.g., to the port of the aspiration connector 604) and can be connected to a fitting (e.g., a barbed fitting) on the aspiration source 626.

The removal sheath 179 can be inserted into the body space 8, for example, in the same way as the sheath 508 of the device 50. For example, can enter a wall (e.g., the anterior wall) of the vas deferens 3. In practice, the anterior wall can be opened by the physician with the distal tip of the dissecting forceps in the same fashion that the scrotal skin is opened via blunt dissection in a no-scalpel technique. The anterior wall entry can be, for example, 3 cm proximal to the entry location used for placement of the device 50. The distal end of the device 180 can have a dual lumen for irrigation and aspiration. The removal sheath 179 can have the same or different distal profile (e.g., the same 2 Fr distal profile) as the sheath 508 of the device 50.

The distal tip of the device 180 (e.g., the irrigation lumen 181 and/or the aspiration lumen 192 of the removal sheath 179) can be inserted into the vas deferens and the probe 620 (e.g., a 0.014″ guidewire) can be used to confirm placement of the removal sheath 179 in the vas deferens lumen 8v. The probe 620 can be advanced the threshold distance D6 (e.g., minimum of 3 cm) to signify proper location of the removal sheath 179. After confirmation of access to the vas deferens lumen 8v is confirmed, the removal sheath 179 can be advanced over-the-probe (e.g., over-the-guidewire) to a distance of 2.0 cm-3.0 cm in the vas deferens lumen 8v.

Before, after, or while the removal sheath 179 is advanced over-the-probe (e.g., over-the-wire), the source of the material 160b (e.g., a 8.4% USP, sterile for injection sodium bicarbonate solution), for example, the irrigation source 630, can be connected the irrigation port of the device 180 (e.g., to the port of irrigation connector 605) with the irrigation tubing 632. The irrigation source 630 (e.g., a 50 cc bottle of 8.4% USP, sterile for injection sodium bicarbonate solution) can be placed on an IV pole above the patient and the stopcock can be opened to allow the flow of the material 160b (e.g., the sodium bicarbonate solution) into the body space (e.g., into the vas deferens lumen 8v). The pressure regulator can allow only a maximum of 1.0 psi to 6.0 psi, or more narrowly, 2.0 psi to 4.0 psi, including every 0.1 psi increment within these ranges (e.g., 3.0 psi) of pressure within the device 180.

The aspiration source 626 can be attached to the aspiration port of the device 180 (e.g., to the port of aspiration connector 604). The stopcock can be opened for aspiration (e.g., continuous aspiration) during the reversal procedure. With the aspiration source 626 on and the irrigation of the material 160b (e.g., 8.4% USP, sterile for injection sodium bicarbonate solution), the probe 620, followed by the delivery sheath 179, can be advanced and retracted through the occlusion site (e.g., the site of the material 160a) to facilitate removal of the material 160a (e.g., a hydrogel such as Vasalgel®).

FIGS. 56A-56B illustrate a variation of a process of accessing the body space 8 (e.g., the vas deferens lumen 8v) having an occlusion (e.g., the material 160a), for example, with the device 180. FIGS. 56A-56B illustrate a variation of a process of removing the occlusion (e.g., the material 160a) from the body space 8, for example, by delivering the material 160b into the body space 8. For example, FIGS. 56A-56B illustrate the steps shown to access the body space 8 and remove the occlusion (e.g., the material 160a) from the body space 8, for example, to reopen the body space 8. FIGS. 56A-56B illustrate a cross-sectional view of the vas deferens 3.

FIGS. 56A-56B illustrate that the body space (e.g., the vas deferens lumen 8v) can be accessed using the device 180, for example, using the same or similar access procedure as to how the body space 8 can be accessed using the device 50 (e.g., via the conduit 52, via the sheath assembly 502). For example, FIG. 56A illustrates that using standard no-scalpel vasectomy procedures, the vas deferens 3 can be locally anesthetized and exposed outside the scrotum 1 through a scrotal skin opening 6. Using the forceps 22 (e.g., as shown in FIG. 1C), FIG. 56A illustrates that a puncture 566 (e.g., as shown in FIG. 53A1) can be placed in a wall of the vas deferens 3 (e.g., the vas deferens anterior wall) for access to the vas deferens lumen 8v (e.g., as shown in FIG. 1C). FIG. 56A illustrates, for example, that the device 180 may not have a needle (e.g., the needle 54). As another example, the device 180 (e.g., the device 180 shown in FIG. 56A) can have a needle (e.g., the needle 54).

FIG. 56A illustrates that the removal sheath 179 can be pushed through the puncture 566 into the vas deferens lumen 8v, for example, to the position shown. The irrigation lumen 181 and/or the aspiration lumen 192 can be pushed through the puncture 566 into the vas deferens lumen 8v. For example, FIG. 56A illustrates that the irrigation lumen 181 can be pushed through the puncture 566 into the vas deferens lumen 181. The lumen having the probe 620 can be pushed through the puncture 566 into the vas deferens lumen 8v. For example, FIG. 56A illustrates that the irrigation lumen 181 can have the probe 620 such that FIG. 56A illustrates that the irrigation lumen 181 can be pushed through the puncture 566 into the vas deferens lumen 8v. When the removal sheath 179 is pushed through the puncture 566 into the vas deferens lumen 8v, the probe 620 may or may not be in the removal sheath 179. For example, the portion of the removal sheath 179 in the vas deferens lumen 8v shown in FIG. 56A may or may not have the probe 620. For example, FIG. 56A illustrates that the portion of the removal sheath 179 in the vas deferens lumen 8v can have the probe 620 such that FIG. 56A illustrates that the removal sheath 179 (e.g., the irrigation lumen 181 of the removal sheath 179) and the probe 620 (e.g., the distal end of the probe 620) can be advanced through the puncture 566 together (e.g., at the same time) while the probe 620 is in a lumen (e.g., the irrigation lumen 181) of the removal sheath 179. FIG. 56A illustrates, for example, that the removal sheath 179 (e.g., the irrigation lumen 181) and the probe 620 can be simultaneously pushed through the puncture 566 and into the vas deferens lumen 8v. FIG. 56A illustrates that the removal sheath 179 and the probe 620 can be pushed through the puncture 566 while the probe 620 is in a retracted configuration (e.g., in a fully retracted configuration). FIG. 56A illustrates that when the probe 620 is in a fully retracted configuration, the distal end of the probe 620 can be inside a distal end of the removal sheath 179, for example, in the irrigation lumen 181. As another example, the lumen of the removal sheath 179 (e.g., the irrigation lumen 181 or the aspiration lumen 192) through which the probe 620 can be advanceable and retractable can be pushed through the puncture 566 into the vas deferens lumen 8v before the probe 620 is in the removal sheath 179, for example, before the probe 620 is in the irrigation lumen 181 or the aspiration lumen 192. As another example, the removal sheath 179 can be pushed through the puncture 566 into the vas deferens lumen 8v while the probe 620 extends distally out of the removal sheath 179 (e.g., out of an irrigation hole 182, for example, distal opening 183 of the irrigation lumen 181).

FIG. 56A illustrates that the probe 620 can be advanced a distance D5 out of the removal sheath 179 by moving (e.g., pushing) the probe knob 614 and/or the probe proximal tube 618 toward the removal sheath 179. The probe 620 can be advanced out of the removal sheath 179 a distance D5 less than, equal to, or greater than the threshold distance D6 into the body space 8. As another example, the maximum distance D5 can be the threshold distance D6. When removing of the material 160a (e.g., a hydrogel such as Vasalgel®) from the body space 8, the threshold D6 can be less than, equal to, or greater than the threshold distance D6 when delivering the material 160a to the body space 8. For example, FIG. 56A illustrates that the threshold distance D6 during removal of the material 160 can be, for example, 1.0 cm-3.0 cm, or more narrowly 2.0 cm-3.0 cm, including every 0.1 cm increment within these ranges (e.g., 1.0 cm, 2.0 cm, 2.5 cm, 3.0 cm). FIG. 56A illustrates that the probe 620 can be advanced into the body space a distance D5 that equals or is greater than the threshold distance D6 to confirm that the removal sheath 179 is in the body space 8 (e.g., the vas deferens lumen 8v). FIG. 56A illustrates that a partially advanced position of the probe 620 can correspond to the distance D5 reaching (e.g., equaling) the threshold distance D6, for example, as measured from a distal terminal end of the removal sheath 179. FIGS. 56A-56B illustrate, for example, that the threshold distance D6 can be less than the maximum distance D5 that the probe 620 can be advanced out of the removal sheath 179 (e.g., out of the irrigation lumen 181). FIG. 56A illustrates, for example, that when the probe 620 is in a partially advanced configuration, the probe knob 520 may not contact (e.g., may not abut) the guidewire stop 616. As another example, the maximum distance D5 that the probe 620 can be advanceable out of the removal sheath 179 can be the threshold distance D6. FIG. 56A illustrates that for the probe 620 to reach the threshold distance D6 as the probe 620 is advanced into the body space 8 from the distal opening 183 of the removal sheath 179, the probe 620 can travel freely in the body space 8 without resistance and/or with minimal resistance out of the removal sheath 179, for example, along the center of the vas deferens lumen 8v and/or along an inner wall of the vas deferens 3 that defines the vas deferens lumen 8v. FIG. 56A illustrates that when the probe 620 is in an advanced configuration (e.g., a partially advanced configuration), the removal sheath 179 and the probe 620 can be in the body space 8. After confirmation (e.g., after the distance D5 is greater than or equal to the threshold distance D6), the removal sheath 179 can be advanced over the probe 620 to the material 160a (e.g., to the occlusion).

FIG. 56B illustrates that the device 180 can irrigate and aspirate inside the body space 8. For example, the stopcock on the irrigation tubing 632 can be opened, and the aspiration source (e.g., a negative pressure pump) can be turned on. FIG. 56B illustrates that as the material 160b (e.g., sodium bicarbonate) flows out the irrigation holes 182 (e.g., through the two side holes 182 and the distal opening 183), the occlusion (e.g., the material 160a) can be dissolved and removed through the aspiration hole 185 (e.g., through the distal opening 187). For example, FIG. 56B illustrates the material 160b flowing out of the irrigation holes 182 and the dissolved material 160a and the material 160b flowing into the aspiration hole 185 (e.g., into the distal opening 187). The circles labeled with as the material 160a and the material 160b are for illustrative purposes only, for example, to show exemplary locations of these materials during irrigation and/or aspiration.

FIGS. 56A-56B illustrates that as the occlusion (e.g., the material 160a) is dissolved by the material 160b during irrigation and aspiration, the removal sheath 179 can be advanced in the body space (e.g., in the vas deferens lumen 8v) until the occlusion (e.g., the material 160) has been completely removed and/or until the body space 8 has been reopened, which can be confirmed, for example, by unrestricted movement of the removal sheath 179 and/or the probe 620 in the body space 8 (e.g., in the vas deferens lumen 8v). FIGS. 56A-56B illustrate that the removal sheath 179 and the probe 620 can fully cross the material 160a during irrigation and aspiration.

FIGS. 56A-56B illustrate that the body space 8 can be irrigated and aspirated for a minimum time period, for example, of 1-10 minutes (e.g., for 4 minutes), for example, to confirm the material 160a has been fully removed and/or to confirm that the body space 8 has been reopened.

FIGS. 56A-56B illustrate that the body space 8 can be irrigated with the material 160b, for example, to restore partial or full patency of the body space 8.

FIGS. 56A-56B illustrate that the material 160a and/or the material 160b can be agitated with the probe 620. For example, FIGS. 56A-56B illustrate that the material 160a and/or the material 160b can be agitated before, during, and/or after irrigating the material 160b into the body space 8, before, during, and/or after aspirating the material 160a and/or the material 160b from the body space 8, before, during, and/or after irrigating the material 160b into the body space and aspirating the material 160a and the material 160b from the body space 8, or any combination thereof, for example, by moving the probe 620 relative to the material 160a and/or the material 160b. Moving the probe 620 relative to the material 160a and/or the material 160b can include, for example, translating and/or rotating the probe 620 relative to the material 160a and/or the material 160b. Moving the probe 620 relative to the material 160a and/or the material 160b can include, for example, moving the probe 620 into and out of contact with the material 160a, for example, before, during, and/or after irrigation, aspiration, and/or irrigation and aspiration, for example, by advancing and retracting the probe 620. As another example, moving the probe 620 relative to the material 160a and/or the material 160b can include, for example, rotating the probe 620 while the probe 620 is advanced and/or retracted, rotating the probe 620 while the probe 620 is in contact with the material 160a and/or the material 160b, for example, before, during, and/or after irrigation, aspiration, and/or irrigation and aspiration. Moving the probe 620 relative to the material 160a can include for example, keeping the probe 620 in contact with the material 160a, for example, by pushing the probe 620 further into the body space 8 as the material 160b progressively breaks down the occlusion from a first end to a second end of the occlusion. Agitating the material 160a and/or the material 160b can include, for example, mechanically agitating the material 160a and/or the material 160b with the probe 620. FIGS. 56A-56B illustrate that the probe 620 can agitate the material 160a and/or the material 160b according to any technique disclosed herein, including, for example, the methods described with reference to FIGS. 42-45G. The device 180 in FIGS. 56A-56B and the method shown and described with reference to the device 180 (e.g., in FIGS. 56A-56B) can be used in combination with any of the disclosure herein.

Translating and/or rotating the probe 620 in the body space 8 can create turbulence in the material 160b in the body space 8, which can facilitate (e.g., accelerate) the breakdown of the material 160a in the body space 8 by the material 160b. The irrigation and/or suction of the material 160b in the body space 8 and/or a translation and/or rotation of the probe 620 in the body space can create turbulence of the material in the body space 8. The material 160b in the body space 8 can cause pieces or chunks of the material 160a to break off the occlusion (e.g., the material 160a) in the body space 8. For example, an interaction (e.g., a chemical reaction) between the material 160a and the material 160b in the body space 8 can cause pieces or chunks of the material 160a to break off the occlusion in the body space 8. Turbulence of the material 160b, which can be caused by moving the probe 620 (e.g., translating and/or rotating the probe 620) and/or the flow of the material 160b into and/or out of the body space 8, can cause pieces or chunks of the material 160a that have broken off the occlusion to collide with the remaining portion of the occlusion that is still intact in the body space 8. Such collisions can facilitate (e.g., accelerate) the breakdown of the occlusion (e.g., the material 160a) by the material 160b, for example, by the material 160b pieces of chunks of the material 160a that have broken off the occlusion to collide with (e.g., slam into) the remaining portion of the occlusion and/or collide with the wall of the body space (e.g., the wall of the vas deferens 3 defining the vas deferens lumen 8v). The impacts from these collisions can facilitate (e.g., accelerate) the breakdown of the remaining portion of the occlusion, for example, by causing more pieces or chunks of the material 160a to break off from the occlusion and/or by weakening of the remaining portion of the occlusion that is still intact. The pieces or chunks of the material 160a can thereby agitate the remaining portion of the occlusion in the body space 8 that is still intact. For example, creating turbulence in the body space can include forcing a first portion of an occlusion (e.g., a piece or chunk of the material 160a) to collide with a second portion of the occlusion (e.g., a remaining portion of the occlusion that is still intact and/or is larger than the first portion of the occlusion). Creating turbulence in the body space can include creating turbulence in a fluid (e.g., the material 160b) that comprises the first portion of the occlusion.

The device 50 (e.g., including the cartridge 111) can be provided sterile, for example, for single-use only.

The device 180 can be provided sterile, for example, for single-use only.

The device 50 and the device 180 can be used, for example, in healthcare settings where a sterile field can be provided and can be used by healthcare professionals trained to perform male contraception (e.g., vasectomy) and/or female contraception.

As the material 160a is delivered into the vas deferens lumen 8v from the device 50, the material 160a (e.g., a hydrogel such as Vasalgel®) can be a bio-adhesive, that can precipitate into a soft viscoelastic solid material as DMSO is replaced by bodily fluids. The material 160a can gel (e.g., can immediately gel) within the body space 8 (e.g., the vas deferens lumen 8v) with a viscosity of 3,000-10,000 centipoise. The material 160a (e.g., a hydrogel such as Vasalgel®) can be configured to inhibit the passage of sperm, which are approximately 3 micrometers in size.

If elected in the future, the material 160a can be removed from the body space 8 with the administration of the material 160b (e.g., 8.4% USP, sterile for injection sodium bicarbonate solution). Upon the interaction of the material 160a with the material 160b (e.g., sodium bicarbonate solution) in the body space 8, the material 160a (e.g., a hydrogel such as Vasalgel®) can change from a soft solid to a liquid with a viscosity approximately 1.3 centipoise, which can closely match the viscosity of water. The removal procedure for the material 160a can combine delivery of the material 160b (e.g., sodium bicarbonate solution) via the device 180 that enters the body space 8 (e.g., the vas deferens lumen 8v) through the irrigation holes 182 using a similar access procedure as for the device 50. As a safety feature, delivery of the material 160b (e.g., sodium bicarbonate solution) can be performed with pressure-controlled irrigation and continuous aspiration to prevent the internal distension pressure of the vas deferens lumen 8v from exceeding 1.0 psi to 6.0 psi, or more narrowly, 2.0 psi to 4.0 psi, including, for example, every 0.1 psi increment within these ranges (e.g., 3.0 psi).

U.S. Pat. Nos. 9,861,505 and 10,456,292 are incorporated by reference herein in their entireties for all purposes.

A method of confirming intraluminal access of a lumen in a body is disclosed (e.g., as shown in the drawings and/or as described herein). The method can include inserting a sheath into the body, advancing a probe relative to the sheath by an advancing distance into the lumen, and/or confirming the sheath has access to the lumen if the advancing distance is greater than or equal to a threshold distance.

Inserting the sheath into the body can include penetrating tissue with a penetrator. The penetrator can be a distal end of the sheath and/or a needle.

Inserting the sheath into the body can include inserting the sheath through an incision.

Advancing the probe relative to the sheath by the advancing distance into the lumen can include advancing the probe out of the sheath by the advancing distance.

Advancing the probe relative to the sheath by the advancing distance into the body can include advancing the probe out of a port by the advancing distance. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

Confirming the sheath has access to the lumen if the advancing distance is greater than or equal to the threshold distance can include confirming the sheath is in the lumen if the advancing distance is greater than or equal to the threshold distance.

Inserting the sheath into the body can include inserting a port into the body. Confirming the sheath has access to the lumen if the advancing distance is greater than or equal to the threshold distance can include confirming the port has access to the lumen if the advancing distance is greater than or equal to the threshold distance. The sheath or a penetrator have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

Inserting the sheath into the body can include inserting a port into the body. Confirming the sheath has access to the lumen if the advancing distance is greater than or equal to the threshold distance can include confirming the port is in the lumen if the advancing distance is greater than or equal to the threshold distance. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

The method can include confirming the sheath does not have access to the lumen if the advancing distance is less than the threshold distance. Confirming the sheath does not have access to the lumen if the advancing distance is less than the threshold distance can include confirming the sheath is not in the lumen if the advancing distance is less than the threshold distance. Inserting the sheath into the body can include inserting a port into the body. Confirming the sheath does not have access to the lumen if the advancing distance is less than the threshold distance can include confirming the port does not have access to the lumen if the advancing distance is less than the threshold distance. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle. Inserting the sheath into the body can include inserting a port into the body. Confirming the sheath does not have access to the lumen if the advancing distance is less than the threshold distance can include confirming the port is not in the lumen if the advancing distance is less than the threshold distance. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

The method can include delivering a material into the lumen after confirming the sheath has access to the lumen. The material can be an implant. The material can be material of an implant. The material can be implanted material. The implant, the material of the implant, and/or the implanted material can be a hydrogel. The material can form an occlusion. The material can be a hydrogel. The material can be configured to dissolve an implant. The material can be sodium bicarbonate. The material can be configured to alter a physical property and/or a chemical property of an implant.

The method can include delivering a material through the sheath into the lumen after confirming the sheath has access to the lumen.

The method can include delivering a material through a port into the lumen after confirming the port has access to the lumen. The sheath or a penetrator can have the port.

The method can include retracting the probe relative to the sheath. The method can include delivering a material through the sheath into the lumen after confirming the sheath has access to the lumen and after retracting the probe relative to the sheath. The method can include delivering a material through a port into the lumen after confirming the port has access to the lumen and after retracting the probe relative to the sheath. The material can be a hydrogel.

The method can include retracting the probe into the sheath. The method can include delivering a material through the sheath into the lumen after confirming the sheath has access to the lumen and after retracting the probe into the sheath. The method can include delivering a material through a port into the lumen after confirming the port has access to the lumen and after retracting the probe into the sheath. Retracting the probe into the sheath can include retracting the probe into the port.

The method can include delivering a material into the lumen.

The method can include removing a material from the lumen after confirming the sheath has access to the lumen. The material can be an implant. The material can be material of an implant. The material can be implanted material. The implant, material of the implant, and/or the implanted material can be a hydrogel. The material can form an occlusion. The material can be a hydrogel. The material can be configured to dissolve an implant. The material can be sodium bicarbonate. The material can be configured to alter a physical property and/or a chemical property of an implant. Removing the material from the lumen after confirming the sheath has access to the lumen can include aspirating the material from the lumen after confirming the sheath has access to the lumen.

The method can include removing a material from the lumen into the sheath after confirming the sheath has access to the lumen. Removing the material from the lumen into the sheath after confirming the sheath has access to the lumen can include aspirating the material from the lumen into the sheath after confirming the sheath has access to the lumen.

The method can include removing a material from the lumen through a port after confirming the port has access to the lumen. The sheath or a penetrator can have the port. Removing the material from the lumen through the port after confirming the port has access to the lumen can include aspirating the material from the lumen through the port after confirming the port has access to the lumen.

The method can include moving a material from the lumen into the sheath after confirming the sheath has access to the lumen. Moving the material from the lumen into the sheath after confirming the sheath has access to the lumen can include aspirating the material from the lumen into the sheath after confirming the sheath has access to the lumen.

The method can include retracting the probe relative to the sheath. The method can include removing a material from the lumen into the sheath after confirming the sheath has access to the lumen and after retracting the probe relative to the sheath. The method can include removing a material from the lumen through a port after confirming the port has access to the lumen and after retracting the probe relative to the sheath. Retracting the probe relative the sheath can include retracting the probe into the port.

The method can include retracting the probe into the sheath. The method can include removing a material from the lumen into the sheath after confirming the sheath has access to the lumen and after retracting the probe into the sheath. The method can include removing a material from the lumen through a port after confirming the port has access to the lumen and after retracting the probe into the sheath. Retracting the probe into the sheath can include retracting the probe into the port.

The method can include removing a material from the lumen.

The method can include delivering a first material into the lumen after confirming the sheath has access to the lumen. The first material can be configured to dissolve a second material. The first material can be sodium bicarbonate. The first material can be configured to alter a physical property and/or a chemical property of a second material. The method can include removing a second material from the lumen. The second material can be an implant. The second material can be material of an implant. The second material can be implanted material. The implant, the material of the implant, and/or the implanted material can be a hydrogel. The second material can be an occlusion. The second material can be a hydrogel. Removing the second material from the lumen can include aspirating the second material from the lumen. The method can include removing the first material from the lumen. Removing the first material from the lumen can include aspirating the first material from the lumen. Removing the second material from the lumen can include aspirating the second material from the lumen. The method can include simultaneously removing the first material and the second material from the lumen.

The method can include delivering a first material into the lumen after confirming the sheath has access to the lumen. The method can include changing a physical property of a second material in the lumen via the first material. The method can include removing the first material and/or the second material from the lumen. Removing the first material and/or the second material from the lumen can include aspirating the first material and/or the second material from the lumen.

The method can include delivering a first material into the lumen after confirming the sheath has access to the lumen. The method can include agitating a second material in the lumen with the probe. The method can include agitating the first material in the lumen with the probe.

The method can include delivering a first material into the lumen after confirming the sheath has access to the lumen. The method can include agitating the first material and/or a second material in the lumen with the probe.

The probe can be a first probe. The method can include agitating a second material in the lumen with a second probe. The method can include agitating the first material in the lumen with the second probe.

The probe be a first probe. The method can include agitating the first material and/or a second material in the lumen with a second probe.

The method can include agitating a first material and/or a second material with the probe. The first material can be a fluid. The second material can be an implant. The second material can be material of an implant. The second material can be implanted material. The implant, the material of the implant, and/or the implanted material can be a hydrogel. The first material can be sodium bicarbonate. The implant can be a hydrogel. The method can include removing the first material and/or the second material from the lumen. Removing the first material and/or the second material from the lumen can include aspirating the first material and/or the second material from the lumen.

The probe can be a first probe. The method can include agitating a first material and/or a second material with a second probe. The first material can be a fluid. The second material can be material of an implant. The second material can be implanted material. The implant, the material of the implant, and/or the implanted material can be a hydrogel. The first material can be sodium bicarbonate. The implant can be a hydrogel. The method can include removing the first material and/or the second material from the lumen. Removing the first material and/or the second material from the lumen can include aspirating the first material and/or the second material from the lumen.

The method can include delivering a first material into the lumen after confirming the sheath has access to the lumen. The lumen can have a second material. The method can include creating a third material in the lumen via a chemical reaction between the first material and the second material. The probe can be a first probe. The method can include removing the first material, the second material, and/or the third material from the lumen and/or the method can include agitating the first material, the second material, and/or the third material in the lumen with the first probe and/or with a second probe. Removing the first material, the second material, and/or the third material from the lumen can include aspirating the first material, the second material, and/or the third material from the lumen.

The threshold distance can be 1.0 cm to 15.0 cm. The threshold distance can be 1.0 cm to 10.0 cm. The threshold distance can be 1.0 cm to 5.0 cm. The threshold distance can be 5.0 cm, 5.0 cm or less, 10.0 cm or less, or 15.0 cm or less.

The lumen can be a lumen of a reproductive tract. The lumen can be a lumen of a vas deferens. The lumen can be a lumen of a blood vessel.

The method can include preventing delivery of a material into the lumen until confirming the sheath has access to the lumen.

The method can include unlocking a lock upon or after confirming the sheath has access to the lumen.

The sheath can be a tube. The sheath can be a catheter. The sheath can have a needle. A distal tip of the sheath can have a needle. The probe can be a guidewire.

Advancing the probe can include advancing the probe via a spring or a spring-loaded retractable lancet system.

Advancing the probe relative to the sheath by the advancing distance into the lumen can include advancing the probe out of the sheath by the advancing distance.

Confirming the sheath has access to the lumen if the advancing distance is greater than or equal to the threshold distance can include confirming the sheath is in the lumen if the advancing distance is greater than or equal to the threshold distance.

Inserting the sheath into the body can include inserting a port into the body. Confirming the sheath has access to the lumen if the advancing distance is greater than or equal to the threshold distance can include confirming the port has access to the lumen if the advancing distance is greater than or equal to the threshold distance.

The method can include delivering a material into the lumen after confirming the sheath has access to the lumen. The material can be a hydrogel or can be configured to alter a physical property and/or a chemical property of an implant.

The method can include removing a material from the lumen after confirming the sheath has access to the lumen. The material can be a hydrogel or can be configured to alter a physical property and/or a chemical property of an implant.

The lumen can be a lumen of a reproductive tract.

A device is disclosed (e.g., as shown in the drawings and/or as described herein). The device can have a sheath and a probe. The probe can be advanceable from a retracted configuration to an advanced configuration relative to the sheath. When the probe is in the retracted configuration, a first distance can be between the probe and the sheath. When the probe is in the advanced configuration, a second distance greater than the first distance can be between the probe and the sheath. The first distance can be less than a threshold distance. When the second distance is greater than or equal to the threshold distance, the advanced configuration of the probe can be an indicator to a user that the device has access to a target site.

When the second distance is greater than or equal to the threshold distance, the advanced configuration of the probe can be an indicator to a user that the device is in the target site.

When the second distance is greater than or equal to the threshold distance, the advanced configuration of the probe can be an indicator to a user that the sheath has access to the target site.

When the second distance is greater than or equal to the threshold distance, the advanced configuration of the probe can be an indicator to a user that the sheath is in the target site.

When the second distance is greater than or equal to the threshold distance, the advanced configuration of the probe can be an indicator to the user that a port has access to the target site. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

When the second distance is greater than or equal to the threshold distance, the advanced configuration of the probe can be an indicator to the user that a port is in the target site. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

When the second distance is less than the threshold distance, the advanced configuration of the probe can be an indicator to the user that the device does not have access to the target site.

When the second distance is less than the threshold distance, the advanced configuration of the probe can be an indicator to a user that the device is outside of the target site.

When the second distance is less than the threshold distance, the advanced configuration of the probe can be an indicator to a user that the sheath does not have access to the target site.

When the second distance is less than the threshold distance, the advanced configuration of the probe can be an indicator to a user that the sheath is outside of the target site.

When the second distance is less than the threshold distance, the advanced configuration of the probe can be an indicator to the user that a port does not have access to the target site. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

When the second distance is greater than or equal to the threshold distance, the advanced configuration of the probe can be an indicator to the user that a port is outside of the target site. The sheath or a penetrator can have the port. The port can be a distal terminal opening of the sheath or an opening on a side of the sheath. The port can be an opening of the penetrator. The port can be proximal a distal tip of the sheath. The port can be proximal a distal tip of the penetrator. The penetrator can be a needle.

The target site can be a lumen. The target site can be a lumen of a reproductive tract. The target site can be a lumen of a vas deferens. The target site can be a lumen of a blood vessel.

The sheath can have a sheath lumen. When the probe is in the retracted configuration, a distal end of the probe can be inside the sheath lumen. When the probe is in the advanced configuration, the distal end of the probe can be outside the sheath lumen. When the probe is in the retracted configuration, a distal terminal end of the device can be the sheath. When the probe is in the advanced configuration, the distal terminal end of the device can be the probe. When the probe is in the retracted configuration, the sheath can form a distal terminal end of the device. When the probe is in the advanced configuration, the probe can form the distal terminal end of the device. When the probe is in the retracted configuration, the distal terminal end of the device can be a distal terminal end of the sheath. When the probe is in the advanced configuration, the distal terminal end of the device can be a distal terminal end of the probe. When the probe is in the retracted configuration, a distal terminal end of the sheath can form the distal terminal end of the device. When the probe is in the advanced configuration, a distal terminal end of the probe can form the distal terminal end of the device.

The sheath can have a sheath lumen. More of the probe can be distal the sheath lumen when the probe is in the advanced configuration than when the probe is in the retracted configuration. Some or none of the probe can be distal the sheath lumen when the probe is in the retracted configuration.

More of the probe can be outside the sheath when the probe is in the advanced configuration than when the probe is in the retracted configuration. Some or none of the probe can be outside the sheath when the probe is in the retracted configuration.

When the probe is in the retracted configuration, a distal terminal end of the probe can be proximal a distal terminal end of the sheath.

When the probe is in the retracted configuration, a distal terminal end of the probe can be distal a distal terminal end of the sheath. The device can have a penetrator. The penetrator can be the distal terminal end of the probe and/or the distal terminal end of the sheath.

When the probe is in the retracted configuration, a distal terminal end of the device can be a distal terminal end of the probe and a distal terminal end of the sheath.

When the probe is in the retracted configuration, a distal terminal end of the probe can be collinear with a distal terminal end of the sheath.

The device can have a material in a reservoir. Before the second distance becomes greater than or equal to the threshold distance, a flow path that extends between the reservoir and a distal end of the sheath can have a closed configuration. When and/or after the second distance becomes greater than or equal to the threshold distance, the flow path that extends between the reservoir and the distal end of the sheath can have an open configuration. A lumen of the sheath can be the flow path. The device can have a barrier. When the flow path has the closed configuration, the barrier can be in the flow path. When the flow path has the open configuration, the barrier can be outside of the flow path. Less of the barrier can be in the flow path when the flow path has the open configuration than when the flow path has the closed configuration. The barrier can have a valve and/or a trigger lock.

The device can have a lock. When the second distance is less than the threshold distance, the lock can be in a locked state. When the second distance is greater than or equal to the threshold distance, the lock can be in an unlocked state. When the lock is in the locked state, the device can prevent delivery of a material through the sheath. When the lock is in the unlocked state, the device can allow delivery of the material through the sheath.

The device can have a material. Before the second distance is greater than or equal to the threshold distance, the material can be outside the sheath and/or can be proximal a distal terminal end of the sheath. After the second distance is greater than or equal to the threshold distance, the material can be inside the sheath and/or can be distal the distal terminal end of the sheath. The material can be an implant (e.g., material of an implant, the entire implant, a portion of the implant), form an occlusion, be a hydrogel, be configured to dissolve an implant, or any combination thereof. The material can be sodium bicarbonate. The material can be configured to alter a physical property and/or a chemical property of an implant.

The device can have a material and a material reservoir. A flow path can extend between the material reservoir and a distal end of the sheath. The device can have a first configuration, a second configuration, and a third configuration. Before the second distance becomes greater than or equal to the threshold distance, the device can have the first configuration. When the second distance becomes greater than or equal to the threshold distance, the device can have the second configuration or the device can be changeable from the first configuration to the second configuration. After the second distance becomes greater than or equal to the threshold distance, the device can have the third configuration. When the device is in the first configuration, the material can be in the material reservoir and the flow path can be open or closed. When the device is in the second configuration, the material can be between the material reservoir and a distal terminal end of the sheath and the flow path can be open or closed. When the device is in the third configuration, the material can be distal a distal terminal end of the sheath and the flow path can be open or closed. The reservoir can be a first portion of a lumen of the sheath, and/or the flow path can be a second portion of the lumen of the sheath. The reservoir can be insertable into a lumen of the sheath. When the device is in the first configuration, the material can be outside of a lumen of the sheath. When the device is in the second configuration, the material can be inside the lumen of the sheath. When the device is in the second configuration, the material can be in contact with the probe and/or is distal a distal terminal end of the probe. When the device is in the second configuration, the probe can be in the retracted configuration. The material can be an implant (e.g., material of an implant, the entire implant, a portion of the implant), form an occlusion, be a hydrogel, be configured to dissolve an implant, or any combination thereof. The material can be sodium bicarbonate. The material can be configured to alter a physical property and/or a chemical property of an implant.

After the second distance becomes greater than or equal to the threshold distance, a material can be deliverable through the sheath. Before the second distance becomes greater than or equal to the threshold distance, the material may not be deliverable through the sheath. Before the second distance becomes greater than or equal to the threshold distance, the device can prevent delivery of a material through the sheath. The material can be an implant (e.g., material of an implant, the entire implant, a portion of the implant), form an occlusion, be a hydrogel, be configured to dissolve an implant, or any combination thereof. The material can be sodium bicarbonate. The material can be configured to alter a physical property and/or a chemical property of an implant.

The device can have a first material and a second material. Before the second distance is greater than or equal to the threshold distance, the first material can be outside the sheath and/or can be proximal a distal terminal end of the sheath. After the second distance is greater than or equal to the threshold distance, the first material can be inside the sheath and/or can be distal the distal terminal end of the sheath. Before the second distance is greater than or equal to the threshold distance, the second material can be outside the sheath. After the second distance is greater than or equal to the threshold distance, the second material can be inside the sheath and/or can be distal the distal terminal end of the sheath. The first material can be a fluid. The first material can be configured to dissolve the second material. The first material can be sodium bicarbonate. The first material can be configured to alter a physical property and/or a chemical property of the second material The second material can be an implant. The second material can be material of an implant. The second material can be implanted material. The implant, the material of the implant, and/or the implanted material can be a hydrogel.

The device can have a first material and a second material. The device can have a first configuration and a second configuration. When the device is in the first configuration, the first material can be proximal a distal terminal end of the sheath and the second material can be distal the distal terminal end of the sheath. When the device is in the second configuration, the first material and the second material can be distal the distal terminal end of the sheath. The device can have a third configuration. When the device is in the third configuration, the first material, the second material, and/or a third material can be proximal the distal terminal end of the sheath. The sheath can have a sheath lumen. When the device is in the first configuration, the first material can be in a reservoir and/or inside the sheath lumen and the second material can be outside the sheath lumen. When the device is in the second configuration, the first material and the second material can be outside the sheath lumen. When the device is in the second configuration, a third material can be outside the sheath lumen distal the distal terminal end of the sheath. An interaction between the first material and the second material outside the sheath can be configured to produce the third material. The device can have a third configuration. When the device is in the third configuration, the first material, the second material, and/or a third material can be inside the sheath lumen. An interaction between the first material and the second material can be configured to produce the third material outside the sheath lumen distal the distal terminal end of the sheath when the device is in the second configuration. The first material can be a fluid, the second material can be an implant (e.g., material of an implant, the entire implant, a portion of the implant), and the third material can be a mixture of the first material and the second material. The first material can be a fluid, the second material can be an implant (e.g., material of an implant, the entire implant, the portion of an implant), and the third material can be a material different than the first material and the second material. When the device is in the second configuration, an interaction between the first material and the second material can be configured to produce the third material outside the sheath lumen distal the distal terminal end of the sheath. The device can have a third configuration. When the device is in the third configuration, the first material, the second material, and/or the third material can be inside the sheath lumen.

The device can have a first material, a second material, a reservoir, and a receptacle. The reservoir can be configured to hold the first material. The receptacle can be configured to hold the first material, the second material, and/or a third material. The sheath can have a sheath lumen. The reservoir can include the sheath lumen. The receptacle can include the sheath lumen.

The device can have an irrigator and/or an aspirator. A distal end of the sheath can have a port. When the irrigator is irrigating, the port can be in fluid communication with the irrigator. When the aspirator is aspirating, the port can be in fluid communication with the aspirator. The irrigator can have a first manual pump and/or a first electric pump. The aspirator can have a second manual pump and/or a second electric pump. When the device is in a first configuration, a first material can flow out of the port. When the device is in a second configuration, the first material, a second material, and/or a third material can be configured to flow into the port. The sheath can have a sheath lumen. A distal terminal end of the sheath lumen can have the port.

The device can have an irrigator and/or an aspirator. A distal end of the sheath can have an outflow port and an inflow port. When the irrigator is irrigating, the outflow port can be in fluid communication with the irrigator. When the aspirator is aspirating, the inflow port can be in fluid communication with the aspirator. The irrigator can have a first manual pump and/or a first electric pump. The aspirator can have a second manual pump and/or a second electric pump. When the device is in a first configuration, a first material can be configured to flow out of the outflow port. When the device is in a second configuration, the first material, a second material, and/or a third material can be configured to flow into the inflow port. The sheath can have an inflow channel and an outflow channel. A distal terminal end of the inflow channel can have the inflow port. A distal terminal end of the outflow channel can have the outflow port.

The device can have a first material. More of the first material can be distal the sheath and the probe after the second distance is greater than or equal to the threshold distance than before the second distance is greater than or equal to the threshold distance. The first material can be configured to dissolve a second material. The first material can be sodium bicarbonate. The first material can be configured to alter a physical property and/or a chemical property of a second material. The device can have a second material. More of the second material can be proximal a distal terminal end of the sheath after the second distance is greater than or equal to the threshold distance than before the second distance is greater than or equal to the threshold distance. The second material can be an implant. The second material can be an occlusion. The second material can be a hydrogel. The device can have an aspirator. The aspirator can be configured to aspirate the first material and/or the second material into the sheath. The aspirator can be configured to simultaneously aspirate the first material and the second material into the sheath. The probe can be configured to agitate a second material outside the sheath and/or inside a lumen of the sheath. The probe can be configured to agitate the first material outside the sheath and/or inside the lumen of the sheath. The probe can be configured to agitate the first material and/or the second material outside the sheath and/or inside a lumen of the sheath. The probe can be a first probe. The device can have a second probe. The second probe can be configured to agitate a second material outside the sheath and/or inside a lumen of the sheath. The second probe can be configured to agitate the first material outside the sheath and/or inside the lumen of the sheath.

The probe can be a first probe. The device can have a second probe. The second probe can be configured to agitate the first material and/or a second material outside the sheath and/or inside a lumen of the sheath.

The probe can be a first probe The device can have a second probe. The first probe and/or the second probe can be configured to agitate a first material and/or a second material outside the sheath and/or inside a lumen of the sheath. The first material can be a fluid. The second material can be an implant. The implant can be a hydrogel. The device can have an aspirator. The aspirator can be configured to aspirate the first material and/or the second material into the sheath.

The probe can be a first probe. The device can have a second probe. The first probe and/or the second probe can be configured to agitate a first material, a second material, and/or a third material outside the sheath and/or inside a lumen of the sheath. The device can have an aspirator. The aspirator can be configured to aspirate the first material and/or the second material into the sheath. The first material can be a fluid. The second material can be an implant. The implant can be a hydrogel.

The device can have a spring. The probe can be advanceable from the retracted configuration to the advanced configuration via the spring. The probe can be retractable from the advanced configuration to the retracted configuration via the spring.

The first distance can be measured along a straight line or a curved line between a tip of the sheath and a tip of the probe.

The second distance can be measured along a straight line and/or a curved line between a tip of the sheath and a tip of the probe.

The second distance can be measured along the same straight line and/or the same curved line as the first distance can be measured along.

The first distance can be a longitudinal distance between a tip of the sheath and a tip of the probe.

The first distance can be a distance of zero (e.g., 0.0 cm, 0.0 mm) between a tip of the sheath and a tip of the probe.

The probe can be retractable from the advanced configuration to the retracted configuration relative to the sheath. The probe can be advanceable from the retracted configuration to the advanced configuration inside a lumen of the sheath. The probe can be retractable from the advanced configuration to the retracted configuration inside the lumen of the sheath.

The threshold distance can be 1.0 cm to 15.0 cm. The threshold distance can be 1.0 cm to 10.0 cm. The threshold distance can be 1.0 cm to 5.0 cm. The threshold distance can be 5.0 cm, 5.0 cm or less, 10.0 cm or less, or 15.0 cm or less.

The sheath can be a tube. The sheath can be a catheter.

A distal end of the sheath can be configured to puncture tissue.

The sheath can be a needle.

A distal end of the sheath can be a needle.

The sheath can have a needle.

The device can have a needle.

The probe can be a guidewire.

The first probe and/or the second probe can be a guidewire.

The second probe can be the same as the first probe, for example, such that the first probe and the second probe are identical probes but part of two different devices (e.g., two different identical devices).

When the second distance is greater than or equal to the threshold distance, the advanced configuration of the probe can be an indicator to a user that the sheath and/or a port has access to the target site.

The sheath can have a sheath lumen. More of the probe can be distal the sheath lumen when the probe is in the advanced configuration than when the probe is in the retracted configuration.

The device can have a material and/or a material reservoir. A flow path can extend between the material reservoir and a distal end of the sheath. The device can have a first configuration, a second configuration, and a third configuration. Before the second distance becomes greater than or equal to the threshold distance, the device can have the first configuration. When the second distance becomes greater than or equal to the threshold distance, the device can have the second configuration or the device can be changeable from the first configuration to the second configuration. After the second distance becomes greater than or equal to the threshold distance, the device can have the third configuration. When the device is in the first configuration, the material can be in the material reservoir and the flow path can be open or closed. When the device is in the second configuration, the material can be between the material reservoir and a distal terminal end of the sheath and the flow path can be open or closed. When the device is in the third configuration, the material can be distal a distal terminal end of the sheath and the flow path can be open or closed.

The device can have an irrigator and/or an aspirator. A distal end of the sheath can have a port. When the irrigator is irrigating, the port can be in fluid communication with the irrigator. When the aspirator is aspirating, the port can be in fluid communication with the aspirator.

The systems, devices, and/or methods disclosed herein can have any combination of features shown in the drawings and/or described herein (e.g., any combination of features between “A method of confirming intraluminal access of a lumen in a body is disclosed (e.g., as shown in the drawings and/or as described herein)” and “The second probe can be the same as the first probe, for example, such that the first probe and the second probe are identical probes but part of two different devices (e.g., two different identical devices)” above).

A method of removing a first material from a body lumen is disclosed (e.g., as shown in the drawings and/or as described herein). The method can include inserting a sheath into the body lumen, advancing a probe into the body lumen relative to the sheath, agitating the first material in the body lumen with the probe, irrigating a second material into the body lumen, and/or removing the first material from the body lumen.

Inserting the sheath into the body can include penetrating tissue with a penetrator. The penetrator can be a distal end of the sheath and/or a needle.

Inserting the sheath into the body can include inserting the sheath through an incision.

Agitating the first material in the body lumen with the probe can include mechanically agitating the first material in the body lumen with the probe. The first material can be an implant. The first material can be material of an implant. The first material can be implanted material. The implant, the material of the implant, and/or the implanted material can be a hydrogel. The second material can be a fluid (e.g., sodium bicarbonate). The second material can change a chemical property and/or a physical property of the first material.

Agitating the first material in the body lumen with the probe can include moving the probe relative to the sheath in the body lumen. Moving the probe relative to the sheath in the body lumen can include translating, rotating, and/or vibrating the probe in the body lumen. Moving the probe in the body lumen can include a user and/or a motion source moving the probe. The motion source can include a vibratory motion source, an ultrasonic motion source, a translation motion source, and/or a rotational motion source. Moving the probe in the body lumen can include applying vibratory motion, a dottering motion, an ultrasonic motion, a translational motion, and/or a rotational motion to the probe.

Agitating the first material in the body lumen with the probe can include agitating the first material in the body lumen while the probe is in contact with the first material.

Agitating the first material in the body lumen with the probe can include moving the probe into and out of contact with the first material.

Agitating the first material in the body lumen with the probe can include moving the probe into and out of contact with the first material while the second material is in contact with the first material.

Agitating the first material in the body lumen with the probe can include agitating the first material in the body lumen while the probe is in contact with the second material.

Agitating the first material in the body lumen with the probe can include moving the first material in the body lumen via the probe while the second material is in contact with the first material and/or the probe in the body lumen. The method can include moving the first material in the body lumen via irrigating the second material into the body lumen. The method can include removing the second material from the body lumen. The method can include removing the second material from the body lumen into the sheath. The method can include moving the first material in the body lumen via removing the second material from the body lumen into the sheath. The method can include moving the first material in the body lumen via irrigating the second material into the body lumen and/or removing the second material from the body lumen. The method can include moving the first material in the body lumen via simultaneously irrigating the second material into the body lumen and removing the second material from the body lumen into the sheath.

The method can include moving the first material in the body lumen via a flow of the second material in the body lumen toward and/or away from the sheath.

Agitating the first material in the body lumen with the probe can include moving the first material relative to the sheath and/or the second material.

Agitating the first material in the body lumen with the probe can include moving the first material toward and/or away from the sheath in the body lumen.

Agitating the first material in the body lumen with the probe can include moving the first material toward and/or away from the second material in the body lumen.

Agitating the first material in the body lumen with the probe can include mechanically breaking down the first material with the probe.

Agitating the first material in the body lumen with the probe can include mixing the first material and the second material together in the body lumen. The first material can be an implant. The first material can be material of an implant. The first material can be implanted material. The implant, the material of the implant, and/or the implanted material can be a hydrogel. The second material can be a fluid (e.g., sodium bicarbonate). The second material can change a chemical property and/or a physical property of the first material.

Agitating the first material in the body lumen with the probe can include forcing the first material into the second material and/or forcing the second material into the first material.

Agitating the first material in the body lumen with the probe can include mixing the first material into the second material and/or mixing the second material into the first material.

The method can include forming, via the first material and the second material interacting with each other in the body lumen, a solution and/or a mixture in the body lumen. The first material and the second material interacting with each other in the body lumen can include the first material and the second material mixing in the body lumen and/or a chemical reaction between the first material and the second material in the body lumen. The first material can be an implant. The first material can be material of an implant. The first material can be implanted material. The implant, the material of the implant, and/or the implanted material can be a hydrogel. The second material can be a fluid.

The method can include forming, from the first material and/or the second material in the body lumen, a solution and/or a mixture in the body lumen. The solution and/or the mixture can be a third material. The solution and/or the mixture can be a dissolved form of the first material and/or a disassociated form of the first material.

The method can include forming a dissolved form of the first material and/or a disassociated form of the first material in the body lumen.

The method can include forming a dissolved form of the first material and/or a disassociated form of the first material via an interaction between the first material and the second material in the body lumen.

Agitating the first material in the body lumen with the probe can include forming, from the first material and/or the second material in the body lumen, a solution and/or a mixture in the body lumen. Agitating the first material in the body lumen with the probe can include forming a third material in the body lumen. The solution and/or the mixture can be the third material. The third material can have a chemical property that is different from the first material and/or the second material.

The method can include forming, via the second material and a third material interacting with each other in the body lumen, the first material in the body lumen. The first material can be a solution and/or a mixture. The second material can be a fluid. The third material can be an implant. The third material can be material of an implant. The third material can be implanted material. The implant, the material of the implant, and/or the implanted material can be a hydrogel. The solution and/or the mixture can be a portion of the implant. The solution and/or the mixture can be a piece of the implant. The solution and/or the mixture can be a dissolved form of the third material and/or a disassociated form of the third material. The second material and the third material interacting with each other in the body lumen can include the second material and the third material mixing in the body lumen and/or a chemical reaction between the second material and the third material in the body lumen.

The method can include forming, from the second material and/or a third material in the body lumen, the first material in the body lumen. The first material can be a dissolved form of the third material and/or a disassociated form of the third material. The second material can be a fluid. The third material can be an implant. The third material can be material of an implant. The third material can be implanted material. The implant, the material of the implant, and/or the implanted material can be a hydrogel.

Agitating the first material in the body lumen with the probe can include forming, from the second material and/or a third material in the body lumen, the first material in the body lumen. The second material can be a fluid. The third material can be an implant. The third material can be material of an implant. The third material can be implanted material. The implant, the material of the implant, and/or the implanted material can be a hydrogel. The first material can be a mixture and/or a solution having the second material and/or the third material. The first material can have a chemical property that is different from a chemical property of the first material and/or a chemical property of the second material.

The method can include agitating the second material in the body lumen with the probe. Agitating the second material in the body lumen with the probe can include mechanically agitating the second material in the body lumen with the probe. Agitating the second material in the body lumen with the probe can include moving the probe in the body lumen. Moving the probe in the body lumen can include translating, rotating, and/or vibrating the probe in the body lumen. Moving the probe in the body lumen can include a user and/or a motion source moving the probe. The motion source can be a vibratory motion source, an ultrasonic motion source, a translation motion source, and/or a rotational motion source. Moving the probe in the body lumen can include applying vibratory motion, a dottering motion, an ultrasonic motion, a translational motion, and/or a rotational motion to the probe. Agitating the second material in the body lumen with the probe can include agitating the second material in the body lumen while the probe is in contact with the first material and/or the second material. Agitating the first material in the body lumen with the probe can include moving the probe into and out of contact with the first material. Agitating the second material in the body lumen with the probe can include moving the second material while the second material is in contact with the first material in the body lumen. Agitating the second material in the body lumen with the probe can include agitating the second material in the body lumen while the probe is in contact with the second material. Agitating the second material in the body lumen with the probe can include moving the second material relative to the sheath and/or the first material. Agitating the second material in the body lumen with the probe can include moving the second material toward and/or away from the sheath in the body lumen. Agitating the second material in the body lumen with the probe can include moving the second material toward and/or away from the first material in the body lumen. Agitating the first material in the body lumen with the probe and agitating the second material in the body lumen with the probe can include simultaneously agitating the first material and the second material in the body lumen with the probe. Agitating the second material in the body lumen with the probe can include mixing the first material and the second material together in the body lumen. Agitating the second material in the body lumen with the probe can include forcing the first material into the second material and/or forcing the second material into the first material. Agitating the second material in the body lumen with the probe can include mixing the first material into the second material and/or mixing the second material into the first material. Agitating the second material in the body lumen with the probe can include creating turbulence and/or flow in the second material in the body lumen. The second material can be a fluid. The second material can be a liquid and/or a gas.

The method can include agitating the second material and/or a third material in the body lumen with the probe. Agitating the second material and/or the third material in the body lumen with the probe can include mechanically agitating the second material and/or the third material in the body lumen with the probe. Agitating the second material and/or the third material in the body lumen with the probe can include moving the probe in the body lumen. Moving the probe in the body lumen can include translating, rotating, and/or vibrating the probe in the body lumen. Moving the probe in the body lumen can include a user and/or a motion source moving the probe. The motion source can be a vibratory motion source, an ultrasonic motion source, a translation motion source, and/or a rotational motion source. Moving the probe in the body lumen can include applying vibratory motion, a dottering motion, an ultrasonic motion, a translational motion, and/or a rotational motion to the probe. Agitating the second material and/or the third material in the body lumen with the probe can include forming the third material in the body lumen by mixing the first material and the second material together in the body lumen. Agitating the second material and/or the third material in the body lumen with the probe can include agitating the second material and/or the third material in the body lumen while the probe is in contact with the second material and/or the third material. Agitating the first material in the body lumen with the probe can include moving the probe into and out of contact with the first material. Agitating the second material and/or the third material in the body lumen with the probe can include mixing the first material and the second material together in the body lumen. Agitating the second material and/or the third material in the body lumen with the probe can include forcing the first material into the second material and/or forcing the second material into the first material. Agitating the second material and/or the third material in the body lumen with the probe can include mixing the first material into the second material and/or mixing the second material into the first material. Agitating the second material and/or the third material in the body lumen with the probe can include creating turbulence and/or flow in the second material and/or the third material in the body lumen. The second material can be a first fluid. The third material can be a second fluid. The third material can be a mixture of the first material and the second material and/or can be a different material than the first material and/or the second material. Agitating the second material and/or the third material in the body lumen with the probe can include moving the second material and/or the third material relative to the sheath and/or the first material. Agitating the second material and/or the third material in the body lumen with the probe can include moving the second material and/or the third material toward and/or away from the sheath in the body lumen. Agitating the second material and/or the third material in the body lumen with the probe can include moving the second material and/or the third material toward and/or away from the first material in the body lumen. The method can include simultaneously agitating the first material, the second material, and the third material in the body lumen with the probe. Agitating the second material and/or the third material in the body lumen with the probe can include moving the second material and/or the third material while the second material is in contact with the first material in the body lumen. Agitating the second material and/or the third material in the body lumen with the probe can include agitating the second material and/or the third material in the body lumen while the probe is in contact with the second material and/or the third material. Agitating the second material and/or the third material in the body lumen with the probe can include agitating the second material and/or the third material in the body lumen while the probe is in contact with the first material, the second material, and the third material.

The body lumen can be a lumen of a reproductive tract. The body lumen can be a lumen of a vas deferens. The body lumen can be a lumen of a blood vessel.

The first material can be an implanted material or an implant. The first material can form an occlusion. The first material can include a hydrogel. The first material can include a fluid and an implant. The first material can be a mixture and/or a solution having the second material and/or a third material.

The second material can be a dissolving material. The second material can be a sodium bicarbonate solution. The second material can be configured to dissolve and/or disassociate the first material. The second material can be a liquid and/or a gas.

The method can include a third material or having a third material. The third material can be a fluid and/or an implant. The third material can be a mixture and/or a solution comprising the first material and/or the second material.

The method can include forming a third material via an interaction between the first material and the second material.

The method can include forming a third material in the body lumen via an interaction between the first material and the second material in the body lumen.

The method can include forming a third material via an interaction between the first material and the second material distal a distal terminal end of the sheath. The method can include forming the third material via an interaction between the first material and the second material distal a distal terminal end of the probe. Forming the third material via the interaction between the first material and the second material distal the distal terminal end of the sheath can include forming the third material forming the third material via the interaction between the first material and the second material between the distal terminal end of the sheath and a distal terminal end of the probe.

The method can include forming the third material via an interaction between the first material and the second material in the body lumen outside the sheath. The third material can be a solution comprising the first material and the second material. The third material can include a mixture of the first material and the second material. The third material can have a different physical property or a different chemical property than the first material and/or the second material.

The method can include forming a third material via the second material dissolving and/or disassociating the first material.

The method can include forming a third material via the second material dissolving and/or disassociating the first material distal a distal terminal end of the sheath. The method can include forming the third material via the second material dissolving and/or disassociating the first material distal a distal terminal end of the probe. Forming the third material via the second material dissolving and/or disassociating the first material distal the distal terminal end of the sheath can include forming the third material via the second material dissolving and/or disassociating the first material between the distal terminal end of the sheath and a distal terminal end of the probe.

The method can include forming a third material via the second material dissolving and/or disassociating the first material in the body lumen outside the sheath. The third material can be a solution comprising the first material and the second material. The third material can comprise a mixture of the first material and the second material. The third material can have a different physical property or a different chemical property than the first material and/or the second material.

The method can include forming a third material by altering a physical property and/or a chemical property of the first material via the second material.

The method can include forming a third material by altering a physical property and/or a chemical property of the first material via the second material distal a distal terminal end of the sheath. The method can include forming the third material by altering the physical property and/or the chemical property of the first material via the second material distal a distal terminal end of the probe. Forming the third material by altering the physical property and/or the chemical property of the first material via the second material distal the distal terminal end of the sheath can include forming the third material by altering the physical property and/or the chemical property of the first material via the second material between the distal terminal end of the sheath and a distal terminal end of the probe.

The method can include forming a third material by altering a physical property and/or a chemical property of the first material via the second material in the body lumen outside the sheath. The third material can be a solution comprising the first material and the second material. The third material can be a mixture of the first material and the second material.

The method can include dissolving and/or disassociating the first material with the second material.

The method can include dissolving and/or disassociating the first material with the second material in the body lumen. Dissolving and/or disassociating the first material with the second material in the body lumen can form a dissolved and/or disassociated first material in the body lumen. Removing the first material from the body lumen can include removing the dissolved and/or disassociated first material from the body lumen. Dissolving and/or disassociating the first material with the second material in the body lumen can form a mixture of the first material and the second material. Removing the first material from the body lumen can include removing the mixture of the first material and the second material from the body lumen. Dissolving and/or disassociating the first material with the second material in the body lumen can form a third material in the body lumen. The third material can be a solution comprising the first material and the second material. The third material can be a mixture of the first material and the second material. The third material can have a different physical property or a different chemical property than the first material and/or the second material. Removing the first material from the body lumen can include removing the third material from the body lumen. Removing the first material from the body lumen can include first forming the third material by dissolving and/or disassociating the first material with the second material in the body lumen and then removing the third material from the body lumen.

The method can include simultaneously irrigating the second material, agitating the first material, and removing the first material and the second material.

The method can include simultaneously removing the first material, the second material, and/or a third material from the body lumen.

The probe can have a protrusion, a bristle, a brush, an indentation, a coil, a loop, and/or an angulation. Agitating the first material in the body lumen with the probe can include agitating the first material in the body lumen with the protrusion, the bristle, the brush, the indentation, the coil, the loop, and/or the angulation.

The probe can have protrusions, bristles, brushes, indentations, coils, loops, and/or angulations. Agitating the first material in the body lumen with the probe can include agitating the first material in the body lumen with the protrusions, the bristles, the brushes, the indentations, the coils, the loops, and/or the angulations.

The probe can have one or multiple protrusions, one or multiple bristles, one or multiple brushes, one or multiple indentations, one or multiple coils, one or multiple loops, and/or one or multiple angulations. Agitating the first material in the body lumen with the probe can include agitating the first material in the body lumen with the protrusions, the bristles, the brushes, the indentations, the coils, the loops, and/or the angulations.

Advancing the probe into the body lumen relative to the sheath can include advancing the probe out of the sheath and/or the method can include retracting the probe into the sheath.

Advancing the probe into the body lumen relative to the sheath can include advancing the probe into the body lumen relative to the sheath after inserting the sheath into the body lumen.

The method can include agitating the first material inside the body lumen from outside the body lumen with an agitator. The method can include applying the agitator to an external surface outside of the body lumen. The external surface can include an external surface of a scrotum. The agitator can be a vibrator.

Removing the first material from the body lumen can include removing the first material from the body lumen into the sheath.

The method can include irrigating the second material into the body lumen when the first material is distal a distal terminal end of the sheath and/or when the second material is 15.0 cm or less, 10.0 cm or less, or 5.0 cm or less from the distal terminal end of the sheath.

The method can include irrigating the second material into the body lumen when the first material is between a distal terminal end of the sheath and a distal terminal end of the probe.

Removing the first material from the body lumen can include aspirating the first material from the body lumen.

The method can include removing the second material from the body lumen. Removing the first material from the body lumen can include aspirating the first material from the body lumen. Removing the second material from the body lumen can include aspirating the second material from the body lumen. Removing the first material from the body lumen and removing the second material from the body lumen can include simultaneously aspirating the first material and the second material from the body lumen.

The method can include removing the second material and/or a third material from the body lumen. The third material can be a solution comprising the first material and the second material. The third material can be a mixture of the first material and the second material. The third material can have a different physical property or a different chemical property than the first material and/or the second material. The sheath can have a port. Irrigating the second material into the body lumen can include irrigating the second material into the body lumen through the port. Removing the first material, the second material, and/or the third material from the body lumen can include removing the first material, the second material, and/or the third material from the body lumen through the port. Removing the first material, the second material, and/or the third material from the body lumen can include aspirating the first material, the second material, and/or the third material from the body lumen through the port. The port can be a distal terminal opening of the sheath or an opening on a side of the sheath. The sheath can have an outflow port and an inflow port. Irrigating the second material into the body lumen can include irrigating the second material into the body lumen through the inflow port. Removing the first material, the second material, and/or the third material from the body lumen can include removing the first material, the second material, and/or the third material from the body lumen through the outflow port. Removing the first material, the second material, and/or the third material from the body lumen can include aspirating the first material, the second material, and/or the third material from the body lumen through the outflow port. The sheath can have an inflow channel and an outflow channel. A distal terminal end of the inflow channel can have the inflow port. A distal terminal end of the outflow channel can have the outflow port. The outflow port can be a first distal terminal opening of the sheath and/or a first opening on a side of the sheath. The inflow port can be a second distal terminal opening of the sheath and/or a second opening on a side of the sheath.

The sheath can have a first port. The probe can have a second port. Irrigating the second material into the body lumen can include irrigating the second material into the body lumen through the first port. Removing the first material, the second material, and/or the third material from the body lumen can include removing the first material, the second material, and/or the third material from the body lumen through the second port.

The sheath can have a first port. The probe can have a second port. Irrigating the second material into the body lumen can include irrigating the second material into the body lumen through the second port. Removing the first material, the second material, and/or the third material from the body lumen can include removing the first material, the second material, and/or the third material from the body lumen through the first port.

The probe can have an outflow port and/or an inflow port. Irrigating the second material into the body lumen can include irrigating the second material into the body lumen through the inflow port. Removing the first material, the second material, and/or the third material from the body lumen can include removing the first material, the second material, and/or the third material from the body lumen through the outflow port. Removing the first material, the second material, and/or the third material from the body lumen can include aspirating the first material, the second material, and/or the third material from the body lumen through the outflow port. The probe can have an inflow channel and an outflow channel. A distal terminal end of the inflow channel can have the inflow port. A distal terminal end of the outflow channel can have the outflow port. The outflow port can be a first distal terminal opening of the probe and/or a first opening on a side of the probe. The inflow port can be a second distal terminal opening of the probe and/or a second opening on a side of the probe.

A method of removing a first material from a body lumen is disclosed (e.g., as shown in the drawings and/or as described herein). The method can include inserting a sheath into the body lumen, inserting a probe into the body lumen, agitating the first material in the body lumen with the probe, irrigating a second material into the body lumen, and/or aspirating the first material and the second material from the body lumen.

Agitating the first material in the body lumen with the probe can include moving the probe in the body lumen. Moving the probe in the body lumen can include translating, rotating, and/or vibrating the probe in the body lumen.

Agitating the first material in the body lumen with the probe can include mechanically breaking down the first material with the probe.

The body lumen can be a lumen of a reproductive tract. The body lumen can be a lumen of a vas deferens.

The first material can be an implanted material. The first material can form an occlusion. The first material can be a hydrogel.

The second material can be a dissolving material. The second material can be configured to dissolve another material. The second material can be a sodium bicarbonate solution.

The method can include agitating the second material in the body lumen with the probe.

The method can include advancing the probe out of the sheath and/or retracting the probe into the sheath. The method can include agitating the first material in the body lumen with the probe after advancing the probe out of the sheath. Agitating the first material in the body lumen with the probe can be different from advancing the probe out of the sheath and/or retracting the probe into the sheath. Agitating the first material in the body lumen with the probe can include moving the probe in the body lumen relative to the sheath. The method can include moving the probe in the body lumen relative to the sheath after advancing the probe out of the sheath. Agitating the first material in the body lumen with the probe can include moving the probe in the body lumen relative to the sheath. Moving the probe in the body lumen relative to the sheath can be a different step from advancing the probe out of the sheath and/or retracting the probe into the sheath.

Agitating the first material in the body lumen with the probe can include moving the probe relative to the sheath in the body lumen. Moving the probe relative to the sheath in the body lumen can include translating, dottering, rotating, and/or vibrating the probe in the body lumen.

Agitating the first material in the body lumen with the probe can include mechanically breaking down the first material with the probe.

The lumen can be a lumen of a reproductive tract.

The method can include agitating the second material in the body lumen with the probe.

A method of removing a first material, a second material, and/or a third material from a body lumen is disclosed (e.g., as shown in the drawings and/or as described herein). The method can include inserting a sheath into the body lumen, inserting a probe into the body lumen, agitating the first material in the body lumen with the probe, irrigating the second material into the body lumen, and/or removing the first material, the second material, and/or the third material from the body lumen.

The method can include agitating the second material in the body lumen with the probe.

The method can include agitating the second material and the third material in the body lumen with the probe. Agitating the first material, the second material, and the third material in the body lumen with the probe can include mechanically agitating the first material, second material and the third material in the body lumen with the probe. Agitating the first material, the second material, and the third material in the body lumen with the probe can include moving the probe in the body lumen. Moving the probe in the body lumen can include translating, rotating, and/or vibrating the probe in the body lumen. Moving the probe in the body lumen can include a user and/or a motion source moving the probe. The motion source can be a vibratory motion source, an ultrasonic motion source, a translation motion source, and/or a rotational motion source. Moving the probe in the body lumen can include applying vibratory motion, a dottering motion, an ultrasonic motion, a translational motion, and/or a rotational motion to the probe.

Agitating the first material, the second material, and the third material in the body lumen with the probe can include forming the third material in the body lumen by mixing the first material and the second material together in the body lumen.

Agitating the first material, the second material, and the third material in the body lumen with the probe can include agitating the first material, the second material, and the third material in the body lumen while the probe is in contact with the first material, the second material, and/or the third material. Agitating the first material in the body lumen with the probe can include moving the probe into and out of contact with the first material.

Agitating the first material, the second material, and the third material in the body lumen with the probe can include mixing the first material and the second material together in the body lumen.

Agitating the first material, the second material, and the third material in the body lumen with the probe can include forcing the first material into the second material and/or forcing the second material into the first material.

Agitating the first material, the second material, and the third material in the body lumen with the probe can include mixing the first material into the second material and/or mixing the second material into the first material.

Agitating the first material, the second material, and the third material in the body lumen with the probe can include creating turbulence and/or flow in the second material and/or the third material in the body lumen. The second material can be a first fluid. The third material can be a second fluid. The third material can be a mixture of the first material and the second material and/or can comprise a different material than the first material and/or the second material.

Agitating the first material, the second material, and the third material in the body lumen with the probe can include moving the first material and/or the second material relative to the third material, moving the second material and/or the third material relative to the first material, and/or moving the first material and/or the third material relative to the second material.

Agitating the first material, the second material, and the third material in the body lumen with the probe can include moving the second material and/or the third material toward and/or away from the first material in the body lumen.

Agitating the first material, the second material, and the third material in the body lumen with the probe can include moving the second material and/or the third material while the second material is in contact with the first material in the body lumen. Agitating the first material, the second material, and the third material in the body lumen with the probe can include agitating the second material and/or the third material in the body lumen while the probe is in contact with the second material and/or the third material. Agitating the first material, the second material, and the third material in the body lumen with the probe can include agitating the second material and/or the third material in the body lumen while the probe is in contact with the first material, the second material, and the third material.

The first material can be an implanted material, an implant, and/or a hydrogel. The first material can form an occlusion.

The second material can be configured to dissolve and/or disassociate the first material.

The method can include forming the third material via an interaction between the first material and the second material in the body lumen. The third material can be a solution comprising the first material and the second material. The third material can comprise a mixture of the first material and the second material. The third material can have a different physical property or a different chemical property than the first material and/or the second material.

The method can include irrigating the second material into the body lumen when the first material is distal a distal terminal end of the sheath and/or when the second material is 15.0 cm or less, 10.0 cm or less, or 5.0 cm or less from the distal terminal end of the sheath.

The method can include irrigating the second material into the body lumen when the first material is between a distal terminal end of the sheath and a distal terminal end of the probe.

Removing the first material, the second material, and/or the third material from the body lumen can include aspirating the first material, the second material, and/or the third material from the body lumen.

The sheath can have a port. Irrigating the second material into the body lumen can include irrigating the second material into the body lumen through the port. Removing the first material, the second material, and/or the third material from the body lumen can include removing the first material, the second material, and/or the third material from the body lumen through the port. Removing the first material, the second material, and/or the third material from the body lumen can include aspirating the first material, the second material, and/or the third material from the body lumen through the port. The port can be a distal terminal opening of the sheath and/or an opening on a side of the sheath.

The sheath can have an outflow port and an inflow port. Irrigating the second material into the body lumen can include irrigating the second material into the body lumen through the inflow port. Removing the first material, the second material, and/or the third material from the body lumen can include removing the first material, the second material, and/or the third material from the body lumen through the outflow port. Removing the first material, the second material, and/or the third material from the body lumen can include aspirating the first material, the second material, and/or the third material from the body lumen through the outflow port. The sheath can have an inflow channel and an outflow channel. A distal terminal end of the inflow channel can have the inflow port. A distal terminal end of the outflow channel can have the outflow port. The outflow port can be a first distal terminal opening of the sheath and/or a first opening on a side of the sheath. The inflow port can be a second distal terminal opening of the sheath and/or a second opening on a side of the sheath.

The sheath can have a first port. The probe can have a second port. Irrigating the second material into the body lumen can include irrigating the second material into the body lumen through the first port. Removing the first material, the second material, and/or the third material from the body lumen can include removing the first material, the second material, and/or the third material from the body lumen through the second port.

The sheath can have a first port. The probe can have a second port. Irrigating the second material into the body lumen can include irrigating the second material into the body lumen through the second port. Removing the first material, the second material, and/or the third material from the body lumen can include removing the first material, the second material, and/or the third material from the body lumen through the first port.

The probe can have an outflow port and/or an inflow port. Irrigating the second material into the body lumen can include irrigating the second material into the body lumen through the inflow port. Removing the first material, the second material, and/or the third material from the body lumen can include removing the first material, the second material, and/or the third material from the body lumen through the outflow port. Removing the first material, the second material, and/or the third material from the body lumen can include aspirating the first material, the second material, and/or the third material from the body lumen through the outflow port. The probe can have an inflow channel and an outflow channel. A distal terminal end of the inflow channel can have the inflow port. A distal terminal end of the outflow channel can have the outflow port. The outflow port can be a first distal terminal opening of the probe and/or a first opening on a side of the probe. The inflow port can be a second distal terminal opening of the probe and/or a second opening on a side of the probe.

A device is disclosed (e.g., as shown in the drawings and/or as described herein). The device can have a sheath, a probe, a first material, and/or a first opening. The device can have a first configuration and a second configuration. More of the first material can be distal the first opening when the device is in the second configuration than when the device is in the first configuration. The first material can be closer to a second material when the device is in the second configuration than when the device is in the first configuration. The probe can have a retracted configuration and an advanced configuration. More of the probe can be distal the first opening when the probe is in the advanced configuration than when the probe is in the retracted configuration. When the probe is in the advanced configuration, the probe can be movable from a first position to a second position. When the device is in the second configuration and the probe is in the first position, the probe can be in contact with the first material. When the device is in the second configuration and the probe is in the second position, the probe can be in contact with the first material or a gap can be between the probe and the first material. A distal terminal end of the probe can be the same distance or a different distance from the first opening when the probe is in the second position than when the probe is in the first position.

The first material can be an irrigant.

The sheath can have the first opening.

More of the first material can be in contact with the second material when the device is in the second configuration than when the device is in the first configuration.

The first material can be in contact with the second material when the device is in the second configuration.

Some or none of the first material can be distal the first opening when the device is in the first configuration.

Some or none of the first material can be in contact with the second material when the device is in the first configuration.

More of a distal end of the probe can be inside of the sheath when the probe is in the retracted configuration than when the probe is in the advanced configuration.

When the probe is in the retracted configuration, a first distance can be between the sheath and the distal terminal end of the probe and the first opening. When the probe is in the advanced configuration, a second distance greater than the first distance can be between the distal terminal end of the probe and first opening. The second distance can be 1.0 cm to 15.0 cm greater than the first distance. When the probe is in the advanced configuration, 1.0 cm to 15.0 cm of the probe can be distal the first opening.

When the probe is in the advanced configuration, the probe can be movable from the second position to the first position.

When the probe is in the advanced configuration, the probe can be translatable and/or rotatable from the first position to the second position.

When the probe is in the advanced configuration, the probe can be translatable and/or rotatable back and forth between the first position and the second position.

When the probe is in the advanced configuration, the probe can be configured to oscillate back and forth between the first position and the second position.

When the probe is in the advanced configuration, the distal terminal end of the probe can be 0.01 mm to 5.00 mm farther from the first opening when the probe is in the second position than when the probe is in the first position.

When the probe is in the advanced configuration, the distal terminal end of the probe can be 0.1 mm to 5.0 mm farther from the first opening when the probe is in the second position than when the probe is in the first position.

When the probe is in the advanced configuration, the distal terminal end of the probe can be 0.5 mm to 5.0 mm farther from the first opening when the probe is in the second position than when the probe is in the first position.

When the device is in the second configuration and the probe is in the advanced configuration, the first material can surround the probe. When the device is in the second configuration and the probe is in the advanced configuration, the probe can be surrounded (e.g., circumferentially surrounded) by the first material.

When the device is in the second configuration and the probe is in the first position, the probe can be in contact with the second material.

When the device is in the second configuration and the probe is in the second position, the probe can be in contact with the second material.

The device can have a first material reservoir. The first material reservoir can be filled with the first material. The first material reservoir can have the first material. Less of the first material can be in the first material reservoir when the device is in the second configuration than when the device is in the first configuration. The sheath can have the first material reservoir. The first material reservoir can include a lumen of the sheath (e.g., an inflow lumen of the sheath).

The device can have a first material reservoir. The first material reservoir can be filled with the first material. The first material reservoir can have the first material. When the device is irrigating the first material, the first opening and/or a second opening can be in fluid communication with the first material reservoir. The first opening can be distal the second opening. The sheath can have the first opening and/or the second opening. The sheath can have a lumen. A first end of the lumen can have the first opening. A second end of the lumen can have the second opening.

The device can have a first material reservoir. The first material reservoir can be filled with the first material. The first material reservoir can have the first material. When the device is irrigating the first material, the first opening and/or a second opening can be in fluid communication with the first material reservoir.

The first configuration can be a first deployed configuration of the device and the second configuration can be a second deployed configuration of the device.

The device can have a third configuration. When the device is in the second configuration, the first material distal the first opening can be an irrigated first material. More of the irrigated first material can be proximal the first opening when the device is in the third configuration than when the device is in the second configuration.

The device can have a third configuration. When the device is in the second configuration, the first material distal the first opening can be an irrigated first material. More of the irrigated first material can be in a lumen of the sheath when the device is in the third configuration than when the device is in the second configuration.

The device can have a remover. The first material, the second material, and/or a third material can be closer to the first opening when the device is in a third configuration than when the device is in the second configuration. The remover can be an aspirator. The device can have a third configuration. More of the second material and/or a third material can be in a lumen of the sheath when the device is in the third configuration than when the device is in the second configuration.

The first configuration can be a first deployed configuration of the device, the second configuration can be a second deployed configuration of the device, and the third configuration can be a third deployed configuration of the device.

The device can have a first reservoir and a second reservoir. When the device is delivering the first material to a space distal the first opening, the first opening can be in fluid communication with the first material reservoir. When the device is removing the first material, the second material, and/or a third material from the space distal the first opening, a second opening can be in fluid communication with the second reservoir. The sheath can have the first reservoir and/or the second reservoir. The first reservoir can include a first lumen of the sheath (e.g., an inflow lumen of the sheath) and/or the second reservoir can include a second lumen of the sheath (e.g., an outflow lumen sheath).

The device can have a first reservoir and a second reservoir. When the device is delivering the first material to a space distal the first opening, the first opening can be in fluid communication with the first material reservoir. When the device is removing the first material, the second material, and/or a third material from the space distal the first opening, the first opening can be in fluid communication with the second reservoir.

The device can have a mechanical agitation source connected to the probe. The mechanical agitation source can be a vibratory motion source, a dottering motion source, an ultrasonic motion source, a translational motion source, and/or a rotational motion source.

The probe can have a protrusion, a bristle, a brush, an indentation, a coil, a loop, and/or an angulation anywhere on the probe, for example, between a first end of the probe and a second end of the probe.

The probe can have protrusions, bristles, brushes, indentations, coils, loops, and/or angulations anywhere on the probe, for example, between a first end of the probe and a second end of the probe.

The probe can have one or multiple protrusions, one or multiple bristles, one or multiple brushes, one or multiple indentations, one or multiple coils, one or multiple loops, and/or one or multiple angulations anywhere on the probe, for example, between a first end of the probe and the second end of the probe.

The sheath can be a tube. The sheath can be a catheter.

The device can have a remover. The first material, the second material, and/or a third material can be closer to the first opening when the device is in a third configuration than when the device is in the second configuration.

The systems, devices, and/or methods disclosed herein can have any combination of features shown in the drawings and/or described herein (e.g., any combination of features between “A method of removing a first material from a body lumen is disclosed (e.g., as shown in the drawings and/or as described herein)” and “The probe can have one or multiple protrusions, one or multiple bristles, one or multiple brushes, one or multiple indentations, one or multiple coils, one or multiple loops, and/or one or multiple angulations anywhere on the probe, for example, between a first end of the probe and the second end of the probe” above).

A method for performing the delivery and/or removal of occlusion devices from a body space is disclosed (e.g., as shown in the drawings and/or as described herein). The method can include positioning a delivery and/or removal system within the body space. The delivery and/or removal system can have a probe within a penetrating element and a catheter configured to fit on the probe. The catheter can have a catheter lumen and a distal port at the distal end of the catheter lumen. A handle can be attached to the catheter. The method can include advancing the probe within the body space a distance of 1 cm to 15 cm, advancing the catheter over the probe once the translation of the probe reaches 1 cm to 15 cm, and delivering and/or removing an occlusion device within the body space through the catheter.

The body space can be, for example, a reproductive tract, a blood vessel, or a gastrointestinal tract.

The body space can be a body lumen, for example, a lumen of reproductive tract, a lumen of a blood vessel, or a lumen of a gastrointestinal tract.

The distance can be 5.0 cm.

The delivery and/or removal of the occlusion device can occur after the injection of media within the body space.

The delivery and/or removal of the occlusion device can occur after the removal of the probe from the body space.

The delivery and/or removal of the occlusion device can be followed by the injection of media to confirm the presence and/or absence of the occlusion device from the body space.

The advancement of the probe for confirmation of body space access (e.g., intraluminal access) can be a range from 1 cm to 10 cm.

The advancement of the probe for confirmation of body space access (e.g., intraluminal access) can be a range from 5 cm to 10 cm.

A system for performing the delivery and/or removal of occlusion devices from a body space is disclosed (e.g., as shown in the drawings and/or as described herein). The system can have a probe within a penetrating element. The penetrating element can have a needle or a component of a needle. The system can have a catheter configured to fit on the probe and a needle (e.g., a half needle). The catheter can have a catheter lumen and a distal port at the distal end of the catheter lumen. A handle can be attached to the catheter. The advancement of the probe within the body space a predetermined distance can allow for the remaining steps of delivery and/or removal to occur in the body space.

The penetrating element can have a half needle. The penetrating element can be a half needle.

The system can have a half needle. The half needle can be a component of a needle.

The body space can be, for example, a reproductive tract, a blood vessel, or a gastrointestinal tract.

The body space can be a body lumen, for example, a lumen of reproductive tract, a lumen of a blood vessel, or a lumen of a gastrointestinal tract.

The predetermined distance of the probe can be a translation of 5 cm of the probe, for example, relative to the catheter.

The predetermined distance of the probe can be a translation of 1 cm to 15 cm of the probe, for example, relative to the catheter.

A system for performing the removal of occlusion devices from a body space is disclosed (e.g., as shown in the drawings and/or as described herein). The system can have a catheter configured to fit on the probe and a needle or a component of a needle. The catheter can have a catheter lumen and a distal port at the distal end of the catheter lumen. A handle can be attached to the catheter. The system can have a probe with an irrigation source and an aspiration source. The irrigation source can have a dissolution media.

The needle or the component of the needle can be a half needle.

The system can have a half needle. The half needle can be a component of a needle.

The catheter can be configured to fit on the probe and a half needle.

Mechanical agitation can be applied to the probe to remove the occlusive device from the reproductive tract.

The mechanical agitation can include vibratory, dottering, ultrasonic motion, translational motion, and/or rotary motion.

The systems, devices, and/or methods disclosed herein can have any combination of features shown in the drawings and/or described herein (e.g., any combination of features between “A method for performing the delivery and/or removal of occlusion devices from a body space is disclosed (e.g., as shown in the drawings and/or as described herein)” and “The mechanical agitation can include vibratory, dottering, ultrasonic motion, translational motion, and/or rotary motion” above).

Delivery and/or removal systems 48 for providing and/or removing material (e.g., one or multiple materials 160) into and/or from the body space 8, including but not limited to a reproductive tract such as the vas deferens 3 or fallopian tubes 10 for contraception and/or for reversing a contraception procedure is described herein. The delivery and/or removal systems 48 can confirm proper placement of the system in the body lumen prior to the delivery of occlusive material (e.g., the material 160a), for example, by advancing a probe (e.g., the probe 55, the probe 516, the probe 620) the threshold distance D6. The systems 48 can confirm that the body space 8 is blocked by the material 160a following the deposition of the material 160a. The removal device 180 can deliver the material 160b (e.g., a dissolving material), agitate the occlusion (e.g., the material 160a) and/or the material 160b, irrigate the body space 8 (e.g., with the material 160b), aspirate the body space 8, confirm the patency of the body space 8 following the removal of the material 160a from the body space 8, or any combination thereof. The removal procedure performable using the device 180 can be an elective procedure that a patient can choose to have performed, for example, if the patient desires a return to fertility.

Any elements described herein as singular can be pluralized (i.e., anything described as “one” can be more than one). Any species element of a genus element can have the characteristics or elements of any other species element of that genus. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

The features of the systems and devices in the figures can have the relative positions shown, the relative sizes shown, and/or the relative positions and relative sizes shown. As another example, the systems and devices in the figures may not have the relative positions shown in the figures, the relative sizes shown in the figures, and/or the relative positions and sizes shown in the figures. For example, the drawings of this disclosure can be drawn to scale. The drawings of this disclosure can be considered, for example, to be drawn to scale. As another example, the drawings of this disclosure may not be drawn to scale. The drawings of this disclosure can be considered, for example, to not be drawn to scale.

Changes and modifications can be made to this disclosure, and equivalents employed, or combinations of any of the disclosed elements, characteristics, features, devices, tools, steps, or methods without departing from the spirit and scope of the disclosure. Any of the disclosed elements, characteristics, features, devices, tools, steps, or methods can be present as a singular or as a plurality regardless of whether the elements, characteristics, features, devices, steps, or methods are explicitly disclosed herein as being singular or as a plurality. Elements shown with any variation are exemplary for the specific variation and can be used on other variations within this disclosure. The terms about and approximately can include the exact values following such terms and can include, for example, a tolerance of plus or minus 1% of any such values, a tolerance of plus or minus 5%, or any other tolerance that one of ordinary skill in the art would understand. Any phrase involving an “A and/or B” construction or similar construction can mean (1) A alone, (2) B alone, (3) A and B together. Any range disclosed can include any subrange of the range disclosed, for example, a range of 1-10 units can include 2-10 units, 8-10 units, or any other subrange. The words “may” and “can” are interchangeable (e.g., “may” can be replaced with “can” and “can” can be replaced with “may”). All systems, devices, and methods described herein can be used for medical (e.g., diagnostic, therapeutic, or rehabilitative) or non-medical purposes. The above-described configurations, elements or complete assemblies and methods and their elements can be combined and modified with each other in any combination.

1. A device comprising:
a sheath;
a probe;
a first material; and
a first opening
wherein the device has a first configuration and a second configuration, wherein more of the first material is distal the first opening when the device is in the second configuration than when the device is in the first configuration, wherein the first material is closer to a second material when the device is in the second configuration than when the device is in the first configuration,
wherein the probe has a retracted configuration and an advanced configuration, wherein more of the probe is distal the first opening when the probe is in the advanced configuration than when the probe is in the retracted configuration, wherein when the probe is in the advanced configuration, the probe is movable from a first position to a second position,
wherein when the device is in the second configuration and the probe is in the first position, the probe is in contact with the first material,
wherein when the device is in the second configuration and the probe is in the second position, the probe is in contact with the first material or a gap is between the probe and the first material, and
wherein a distal terminal end of the probe is the same distance or a different distance from the first opening when the probe is in the second position than when the probe is in the first position.

2. The device of claim 1, further comprising a remover, wherein the first material, the second material, and/or a third material is closer to the first opening when the device is in a third configuration than when the device is in the second configuration.

3. A device comprising:
a sheath; and
a probe,
wherein the probe is advanceable relative to the sheath,
wherein when the probe is in an advanced position, the probe is an advanced distance from the sheath, and
wherein when the advanced distance is greater than or equal to a threshold distance, the advanced position of the probe comprises an indicator to a user that the device has access to a target site.

4. The device of claim 3, wherein when the advanced distance is greater than or equal to the threshold distance, the advanced position of the probe comprises an indicator to the user that the sheath and/or a port has access to the target site.

5. The device of claim 3, wherein the sheath comprises a sheath lumen, and wherein more of the probe is distal the sheath lumen when the probe is in the advanced configuration than when the probe is in a retracted configuration.

6. The device of claim 3, wherein the advanced distance is measured along a straight line between a distal terminal end of the sheath and a distal terminal end of the probe.

7. The device of claim 3, wherein the advanced distance is measured between a distal terminal end of the sheath and a distal terminal end of the probe.

8. The device of claim 3, wherein the advanced distance is measured along a straight line between a distal terminal opening of the sheath and a distal terminal end of the probe.

9. The device of claim 3, wherein the advanced distance is measured between a distal terminal opening of the sheath and a distal terminal end of the probe along a center longitudinal axis of the probe.

10. The device of claim 3, wherein the probe is retractable relative to the sheath.

11. A system comprising:
a sheath;
a cartridge comprising a material; and
a handle comprising a first plunger and/or a second plunger.

12. The system of claim 11, wherein the first plunger has a retracted configuration and an advanced configuration, wherein less of the material is in the cartridge when the first plunger is in the advanced configuration than when the first plunger is in the retracted configuration.

13. The system of claim 11, further comprising a plunger tip, wherein the cartridge comprises a cartridge space, wherein the first plunger has a retracted configuration and an advanced configuration, wherein when the first plunger is in the retracted configuration, the plunger tip is outside the cartridge space, and wherein when the first plunger is in the advanced configuration, the plunger tip is inside the cartridge space.

14. The system of claim 11, wherein the material comprises a hydrogel.

15. The system of claim 11, further comprising a probe.

16. The system of claim 15, wherein the probe comprises a guidewire.

17. The system of claim 11, further comprising a sheath assembly and a probe assembly, wherein the sheath assembly comprises the sheath, wherein the probe assembly comprises a probe, and wherein when the sheath assembly and the probe assembly are removably connected to each other and the probe is in an advanced position, the probe is distal the sheath.

18. The system of claim 11, further comprising a sheath assembly and a probe assembly, wherein the sheath assembly comprises the sheath and a sheath hub, wherein the probe assembly comprises a probe and a probe hub, and wherein when the sheath hub and the probe hub are removably connected to each other, more of the probe is distal the sheath hub when the probe is in an advanced configuration than when the probe is in a retracted configuration.

19. The system of claim 11, further comprising a sheath assembly comprising the sheath and a sheath hub, wherein when the sheath assembly and the handle are connected to each other, the cartridge is in the sheath hub and/or in a housing of the handle.

20. The system of claim 11, wherein the handle comprises the first plunger and the second plunger.
